(19) United States
(12) Patent Application Publication
(10) Pub. No.: US 2019/0091322 A1
(43) Pub. Date: Mar. 28, 2019

(54) DNA ANTIBODY CONSTRUCTS AND METHOD OF USING SAME

(71) Applicants: David B. Weiner, Merion, PA (US); Karuppiah Muthumani, Cherry Hill, NJ (US); Seleeke Flingai, Minneapolis, MN (US); Niranjan Sardesai, Blue Bell, PA (US); Sarah Elliott, Pullman, WA (US); Jian Yan, Wallingford, PA (US); Ami Patel, Philadelphia, PA (US)

(72) Inventors: David B. Weiner, Merion, PA (US); Karuppiah Muthumani, Cherry Hill, NJ (US); Seleeke Flingai, Minneapolis, MN (US); Niranjan Sardesai, Blue Bell, PA (US); Sarah Elliott, Pullman, WA (US); Jian Yan, Wallingford, PA (US); Ami Patel, Philadelphia, PA (US)

(21) Appl. No.: 16/087,146

(22) PCT Filed: Mar. 21, 2017
(86) PCT No.: PCT/US17/23479

§ 371 Date: Sep. 21, 2018

Related U.S. Application Data
(60) Provisional application No. 62/311,316, filed on Mar. 21, 2016.
(60) Provisional application No. 62/332,381, filed on May 5, 2016.
(60) Provisional application No. 62/376,162, filed on Aug. 17, 2016.
(60) Provisional application No. 62/396,748, filed on Sep. 19, 2016.
(60) Provisional application No. 62/396,750, filed on Sep. 19, 2016.
(60) Provisional application No. 62/417,093, filed on Nov. 3, 2016.
(60) Provisional application No. 62/429,454, filed on Dec. 2, 2016.
(60) Provisional application No. 62/429,473, filed on Dec. 2, 2016.

(51) Int. Cl. A61K 39/12; A61K 39/42; C07K 16/10

(52) U.S. Cl. CPC A61K 39/12; A61K 2039/53; C07K 16/1081; A61K 39/42

(57) ABSTRACT

Disclosed herein is a composition comprising the combination of a nucleic acid sequence encoding a desired polypeptide that elicits an immune response in a mammal and a nucleic acid sequence encoding an antibody, a fragment thereof, a variant thereof, or a combination thereof.

### CROSS REFERENCE TO RELATED APPLICATION

This application claims priority to U.S. Provisional Application No. 62/311,316, filed Mar. 21, 2016, U.S. Provisional Application No. 62/396,748, filed Sep. 19, 2016, U.S. Provisional Application No. 62/396,750, filed Sep. 19, 2016, U.S. Provisional Application No. 62/417,093, filed Nov. 3, 2016, U.S. Provisional Application No. 62/332,381, filed May 4, 2016, U.S. Provisional Application No. 62/376,162, filed Aug. 17, 2016, U.S. Provisional Application No. 62/429,454, filed Dec. 2, 2016, and U.S. Provisional Application No. 62/429,473, filed Dec. 2, 2016, each of which is hereby incorporated by reference in its entirety

### TECHNICAL FIELD

The present invention relates to a combination of a DNA vaccine with a composition comprising a recombinant nucleic acid sequence for generating one or more synthetic antibodies, and functional fragments thereof, in vivo. The compositions of the invention provide improved methods for inducing immune responses, and for prophylactically and/or therapeutically immunizing individuals against an antigen.

### BACKGROUND

The immunoglobulin molecule comprises two of each type of light (L) and heavy (H) chain, which are covalently linked by disulphide bonds (shown as S—S) between cysteine residues. The variable domains of the heavy chain (VH) and the light chain (VL) contribute to the binding site of the antibody molecule. The heavy-chain constant region is made up of three constant domains (CH1, CH2 and CH3) and the (flexible) hinge region. The light chain also has a constant domain (CL). The variable regions of the heavy and light chains comprise four framework regions (FRs; FR1, FR2, FR3 and FR4) and three complementarity-determining regions (CDRs; CDR1, CDR2 and CDR3). Accordingly, these are very complex genetic systems that have been difficult to assemble in vivo.

Targeted monoclonal antibodies (mAbs) represent one of the most important medical therapeutic advances of the last 25 years. This type of immune based therapy is now used routinely against a host of autoimmune diseases, treatment of cancer as well as infectious diseases. For malignancies, many of the immunoglobulin (Ig) based therapies currently used are in combination with cytotoxic chemotherapy regimens directed against tumors. This combination approach has significantly improved overall survival. Multiple mAb preparations are licensed for use against specific cancers, including Rituxan (Rituximab), a chimeric mAb targeting CD20 for the treatment of Non-Hodgkins lymphoma and Ipilimumab (Yervoy), a human mAb that blocks CTLA-4 and which has been used for the treatment of melanoma and other malignancies. Additionally, Bevacizumab (Avastin) is another prominent humanized mAb that targets VEGF and tumor neovascularization and has been used for the treatment of colorectal cancer. Perhaps the most high profile mAb for treatment of a malignancy is Trastuzumab (Herceptin), a humanized preparation targeting Her2/neu that has been demonstrated to have considerable efficacy against breast cancer in a subset of patients. Furthermore, a host of mAbs are in use for the treatment of autoimmune and specific blood disorders.

In addition to cancer treatments, passive transfer of polyclonal Igs mediate protective efficacy against a number of infectious diseases including diphtheria, hepatitis A and B, rabies, tetanus, chicken-pox and respiratory syncytial virus (RSV). In fact, several polyclonal Ig preparations provide temporary protection against specific infectious agents in individuals traveling to disease endemic areas in circumstances when there is insufficient time for protective Igs to be generated through active vaccination. Furthermore, in children with immune deficiency the Palivizumab (Synagis), a mAb, which targets RSV infection, has been demonstrated to clinically protect against RSV.

Currently available therapeutic antibodies that exist in the market are human IgG1 isotypes. These antibodies include glycoproteins bearing two N-linked biantennary complex-type oligosaccharides bound to the antibody constant region (Fc), in which a majority of the oligosaccharides are core-fucosylated. It exercises effector functions of antibody-dependent cellular toxicity (ADCC) and complement-dependent cytotoxicity (CDC) through the interaction of the Fc with either leukocyte receptors (FcγRs) or complement. There is a phenomena of reduced in vivo efficacy of therapeutic antibodies (versus in vitro), thus resulting in the need for large doses of therapeutic antibodies—sometimes weekly doses of several hundred milligrams. This is mainly due to the competition between serum IgG and therapeutic antibodies for binding to FcγRIIIa on natural killer (NK) cells. Endogenous human serum IgG inhibits ADCC induced by therapeutic antibodies. Thus, there can be enhanced efficacy of non-fucosylated therapeutic antibodies in humans. Non-fucosylated therapeutic antibodies have much higher binding affinity for FcγRIIIa than fucosylated human serum IgG, which is a preferable character to conquer the interference by human plasma IgG.

Antibody based treatments are not without risks. One such risk is antibody-dependent enhancement (ADE), which occurs when non-neutralising antiviral proteins facilitate virus entry into host cells, leading to increased infectivity in the cells. Some cells do not have the usual receptors on their surfaces that viruses use to gain entry. The antiviral proteins (i.e., the antibodies) bind to antibody Fc receptors that some of these cells have in the plasma membrane. The viruses bind to the antigen binding site at the other end of the antibody. This virus can use this mechanism to infect human macrophages, causing a normally mild viral infection to become life-threatening. The most widely known example of ADE occurs in the setting of infection with the dengue virus (DENV). It is observed when a person who has previously been infected with one serotype of DENV becomes infected many months or years later with a different serotype. In such cases, the clinical course of the disease is more severe, and these people have higher viremia compared with those in whom ADE has not occurred. This explains the observation that while primary (first) infections cause mostly minor disease (DF) in children, secondary infection (re-infection at a later date) is more likely to be associated with severe disease (DHF and/or DSS) in both children and adults. There are four antigenically different serotypes of DENV (DENV-1-DENV-4). Infection with DENV induces the production of neutralizing homotypic immunoglobulin G (IgG) antibodies which provide lifelong immunity against the infecting serotype. Infection with DENV also produces some degree of cross-protective immunity against the other three serotypes. In addition to inducing neutralizing heterotypic antibodies, infection with DENV can also induce heterotypic antibodies which neutralize the virus only partially or not at all. The production of such cross-reactive but non-neutralizing antibodies could be the reason for more severe secondary infections. Once inside the white blood cell, the virus replicates undetected, eventually generating very high virus titers which cause severe disease.

The clinical impact of mAb therapy is impressive. However, issues remain that limit the use and dissemination of this therapeutic approach. Some of these include the high cost of production of these complex biologics that can limit their use in the broader population, particularly in the developing world where they could have a great impact. Furthermore, the frequent requirement for repeat administrations of the mAbs to attain and maintain efficacy can be an impediment in terms of logistics and patient compliance. New antibodies that would reduce or eliminate the low in vivo efficacy of therapeutic antibodies due to competition with serum IgGs are needed. New antibodies that can eliminate antibody dependent enhancement in viruses like Dengue, HIV, RSV and others are needed. Bispecific antibodies, bifunctional antibodies, and antibody cocktails are needed to perform several functions that could prove therapeutic or prophylactic. Combination therapies are needed as well that can utilize the synthetic antibodies described herein along with immunostimulating a host system through immunization with a vaccine, including a DNA based vaccine. Additionally, the long-term stability of these antibody formulations is frequently short and less than optimal. Thus, there remains a need in the art for a synthetic antibody molecule that can be delivered to a subject in a safe and cost effective manner.

### SUMMARY

The present invention provides a combination of a composition that elicits an immune response in a mammal against an antigen with a composition comprising a recombinant nucleic acid sequence encoding an antibody, a fragment thereof, a variant thereof, or a combination thereof.

One aspect of the present invention provides nucleic acid constructs capable of expressing a polypeptide that elicits an immune response in a mammal against an antigen. The nucleic acid constructs are comprised of an encoding nucleotide sequence and a promoter operably linked to the encoding nucleotide sequence. The encoding nucleotide sequence expresses the polypeptide, wherein the polypeptide includes consensus antigens. The promoter regulates expression of the polypeptide in the mammal.

Another aspect of the present invention provides DNA plasmid vaccines that are capable of generating in a mammal an immune response against an antigen. The DNA plasmid vaccines are comprised of a DNA plasmid capable of expressing a consensus antigen in the mammal and a pharmaceutically acceptable excipient. The DNA plasmid is comprised of a promoter operably linked to a coding sequence that encodes the consensus antigen.

Another aspect of the present invention provides methods of eliciting an immune response against an antigen in a mammal, comprising delivering a DNA plasmid vaccine to tissue of the mammal, the DNA plasmid vaccine comprising a DNA plasmid capable of expressing a consensus antigen in a cell of the mammal to elicit an immune response in the mammal, and electroporating cells of the tissue to permit entry of the DNA plasmids into the cells.

The present invention is directed to a method of generating a synthetic antibody in a subject. The method can comprise administering to the subject a composition comprising a recombinant nucleic acid sequence encoding an antibody or fragment thereof. The recombinant nucleic acid sequence can be expressed in the subject to generate the synthetic antibody.

The generated synthetic antibody may be defucosylated. The generated synthetic antibody may include two leucine to alanine mutations in a CH2 region of a Fc region.

The antibody can comprise a heavy chain polypeptide, or fragment thereof, and a light chain polypeptide, or fragment thereof. The heavy chain polypeptide, or fragment thereof, can be encoded by a first nucleic acid sequence and the light chain polypeptide, or fragment thereof, can be encoded by a second nucleic acid sequence. The recombinant nucleic acid sequence can comprise the first nucleic acid sequence and the second nucleic acid sequence. The recombinant nucleic acid sequence can further comprise a promoter for expressing the first nucleic acid sequence and the second nucleic acid sequence as a single transcript in the subject. The promoter can be a cytomegalovirus (CMV) promoter.

The recombinant nucleic acid sequence can further comprise a third nucleic acid sequence encoding a protease cleavage site. The third nucleic acid sequence can be located between the first nucleic acid sequence and second nucleic acid sequence. The protease of the subject can recognize and cleave the protease cleavage site.

The recombinant nucleic acid sequence can be expressed in the subject to generate an antibody polypeptide sequence. The antibody polypeptide sequence can comprise the heavy chain polypeptide, or fragment thereof, the protease cleavage site, and the light chain polypeptide, or fragment thereof. The protease produced by the subject can recognize and cleave the protease cleavage site of the antibody polypeptide sequence thereby generating a cleaved heavy chain polypeptide and a cleaved light chain polypeptide. The synthetic antibody can be generated by the cleaved heavy chain polypeptide and the cleaved light chain polypeptide.

The recombinant nucleic acid sequence can comprise a first promoter for expressing the first nucleic acid sequence as a first transcript and a second promoter for expressing the second nucleic acid sequence as a second transcript. The first transcript can be translated to a first polypeptide and the second transcript can be translated into a second polypeptide. The synthetic antibody can be generated by the first and second polypeptide. The first promoter and the second promoter can be the same. The promoter can be a cytomegalovirus (CMV) promoter.

The heavy chain polypeptide can comprise a variable heavy region and a constant heavy region 1. The heavy chain polypeptide can comprise a variable heavy region, a constant heavy region 1, a hinge region, a constant heavy region 2 and a constant heavy region 3. The light chain polypeptide can comprise a variable light region and a constant light region.

The recombinant nucleic acid sequence can further comprise a Kozak sequence. The recombinant nucleic acid sequence can further comprise an immunoglobulin (Ig) signal peptide. The Ig signal peptide can comprise an IgE or IgG signal peptide.

The recombinant nucleic acid sequence can comprise a nucleic acid sequence encoding at least one amino acid sequence of SEQ ID NOs:1, 2, 5, 41, 43, 45, 46, 47, 48, 49, 51, 53, 55, 57, 59, 61, and 80. The recombinant nucleic acid sequence can comprise at least one nucleic acid sequence of SEQ ID NOs:3, 4, 6, 7, 40, 42, 44, 50, 52, 54, 56, 58, 60, 62, 63, and 79.

The present invention is also directed to a method of generating a synthetic antibody in a subject. The method can comprise administering to the subject a composition comprising a first recombinant nucleic acid sequence encoding a heavy chain polypeptide, or fragment thereof, and a second recombinant nucleic acid sequence encoding a light chain polypeptide, or fragment thereof. The first recombinant nucleic acid sequence can be expressed in the subject to generate a first polypeptide and the second recombinant nucleic acid can be expressed in the subject to generate a second polypeptide. The synthetic antibody can be generated by the first and second polypeptides.

The first recombinant nucleic acid sequence can further comprise a first promoter for expressing the first polypeptide in the subject. The second recombinant nucleic acid sequence can further comprise a second promoter for expressing the second polypeptide in the subject. The first promoter and second promoter can be the same. The promoter can be a cytomegalovirus (CMV) promoter.

The heavy chain polypeptide can comprise a variable heavy region and a constant heavy region 1. The heavy chain polypeptide can comprise a variable heavy region, a constant heavy region 1, a hinge region, a constant heavy region 2 and a constant heavy region 3. The light chain polypeptide can comprise a variable light region and a constant light region.

The first recombinant nucleic acid sequence and the second recombinant nucleic acid sequence can further comprise a Kozak sequence. The first recombinant nucleic acid sequence and the second recombinant nucleic acid sequence can further comprise an immunoglobulin (Ig) signal peptide. The Ig signal peptide can comprise an IgE or IgG signal peptide.

The present invention is further directed to method of preventing or treating a disease in a subject. The method can comprise generating a synthetic antibody in a subject according to one of the above methods. The synthetic antibody can be specific for a foreign antigen. The foreign antigen can be derived from a virus. The virus can be Human immunodeficiency virus (HIV), Chikungunya virus (CHIKV) or Dengue virus.

The virus can be HIV. The recombinant nucleic acid sequence can comprise a nucleic acid sequence encoding at least one amino acid sequence of SEQ ID NOs:1, 2, 5, 46, 47, 48, 49, 51, 53, 55, and 57. The recombinant nucleic acid sequence can comprise at least one nucleic acid sequence of SEQ ID NOs: 3, 4, 6, 7, 50, 52, 55, 56, 62, and 63.

The virus can be CHIKV. The recombinant nucleic acid sequence can comprise a nucleic acid sequence encoding at least one amino acid sequence of SEQ ID NOs:59 and 61. The recombinant nucleic acid sequence can comprise at least one nucleic acid sequence of SEQ ID NOs: 58, 60, 97, 98, 99 and 100.

The virus can be Zika. The recombinant nucleic acid sequence can comprise a nucleic acid sequence encoding at least one amino acid sequence of SEQ ID NOs: 101, 102, 103, 104, 105, 106, 107, 108, 109, 110, 111, 112, 113, 114, 115, 116, 117, 118, 121, 122, 123, 125, 127, 129, 131, or 133. The recombinant nucleic acid sequence can comprise at least one nucleic acid sequence of SEQ ID NOs: 124, 126, 128, 130, or 132.

The virus can be Dengue virus. The recombinant nucleic acid sequence can comprise a nucleic acid sequence encoding at least one amino acid sequence of SEQ ID NO:45. The recombinant nucleic acid sequence comprises at least one nucleic acid sequence of SEQ ID NO:44.

The synthetic antibody can be specific for a self-antigen. The self-antigen can be Her2. The recombinant nucleic acid sequence can comprise a nucleic acid sequence encoding at least one amino acid sequence of SEQ ID NOs:41 and 43. The recombinant nucleic acid sequence can comprise at least one nucleic acid sequence of SEQ ID NOs:40 and 42.

The synthetic antibody can be specific for a self-antigen. The self-antigen can be PSMA. The recombinant nucleic acid sequence can comprise a nucleic acid sequence encoding at least one amino acid sequence of SEQ ID NO:80. The recombinant nucleic acid sequence can comprise at least one nucleic acid sequence of SEQ ID NO:79.

The present invention is also directed to a product produced by any one of the above-described methods. The product can be a single DNA plasmid capable of expressing a functional antibody. The product can be comprised of two or more distinct DNA plasmids capable of expressing components of a functional antibody that combine in vivo to form a functional antibody.

The present invention is also directed to a method of treating a subject from infection by a pathogen, comprising: administering a nucleotide sequence encoding a synthetic antibody specific for the pathogen. The method can further comprise administering an antigen of the pathogen to generate an immune response in the subject.

The present invention is also directed to a method of treating a subject from cancer, comprising: administering a nucleotide sequence encoding a cancer marker to induce ADCC.

The present invention is also directed to a nucleic acid molecule encoding a synthetic antibody comprising a nucleic acid sequence having at least about 95% identity over an entire length of the nucleic acid sequence set forth in SEQ ID NO:79.

The present invention is also directed to a nucleic acid molecule encoding a synthetic antibody comprising a nucleic acid sequence as set forth in SEQ ID NO:79.

The present invention is also directed to a nucleic acid molecule encoding a synthetic antibody comprising a nucleic acid sequence encoding a protein having at least about 95% identity over an entire length of the amino acid sequence set forth in SEQ ID NO:80.

The present invention is also directed to a nucleic acid molecule encoding a synthetic antibody comprising a nucleic acid sequence encoding a protein comprising an amino acid sequence as set forth in SEQ ID NO:80.

Any one of the above-described nucleic acid molecules may comprise an expression vector.

The present invention is also directed to a composition comprising one or more of the above-described nucleic acid molecules. The composition may also include a pharmaceutically acceptable excipient.

### BRIEF DESCRIPTION OF THE DRAWINGS

FIG. 1, comprising FIG. 1A through FIG. 1D, depicts CVM1-immunoglobulin G (IgG) and CVM-1-Fab dMAb plasmid design and expression. FIG. 1A depicts in vitro expression of CVM1-Fab. The CVM1-Fab, CVM1-variable heavy chain (VH), and CVM1-variable light chain (VL) constructs were transfected into 293T cells to determine in vitro expression through binding enzyme-linked immunosorbent assays (ELISAs). Samples were analyzed at 0, 24, and 48 hours post-transfection. Cells transfected with an empty backbone pVax1 plasmid served as a negative control. FIG. 1B depicts In vitro expression of CVM1-IgG. The CVM1-IgG was transfected into 293T cells to determine in vitro expression through binding enzyme-linked immunosorbent assays (ELISAs). Samples were analyzed at 0, 24, and 48 hours post-transfection. Cells transfected with an empty backbone pVax1 plasmid served as a negative control. FIG. 1C depicts in vivo expression of CVM1-IgG and CVM1-Fab. Mice (B6.Cg-Foxn1nu/J) aged 5-6 weeks received a single, 100-μg intramuscular injection of CVM1-IgG, CVM1-VH, CVM1-VL, or CVM1-Fab plasmids, followed by electroporation (5 mice per group). Injection of a pVax1 vector was used a negative control. Sera IgG levels were measured at various time points in mice injected intramuscularly. FIG. 1D depicts experimental results demonstrating sera from CVM1-IgG-administered mice binds chikungunya virus (CHIKV) envelope protein (Env). ELISA plates were coated with recombinant CHIKV envelope or human immunodeficiency virus type 1 (HIV-1) (subtype B; MN) envelope protein, and sera obtained on day 15 from mice given a single injection of CVM1-IgG, CVM1-Fab, or pVax1 were tested.

FIG. 2, comprising FIG. 2A through FIG. 2D, depicts binding analyses and neutralization activity of CVM1-immunoglobulin G (IgG) antibodies. FIG. 2A depicts an immunofluorescence assay demonstrating that IgG generated from CVM1-IgG-administered mice was capable of binding to chikungunya virus (CHIKV) envelope protein (Env). CHIKV-infected Vero cells were fixed 24 hours after infection and evaluated by an immunofluorescence assay to detect CHIKV Env antigen expression (green). Cell nuclei were stained with DAPI (blue). Sera from control mice injected with pVax1 were used as a negative control. FIG. 2B depicts binding affinity of sera from CVM1-IgG-injected mice (day 15) to target proteins. Binding was tested by Western blot, using cell lysates from CHIKV- or mock-infected cells. Protein transferred membranes were re-probed with antibody against β-actin as a loading control. The image presented here was cropped from an original image and is representative of several gels. FIG. 2C depicts fluorescence-activated cell-sorting analysis of the binding of sera from plasmid-injected mice to CHIKV-infected cells. The x-axis indicates green fluorescent protein (GFP) staining, using the lentiviral GFP pseudovirus complemented with CHIKV Env. The y-axis demonstrates staining of infected cells by human IgG produced in mice 15 days after injection with CVM1-IgG. Staining with a control anti-CHIKV antibody (Env antibody) is also shown, as well as staining with no antibodies and pVax1. The presence and number of double-positive cells indicate presence and level of sera binding to the CHIKV-infected cells. FIG. 2D depicts sera from mice injected with CVM1-IgG via electroporation possess neutralizing activity against multiple CHIKV strains (ie, Ross, LR2006-OPY1, IND-63-WB1, PC-08, DRDE-06, and SL-CH1). Neutralizing antibody titers are plotted, and 50% inhibitory concentrations (IC50 values; parenthesis) were calculated with Prism GraphPad software. Similar results were observed in 2 independent experiments with at least 10 mice per group for each experiment.

FIG. 3, comprising FIG. 3A through FIG. 3D, depicts the characterization of in vivo immune protection conferred by CVM1-Fab and CVM1-immunoglobulin G (IgG). FIG. 3A depicts BALB/c mice were injected with 100 μg of pVax1 (negative control), CVM1-IgG, CVM1-variable heavy chain, and CVM1-variable light chain on day 0 and challenged on day 2 with chikungunya virus (CHIKV). Mice were monitored daily, and survival rates were recorded for 20 days after viral challenge. FIG. 3B depicts BALB/c mice were injected with 100 μg of pVax1 (negative control), CVM1-IgG, CVM1-variable heavy chain, and CVM1-variable light chain on day 0 and challenged on day 30 with chikungunya virus (CHIKV). Mice were monitored daily, and survival rates were recorded for 20 days after viral challenge. FIG. 3C depicts protection of mice from different routes of CHIKV challenge. Two groups of mice were injected with 100 μg of CVM1-IgG by the intramuscular route, followed by viral challenge on day 2 with subcutaneous inoculation. Mice were monitored daily, and survival rates were recorded for 20 days after the viral challenge. The black arrow indicates plasmid injections; the red arrow indicates the time of viral challenge. Each group consisted of 10 mice, and the results were representative of 2 independent experiments. FIG. 3D depicts protection of mice from different routes of CHIKV challenge. Two groups of mice were injected with 100 μg of CVM1-IgG by the intramuscular route, followed by viral challenge on day 2 with intranasal inoculation. Mice were monitored daily, and survival rates were recorded for 20 days after the viral challenge. The black arrow indicates plasmid injections; the red arrow indicates the time of viral challenge. Each group consisted of 10 mice, and the results were representative of 2 independent experiments.

FIG. 4, comprising FIG. 4A through FIG. 4D, depicts comparative and combination studies with CVM1-immunoglobulin G (IgG) and the chikungunya virus (CHIKV) envelope protein (Env) DNA vaccine. FIG. 4A depicts a survival analysis of BALB/c mice were injected with 100 μg of CVM1-IgG, 100 μg of pVax1 (negative control), or 25 μg of CHIKV-Env DNA on day 0 and challenged on day 2 with CHIKV Del-03 (JN578247; 1×10⁷ plaque-forming units in a total volume of 25 μL). Mice were monitored for 20 days after challenge, and survival rates were recorded. FIG. 4B depicts a survival analysis of BALB/c mice were administered either a single injection of 100 μg of CVM1-IgG on day 0 or 3 immunizations of 25 μg of CHIKV Env DNA on day 0, day 14, and day 28 and then challenged on day 35 under the same conditions and with the same CHIKV isolate. Mice were monitored for 20 days after challenge, and survival rates were recorded. FIG. 4C depicts a survival analysis of Groups of 20 BALB/c mice were administered a single 100 μg injection of CVM1-IgG on day 0 and 3 immunizations with CHIKV-Env DNA (25 μg) on day 0, day 14, and day 28. Half of the mice were then challenged on day 2, and the remaining half were challenged on day 35 under the same conditions and with the same CHIKV isolate challenge described above. The black arrow indicates plasmid injection, and the red arrow indicates the time of viral challenge. Mice were monitored for 20 days after challenge, and survival rates were recorded. FIG. 4D depicts experimental results demonstrating induction of persistent and systemic anti-CHIKV Env antibodies following a single CVM1-IgG (human anti-CHIKV Env) injection and CHIKV-Env immunization (mouse anti-CHIKV Env) 1 week after the second immunization in mice.

FIG. 5, comprising FIG. 5A through FIG. 5C, depicts characterization of pathologic footpad swelling and changes in weight in viral-challenged mice vaccinated with CVM1-immunoglobulin G (IgG) and/or chikungunya virus (CHIKV) envelope protein (Env) DNA. FIG. 5A depicts viral titers 1 week after CHIKV challenge in mice that received CVM1-IgG, CHIKV-Env, CVM1-IgG plus CHIKV-Env, or pVax1 (control). Each data point represents the average viral titers from 10 mice. Error bars indicate standard errors of the means. FIG. 5B depicts mean daily weight gain (±standard deviation [SD]) after subcutaneous inoculation with the CHIKV isolate among mice that received CVM1-IgG, CHIKV-Env, CVM1-IgG plus CHIKV-Env, or pVax1. Mice were weighed on the specified days after inoculation. Results are presented as mean body weights (±SD). FIG. 5C depicts swelling of the hind feet quantified using calipers on the specified days among mice that received CVM1-IgG, CHIKV-Env, CVM1-IgG plus CHIKV-Env, or pVax1. Data are mean values (±SD).

FIG. 6, comprising FIG. 6A and FIG. 6B, depicts cellular immune analysis in viral challenged CVM1-IgG and/or CHIKV-Env DNA vaccinated mice. FIG. 6A depicts concentrations of anti-CHIKV human IgG levels were measured from the mice that were injected with CVM1-IgG plus CHIKV-Env and then challenged on day 35 under the same conditions with the CHIKV isolate. Concentrations of anti-CHIKV human IgG levels were measured at indicated time points following injection. FIG. 6B depicts T-cell responses in splenocytes of mice injected with CVM1-IgG plus CHIKV-Env after stimulation with CHIKV-specific peptides. IFN-γ ELISPOTs were performed on day 35 samples. The data indicated are representative of at least 2 separate experiments.

FIG. 7 depicts characterization of serum pro-inflammatory cytokines levels from CHIKV infected mice. Cytokine (TNF-α, IL-1β and IL-6) levels were measured in mice at one week post-challenge by specific ELISA assays. Mice injected with CHIKV IgG and CHIKV-Env had similar and significantly lower sera levels of TNF-α, IL-10 and IL-6 levels. Data represent the average of 3 wells per mouse (n=10 per group).

FIG. 8 depicts experimental results demonstrating the induction of persistent and systemic anti-Zika virus-Env antibodies. Anti-ZIKV antibody responses are induced by ZIKV-prME +ZV-DMAb immunization. A129 mice (n=4) were immunized i.m. three times with 25 μg of ZIKV-prME plasmid at 2-week intervals or one time with ZIKV-DMAb. Binding to recombinant ZIKV-Envelope was analyzed with sera from animals at different time points as indicated. Induction of persistent and systemic anti-ZIKV Env antibodies following a single ZV-IgG (human anti-ZIKV) injection and ZIKV-prME immunization (mouse anti-ZIKV Envelope). The data shown are representative of at least two separate experiments and mean OD450 values are shown ±SD.

FIG. 9 depicts the structure of the ZIKV-E protein.

FIG. 10 depicts the workflow for development and characterization of Zika dMABs.

FIG. 11 depicts the binding ELISA for ZIKV-Env specific monoclonal antibodies.

FIG. 12 depicts a western blot of ZV Env and ZV mAB. 2 μg of rZV envelope protein loaded; 1:250 dilution were used for ZV monoclonal antibody.

FIG. 13 depicts ZIKA mAb VH and VL alignments.

FIG. 14 depicts ZIKA mAb VH and VL alignments and identity and RMSD matrices.

FIG. 15 depicts mAb model superpositions.

FIG. 16 depicts a comparison of model CDR regions

FIG. 17 depicts mAB 1C2A6, 8D10F4, and 8A9F9 VH and VL alignments.

FIG. 18 depicts a model of 1C2A6 Fv.

FIG. 19 depicts a summary of Fv biophysical features for 8D10F4, 1C2A6, 8A9F9, 3F12E9, and 1D4G7.

FIG. 20, comprising FIG. 20A through FIG. 20E depicts experimental results demonstrating the construction of the ZIKV-prME consensus DNA vaccine. FIG. 20A depicts a diagrammatic representation of the ZIKV-prME DNA vaccine indicating the cloning of rME into the pVax1 mammalian expression vector. A consensus design strategy was adopted for the ZIKV-prME consensus sequence. Codon-optimized synthetic genes of the prME construct included a synthetic IgE leader sequence. The optimized gene construct was inserted into the BamH1 and Xho1 sites of a modified pVax1 vector under the control of the CMV promoter. FIG. 20B depicts a model building of the ZIKV-E proteins demonstrates overlap of the vaccine target with potentially relevant epitope regions. Several changes made for vaccine design purpose are located in domains II and III (located within dashed lines of inset, middle left). Vaccine-specific residue changes in these regions are shown in violet CPK format on a ribbon backbone representation of an E (envelope) protein dimer (each chain in light and dark green, respectively). Regions corresponding to the defined EDE are indicated in cyan, and the fusion loop is indicated in blue. Residue Ile156 (T156I) of the vaccine E protein, modelled as exposed on the surface of the 150 loop, is part of an N-linked glycosylation motif NXS/T in several other ZIKV strains as well as in multiple dengue virus strains. FIG. 20C depicts expression analysis by SDS-PAGE of ZIKV-prME protein expression in 293T cells using western blot analysis. The 293T cells were transfected with the ZIKV-prME plasmid and the cell lysates and supernatants were analyzed for expression of the vaccine construct with pan-flavivirus immunized sera. Protein molecular weight markers (kDa); cell lysate and supernatant from ZIKV-prME transfected cells and rZIKV-E positive control were loaded as indicated. FIG. 20D depicts expression analysis by SDS-PAGE of ZIKV-prME protein expression in 293T cells using western blot analysis. The 293T cells were transfected with the ZIKV-prME plasmid and the cell lysates and supernatants were analyzed for expression of the vaccine construct with ZIKV-prME immunized sera. Protein molecular weight markers (kDa); cell lysate and supernatant from ZIKV-prME transfected cells and rZIKV-E positive control were loaded as indicated. FIG. 20E depicts Immunofluorescence assay (IFA) analysis for ZIKV-prME protein expression in 293T cells. The cells were transfected with 5 μg of the ZIKVprME plasmid. Twenty-four hours post transfection, immunofluorescence labelling was performed with the addition of sera (1:100) from ZIKV-prME immunized mice followed by the addition of the secondary anti-mouse IgG-AF488 antibody for detection. Staining with sera from ZIKV-prME and pVax1 immunized mice is shown. DAPI panels show control staining of cell nuclei. Overlay panels are combinations of antimouse IgG-AF488 and DAPI staining patterns. DAPI, 4′,6-diamidino-2-phenylindole; ZIKV-prME, precursor membrane and envelope of Zika virus.

FIG. 21, comprising FIG. 21A through FIG. 21D depicts experimental results demonstrating the characterization of cellular immune responses in mice following vaccination with the ZIKV-prME DNA vaccine. FIG. 21A depicts a timeline of vaccine immunizations and immune analysis used in the study. FIG. 21B depicts ELISpot analysis measuring IFN-γ secretion in splenocytes in response to ZIKV-prME immunization. C57BL/6 mice (n=4/group) were immunized i.m. three times with 25 μg of either pVax1 or the ZIKV-prME DNA vaccine followed by electroporation. IFN-γ generation, as an indication of induction of cellular immune responses, was measured by an IFN-γ ELISpot assay. The splenocytes harvested 1 week after the third immunization were incubated in the presence of one of the six peptide pools spanning the entire prM and Envelope proteins. Results are shown in stacked bar graphs. The data represent the average numbers of SFU (spot-forming units) per million splenocytes with values representing the mean responses in each±s.e.m. FIG. 21C depicts the epitope composition of the ZIKVprME-specific IFN-γ response as determined by stimulation with matrix peptide pools 1 week after the third immunization. The values represent mean responses in each group±s.e.m. The experiments were performed independently at least three times with similar results. FIG. 21D depicts flow cytometric analysis of T-cell responses. Immunisation with ZIKV-prME induces higher number of IFN-γ and TNF-α secreting cells when stimulated by ZIKV peptides. One week after the last immunization with the ZIKV-prME vaccine, splenocytes were cultured in the presence of pooled ZIKV peptides (5 μM) or R10 only. Frequencies of ZIKV peptide-specific IFN-γ and TNF-α secreting cells were measured by flow cytometry. Single function gates were set based on negative control (unstimulated) samples and were placed consistently across samples. The percentage of the total CD8⁺ T-cell responses are shown. These data are representative of two independent immunization experiments. IFN, interferon; TNF, tumour necrosis factor; ZIKV-prME, precursor membrane and envelope of Zika virus.

FIG. 22, comprising FIG. 22A through FIG. 22E depicts experimental results demonstrating that anti-ZIKV antibody responses are induced by ZIKV-prME vaccination. FIG. 22A depicts ELISA analysis measuring binding antibody production (measured by OD450 values) in immunized mice. The C57BL/6 mice (n=4) were immunized i.m. three times with 25 μg of ZIKV-prME plasmid or pVax1 at 2-week intervals. Binding to rZIKV-E was analyzed with sera from animals at different time points (days 21, 35 and 50) post immunization at various dilutions. The data shown are representative of at least three separate experiments. FIG. 22B depicts End point binding titer analysis. Differences in the anti-ZIKV end point titers produced in response to the ZIKV-prME immunogen were analyzed in sera from immunized animals after each boost. FIG. 22C depicts Western blot analysis of rZIKV-E specific antibodies induced by ZIKV-prME immunization. The rZIKV-E protein was electrophoresed on a 12.5% SDS polyacrylamide gel and analyzed by western blot analysis with pooled sera from ZIKV-prME immunized mice (day 35). Binding to rZIKV-E is indicated by the arrowhead. FIG. 22D depicts immunofluorescence analysis of ZIKV specific antibodies induced by ZIKV-prME immunization. The Vero cells infected with either ZIKV-MR766 or mock infected were stained with pooled sera from ZIKV-prME immunized mice (day 35) followed by an anti-mouse-AF488 secondary antibody for detection. FIG. 22E depicts plaque-reduction neutralization (PRNT) assay analysis of neutralizing antibodies induced by ZIKV-prME immunization. The serum samples from the ZIKV-prME immunized mice were tested for their ability to neutralize ZIKV infectivity in vitro. PRNT50 was defined as the serum dilution factor that could inhibit 50% of the input virus. The values in parentheses indicate the PRNT50. Control ZIKV-Cap (DNA vaccine expressing the ZIKV capsid protein) and pVax1 sera were used as negative controls. ZIKV-prME, precursor membrane and envelope of Zika virus.

FIG. 23, comprising FIG. 23A through FIG. 23E depicts experimental results demonstrating Induction of ZIKV specific cellular immune responses following ZIKV-prME vaccination of non-human primates (NHPs). FIG. 23A depicts ELISpot analysis measuring IFN-γ secretion in peripheral blood mononuclear cells (PBMCs) in response to ZIKV-prME immunization. Rhesus macaques were immunized intradermally with 2 mg of ZIKV-prME plasmid at weeks 0 and 4 administered as 1 mg at each of two sites, with immunization immediately followed by intradermal electroporation. PBMCs were isolated pre-immunization and at week 6 and were used for the ELISPOT assay to detect IFN-γ-secreting cells in response to stimulation with ZIKV-prME peptides as described in the ‘Materials and Methods’ section. The number of IFN-γ producing cells obtained per million PBMCs against six peptide pools encompassing the entire prME protein is shown. The values represent mean responses in each group (n=5)±s.e.m. FIG. 23B depicts the detection of ZIKV-prME-specific antibody responses following DNA vaccination. Anti-ZIKV IgG antibodies were measured pre-immunization and at week 6 by ELISA. FIG. 23C depicts end point ELISA titers for anti ZIKV-envelope antibodies are shown following the first and second immunizations. FIG. 23D depicts western blot analysis using week 6 RM immune sera demonstrated binding to recombinant envelope protein. FIG. 23E depicts PRNT activity of serum from RM immunized with ZIKV-prME. Pre-immunization and week 6 immune sera from individual monkeys were tested by plaque-reduction neutralization (PRNT) assay for their ability to neutralize ZIKV infectivity in vitro. PRNT50 was defined as the serum dilution factor that could inhibit 50% of the input virus. Calculated (PRNT50) values are listed for each monkey. IFN, interferon; ZIKV-prME, precursor membrane and envelope of Zika virus.

FIG. 24, comprising FIG. 24A through FIG. 24F depicts experimental results demonstrating survival data for immunized mice lacking the type I interferon α, β receptor following ZIKV infection. FIG. 24A depicts survival of IFNAR^{-/-} mice after ZIKV infection. Mice were immunized twice with 25 μg of the ZIKV-prME DNA vaccine at 2-week intervals and challenged with ZIKV-PR209 virus 1 week after the second immunization with 1×10⁶ plaque-forming units FIG. 24B depicts survival of IFNAR^{-/-} mice after ZIKV infection. Mice were immunized twice with 25 μg of the ZIKV-prME DNA vaccine at 2-week intervals and challenged with ZIKV-PR209 virus 1 week after the second immunization with 2×10⁶ plaque-forming units FIG. 24C depicts the weight change of animals immunized with 1×10⁶ plaque-forming units. FIG. 24D depicts the weight change of animals immunized with 2×10⁶ plaque-forming units. FIG. 24E depicts the clinical scores of animals immunized with 1×10⁶ plaque-forming units. FIG. 24F depicts the clinical scores of animals immunized with 2×10⁶ plaque-forming units. The designation for the clinical scores is as follows: 1: no disease, 2: decreased mobility; 3: hunched posture and decreased mobility; 4: hind limb knuckle walking (partial paralysis); 5: paralysis of one hind limb; and 6: paralysis of both hind limbs. The data reflect the results from two independent experiments with 10 mice per group per experiment. ZIKV-prME, precursor membrane and envelope of Zika virus.

FIG. 25, comprising FIG. 25A through FIG. 25d depicts experimental results demonstrating single immunization with the ZIKV-prME vaccine provided protection against ZIKV challenge in mice lacking the type I interferon α, β receptor. The mice were immunized once and challenged with 2×10⁶ plaque-forming units of ZIKV-PR209, 2 weeks after the single immunization. The survival curves depict 10 mice per group per experiment FIG. 25A demonstrates that the ZIKV-prME vaccine prevented ZIKA-induced neurological abnormalities in the mouse brain FIG. 25B depicts brain sections from pVax1 and ZIKV-prME vaccinated groups were collected 7-8 days after challenge and stained with H&E (haematoxylin and eosin) for histology. The sections taken from representative, unprotected pVax1 control animals shows pathology. (i): nuclear fragments within neuropils of the cerebral cortex (inset shows higher magnification and arrows to highlight nuclear fragments); (ii): perivascular cuffing of vessels within the cortex, lymphocyte infiltration and degenerating cells; (iii): perivascular cuffing, cellular degeneration and nuclear fragments within the cerebral cortex; and (iv): degenerating neurons within the hippocampus (arrows). An example of normal tissue from ZIKV-prME vaccinated mice appeared to be within normal limits (v and vi). FIG. 25C depicts levels of ZIKV RNA in the plasma samples from mice following vaccination and viral challenge at the indicated day post infection. The results are indicated as the genome equivalents per milliliter of plasma. FIG. 25D depicts levels of ZIKV-RNA in the brain tissues were analyzed at day 28 post infection. The results are indicated as the genome equivalent per gram of tissue. ZIKV-prME, precursor membrane and envelope of Zika virus.

FIG. 25, comprising FIG. 26A and FIG. 26B, depicts experimental results demonstrating protection of mice lacking the type I interferon α, β receptor following passive transfer of anti-ZIKV immune sera following ZIKV challenge. Pooled NHP anti-ZIKV immune sera, titred for anti-ZIKA virus IgG, was administered i.p. (150 μl/mouse) to mice 1 day after s.c. challenge with a ZIKA virus (10⁶ plaque-forming units per mouse). As a control, normal monkey sera and phosphate-buffered saline (PBS) were administered (150 μl/mouse) to age-matched mice as controls. FIG. 26A depicts the mouse weight change during the course of infection and treatment. Each point represents the mean and standard error of the calculated percent pre-challenge (day 0) weight for each mouse. FIG. 26B depicts the survival of mice following administration of the NHP immune sera. ZIKV-prME, precursor membrane and envelope of Zika virus.

FIG. 27, comprising FIG. 27A through FIG. 27D, depicts experimental results demonstrating the characterization of immune responses of ZIKV-prME-MR766 or ZIKV-prME Brazil vaccine in C57BL/6 mice. FIG. 27A depicts ELISpot and ELISA analysis measuring cellular and antibody responses after vaccination with either ZIKV-prME-MR766 and ZIKV-prME-Brazil DNA vaccines. C57BL/6 mice (n=4/group) were immunized intramuscularly three times with 25 μg of ZIKV-prME-MR766 followed by in vivo EP. IFN-γ generation, as an indication of cellular immune response induction, was measured by IFN-γ ELISpot. Splenocytes harvested one week after the third immunization were incubated in the presence of one of six peptide pools spanning the entire prM and E proteins. Results are shown in stacked bar graphs. The data represent the average numbers of SFU (spot forming units) per million splenocytes with values representing the mean responses in each±SEM. FIG. 27B depicts ELISpot and ELISA analysis measuring cellular and antibody responses after vaccination with either ZIKV-prME-MR766 and ZIKV-prME-Brazil DNA vaccines. C57BL/6 mice (n=4/group) were immunized intramuscularly three times with 25 μg of ZIKV prME-Brazil followed by in vivo EP. IFN-γ generation, as an indication of cellular immune response induction, was measured by IFN-γ ELISpot. Splenocytes harvested one week after the third immunization were incubated in the presence of one of six peptide pools spanning the entire prM and E proteins. Results are shown in stacked bar graphs. The data represent the average numbers of SFU (spot forming units) per million splenocytes with values representing the mean responses in each±SEM. FIG. 27C depicts ELISA analysis measuring binding antibody production in immunized C57BL/6 mice. Binding to rZIKV-E was analyzed with sera from mice at day 35 post immunization at various dilutions. FIG. 27D depicts ELISA analysis measuring binding antibody production in immunized C57BL/6 mice. Binding to rZIKV-E was analyzed with sera from mice at day 35 post immunization at various dilutions.

FIG. 28, comprising FIG. 28A through FIG. 28D, depicts experimental results demonstrating the expression, purification, and characterization of ZIKV-Envelope protein. FIG. 28A depicts the cloning plasmid for rZIKV E expression. FIG. 28B depicts the characterization of the recombinant ZIKV-E (rZIKV-E) protein by SDS-PAGE and Western blot analysis. Lane 1-BSA control; Lane 2-lysates from E. coli cultures transformed with pET-28a vector plasmid, was purified by nickel metal affinity resin columns and separated by SDS-PAGE after IPTG induction. Lane 3, 37 recombinant ZV-E purified protein was analyzed by Western blot with anti-His tag antibody. Lane M, Protein molecular weight marker. FIG. 28C depicts the purified rZIKV-E protein was evaluated for its antigenicity. ELISA plates were coated with rZIKV-E and then incubated with various dilutions of immune sera from the mice immunized with ZIKV-prME vaccine or Pan-flavivirus antibody as positive control. Bound IgG was detected by the addition of peroxidase-conjugated anti-mouse antibody followed by tetramethylbenzidine substrate as described in Experimental Example. FIG. 28D depicts western blot detection of purified rZIKV-E protein with immune sera from ZIKV prME immunized mice. Various concentrations of purified rZIKV-E protein were loaded onto an SDS-PAGE gel as described. A dilution of 1:100 immune sera, and goat anti-mouse at 1:15,000 were used for 1 hour at room temperature. After washing, the membranes were imaged on the Odyssey infrared imager. Odyssey protein molecular weight standards were used. The arrows indicate the position of rZIKV-E protein.

FIG. 29, comprising FIG. 29A through FIG. 29C, depicts experimental results demonstrating the characterization of immune responses ZIKA-prME in IFNAR^{-/-} mice. ELISpot and ELISA analysis measuring cellular and antibody responses to ZIKV-prME in IFNAR^{-/-} mice. Mice (n=4/group) were immunized intramuscularly three times with 25 μg of ZIKV-prME followed by in vivo EP. FIG. 29A depicts IFN-γ generation, as an indication of cellular immune response induction, was measured by IFN-γ ELISPOT. FIG. 29B depicts ELISA analysis measuring binding antibody production in immunized IFNAR^{-/-} mice. Binding to rZIKV-E was analyzed with sera from mice at various time points post immunization. FIG. 29C depicts endpoint titer analysis of anti-ZIKV antibodies produced in immunized IFNAR^{-/-} mice.

FIG. 30, comprising FIG. 30A through FIG. 30D, depicts experimental results demonstrating the neutralization activity of immune sera from Rhesus Macaques immunized against ZIKV-prME. SK-N-SH and U87MG cells were mock infected or infected with MR766 at an MOI of 0.01 PFU/cell in the presence of pooled NHP sera immunized with ZIKV-prME vaccine (Wk 6). Zika viral infectivity were analyzed 4 days post infection by indirect immunofluorescence assay (IFA) using sera from ZIKV-prME vaccinated NHPs. FIG. 30A depicts photographs of stained tissue sample slices taken with a 20× objective demonstrating inhibition of infection by ZIKV viruses MR766 and PR209 in Vero, SK-N-SH and U87MG FIG. 30B depicts photographs of stained tissue sample slices taken with a 20× objective demonstrating inhibition of infection by ZIKV viruses SK-N-SH and U87MG in Vero, SK-N-SH and U87MG FIG. 30C depicts a bar graph shows the percentage of infected (GFP positive cells) demonstrating the inhibition of infection by ZIKV viruses MR766 and PR209 in Vero, SK-N-SH and U87MG FIG. 30D depicts a bar graph showing the percentage of infected (GFP positive cells) demonstrating the inhibition of infection by ZIKV viruses SK-N-SH and U87MG in Vero, SK-N-SH and U87MG

FIG. 31, comprising FIG. 31A through FIG. 31D, depicts experimental results demonstrating ZIKV is virulent to IFNAR^{-/-} mice. These data confirm that ZIKV is virulent in IFNAR^{-/-} resulting in morbidity and mortality. FIG. 31A depicts Kaplan-Meier survival curves of IFNAR^{-/-} mice inoculated via intracranial with 10⁶ pfu ZIKV-PR209 virus. FIG. 31B depicts Kaplan-Meier survival curves of IFNAR^{-/-} mice inoculated via intravenously with 10⁶ pfu ZIKV-PR209 virus. FIG. 31C depicts Kaplan-Meier survival curves of IFNAR^{-/-} mice inoculated via intraperitoneal with 10⁶ pfu ZIKV-PR209 virus. FIG. 31D depicts Kaplan-Meier survival curves of IFNAR^{-/-} mice inoculated via subcutaneously with 10⁶ pfu ZIKV-PR209 virus. FIG. 31A depicts the mouse weight change during the course of infection for all the routes.

### DETAILED DESCRIPTION

In one embodiment, the invention provides composition comprising one or more nucleotide sequences encoding one or more antigens and one or more nucleotide sequences encoding one or more antibodies or fragments thereof.

In one embodiment, the invention provides a composition comprising a combination of a composition that elicits an immune response in a mammal against a desired target and a composition comprising a recombinant nucleic acid sequence encoding an antibody, a fragment thereof, a variant thereof, or a combination thereof.

In one embodiment, the recombinant nucleic acid sequence encoding an antibody comprises sequences that encode a heavy chain and light chain. In particular, the heavy chain and light chain polypeptides expressed from the recombinant nucleic acid sequences can assemble into the synthetic antibody. The heavy chain polypeptide and the light chain polypeptide can interact with one another such that assembly results in the synthetic antibody being capable of binding the antigen, being more immunogenic as compared to an antibody not assembled as described herein, and being capable of eliciting or inducing an immune response against the antigen.

Additionally, these synthetic antibodies are generated more rapidly in the subject than antibodies that are produced in response to antigen induced immune response. The synthetic antibodies are able to effectively bind and neutralize a range of antigens. The synthetic antibodies are also able to effectively protect against and/or promote survival of disease.

Another aspect of the present invention provides DNA plasmid vaccines that are capable of generating in a mammal an immune response against a desired target (e.g. an antigen). The DNA plasmid vaccines are comprised of a DNA plasmid capable of expressing a consensus antigen in a mammal and a pharmaceutically acceptable excipient. The DNA plasmid is comprised of a promoter operably linked to a coding sequence that encodes the consensus antigen.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOSITION

In one aspect, the present invention provides a combination of a composition that elicits an immune response in a mammal against an antigen with a composition comprising a recombinant nucleic acid sequence encoding an antibody, a fragment thereof, a variant thereof, or a combination thereof. The composition can be administered to a subject in need thereof to facilitate in vivo expression and formation of a synthetic antibody.

In one embodiment, the present invention relates to a combination of a first composition that elicits an immune response in a mammal against an antigen and a second composition comprising a recombinant nucleic acid sequence encoding an antibody, a fragment thereof, a variant thereof, or a combination thereof. In one embodiment, the first composition comprises a nucleic acid encoding one or more antigens. In one embodiment, the first composition comprises a DNA vaccine.

The present invention relates to a composition comprising a recombinant nucleic acid sequence encoding an antibody, a fragment thereof, a variant thereof, or a combination thereof. The composition, when administered to a subject in need thereof, can result in the generation of a synthetic antibody in the subject. The synthetic antibody can bind a target molecule (i.e., an antigen) present in the subject. Such binding can neutralize the antigen, block recognition of the antigen by another molecule, for example, a protein or nucleic acid, and elicit or induce an immune response to the antigen.

The synthetic antibody can treat, prevent, and/or protect against disease in the subject administered the composition. The synthetic antibody by binding the antigen can treat, prevent, and/or protect against disease in the subject administered the composition. The synthetic antibody can promote survival of the disease in the subject administered the composition. The synthetic antibody can provide at least about 50%, 55%, 60%, 65%, 70%, 75%, 80%, 85%, 90%, 95%, or 100% survival of the disease in the subject administered the composition. In other embodiments, the synthetic antibody can provide at least about 65%, 66%, 67%, 68%, 69%, 70%, 71%, 72%, 73%, 74%, 75%, 76%, 77%, 78%, 79%, or 80% survival of the disease in the subject administered the composition.

The composition can result in the generation of the synthetic antibody in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody in the subject within at least about 1 day, 2 days, 3 days, 4 days, 5 days, 6 days, 7 days, 8 days, 9 days, or 10 days of administration of the composition to the subject. The composition can result in generation of the synthetic antibody in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic antibody in the subject more quickly than the generation of an endogenous antibody in a subject who is administered an antigen to induce a humoral immune response. The composition can result in the generation of the synthetic antibody at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

Another aspect of the present invention provides DNA plasmid vaccines that are capable of generating in a mammal an immune response against an antigen. The DNA plasmid vaccines are comprised of a DNA plasmid capable of expressing a consensus antigen in the mammal and a pharmaceutically acceptable excipient. The DNA plasmid is comprised of a promoter operably linked to a coding sequence that encodes the consensus antigen.

In some embodiments, the DNA sequences herein can have removed from the 5′ end the IgE leader sequence, and the protein sequences herein can have removed from the N-terminus the IgE leader sequence.

In some embodiments, the DNA plasmid includes and encoding sequence that encodes for a antigen minus an IgE leader sequence on the N-terminal end of the coding sequence. In some embodiments, the DNA plasmid further comprises an IgE leader sequence attached to an N-terminal end of the coding sequence and operably linked to the promoter.

The DNA plasmid can further include a polyadenylation sequence attached to the C-terminal end of the coding sequence. Preferably, the DNA plasmid is codon optimized.

In some embodiments, the pharmaceutically acceptable excipient is an adjuvant. Preferably, the adjuvant is selected from the group consisting of: IL-12 and IL-15. In some embodiments, the pharmaceutically acceptable excipient is a transfection facilitating agent. Preferably, the transfection facilitating agent is a polyanion, polycation, or lipid, and more preferably poly-L-glutamate. Preferably, the poly-L-glutamate is at a concentration less than 6 mg/ml. Preferably, the DNA plasmid vaccine has a concentration of total DNA plasmid of 1 mg/ml or greater.

In some embodiments, the DNA plasmid comprises a plurality of unique DNA plasmids, wherein each of the plurality of unique DNA plasmids encodes a polypeptide comprising a consensus antigen.

In some embodiments of the present invention, the DNA plasmid vaccines can further include an adjuvant. In some embodiments, the adjuvant is selected from the group consisting of: alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-1, MIP-1-alpha, MIP-1p, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof. In some preferred embodiments, the adjuvant is selected from IL-12, IL-15, CTACK, TECK, or MEC.

In some embodiments, methods of eliciting an immune response in mammals against a consensus antigen include methods of inducing mucosal immune responses. Such methods include administering to the mammal one or more of CTACK protein, TECK protein, MEC protein and functional fragments thereof or expressible coding sequences thereof in combination with a DNA plasmid including a consensus antigen, described above. The one or more of CTACK protein, TECK protein, MEC protein and functional fragments thereof may be administered prior to, simultaneously with or after administration of the DNA plasmid vaccines provided herein. In some embodiments, an isolated nucleic acid molecule that encodes one or more proteins of selected from the group consisting of: CTACK, TECK, MEC and functional fragments thereof is administered to the mammal.

### 3. DNA VACCINE

As described above, the composition can comprise immunogenic compositions, such as vaccines, comprising one or more antigens. The vaccine can be used to protect against any number of antigens, thereby treating, preventing, and/or protecting against antigen based pathologies. The vaccine can significantly induce an immune response of a subject administered the vaccine, thereby protecting against and treating infection by the antigen.

The vaccine can be a DNA vaccine, a peptide vaccine, or a combination DNA and peptide vaccine. The DNA vaccine can include a nucleic acid sequence encoding the antigen. The nucleic acid sequence can be DNA, RNA, cDNA, a variant thereof, a fragment thereof, or a combination thereof. The nucleic acid sequence can also include additional sequences that encode linker, leader, or tag sequences that are linked to the antigen by a peptide bond. The peptide vaccine can include a antigenic peptide, a antigenic protein, a variant thereof, a fragment thereof, or a combination thereof. The combination DNA and peptide vaccine can include the above described nucleic acid sequence encoding the antigen and the antigenic peptide or protein, in which the antigenic peptide or protein and the encoded antigen have the same amino acid sequence.

The vaccine can induce a humoral immune response in the subject administered the vaccine. The induced humoral immune response can be specific for the antigen. The induced humoral immune response can be reactive with the antigen. The humoral immune response can be induced in the subject administered the vaccine by about 1.5-fold to about 16-fold, about 2-fold to about 12-fold, or about 3-fold to about 10-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by the vaccine can include an increased level of neutralizing antibodies associated with the subject administered the vaccine as compared to a subject not administered the vaccine. The neutralizing antibodies can be specific for the antigen. The neutralizing antibodies can be reactive with the antigen. The neutralizing antibodies can provide protection against and/or treatment of infection and its associated pathologies in the subject administered the vaccine.

The humoral immune response induced by the vaccine can include an increased level of IgG antibodies associated with the subject administered the vaccine as compared to a subject not administered the vaccine. These IgG antibodies can be specific for the antigen. These IgG antibodies can be reactive with the antigen. Preferably, the humoral response is cross-reactive against two or more strains of the antigen. The level of IgG antibody associated with the subject administered the vaccine can be increased by about 1.5-fold to about 16-fold, about 2-fold to about 12-fold, or about 3-fold to about 10-fold as compared to the subject not administered the vaccine. The level of IgG antibody associated with the subject administered the vaccine can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the subject not administered the vaccine.

The vaccine can induce a cellular immune response in the subject administered the vaccine. The induced cellular immune response can be specific for the antigen. The induced cellular immune response can be reactive to the antigen. Preferably, the cellular response is cross-reactive against two or more strains of the antigen. The induced cellular immune response can include eliciting a CD8⁺ T cell response. The elicited CD8⁺ T cell response can be reactive with the antigen. The elicited CD8⁺ T cell response can be polyfunctional. The induced cellular immune response can include eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

The induced cellular immune response can include an increased CD8⁺ T cell response associated with the subject administered the vaccine as compared to the subject not administered the vaccine. The CD8⁺ T cell response associated with the subject administered the vaccine can be increased by about 2-fold to about 30-fold, about 3-fold to about 25-fold, or about 4-fold to about 20-fold as compared to the subject not administered the vaccine. The CD8⁺ T cell response associated with the subject administered the vaccine can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered the vaccine.

The induced cellular immune response can include an increased frequency of CD3⁺CD8⁺ T cells that produce IFN-γ. The frequency of CD3⁺CD8⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD8⁺ T cells that produce TNF-α. The frequency of CD3⁺CD8⁺ TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to the subject not administered the vaccine.

The induced cellular immune response can include an increased frequency of CD3⁺CD8⁺ T cells that produce IL-2. The frequency of CD3⁺CD8⁺IL-2⁺ T cells associated with the subject administered the vaccine can be increased by at least about 0.5-fold, 1.0-fold, 1.5-fold, 2.0-fold, 2.5-fold, 3.0-fold, 3.5-fold, 4.0-fold, 4.5-fold, or 5.0-fold as compared to the subject not administered the vaccine.

The induced cellular immune response can include an increased frequency of CD3⁺CD8⁺ T cells that produce both IFN-γ and TNF-α. The frequency of CD3⁺CD8⁺IFN-γ⁺ TNF-α⁺ T cells associated with the subject administered the vaccine can be increased by at least about 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 160-fold, 170-fold, or 180-fold as compared to the subject not administered the vaccine.

The cellular immune response induced by the vaccine can include eliciting a CD4⁺ T cell response. The elicited CD4⁺ T cell response can be reactive with the desired antigen. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD3⁺CD4⁺ T cells that produce IFN-γ. The frequency of CD3⁺CD4⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD4⁺ T cells that produce TNF-α. The frequency of CD3⁺CD4⁺ TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to the subject not administered the vaccine.

The induced cellular immune response can include an increased frequency of CD3⁺CD4⁺ T cells that produce IL-2. The frequency of CD3⁺CD4⁺IL-2⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 45-fold, 50-fold, 55-fold, or 60-fold as compared to the subject not administered the vaccine.

The induced cellular immune response can include an increased frequency of CD3⁺CD4⁺ T cells that produce both IFN-γ and TNF-α. The frequency of CD3⁺CD4⁺IFN-γ⁺ TNF-α⁺ associated with the subject administered the vaccine can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to the subject not administered the vaccine.

The vaccine of the present invention can have features required of effective vaccines such as being safe so the vaccine itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The vaccine can further induce an immune response when administered to different tissues such as the muscle or skin. The vaccine can further induce an immune response when administered via electroporation, or injection, or subcutaneously, or intramuscularly.

a. Vaccine Constructs and Plasmids

The vaccine can comprise nucleic acid constructs or plasmids that encode the one or more antigens. The nucleic acid constructs or plasmids can include or contain one or more heterologous nucleic acid sequences. Provided herein are genetic constructs that can comprise a nucleic acid sequence that encodes the antigens. The genetic construct can be present in the cell as a functioning extrachromosomal molecule. The genetic construct can be a linear minichromosome including centromere, telomeres or plasmids or cosmids. The genetic constructs can include or contain one or more heterologous nucleic acid sequences.

The genetic constructs can be in the form of plasmids expressing the antigen in any order.

The genetic construct can also be part of a genome of a recombinant viral vector, including recombinant adenovirus, recombinant adenovirus associated virus and recombinant vaccinia. The genetic construct can be part of the genetic material in attenuated live microorganisms or recombinant microbial vectors which live in cells.

The genetic constructs can comprise regulatory elements for gene expression of the coding sequences of the nucleic acid. The regulatory elements can be a promoter, an enhancer an initiation codon, a stop codon, or a polyadenylation signal.

The nucleic acid sequences can make up a genetic construct that can be a vector. The vector can be capable of expressing the antigen in the cell of a mammal in a quantity effective to elicit an immune response in the mammal. The vector can be recombinant. The vector can comprise heterologous nucleic acid encoding the antigen. The vector can be a plasmid. The vector can be useful for transfecting cells with nucleic acid encoding the antigen, which the transformed host cell is cultured and maintained under conditions wherein expression of the antigen takes place.

Coding sequences can be optimized for stability and high levels of expression. In some instances, codons are selected to reduce secondary structure formation of the RNA such as that formed due to intramolecular bonding.

The vector can comprise heterologous nucleic acid encoding the antigens and can further comprise an initiation codon, which can be upstream of the one or more cancer antigen coding sequence(s), and a stop codon, which can be downstream of the coding sequence(s) of the antigen. The initiation and termination codon can be in frame with the coding sequence(s) of the antigen. The vector can also comprise a promoter that is operably linked to the coding sequence(s) of the antigen. The promoter operably linked to the coding sequence(s) of the antigen can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene such as human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the contents of which are incorporated herein in its entirety.

The vector can also comprise a polyadenylation signal, which can be downstream of the coding sequence(s) of the antigen. The polyadenylation signal can be a SV40 polyadenylation signal, LTR polyadenylation signal, bovine growth hormone (bGH) polyadenylation signal, human growth hormone (hGH) polyadenylation signal, or human (3-globin polyadenylation signal. The SV40 polyadenylation signal can be a polyadenylation signal from a pCEP4 vector (Invitrogen, San Diego, Calif.).

The vector can also comprise an enhancer upstream of the antigen. The enhancer can be necessary for DNA expression. The enhancer can be human actin, human myosin, human hemoglobin, human muscle creatine or a viral enhancer such as one from CMV, HA, RSV or EBV. Polynucleotide function enhances are described in U.S. Pat. Nos. 5,593,972, 5,962,428, and WO94/016737, the contents of each are fully incorporated by reference.

The vector can also comprise a mammalian origin of replication in order to maintain the vector extrachromosomally and produce multiple copies of the vector in a cell. The vector can be pVAX1, pCEP4 or pREP4 from Invitrogen (San Diego, Calif.), which can comprise the Epstein Barr virus origin of replication and nuclear antigen EBNA-1 coding region, which can produce high copy episomal replication without integration. The vector can be pVAX1 or a pVax1 variant with changes such as the variant plasmid described herein. The variant pVax1 plasmid is a 2998 basepair variant of the backbone vector plasmid pVAX1 (Invitrogen, Carlsbad Calif.). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993.

Based upon the sequence of pVAX1 available from Invitrogen, the following mutations were found in the sequence of pVAX1 that was used as the backbone for plasmids 1-6 set forth herein:

C>G241 in CMV promoter

C>T1942 backbone, downstream of the bovine growth hormone polyadenylation signal (bGHpolyA)

A>−2876 backbone, downstream of the Kanamycin gene

C>T3277 in pUC origin of replication (Ori) high copy number mutation (see Nucleic Acid Research 1985)

G>C 3753 in very end of pUC Ori upstream of RNASeH site

Base pairs 2, 3 and 4 are changed from ACT to CTG in backbone, upstream of CMV promoter.

The backbone of the vector can be pAV0242. The vector can be a replication defective adenovirus type 5 (Ad5) vector.

The vector can also comprise a regulatory sequence, which can be well suited for gene expression in a mammalian or human cell into which the vector is administered. The one or more cancer antigen sequences disclosed herein can comprise a codon, which can allow more efficient transcription of the coding sequence in the host cell.

The vector can be pSE420 (Invitrogen, San Diego, Calif.), which can be used for protein production in Escherichia coli (E. coli). The vector can also be pYES2 (Invitrogen, San Diego, Calif.), which can be used for protein production in Saccharomyces cerevisiae strains of yeast. The vector can also be of the MAXBAC™ complete baculovirus expression system (Invitrogen, San Diego, Calif.), which can be used for protein production in insect cells. The vector can also be pcDNA I or pcDNA3 (Invitrogen, San Diego, Calif.), which maybe used for protein production in mammalian cells such as Chinese hamster ovary (CHO) cells. The vector can be expression vectors or systems to produce protein by routine techniques and readily available starting materials including Sambrook et al., Molecular Cloning and Laboratory Manual, Second Ed., Cold Spring Harbor (1989), which is incorporated fully by reference.

### 4. DNA ENCODED ANTIBODY

As described above, the composition can comprise a recombinant nucleic acid sequence. The recombinant nucleic acid sequence can encode the antibody, a fragment thereof, a variant thereof, or a combination thereof. The antibody is described in more detail below.

The recombinant nucleic acid sequence can be a heterologous nucleic acid sequence. The recombinant nucleic acid sequence can include at least one heterologous nucleic acid sequence or one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

a. Recombinant Nucleic Acid Sequence Construct

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

(1) Heavy Chain Polypeptide

The recombinant nucleic acid sequence construct can include the heterologous nucleic acid encoding the heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

(2) Light Chain Polypeptide

The recombinant nucleic acid sequence construct can include the heterologous nucleic acid sequence encoding the light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

(3) Protease Cleavage Site

The recombinant nucleic acid sequence construct can include the heterologous nucleic acid sequence encoding the protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

(4) Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides.

(5) Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

(6) Intron

The recombinant nucleic acid sequence construct can include one or more introns. Each intron can include functional splice donor and acceptor sites. The intron can include an enhancer of splicing. The intron can include one or more signals required for efficient splicing.

(7) Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

(8) Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

(9) Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

(10) Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, Calif.).

(11) Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

b. Arrangement of the Recombinant Nucleic Acid Sequence Construct

As described above, the recombinant nucleic acid sequence can include one or more recombinant nucleic acid sequence constructs, in which each recombinant nucleic acid sequence construct can include one or more components. The one or more components are described in detail above. The one or more components, when included in the recombinant nucleic acid sequence construct, can be arranged in any order relative to one another. In some embodiments, the one or more components can be arranged in the recombinant nucleic acid sequence construct as described below.

(1) Arrangement 1

In one arrangement, a first recombinant nucleic acid sequence construct can include the heterologous nucleic acid sequence encoding the heavy chain polypeptide and a second recombinant nucleic acid sequence construct can include the heterologous nucleic acid sequence encoding the light chain polypeptide.

The first recombinant nucleic acid sequence construct can be placed in a vector. The second recombinant nucleic acid sequence construct can be placed in a second or separate vector. Placement of the recombinant nucleic acid sequence construct into the vector is described in more detail below.

The first recombinant nucleic acid sequence construct can also include the promoter, intron, transcription termination region, initiation codon, termination codon, and/or polyadenylation signal. The first recombinant nucleic acid sequence construct can further include the leader sequence, in which the leader sequence is located upstream (or 5′) of the heterologous nucleic acid sequence encoding the heavy chain polypeptide. Accordingly, the signal peptide encoded by the leader sequence can be linked by a peptide bond to the heavy chain polypeptide.

The second recombinant nucleic acid sequence construct can also include the promoter, initiation codon, termination codon, and polyadenylation signal. The second recombinant nucleic acid sequence construct can further include the leader sequence, in which the leader sequence is located upstream (or 5′) of the heterologous nucleic acid sequence encoding the light chain polypeptide. Accordingly, the signal peptide encoded by the leader sequence can be linked by a peptide bond to the light chain polypeptide.

Accordingly, one example of arrangement 1 can include the first vector (and thus first recombinant nucleic acid sequence construct) encoding the heavy chain polypeptide that includes VH and CH1, and the second vector (and thus second recombinant nucleic acid sequence construct) encoding the light chain polypeptide that includes VL and CL. A second example of arrangement 1 can include the first vector (and thus first recombinant nucleic acid sequence construct) encoding the heavy chain polypeptide that includes VH, CH1, hinge region, CH2, and CH3, and the second vector (and thus second recombinant nucleic acid sequence construct) encoding the light chain polypeptide that includes VL and CL.

(2) Arrangement 2

In a second arrangement, the recombinant nucleic acid sequence construct can include the heterologous nucleic acid sequence encoding the heavy chain polypeptide and the heterologous nucleic acid sequence encoding the light chain polypeptide. The heterologous nucleic acid sequence encoding the heavy chain polypeptide can be positioned upstream (or 5′) of the heterologous nucleic acid sequence encoding the light chain polypeptide. Alternatively, the heterologous nucleic acid sequence encoding the light chain polypeptide can be positioned upstream (or 5′) of the heterologous nucleic acid sequence encoding the heavy chain polypeptide.

The recombinant nucleic acid sequence construct can be placed in the vector as described in more detail below.

The recombinant nucleic acid sequence construct can include the heterologous nucleic acid sequence encoding the protease cleavage site and/or the linker sequence. If included in the recombinant nucleic acid sequence construct, the heterologous nucleic acid sequence encoding the protease cleavage site can be positioned between the heterologous nucleic acid sequence encoding the heavy chain polypeptide and the heterologous nucleic acid sequence encoding the light chain polypeptide. Accordingly, the protease cleavage site allows for separation of the heavy chain polypeptide and the light chain polypeptide into distinct polypeptides upon expression. In other embodiments, if the linker sequence is included in the recombinant nucleic acid sequence construct, then the linker sequence can be positioned between the heterologous nucleic acid sequence encoding the heavy chain polypeptide and the heterologous nucleic acid sequence encoding the light chain polypeptide.

The recombinant nucleic acid sequence construct can also include the promoter, intron, transcription termination region, initiation codon, termination codon, and/or polyadenylation signal. The recombinant nucleic acid sequence construct can include one or more promoters. The recombinant nucleic acid sequence construct can include two promoters such that one promoter can be associated with the heterologous nucleic acid sequence encoding the heavy chain polypeptide and the second promoter can be associated with the heterologous nucleic acid sequence encoding the light chain polypeptide. In still other embodiments, the recombinant nucleic acid sequence construct can include one promoter that is associated with the heterologous nucleic acid sequence encoding the heavy chain polypeptide and the heterologous nucleic acid sequence encoding the light chain polypeptide.

The recombinant nucleic acid sequence construct can further include two leader sequences, in which a first leader sequence is located upstream (or 5′) of the heterologous nucleic acid sequence encoding the heavy chain polypeptide and a second leader sequence is located upstream (or 5′) of the heterologous nucleic acid sequence encoding the light chain polypeptide. Accordingly, a first signal peptide encoded by the first leader sequence can be linked by a peptide bond to the heavy chain polypeptide and a second signal peptide encoded by the second leader sequence can be linked by a peptide bond to the light chain polypeptide.

Accordingly, one example of arrangement 2 can include the vector (and thus recombinant nucleic acid sequence construct) encoding the heavy chain polypeptide that includes VH and CH1, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A second example of arrangement of 2 can include the vector (and thus recombinant nucleic acid sequence construct) encoding the heavy chain polypeptide that includes VH and CH1, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

A third example of arrangement 2 can include the vector (and thus recombinant nucleic acid sequence construct) encoding the heavy chain polypeptide that includes VH, CH1, hinge region, CH2, and CH3, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A forth example of arrangement of 2 can include the vector (and thus recombinant nucleic acid sequence construct) encoding the heavy chain polypeptide that includes VH, CH1, hinge region, CH2, and CH3, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

c. Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody. In particular, the heavy chain polypeptide and the light chain polypeptide can interact with one another such that assembly results in the synthetic antibody being capable of binding the antigen. In other embodiments, the heavy chain polypeptide and the light chain polypeptide can interact with one another such that assembly results in the synthetic antibody being more immunogenic as compared to an antibody not assembled as described herein. In still other embodiments, the heavy chain polypeptide and the light chain polypeptide can interact with one another such that assembly results in the synthetic antibody being capable of eliciting or inducing an immune response against the antigen.

d. Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

(1) Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

(2) Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAXI, pCEP4 or pREP4 from Invitrogen (San Diego, Calif.), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be p YES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

(3) Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be perM2. The LEC can be perNP. perNP and perMR can be derived from pNP (Puerto Rico/34) and pM2 (New Caledonia/99), respectively.

(4) Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in WO/2008/148010, published Dec. 4, 2008. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### 5. ANTIBODY

As described above, the recombinant nucleic acid sequence can encode the antibody, a fragment thereof, a variant thereof, or a combination thereof. The antibody can bind or react with the antigen, which is described in more detail below.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

The antibody may be modified to reduce or prevent antibody-dependent enhancement (ADE) of disease associated with the antigen as described in more detail below.

a. Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker, including a cancer marker.

b. Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CIVIL).

c. Extension of Antibody Half-Life

As described above, the antibody may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

d. Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, 0-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof e. Reduced ADE Response

The antibody may be modified to reduce or prevent antibody-dependent enhancement (ADE) of disease associated with the antigen, but still neutralize the antigen. For example, the antibody may be modified to reduce or prevent ADE of disease associated with DENV, which is described below in more detail, but still neutralize DENV.

In some embodiments, the antibody may be modified to include one or more amino acid substitutions that reduce or prevent binding of the antibody to FcγR1a. The one or more amino acid substitutions may be in the constant region of the antibody. The one or more amino acid substitutions may include replacing a leucine residue with an alanine residue in the constant region of the antibody, i.e., also known herein as LA, LA mutation or LA substitution. The one or more amino acid substitutions may include replacing two leucine residues, each with an alanine residue, in the constant region of the antibody and also known herein as LALA, LALA mutation, or LALA substitution. The presence of the LALA substitutions may prevent or block the antibody from binding to FcγR1a, and thus, the modified antibody does not enhance or cause ADE of disease associated with the antigen, but still neutralizes the antigen.

### 6. ANTIGEN

The DNA plasmid vaccines encode an antigen or fragment or variant thereof. The synthetic antibody is directed to the antigen or fragment or variant thereof. The antigen can be a nucleic acid sequence, an amino acid sequence, or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof.

The antigen can be from any number of organisms, for example, a virus, a parasite, a bacterium, a fungus, or a mammal. The antigen can be associated with an autoimmune disease, allergy, or asthma. In other embodiments, the antigen can be associated with cancer, herpes, influenza, hepatitis B, hepatitis C, human papilloma virus (HPV), or human immunodeficiency virus (HIV).

In some embodiments, the antigen is foreign. In some embodiments, the antigen is a self-antigen.

a. Foreign Antigens

In some embodiments, the antigen is foreign. A foreign antigen is any non-self substance (i.e., originates external to the subject) that, when introduced into the body, is capable of stimulating an immune response.

(1) Viral Antigens

The foreign antigen can be a viral antigen, or fragment thereof, or variant thereof. The viral antigen can be from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. The viral antigen can be from human immunodeficiency virus (HIV), Chikungunya virus (CHIKV), dengue fever virus, papilloma viruses, for example, human papillomoa virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, influenza virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, or cancer causing virus.

(a) Human Immunodeficiency Virus (HIV) Antigen

The viral antigen may be from Human Immunodeficiency Virus (HIV) virus. In some embodiments, the HIV antigen can be a subtype A envelope protein, subtype B envelope protein, subtype C envelope protein, subtype D envelope protein, subtype B Nef-Rev protein, Gag subtype A, B, C, or D protein, MPol protein, a nucleic acid or amino acid sequences of Env A, Env B, Env C, Env D, B Nef-Rev, Gag, or any combination thereof.

A synthetic antibody specific for HIV can include a Fab fragment comprising the amino acid sequence of SEQ ID NO:48, which is encoded by the nucleic acid sequence of SEQ ID NO:3, and the amino acid sequence of SEQ ID NO:49, which is encoded by the nucleic acid sequence of SEQ ID NO:4. The synthetic antibody can comprise the amino acid sequence of SEQ ID NO:46, which is encoded by the nucleic acid sequence of SEQ ID NO:6, and the amino acid sequence of SEQ ID NO:47, which is encoded by the nucleic acid sequence of SEQ ID NO:7. The Fab fragment comprise the amino acid sequence of SEQ ID NO:51, which is encoded by the nucleic acid sequence of SEQ ID NO:50. The Fab can comprise the amino acid sequence of SEQ ID NO:53, which is encoded by the nucleic acid sequence of SEQ ID NO:52.

A synthetic antibody specific for HIV can include an Ig comprising the amino acid sequence of SEQ ID NO:5. The Ig can comprise the amino acid sequence of SEQ ID NO:1, which is encoded by the nucleic acid sequence of SEQ ID NO:62. The Ig can comprise the amino acid sequence of SEQ ID NO:2, which is encoded by the nucleic acid sequence of SEQ ID NO:63. The Ig can comprise the amino acid sequence of SEQ ID NO:55, which is encoded by the nucleic acid sequence of SEQ ID NO:54, and the amino acid sequence of SEQ ID NO:57, which is encoded by the nucleic acid sequence SEQ ID NO:56.

A DNA vaccine encoding an HIV antigen can include a vaccine encoding a subtype A envelope protein, subtype B envelope protein, subtype C envelope protein, subtype D envelope protein, subtype B Nef-Rev protein, Gag subtype A, B, C, or D protein, MPol protein, a nucleic acid or amino acid sequences of Env A, Env B, Env C, Env D, B Nef-Rev, Gag, or any combination thereof. Examples of DNA vaccines encoding HIV antigens include those described in U.S. Pat. No. 8,168,769 and WO2015/073291, the contents of each are fully incorporated by reference.

(b) Chikungunya Virus

The viral antigen may be from Chikungunya virus. Chikungunya virus belongs to the alphavirus genus of the Togaviridae family. Chikungunya virus is transmitted to humans by the bite of infected mosquitoes, such as the genus Aedes.

A synthetic antibody specific for CHIKV can include a Fab fragment comprising the amino acid sequence of SEQ ID NO:59, which is encoded by the nucleic acid sequence of SEQ ID NO:58, and the amino acid sequence of SEQ ID NO:61, which is encoded by the nucleic acid sequence of SEQ ID NO:60. A synthetic antibody specific for CHIKV can include an Ig encoded by one of SEQ ID NOs: 97-100.

The DNA vaccine may encode a CHIKV antigen. Examples of DNA vaccines encoding CHIKV antigens include those described in U.S. Pat. No. 8,852,609, the contents of which is fully incorporated by reference. A DNA vaccine encoding a CHIKV antigen may include a nucleic acid sequence encoding an amino acid sequence comprising one of SEQ ID NOs: 81-88. The DNA vaccine encoding a CHIKV antigen may include a nucleic acid sequence comprising the sequence SEQ ID NOs: 89-96. For Example, in one embodiment, the DNA vaccine encodes a CHIKV E1 consensus protein. In one embodiment, the CHIKV E1 consensus protein comprises an amino acid sequence of one of SEQ ID NOs: 81 or 84. In one embodiment, the DNA vaccine encoding a CHIKV E1 consensus protein comprises a nucleic acid sequence of SEQ ID NOs:89 or 92. In one embodiment, the DNA vaccine encodes a CHIKV E2 consensus protein. In one embodiment, the CHIKV E2 consensus protein comprises an amino acid sequence of one of SEQ ID NOs: 82 or 85. In one embodiment, the DNA vaccine encoding a CHIKV E2 consensus protein comprises a nucleic acid sequence of SEQ ID NOs: 90 or 93. In one embodiment, the DNA vaccine encodes a CHIKV Capsid consensus protein. In one embodiment, the CHIKV Capsid consensus protein comprises an amino acid sequence of one of SEQ ID NOs: 83 or 86. In one embodiment, the DNA vaccine encoding a CHIKV Capsid consensus protein comprises a nucleic acid sequence of SEQ ID NOs: 91 or 94. In one embodiment, the DNA vaccine encodes a CHIKV Env consensus protein. In one embodiment, the CHIKV Env consensus protein comprises an amino acid sequence of one of SEQ ID NOs: 87 or 88. In one embodiment, the DNA vaccine encoding a CHIKV Env consensus protein comprises a nucleic acid sequence of SEQ ID NOs: 95 or 96.

(c) Dengue Virus

The viral antigen may be from Dengue virus. The Dengue virus antigen may be one of three proteins or polypeptides (C, prM, and E) that form the virus particle. The Dengue virus antigen may be one of seven other proteins or polypeptides (NS1, NS2a, NS2b, NS3, NS4a, NS4b, NS5) which are involved in replication of the virus. The Dengue virus may be one of five strains or serotypes of the virus, including DENV-1, DENV-2, DENV-3 and DENV-4. The antigen may be any combination of a plurality of Dengue virus antigens.

A synthetic antibody specific for Dengue virus can include a Ig comprising the amino acid sequence of SEQ ID NO:45, which is encoded by the nucleic acid sequence of SEQ ID NO:44.

The DNA vaccine may encode a Dengue virus antigen. Examples of DNA vaccines encoding Dengue virus antigens include those described in U.S. Pat. No. 8,835,620 and WO2014/144786, the contents of each are fully incorporated by reference.

(d) Hepatitis Antigen

The viral antigen may include a hepatitis virus antigen (i.e., hepatitis antigen), or a fragment thereof, or a variant thereof. The hepatitis antigen can be an antigen or immunogen from one or more of hepatitis A virus (HAV), hepatitis B virus (HBV), hepatitis C virus (HCV), hepatitis D virus (HDV), and/or hepatitis E virus (HEV).

The hepatitis antigen can be an antigen from HAV. The hepatitis antigen can be a HAV capsid protein, a HAV non-structural protein, a fragment thereof, a variant thereof, or a combination thereof.

The hepatitis antigen can be an antigen from HCV. The hepatitis antigen can be a HCV nucleocapsid protein (i.e., core protein), a HCV envelope protein (e.g., E1 and E2), a HCV non-structural protein (e.g., NS1, NS2, NS3, NS4a, NS4b, NS5a, and NS5b), a fragment thereof, a variant thereof, or a combination thereof.

The hepatitis antigen can be an antigen from HDV. The hepatitis antigen can be a HDV delta antigen, fragment thereof, or variant thereof.

The hepatitis antigen can be an antigen from HEV. The hepatitis antigen can be a HEV capsid protein, fragment thereof, or variant thereof.

The hepatitis antigen can be an antigen from HBV. The hepatitis antigen can be a HBV core protein, a HBV surface protein, a HBV DNA polymerase, a HBV protein encoded by gene X, fragment thereof, variant thereof, or combination thereof. The hepatitis antigen can be a HBV genotype A core protein, a HBV genotype B core protein, a HBV genotype C core protein, a HBV genotype D core protein, a HBV genotype E core protein, a HBV genotype F core protein, a HBV genotype G core protein, a HBV genotype H core protein, a HBV genotype A surface protein, a HBV genotype B surface protein, a HBV genotype C surface protein, a HBV genotype D surface protein, a HBV genotype E surface protein, a HBV genotype F surface protein, a HBV genotype G surface protein, a HBV genotype H surface protein, fragment thereof, variant thereof, or combination thereof.

In some embodiments, the hepatitis antigen can be an antigen from HBV genotype A, HBV genotype B, HBV genotype C, HBV genotype D, HBV genotype E, HBV genotype F, HBV genotype G, or HBV genotype H.

The DNA vaccine may encode a hepatitis antigen. Examples of DNA vaccines encoding hepatitis antigens include those described in U.S. Pat. Nos. 8,829,174, 8,921,536, 9,403,879, 9,238,679, the contents of each are fully incorporated by reference.

(e) Human Papilloma Virus (HPV) Antigen

The viral antigen may comprise an antigen from HPV. The HPV antigen can be from HPV types 16, 18, 31, 33, 35, 45, 52, and 58 which cause cervical cancer, rectal cancer, and/or other cancers. The HPV antigen can be from HPV types 6 and 11, which cause genital warts, and are known to be causes of head and neck cancer.

The HPV antigens can be the HPV E6 or E7 domains from each HPV type. For example, for HPV type 16 (HPV16), the HPV16 antigen can include the HPV16 E6 antigen, the HPV16 E7 antigen, fragments, variants, or combinations thereof. Similarly, the HPV antigen can be HPV 6 E6 and/or E7, HPV 11 E6 and/or E7, HPV 18 E6 and/or E7, HPV 31 E6 and/or E7, HPV 33 E6 and/or E7, HPV 52 E6 and/or E7, or HPV 58 E6 and/or E7, fragments, variants, or combinations thereof.

The DNA vaccine may encode a HPV antigen. Examples of DNA vaccines encoding HPV antigens include those described in WO/2008/014521, published Jan. 31, 2008; U.S. Patent Application Pub. No. 20160038584; U.S. Pat. Nos. 8,389,706 and 9,050,287, the contents of each are fully incorporated by reference.

(f) RSV Antigen

The viral antigen may comprise a RSV antigen. The RSV antigen can be a human RSV fusion protein (also referred to herein as “RSV F,” “RSV F protein,” and “F protein”), or fragment or variant thereof. The human RSV fusion protein can be conserved between RSV subtypes A and B. The RSV antigen can be a RSV F protein, or fragment or variant thereof, from the RSV Long strain (GenBank AAX23994.1). The RSV antigen can be a RSV F protein from the RSV A2 strain (GenBank AAB59858.1), or a fragment or variant thereof. The RSV antigen can be a monomer, a dimer, or trimer of the RSV F protein, or a fragment or variant thereof.

The RSV F protein can be in a prefusion form or a postfusion form. The postfusion form of RSV F elicits high titer neutralizing antibodies in immunized animals and protects the animals from RSV challenge.

The RSV antigen can also be human RSV attachment glycoprotein (also referred to herein as “RSV G,” “RSV G protein,” and “G protein”), or fragment or variant thereof. The human RSV G protein differs between RSV subtypes A and B. The antigen can be RSV G protein, or fragment or variant thereof, from the RSV Long strain (GenBank AAX23993). The RSV antigen can be RSV G protein from the RSV subtype B isolate H5601, the RSV subtype B isolate H1068, the RSV subtype B isolate H5598, the RSV subtype B isolate H1123, or a fragment or variant thereof.

In other embodiments, the RSV antigen can be human RSV non-structural protein 1 (“NS1 protein”), or fragment or variant thereof. For example, the RSV antigen can be RSV NS1 protein, or fragment or variant thereof, from the RSV Long strain (GenBank AAX23987.1). The RSV antigen human can also be RSV non-structural protein 2 (“NS2 protein”), or fragment or variant thereof. For example, the RSV antigen can be RSV NS2 protein, or fragment or variant thereof, from the RSV Long strain (GenBank AAX23988.1). The RSV antigen can further be human RSV nucleocapsid (“N”) protein, or fragment or variant thereof. For example, the RSV antigen can be RSV N protein, or fragment or variant thereof, from the RSV Long strain (GenBank AAX23989.1). The RSV antigen can be human RSV Phosphoprotein (“P”) protein, or fragment or variant thereof. For example, the RSV antigen can be RSV P protein, or fragment or variant thereof, from the RSV Long strain (GenBank AAX23990.1). The RSV antigen also can be human RSV Matrix protein (“M”) protein, or fragment or variant thereof. For example, the RSV antigen can be RSV M protein, or fragment or variant thereof, from the RSV Long strain (GenBank AAX23991.1).

In still other embodiments, the RSV antigen can be human RSV small hydrophobic (“SH”) protein, or fragment or variant thereof. For example, the RSV antigen can be RSV SH protein, or fragment or variant thereof, from the RSV Long strain (GenBank AAX23992.1). The RSV antigen can also be human RSV Matrix protein2-1 (“M2-1”) protein, or fragment or variant thereof. For example, the RSV antigen can be RSV M2-1 protein, or fragment or variant thereof, from the RSV Long strain (GenBank AAX23995.1). The RSV antigen can further be human RSV Matrix protein 2-2 (“M2-2”) protein, or fragment or variant thereof. For example, the RSV antigen can be RSV M2-2 protein, or fragment or variant thereof, from the RSV Long strain (GenBank AAX23997.1). The RSV antigen human can be RSV Polymerase L (“L”) protein, or fragment or variant thereof. For example, the RSV antigen can be RSV L protein, or fragment or variant thereof, from the RSV Long strain (GenBank AAX23996.1).

In further embodiments, the RSV antigen can have an optimized amino acid sequence of NS1, NS2, N, P, M, SH, M2-1, M2-2, or L protein. The RSV antigen can be a human RSV protein or recombinant antigen, such as any one of the proteins encoded by the human RSV genome.

In other embodiments, the RSV antigen can be, but is not limited to, the RSV F protein from the RSV Long strain, the RSV G protein from the RSV Long strain, the optimized amino acid RSV G amino acid sequence, the human RSV genome of the RSV Long strain, the optimized amino acid RSV F amino acid sequence, the RSV NS1 protein from the RSV Long strain, the RSV NS2 protein from the RSV Long strain, the RSV N protein from the RSV Long strain, the RSV P protein from the RSV Long strain, the RSV M protein from the RSV Long strain, the RSV SH protein from the RSV Long strain, the RSV M2-1 protein from the RSV Long strain, the RSV M2-2 protein from the RSV Long strain, the RSV L protein from the RSV Long strain, the RSV G protein from the RSV subtype B isolate H5601, the RSV G protein from the RSV subtype B isolate H1068, the RSV G protein from the RSV subtype B isolate H5598, the RSV G protein from the RSV subtype B isolate H1123, or fragment thereof, or variant thereof.

The DNA vaccine may encode a RSV antigen. Examples of DNA vaccines encoding RSV antigens include those described in U.S. Patent Application Pub. No. 20150079121, the content of which is incorporated by reference.

(g) Influenza Antigen

The viral antigen may comprise an antigen from influenza virus. The influenza antigens are those capable of eliciting an immune response in a mammal against one or more influenza serotypes. The antigen can comprise the full length translation product HA0, subunit HAL subunit HA2, a variant thereof, a fragment thereof or a combination thereof. The influenza hemagglutinin antigen can be derived from multiple strains of influenza A serotype H1, serotype H2, a hybrid sequence derived from different sets of multiple strains of influenza A serotype H1, or derived from multiple strains of influenza B. The influenza hemagglutinin antigen can be from influenza B.

The influenza antigen can also contain at least one antigenic epitope that can be effective against particular influenza immunogens against which an immune response can be induced. The antigen may provide an entire repertoire of immunogenic sites and epitopes present in an intact influenza virus. The antigen may be derived from hemagglutinin antigen sequences from a plurality of influenza A virus strains of one serotype such as a plurality of influenza A virus strains of serotype H1 or of serotype H2. The antigen may be a hybrid hemagglutinin antigen sequence derived from combining two different hemagglutinin antigen sequences or portions thereof. Each of two different hemagglutinin antigen sequences may be derived from a different set of a plurality of influenza A virus strains of one serotype such as a plurality of influenza A virus strains of serotype H1. The antigen may be a hemagglutinin antigen sequence derived from hemagglutinin antigen sequences from a plurality of influenza B virus strains.

In some embodiments, the influenza antigen can be H1 HA, H2 HA, H3 HA, H5 HA, or a BHA antigen.

A synthetic antibody specific for an influenza antigen can include an Ig comprising the amino acid sequence of one of SEQ ID NOs: 155-161. A synthetic antibody specific for an influenza antigen can be encoded by a nucleic acid molecule comprising a nucleic acid sequence of one of SEQ ID NOs:162-170.

The DNA vaccine may encode a influenza antigen. Examples of DNA vaccines encoding influenza antigens include those described in WO/2008/014521, published Jan. 31, 2008; U.S. Pat. Nos. 9,592,285, 8,298,820; U.S. Patent Application Pub. Nos. 20160022806, US 20160175427, the contents of each are fully incorporated by reference.

(h) Ebola Virus

The viral antigen may be from Ebola virus. Ebola virus disease (EVD) or Ebola hemorrhagic fever (EHF) includes any of four of the five known Ebola viruses including Bundibugyo virus (BDBV), Ebola virus (EBOV), Sudan virus (SUDV), and Taï Forest virus (TAFV, also referred to as Cote d'Ivoire Ebola virus (Ivory Coast Ebolavirus, CIEBOV).

A synthetic antibody specific for an Ebola virus antigen. A synthetic antibody specific for Ebola virus can include a Ig comprising the amino acid sequence of SEQ ID NO: 135, SEQ ID NO: 137, SEQ ID NO: 139, SEQ ID NO: 141, SEQ ID NO:143, SEQ ID NO:145, or SEQ ID NO: 147. A synthetic antibody specific for Ebola virus can be encoded by a nucleic acid molecule comprising a nucleic acid sequence of SEQ ID NO: 136, SEQ ID NO: 138, SEQ ID NO: 140, SEQ ID NO: 142, SEQ ID NO:144, SEQ ID NO:146, or SEQ ID NO: 148.

The DNA vaccine may encode an Ebola antigen. Examples of DNA vaccines encoding Ebola antigens include those described in U.S. Patent Application Pub. No. 20150335726, the content of which is incorporated by reference.

(i) Zika Virus

The viral antigen may be from Zika virus. Zika disease is caused by infection with the Zika virus and can be transmitted to humans through the bite of infected mosquitoes or sexually transmitted between humans. The Zika antigen can include a Zika Virus Envelope protein, Zika Virus NS1 protein, or a Zika Virus Capsid protein.

A synthetic antibody specific for a Zika antigen. A synthetic antibody specific for Zika Virus can include an Ig comprising the amino acid sequence of SEQ ID NO:101, SEQ ID NO:102, SEQ ID NO:103, SEQ ID NO: 104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:121, or SEQ ID NO:122.

The DNA vaccine may encode a Zika antigen. A DNA vaccine encoding a Zika antigen may include a nucleic acid sequence encoding an amino acid sequence comprising one of SEQ ID NO: 123, SEQ ID NO: 125, SEQ ID NO: 127, SEQ ID NO: 129, SEQ ID NO: 131, and SEQ ID NO: 133. A DNA vaccine encoding a Zika antigen may include a nucleic acid sequence comprising one of SEQ ID NO: 124, SEQ ID NO: 126, SEQ ID NO: 128, SEQ ID NO: 130, and SEQ ID NO: 132.

(j) Marburg Virus

The viral antigen may be from Marburg virus. Marburgvirus immunogens that can be used to induce broad immunity against multiple subtypes or serotypes of Marburgvirus. The antigen may be derived from a Marburg virus envelope glycoprotein.

The DNA vaccine may encode a Marburg antigen. Examples of DNA vaccines encoding Marburg antigens include those described in U.S. Pat. No. 9,597,388, the contents of which are fully incorporated by reference. A DNA vaccine encoding a Marburg virus antigen may include a nucleic acid sequence encoding an amino acid sequence comprising one of SEQ ID NO: 150, SEQ ID NO: 152, and SEQ ID NO: 154. A DNA vaccine encoding a Marburg virus antigen may include a nucleic acid sequence comprising one of SEQ ID NO: 149, SEQ ID NO: 151, and SEQ ID NO: 153.

(2) Bacterial Antigens

The foreign antigen can be a bacterial antigen or fragment or variant thereof. The bacterium can be from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

The bacterium can be a gram positive bacterium or a gram negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, a psychrophile, an halophile, or an osmophile.

The bacterium can be an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. The bacterium can be a mycobacteria, Clostridium tetani, Yersinia pestis, Bacillus anthracis, methicillin-resistant Staphylococcus aureus (MRSA), or Clostridium difficile. The bacterium can be Mycobacterium tuberculosis.

Examples of DNA vaccines encoding Clostridium difficile antigens include those described in U.S. Patent Application Pub. No. 20140341936, the content of which is incorporated by reference.

Examples of DNA vaccines encoding MRSA antigens include those described in U.S. Patent Application Pub. No. 20140341944, the content of which is incorporated by reference.

(a) Mycobacterium tuberculosis Antigens

The bacterial antigen may be a Mycobacterium tuberculosis antigen (i.e., TB antigen or TB immunogen), or fragment thereof, or variant thereof. The TB antigen can be from the Ag85 family of TB antigens, for example, Ag85A and Ag85B. The TB antigen can be from the Esx family of TB antigens, for example, EsxA, EsxB, EsxC, EsxD, EsxE, EsxF, EsxH, EsxO, EsxQ, EsxR, EsxS, EsxT, EsxU, EsxV, and EsxW.

The DNA vaccine may encode a Mycobacterium tuberculosis antigen. Examples of DNA vaccines encoding Mycobacterium tuberculosis antigens include those described in U.S. Patent Application Pub. No. 20160022796, the content of which is incorporated by reference.

(3) Parasitic Antigens

The foreign antigen can be a parasite antigen or fragment or variant thereof. The parasite can be a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing any one of the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus-lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

(a) Malaria Antigen

The foreign antigen may be a malaria antigen (i.e., PF antigen or PF immunogen), or fragment thereof, or variant thereof. The antigen can be from a parasite causing malaria. The malaria causing parasite can be Plasmodium falciparum. The Plasmodium falciparum antigen can include the circumsporozoite (CS) antigen.

In some embodiments, the malaria antigen can be one of P. falciparum immunogens CS; LSA1; TRAP; CelTOS; and Ama1. The immunogens may be full length or immunogenic fragments of full length proteins.

In other embodiments, the malaria antigen can be TRAP, which is also referred to as SSP2. In still other embodiments, the malaria antigen can be CelTOS, which is also referred to as Ag2 and is a highly conserved Plasmodium antigen. In further embodiments, the malaria antigen can be Ama1, which is a highly conserved Plasmodium antigen. In some embodiments, the malaria antigen can be a CS antigen.

In other embodiments, the malaria antigen can be a fusion protein comprising a combination of two or more of the PF proteins set forth herein. For example, fusion proteins may comprise two or more of CS immunogen, ConLSA1 immunogen, ConTRAP immunogen, ConCelTOS immunogen, and ConAma1 immunogen linked directly adjacent to each other or linked with a spacer or one or more amino acids in between. In some embodiments, the fusion protein comprises two PF immunogens; in some embodiments the fusion protein comprises three PF immunogens, in some embodiments the fusion protein comprises four PF immunogens, and in some embodiments the fusion protein comprises five PF immunogens. Fusion proteins with two PF immunogens may comprise: CS and LSA1; CS and TRAP; CS and CelTOS; CS and Ama1; LSA1 and TRAP; LSA1 and CelTOS; LSA1 and Ama1; TRAP and CelTOS; TRAP and Ama1; or CelTOS and Ama1. Fusion proteins with three PF immunogens may comprise: CS, LSA1 and TRAP; CS, LSA1 and CelTOS; CS, LSA1 and Ama1; LSA1, TRAP and CelTOS; LSA1, TRAP and Ama1; or TRAP, CelTOS and Ama1. Fusion proteins with four PF immunogens may comprise: CS, LSA1, TRAP and CelTOS; CS, LSA1, TRAP and Ama1; CS, LSA1, CelTOS and Ama1; CS, TRAP, CelTOS and Ama1; or LSA1, TRAP, CelTOS and Ama1. Fusion proteins with five PF immunogens may comprise CS or CS-alt, LSA1, TRAP, CelTOS and Ama1.

The DNA vaccine may encode a malaria antigen. Examples of DNA vaccines encoding malaria antigens include those described in U.S. Patent Application Pub. No. 20130273112, the content of which is incorporated by reference.

(4) Fungal Antigens

The foreign antigen can be a fungal antigen or fragment or variant thereof. The fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

b. Self Antigens

In some embodiments, the antigen is a self antigen. A self antigen may be a constituent of the subject's own body that is capable of stimulating an immune response. In some embodiments, a self antigen does not provoke an immune response unless the subject is in a disease state, e.g., an autoimmune disease.

Self antigens may include, but are not limited to, cytokines, antibodies against viruses such as those listed above including HIV and Dengue, antigens affecting cancer progression or development, and cell surface receptors or transmembrane proteins.

(1) WT-1

The self-antigen antigen can be Wilm's tumor suppressor gene 1 (WT1), a fragment thereof, a variant thereof, or a combination thereof. WT1 is a transcription factor containing at the N-terminus, a proline/glutamine-rich DNA-binding domain and at the C-terminus, four zinc finger motifs. WT1 plays a role in the normal development of the urogenital system and interacts with numerous factors, for example, p53, a known tumor suppressor and the serine protease HtrA2, which cleaves WT1 at multiple sites after treatment with a cytotoxic drug. Mutation of WT1 can lead to tumor or cancer formation, for example, Wilm's tumor or tumors expressing WT1.

The DNA vaccine may encode a WT-1 antigen. Examples of DNA vaccines encoding WT-1 antigens include those described in U.S. Patent Application Pub. Nos. 20150328298 and 20160030536, the contents each are incorporated by reference.

(2) EGFR

The self-antigen may include an epidermal growth factor receptor (EGFR) or a fragment or variation thereof. EGFR (also referred to as ErbB-1 and HER1) is the cell-surface receptor for members of the epidermal growth factor family (EGF-family) of extracellular protein ligands. EGFR is a member of the ErbB family of receptors, which includes four closely related receptor tyrosine kinases: EGFR (ErbB-1), HER2/c-neu (ErbB-2), Her 3 (ErbB-3), and Her 4 (ErbB-4). Mutations affecting EGFR expression or activity could result in cancer.

The antigen may include an ErbB-2 antigen. Erb-2 (human epidermal growth factor receptor 2) is also known as Neu, HER2, CD340 (cluster of differentiation 340), or p185 and is encoded by the ERBB2 gene. Amplification or over-expression of this gene has been shown to play a role in the development and progression of certain aggressive types of breast cancer. In approximately 25-30% of women with breast cancer, a genetic alteration occurs in the ERBB2 gene, resulting in the production of an increased amount of HER2 on the surface of tumor cells. This overexpression of HER2 promotes rapid cell division and thus, HER2 marks tumor cells.

A synthetic antibody specific for HER2 can include a Fab fragment comprising an amino acid sequence of SEQ ID NO:41, which is encoded by the nucleic acid sequence of SEQ ID NO:40, and an amino acid sequence of SEQ ID NO:43, which is encoded by the nucleic acid sequence of SEQ ID NO:42.

(3) Cocaine

The self-antigen may be a cocaine receptor antigen. Cocaine receptors include dopamine transporters.

(4) PD-1

The self-antigen may include programmed death 1 (PD-1). Programmed death 1 (PD-1) and its ligands, PD-L1 and PD-L2, deliver inhibitory signals that regulate the balance between T cell activation, tolerance, and immunopathology. PD-1 is a 288 amino acid cell surface protein molecule including an extracellular IgV domain followed by a transmembrane region and an intracellular tail.

The DNA vaccine may encode a PD-1 antigen. Examples of DNA vaccines encoding PD-1 antigens include those described in U.S. Patent Application Pub. No. 20170007693, the content of which is incorporated by reference.

(5) 4-1BB

The self-antigen may include 4-1BB ligand. 4-1BB ligand is a type 2 transmembrane glycoprotein belonging to the TNF superfamily. 4-1BB ligand may be expressed on activated T Lymphocytes. 4-1BB is an activation-induced T-cell costimulatory molecule. Signaling via 4-1BB upregulates survival genes, enhances cell division, induces cytokine production, and prevents activation-induced cell death in T cells.

(6) CTLA4

The self-antigen may include CTLA-4 (Cytotoxic T-Lymphocyte Antigen 4), also known as CD152 (Cluster of differentiation 152). CTLA-4 is a protein receptor found on the surface of T cells, which lead the cellular immune attack on antigens. The antigen may be a fragment of CTLA-4, such as an extracellular V domain, a transmembrane domain, and a cytoplasmic tail, or combination thereof.

(7) IL-6

The self-antigen may include interleukin 6 (IL-6). IL-6 stimulates the inflammatory and auto-immune processes in many diseases including, but not limited to, diabetes, atherosclerosis, depression, Alzheimer's Disease, systemic lupus erythematosus, multiple myeloma, cancer, Behçet's disease, and rheumatoid arthritis.

(8) MCP-1

The self-antigen may include monocyte chemotactic protein-1 (MCP-1). MCP-1 is also referred to as chemokine (C-C motif) ligand 2 (CCL2) or small inducible cytokine A2. MCP-1 is a cytokine that belongs to the CC chemokine family. MCP-1 recruits monocytes, memory T cells, and dendritic cells to the sites of inflammation produced by either tissue injury or infection.

(9) Amyloid Beta

The self-antigen may include amyloid beta (Aβ) or a fragment or a variant thereof. The Aβ antigen can comprise an Aβ(X-Y) peptide, wherein the amino acid sequence from amino acid position X to amino acid Y of the human sequence Aβ protein including both X and Y, in particular to the amino acid sequence from amino acid position X to amino acid position Y of the amino acid sequence

(SEQ ID NO: 171)
DAEFRHDSGYEVHHQKLVFFAEDVGSNKGAIIGLMVGGVVIATVIVI

(corresponding to amino acid positions 1 to 47; the human query sequence) or variants thereof. The Aβ antigen can comprise an Aβ polypeptide of Aβ(X-Y) polypeptide wherein X can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 and Y can be 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, or 15. The Aβ polypeptide can comprise a fragment that is at least 15, at least 16, at least 17, at least 18, at least 19, at least 20, at least 21, at least 22, at least 23, at least 24, at least 25, at least 30, at least 35, at least 36, at least 37, at least 38, at least 39, at least 40, at least 41, at least 42, at least 43, at least 44, at least 45, or at least 46 amino acids.

(10) IP-10

The self-antigen may include interferon (IFN)-gamma-induced protein 10 (IP-10). IP-10 is also known as small-inducible cytokine B10 or C-X-C motif chemokine 10 (CXCL10). CXCL10 is secreted by several cell types, such as monocytes, endothelial cells and fibroblasts, in response to IFN-γ.

(11) PSMA

The self-antigen may include prostate-specific membrane antigen (PSMA). PSMA is also known as glutamate carboxypeptidase II (GCPII), N-acetyl-L-aspartyl-L-glutamate peptidase I (NAALADase I), NAAG peptidase, or folate hydrolase (FOLH). PMSA is an integral membrane protein highly expressed by prostate cancer cells.

In some embodiments, the recombinant nucleic acid sequence encoding an antibody directed against PSMA (anti-PSMA antibody) may be a recombinant nucleic acid sequence including a recombinant nucleic acid sequence construct in arrangement 2.

In still other embodiments, the anti-PSMA antibody encoded by the recombinant nucleic acid sequence may be modified as described herein. One such modification is a defucosylated antibody, which as demonstrated in the Examples, exhibited increased ADCC activity as compared to commercial antibodies. The modification may be in the heavy chain, light chain, or a combination thereof. The modification may be one or more amino acid substitutions in the heavy chain, one or more amino acid substitutions in the light chain, or a combination thereof.

An antibody specific for PSMA and modified to not be fucosylated may be encoded by the nucleic acid sequence set forth in SEQ ID NO:79. SEQ ID NO:79 encodes the amino acid sequence set forth in SEQ ID NO:80.

The DNA vaccine may encode a PSMA antigen. Examples of DNA vaccines encoding PSMA antigens include those described in U.S. Patent Application Pub. No. 20130302361, the content of which is incorporated by reference.

c. Other Antigens

In some embodiments, the antigen is an antigen other than the foreign antigen and/or the self-antigen.

(a) HIV-1 VRC01

The other antigen can be HIV-1 VRC01. HIV-1 VCR01 is a neutralizing CD4-binding site-antibody for HIV. HIV-1 VCR01 contacts portions of HIV-1 including within the gp120 loop D, the CD4 binding loop, and the V5 region of HIV-1.

(b) HIV-1 PG9

The other antigen can be HIV-1 PG9. HIV-1 PG9 is the founder member of an expanding family of glycan-dependent human antibodies that preferentially bind the HIV (HIV-1) envelope (Env) glycoprotein (gp) trimer and broadly neutralize the virus.

(c) HIV-1 4E10

The other antigen can be HIV-1 4E10. HIV-1 4E10 is a neutralizing anti-HIV antibody. HIV-1 4E10 is directed against linear epitopes mapped to the membrane-proximal external region (MPER) of HIV-1, which is located at the C terminus of the gp41 ectodomain.

(d) DV-SF1

The other antigen can be DV-SF1. DV-SF1 is a neutralizing antibody that binds the envelope protein of the four Dengue virus serotypes.

(e) DV-SF2

The other antigen can be DV-SF2. DV-SF2 is a neutralizing antibody that binds an epitope of the Dengue virus. DV-SF2 can be specific for the DENV4 serotype.

(f) DV-SF3

The other antigen can be DV-SF3. DV-SF3 is a neutralizing antibody that binds the EDIII A strand of the Dengue virus envelope protein.

### 7. EXCIPIENTS AND OTHER COMPONENTS OF THE COMPOSITION

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### 8. METHOD OF GENERATING THE SYNTHETIC ANTIBODY

The present invention also relates a method of generating the synthetic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### 9. METHOD OF IDENTIFYING OR SCREENING FOR THE ANTIBODY

The present invention further relates to a method of identifying or screening for the antibody described above, which is reactive to or binds the antigen described above. The method of identifying or screening for the antibody can use the antigen in methodologies known in those skilled in art to identify or screen for the antibody. Such methodologies can include, but are not limited to, selection of the antibody from a library (e.g., phage display) and immunization of an animal followed by isolation and/or purification of the antibody.

### 10. METHOD OF DELIVERY OF THE COMPOSITION

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

a. Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, Pa.) or Elgen electroporator (Inovio Pharmaceuticals, Plymouth Meeting, Pa.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Applications Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U. S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### 11. METHOD OF TREATMENT

Also provided herein is a method of treating, protecting against, and/or preventing disease in a subject in need thereof by generating the synthetic antibody in the subject. The method can include administering the composition to the subject. Administration of the composition to the subject can be done using the method of delivery described above.

Upon generation of the synthetic antibody in the subject, the synthetic antibody can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The method of delivering the vaccine or vaccination may be provided to induce a therapeutic and prophylactic immune response. The vaccination process may generate in the mammal an immune response against the antigen. The vaccine may be delivered to an individual to modulate the activity of the mammal's immune system and enhance the immune response. The delivery of the vaccine may be the transfection of the consensus antigen as a nucleic acid molecule that is expressed in the cell and delivered to the surface of the cell upon which the immune system recognized and induces a cellular, humoral, or cellular and humoral response. The delivery of the vaccine may be used to induce or elicit and immune response in mammals against the antigen by administering to the mammals the vaccine as discussed above.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

The composition can comprise 1 or more, 2 or more, 3 or more, 4 or more, 5 or more, 6 or more, 7 or more, 8 or more, 9 or more, or 10 or more DNA vaccines encoding an antigen. The composition may comprise 1 or more, 2 or more, 3 or more, 4 or more, 5 or more, 6 or more, 7 or more, 8 or more, 9 or more, or 10 or more DNA encoded synthetic antibodies or fragments thereof.

The DNA vaccine and the DMAb may be administered at the same time or at different times. In one embodiment, the DNA vaccine and the DMAb are administered simultaneously. In one embodiment, the DNA vaccine is administered before the DMAb. In one embodiment, the DMAb is administered before the DNA vaccine.

In certain embodiments, the DNA vaccine is administered 1 or more days, 2 or more days, 3 or more days, 4 or more days, 5 or more days, 6 or more days, 7 or more days, 8 or more days, 9 or more days, 10 or more days, 11 or more days, 12 or more days, 13 or more days, or 14 or more days after the DMAb is administered. In certain embodiments, the DNA vaccine is administered 1 or more weeks, 2 or more weeks, 3 or more weeks, 4 or more weeks, 5 or more weeks, 6 or more weeks, 7 or more weeks, 8 or more weeks, 9 or more weeks, or 10 or more weeks after the DMAb is administered. In certain embodiments, the DNA vaccine is administered 1 or more months, 2 or more months, 3 or more months, 4 or more months, 5 or more months, 6 or more months, 7 or more months, 8 or more months, 9 or more months, 10 or more months, 11 or more months, or 12 or more months after the DMAb is administered.

In certain embodiments, the DMAb is administered 1 or more days, 2 or more days, 3 or more days, 4 or more days, 5 or more days, 6 or more days, 7 or more days, 8 or more days, 9 or more days, 10 or more days, 11 or more days, 12 or more days, 13 or more days, or 14 or more days after the DNA vaccine is administered. In certain embodiments, the DMAb is administered 1 or more weeks, 2 or more weeks, 3 or more weeks, 4 or more weeks, 5 or more weeks, 6 or more weeks, 7 or more weeks, 8 or more weeks, 9 or more weeks, or 10 or more weeks after the DNA vaccine is administered. In certain embodiments, the DMAb is administered 1 or more months, 2 or more months, 3 or more months, 4 or more months, 5 or more months, 6 or more months, 7 or more months, 8 or more months, 9 or more months, 10 or more months, 11 or more months, or 12 or more months after the DNA vaccine is administered.

In certain embodiments, the DMAb and DNA vaccine are administered once. In certain embodiments, the DMAb and/or the DNA vaccine are administered more than once. In certain embodiments, administration of the DMAb and DNA vaccine provides a persistent and systemic immune response.

### 12. USE IN COMBINATION WITH ANTIBIOTICS

The present invention also provides a method of treating, protecting against, and/or preventing disease in a subject in need thereof by administering a combination of the synthetic antibody and a therapeutic antibiotic agent.

The synthetic antibody and an antibiotic agent may be administered using any suitable method such that a combination of the synthetic antibody and antibiotic agent are both present in the subject. In one embodiment, the method may comprise administration of a first composition comprising a synthetic antibody of the invention by any of the methods described in detail above and administration of a second composition comprising an antibiotic agent less than 1, less than 2, less than 3, less than 4, less than 5, less than 6, less than 7, less than 8, less than 9 or less than 10 days following administration of the synthetic antibody. In one embodiment, the method may comprise administration of a first composition comprising a synthetic antibody of the invention by any of the methods described in detail above and administration of a second composition comprising an antibiotic agent more than 1, more than 2, more than 3, more than 4, more than 5, more than 6, more than 7, more than 8, more than 9 or more than 10 days following administration of the synthetic antibody. In one embodiment, the method may comprise administration of a first composition comprising an antibiotic agent and administration of a second composition comprising a synthetic antibody of the invention by any of the methods described in detail above less than 1, less than 2, less than 3, less than 4, less than 5, less than 6, less than 7, less than 8, less than 9 or less than 10 days following administration of the antibiotic agent. In one embodiment, the method may comprise administration of a first composition comprising an antibiotic agent and administration of a second composition comprising a synthetic antibody of the invention by any of the methods described in detail above more than 1, more than 2, more than 3, more than 4, more than 5, more than 6, more than 7, more than 8, more than 9 or more than 10 days following administration of the antibiotic agent. In one embodiment, the method may comprise administration of a first composition comprising a synthetic antibody of the invention by any of the methods described in detail above and a second composition comprising an antibiotic agent concurrently. In one embodiment, the method may comprise administration of a first composition comprising a synthetic antibody of the invention by any of the methods described in detail above and a second composition comprising an antibiotic agent concurrently. In one embodiment, the method may comprise administration of a single composition comprising a synthetic antibody of the invention and an antibiotic agent.

Non-limiting examples of antibiotics that can be used in combination with the synthetic antibody of the invention include aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins: carboxypenicillins (e.g., carbenicillin and ticarcillin) and ureidopenicillins (e.g., mezlocillin, azlocillin, and piperacillin), carbapenems (e.g., meropenem, imipenem, doripenem), polymyxins (e.g., polymyxin B and colistin) and monobactams (e.g., aztreonam).

The present invention has multiple aspects, illustrated by the following non-limiting examples.

### 13. EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Rapid and Long-Term Immunity Elicited by DNA Encoded Antibody Prophylaxis and DNA Vaccination Against Chikungunya Virus

Vaccination is known to exhibit a lag phase before generation of immunity; thus, there is a gap of time during infection before an immune response is in effect. The following provides specific novel approaches that utilizes the benefit of vaccines and the native immune response along with a rapid generation of effective immunity using the DNA synthetic antibodies or dMabs.

An antibody-based prophylaxis/therapy entailing the electroporation mediated delivery of synthetic plasmids, encoding biologically active anti-Chikungunya virus envelope mAb (designated dMAb), was designed and evaluated for anti-viral efficacy as well as for the ability to overcome shortcomings inherent with conventional active vaccination by a novel passive immune-based strategy. One intramuscular injection of the CHIKV-dMAb produced antibodies in vivo more rapidly than active vaccination with a CHIKV-DNA vaccine. This dMAb neutralized diverse CHIKV clinical isolates and protected mice from viral challenge. Combinations of both afford rapid as well as long-lived protection.

The results presented herein demonstrate that a DNA based dMAb strategy induces rapid protection against an emerging viral infection, which can be combined with DNA vaccination providing a uniquely both short term and long-term protection against this emerging infectious disease. These studies have implications for pathogen treatment and control strategies.

### Methods

Construction and Expression of CHIKV Specific dMABs

Gene sequence information for an established anti-Env-specific CHIKV neutralizing human mAb were obtained from the National Center for Biotechnology Information database (Wailer et al., 2011, J Immunol 186:3258-64). Human embryonic kidney 293T cells and Vero cells, used for expression confirmation studies, were maintained as described previously (Mallilankaraman et al., 2011, PLoS Negl Trop Dis 5:e928). The variable heavy (VH) and variable light (VL) chain segments for the CHIKV Env dMAb preparation were generated by using synthetic oligonucleotides with several modifications and were constructed as either a full-length immunoglobulin G (IgG; designated “CVM1-IgG”) or Fab fragment (designated “CVM1-Fab”) (Muthumani et al., 2013, Hum Vaccin Immunother 9:2253-62). For cloning of CVM1-IgG, a single open reading frame was assembled containing the heavy and light chain genes, separated by a furin cleavage site coupled with a P2A self-processing peptide sequence. This transgene was cloned into the pVax1 expression vector (Muthumani et al., 2013, Hum Vaccin Immunother 9:2253-62_. The CVM1-Fab VH and VL chains were cloned into separate pVax1 vectors. For tissue culture transfection, 100 μg of pVax1 DNA, CVM1-IgG, or CVM1-Fab (100 μg of each VH and VL construct) was used. The CHIKV Env-based DNA vaccine used in the study was developed and characterized as previously described (Muthumani et al., 2008, Vaccine 26:5128-34; Mallilankaraman et al., 2011, PLoS Negl Trop Dis 5:e928).

### CHIKV-dMAb IgG Quantification and Binding Assays

ELISA assays were performed with sera, collected and measured in duplicate, from mice administered CMV1-IgG or pVax1 to quantify expression kinetics and target antigen binding. These measurements and analyses were performed as previously described (Muthumani et al., 2015, Sci transl Med 7:301ra132).

Western Blot and Immunofluorescence Analysis of dMAb Generated IgG

For Western blot analysis of IgG expression CHIKV (viral isolate PC08) infected cells were lysed two days post infection and evaluated by previously published methods (Mallilankaraman et al., 2011, PLoS Negl Trop Dis 5:e928; Muthumani et al., 2015, Sci transl Med 7:301ra132). For immunofluorescence analysis, chamber slides (Nalgene Nunc, Penfield, N.Y.) were seeded with Vero cells (1×10⁴) and infected for 2 hours with the viral isolate CHIKV PC08 at a multiplicity of infection of 1. Immunofluorescence analysis was performed as previously described (Muthumani et al., 2015, Sci transl Med 7:301ra132), with slides being visually evaluated by confocal microscopy (LSM710; Carl Zeiss). The resulting images were semiquantitatively analyzed using Zen software (Carl Zeiss).

dMAb DNA Plasmid Administration and In Vivo Analysis

CVM1-Fab and CVM1-IgG expression kinetics and functionality were evaluated in B6.Cg-Foxn1nu/J mice (Jackson Laboratory) following intramuscular injection of 100 μg control pVax1, CVM1-IgG, or 100 μg of each plasmid chain of CVM1-Fab. For studies that include the DNA vaccine, 25 μg of the CHIKV Env plasmid were injected 3 times at 2-week intervals. All injections were followed immediately by delivery of CHIKV dMAb DNA plasmid via electroporation (Flingai et al., 2015, Sci Rep 5:12616; Muthumani et al., 2015, Sci transl Med 7:301ra132; Broderick et al., 2014, Methods Mol iol 1143:123-30).

### CHIKV Challenge Study

BALB/c mice received a single (100 μg) electroporation-enhanced intramuscular injection of CVM1-IgG, CMV-Fab (VH and VL), or control pVax1 plasmids. The CHIKV Env DNA vaccine was delivered as described above. Two or 35 days after DNA delivery, mice were challenged with 107 plaque-forming units (25 μL) of the viral isolate CHIKV Del-03 (JN578247) (Muruganandam et al., 2011, Can J Microbiol 57:1073-7) either subcutaneously (in the dorsal side of each hind foot) or intranasally (Mallilankaraman et al., 2011, PLoS Negl Trop Dis 5:e928). Mouse foot swelling (height by breadth) was measured daily up to 14 days after infection. In addition, the animals were monitored daily (for up to 20 days after infection) for survival and signs of infection (ie, changes in body weight and lethargy). Animals losing >30% of their body mass were euthanized, and serum samples were collected for cytokine quantification and other immune analysis. Blood samples were collected from the tail on days 7-14 after infection, and viremia levels were measured by a plaque assay.

### Neutralizing Antibody Analysis

Anti-CHIKV neutralizing antibody titers from mice administered CVM1-IgG were determined by previously described methods (Wang et al., 2008, Vaccine 26:5030-9; Mallilankaraman et al., 2011, PLoS Negl Trop Dis 5:e928), using Vero cells infected with the following CHIKV isolates: LR2006-OPY1 (Indian Ocean Outbreak), IND-63WB1 and SL-CH1 (Asian-clade), Ross (ECSA-clade), and PC08 and DRDE-06 (ECSA-clade). Neutralization titers were calculated as the reciprocal of the highest dilution mediating 100% reduction of the cytopathic effects in the Vero cell monolayer. Data were generated and statistical analyses performed using the GraphPad Prism 5 software package (GraphPad Software). Nonlinear regression fitting with sigmoidal dose response was used to determine the level of antibody mediating 50% inhibition of infection (IC50). CHIKV Env pseudotype production and fluorescence-activated cell-sorting (FACS) analysis were performed as described previously (Muthumani et al., 2013, PLoS One 8:e84234).

### Cytokine Quantitative Analysis

Sera were collected from CVM1-Fab, CVM1-IgG, and CHIKV-Env injected mice as well as CHIKV challenged mice (one week post challenge). TNF-α, IL-1β and IL-6 sera cytokine levels were measured using ELISA kits according to the manufacturer's instructions (R&D Systems).

### Statistical Analysis

A student t-test or a nonparametric Spearman's correlation test, were performed using GraphPad Prism software (Prism Inc.). Correlations between the variables in the control and experimental groups were statistically evaluated using the Spearman rank correlation test, with p values <0.05 for all tests considered to be statistically significant.

### Results

Anti-CHIKV dMAbs Design and Confirmation of Expression

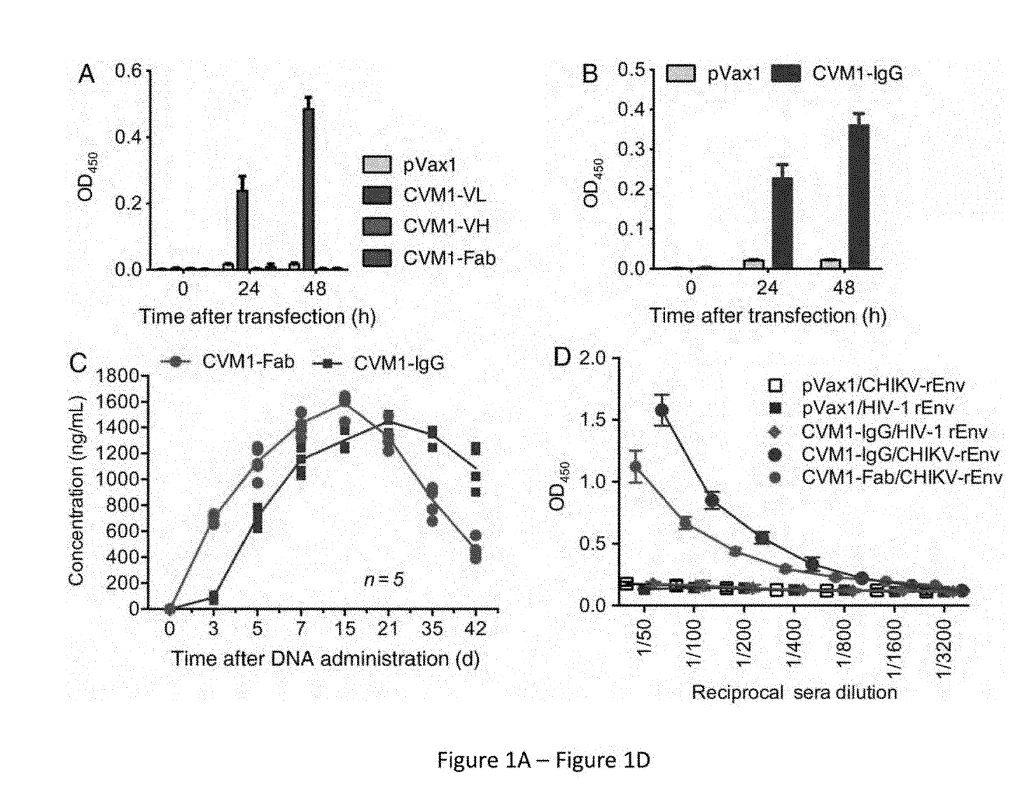

Viral entry into host cells by CHIKV is mediated by Env, against which the majority of neutralizing antibodies are generated (Mallilankaraman et al., 2011, PLoS Negl Trop Dis 5:e928; Sun et al., 2013, eLife 2:e00435). Thus, a DNA plasmid (dMAb) expressing the light and heavy immunoglobulin chains of a neutralizing anti-CHIKV mAb recognizing both E1 and E2 Env proteins was designed (Warter et al., 2011, J Immunol 186:3258-64; Pal et al., 2013, PLoS Pathog 9:e1003312). The complementary DNAs for the coding sequences of the VL and VH immunoglobulin chains for full-length anti-CHIKV dMAb were optimized for increased expression and cloned into a pVax1 vector, using previously described methods (Flingai et al., 2015, Sci Rep 5:12616; Muthumani et al., 2013, Hum Vaccin Immunother 9:2253-62). For the constructs expressing anti-CHIKV-Fab, the VH and VL genes were cloned separately. The optimized synthetic plasmids constructed from the anti-Env-specific CHIKV-neutralizing mAb were designated CVM1-IgG or CVM1-Fab, for the IgG and Fab antibodies, respectively. Human 293T cells were transfected with either the CVM1-IgG plasmid or the CVM1-Fab (VL, VH, or combined) plasmids to validate expression in vitro. As indicated in FIGS. 1A and 1B, anti-CHIKV antibody levels were measured by ELISA with recombinant CHIKV Env used as the binding antigen. These data indicate that the CVM1-Fab and CVM1-IgG expressed antibodies in the muscle that appeared to be properly assembled and biologically functional in vitro.

### In Vivo Expression and Quantification of CVM1-IgG and CVM1-Fab

Following confirmation of in vitro expression, the ability of CVM1-Fab or CVM1-IgG to produce anti-CHIKV antibodies in vivo was measured. B6.Cg-Foxn1^{nu}/J mice aged 5-6 weeks were administered 100 μg of CVM1-IgG (CVM1-IgG is 1 plasmid), 100 μg each of CVM1 VH and VL (CVM1-Fab consists of 2 plasmids), or control vector by a single intramuscular electroporation-mediated injection. Sera were collected at indicated time points, and target antigen binding was measured by IgG quantification, using ELISA. Although mAbs generated from CVM1-Fab appeared more rapidly (ie, within 3 days after injection) than those from CVM1-IgG, both constructs generated similar mAb levels by day 15 (mean sera levels [±SD], 1587.23±73.23 ng/mL of CVM1-Fab and 1341.29±82.07 ng/mL of CVM1-IgG; FIG. 1C). Mice were administered either CVM1-IgG or CVM1-Fab, and sera antibody levels were evaluated through a binding ELISA. Sera collected 15 days after injection from both CVM1-IgG and CVM1-Fab bound to CHIKV Env protein but not to an unrelated control antigen, human immunodeficiency virus type 1 Env (FIG. 1D). These data indicate that in vivo produced anti-CHIKV antibodies from CVM1-IgG or CVM1-Fab constructs have similar biological characteristics to conventionally produced antigen specific antibodies.

In Vivo Specificity and Broadly Neutralizing Activity in Sera from CVM1-IgG Injected Mice

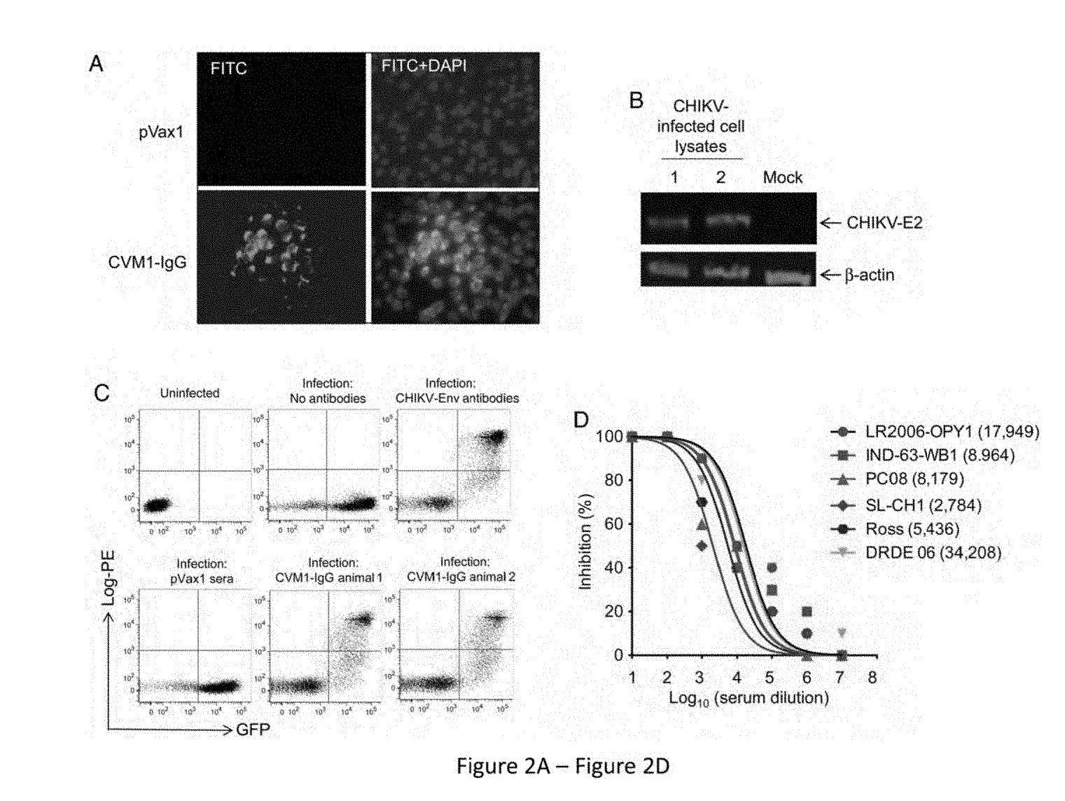

The anti-CHIKV dMAb generated mAbs were tested for binding specificity and anti-CHIKV neutralizing activity. Sera from mice injected with CVM1-IgG were tested against fixed CHIKV PC08-infected Vero cells by immunofluorescence assays. The results indicated binding of the sera antibodies to the CHIKV-infected cells (FIG. 2A). Confirmation of binding of sera from CVM1-IgG-injected mice to target proteins was tested by Western blot analysis. The detection of CHIKV E2 protein (50 kDa) expression in total cell lysate from the CHIKV-infected cells indicates specificity of CVM1-IgG expression (FIG. 2B). The specificity of in vivo-produced CVM1-IgG antibody was further demonstrated through FACS analysis against cells infected with green fluorescent protein-encoded CHIKV (FIG. 2C). Moreover, CVM1-Fab binding, demonstrated by immunohistochemical analysis and FACS analysis, was similar to that of the generated full-length CVM1-IgG (data not shown). Together, these findings indicate a strong specificity of the antibody generated from the CVM1-IgG plasmid.

Furthermore, the anti-CHIKV neutralizing activity in sera from mice that received CVM1-IgG was measured against that in 6 divergent CHIKV strains: LR2006-OPY1 (Indian Ocean Outbreak), IND-63WB1 (Asian-clade), Ross (ECSA-clade), PC08 (ECSA-clade), SL-CH1 (Asian-clade) and DRDE-06 (ECSA-clade) (Sziegler et al., 2007, Am J Trop Med Hyg 79:133-6). IC₅₀ values were determined for each viral isolate. Sera from CVM1-IgG-injected mice effectively neutralized all 6 CHIKV isolates, demonstrating that a single injection can produce significant neutralizing levels of human anti-CHIKV IgG in mice (FIG. 2D). Similar results were observed using sera from CVM1-Fab-injected mice (data not shown). These data indicate that antibodies produced in vivo by CVM1-IgG constructs have relevant biological activity (ie, binding and neutralizing activity against CHIKV)

CVM1-IgG Injection Protects Mice from Lethal CHIKV Challenge

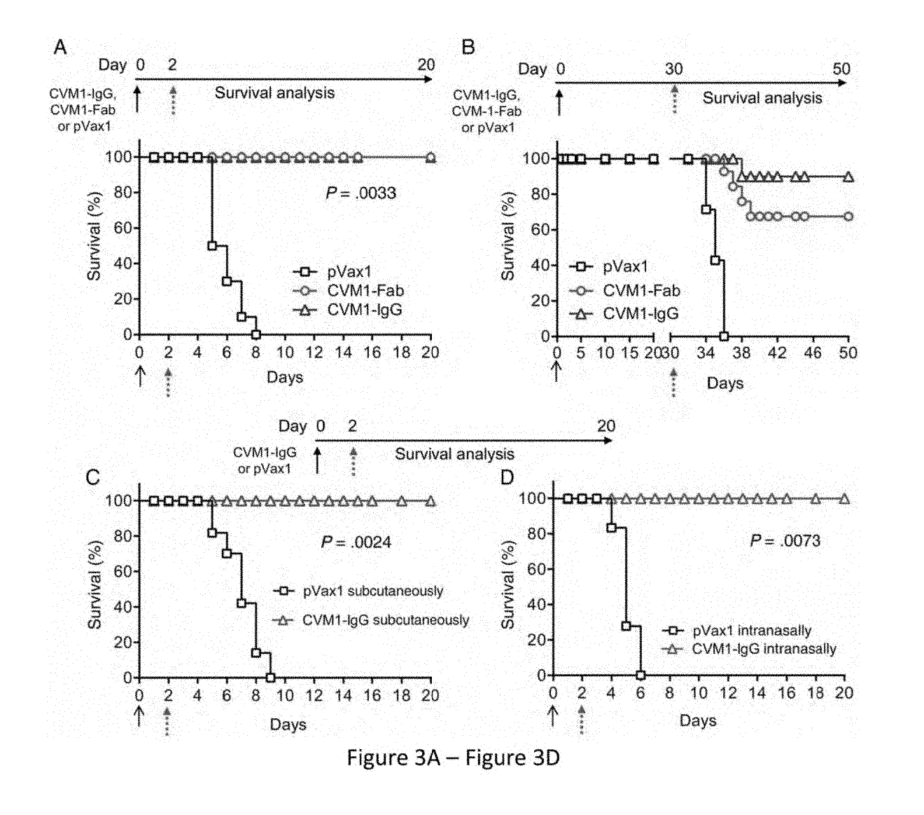

Previous studies demonstrated that early immunity against viruses is a key factor for controlling infections (Barouch et al., 2014, Nat Rev Microbiol 12:765-71; Hudson et al., 2003, Nat Med 9:129-34; Smith et al., 2015, Chikungunya Virus 18:86-95). To determine whether antibodies generated from CVM1-IgG or CVM1-Fab provide protection against early exposure to CHIKV, groups of 10 mice received a single administration of pVax1, CVM1-IgG, or CVM1-Fab on day 0. Each group subsequently was challenged subcutaneously with virus on day 2 to mimic natural CHIKV infection (FIG. 3A). Animal survival and weight changes were subsequently recorded for 20 days. All mice injected with pVax1 control plasmid died within a week of viral challenge. Conversely, 100% survival was observed in mice administered either CVM1-IgG or CVM1-Fab, compared with 0% survival among mice that received pVax1 plasmid (P=0.0033), demonstrating that CVM1-IgG and CVM1-Fab plasmids confer protective immunity within 2 days after delivery.

The longevity of immune protection was next evaluated. A second group of mice (n=10) was challenged with CHIKV 30 days after a single injection with CVM1-IgG, CVM1-Fab, or pVax1 on day 0 (FIG. 3B). Mice were monitored for survival over the next 20 days. Mice injected with CVM1-Fab or CVM1-IgG demonstrated 70% and 90% survival, respectively, compared with no survival among pVax1-injected mice (P=0.0120), indicating that CVM1-IgG provides a more durable degree of immune protection (FIG. 3B).

To assess the ability of the CVM1-IgG plasmid to protect against infection at a mucosal surface, the protective efficacy of CVM1-IgG against subcutaneous versus intranasal viral challenge, previously demonstrated to produce visible CHIKV pathogenesis such as limb muscle weakness, footpad swelling, lethargy, and high mortality within 6-10 days of infection, was evaluated (Mallilankaraman et al., 2011, PLoS Negl Trop Dis 5:e928; Couderc et al., 2008, PLoS Pathog 4:e29). For simplicity, studies focused on the CVM1-IgG construct. Groups of 20 mice received a single administration of pVax1 or CVM1-IgG, with half (ie, 10) being challenged with CHIKV via a subcutaneous or intranasal route 2 days after injection. CVM1-IgG protected mice from both subcutaneous viral challenge (P=0.0024; FIG. 3C) and intranasal viral challenge (P=0.0073; FIG. 3D), compared with pVax1-injected mice, demonstrating that it can protect against systemic and mucosal infection.

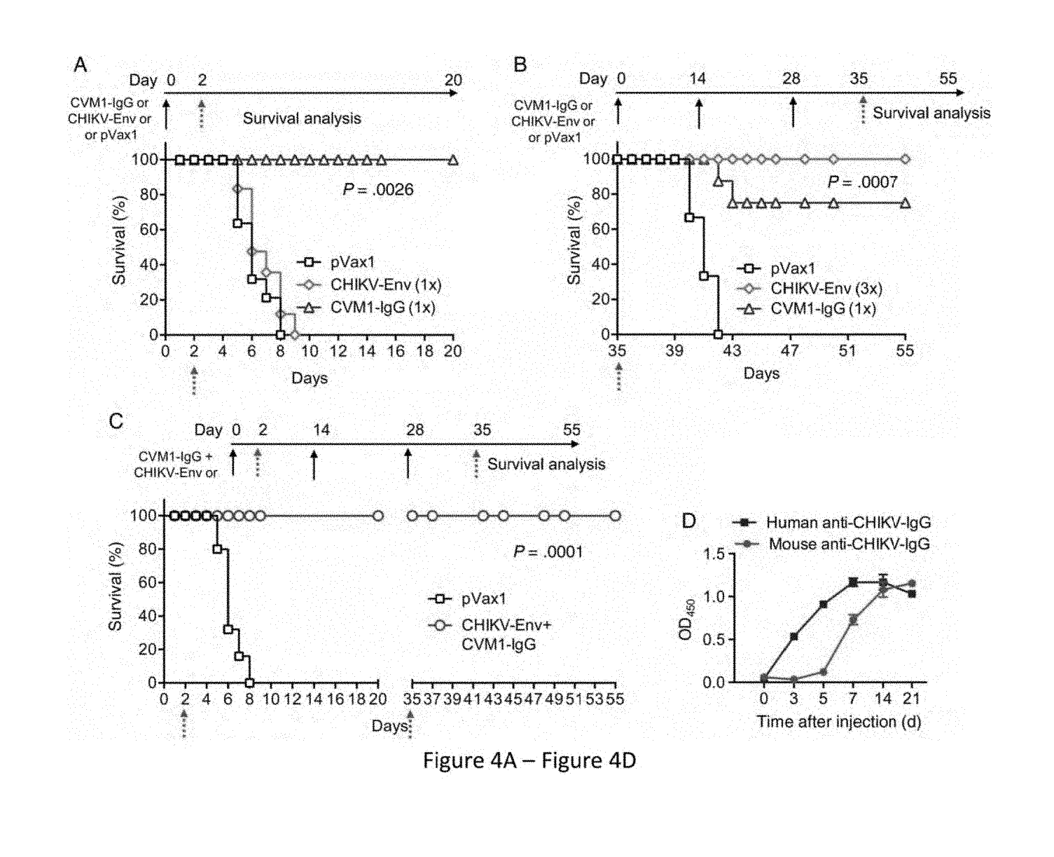

An efficacy study comparing the protective efficacy of CVM1-IgG administration vs a CHIKV Env-expressing DNA vaccine (CHIKV Env) was next performed. A novel consensus-based DNA vaccine was developed by our laboratory and was capable of providing protection against CHIKV challenge in mice. The DNA vaccine also induced both measurable cellular immune responses, as well as potent neutralizing antibody responses in rhesus macaques [11, 12]. Groups of mice were administered a single injection of CVM1-IgG, CHIKV Env, or the pVax1, followed by viral challenge on 2 days after injection. Mice that received a single immunization of CHIKV Env or pVax1 died within 6 days of viral challenge, whereas a single immunization of CVM1-IgG provided 100% protection (FIG. 4A). CVM1-IgG clearly conferred protective immunity more rapidly than the CHIKV Env DNA vaccine (P=0.0026).

### Comparison Between In Vivo Protective Immunity Conferred by CHIKV-IgG Administration and CHIKV-Env DNA Vaccination

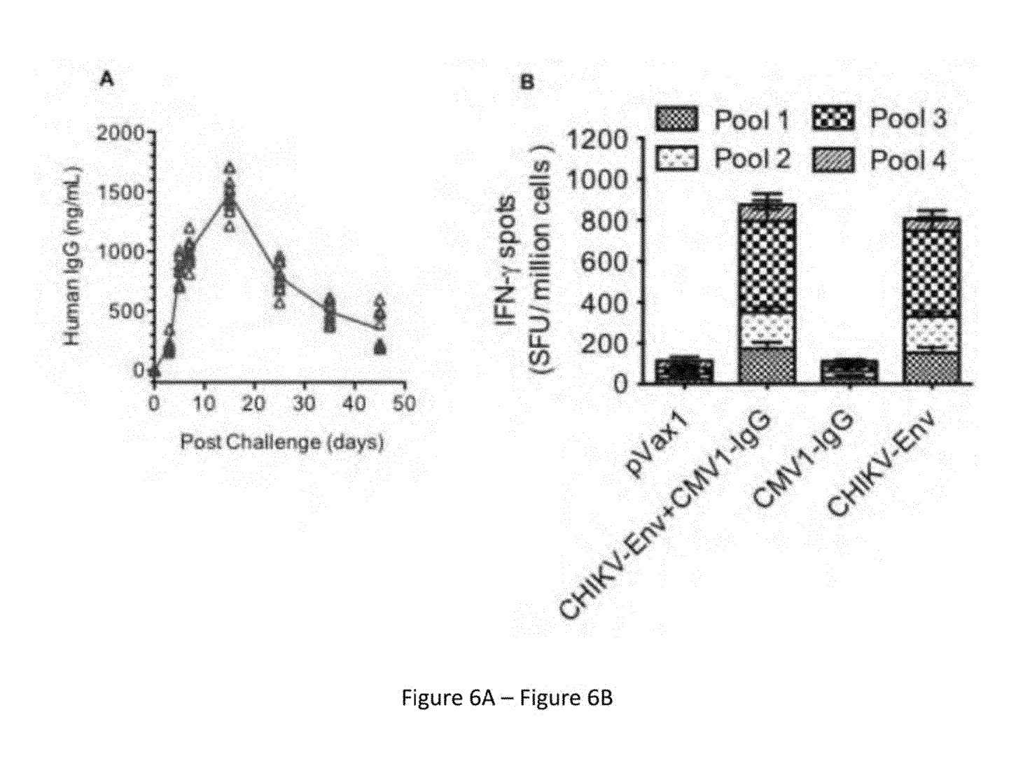

Next, a long-term CHIKV challenge protection study was performed on day 35 following vaccination with the CHIKV Env DNA vaccine or administration of CVM1-IgG on day 0. The multibooster delivery of the CHIKV Env DNA vaccine conferred 100% protection (FIG. 4B), while 80% survival was observed in mice administered CVM1-IgG (P=0.0007). The kinetics of the induced antibody responses was measurable within 2 days of a single injection of CVM1-IgG, with peak levels by day 15 (approximately 1400 ng/mL) and detectable mAb levels maintained for at least 45 days after injection (FIG. 6A). Although there is continued expression, these levels are decreased, compared with peak levels, supporting the partial protection noted in the experiment (FIG. 4B).

### Co-Delivery of CVM1-IgG and the CHIKV-Env DNA Vaccine Produces Systemic Humoral Immunity, Cell-Mediated Immunity, and Protection In Vivo

One potential issue of combining antibody delivery with vaccination approaches is that the antibodies can neutralize many traditional vaccines (Mallilankaraman et al., 2011, PLoS Negl Trop Dis 5:e928; Flingai et al., 2015, Sci Rep 5:12616; Muthumani et al., 2015, Sci transl Med 7:301ra132; Laddy et al., 2008, PLoS One 3:e2517) and thus are incompatible platforms. The effect of co-administration of CVM1-IgG and CHIKV Env on mouse survival in the context of CHIKV challenge was also evaluated. In this experiment, 20 mice were administered at day 0 a single dose of CVM1-IgG and 3 doses of CHIKV Env DNA as described above. Subsequently, half of the animals were challenged with CHIKV at day 2 and the other half at day 35. Survival in these groups was followed as a function of time. Not unexpectedly, both of the challenge groups had 100% long-term survival (FIG. 4C). Specifically, results of the day 2 CHIKV challenge experiment indicated the utility of the CVM1-IgG reagent in mediating protection from infection, with the survival percentage decreasing to approximately 30% by 4 days after challenge in control (pVax1) animals. FIG. 4D indicates levels of anti-CHIKV IgG, by time, generated in mice that received CVM1-IgG and CHIKV Env DNA vaccine; anti-CHIKV human IgG represents antibody produced by the CVM1-IgG plasmid and anti-CHIKV mouse IgG represents antibody induced by the CHIKV Env vaccine. Both human IgG and mouse IgG were detected and exhibited different expression kinetics. By 3 days after initial CHIKV Env DNA vaccination, mouse anti-Env antibody levels were essentially near 0 (mouse anti-CHIKV IgG). Conversely, 3 days after a single CVM1-IgG injection, human anti-Env antibody levels were significant (human anti-CHIKV IgG). These data underscore the importance of CVM1-IgG in mediating rapid protection from infection and death after CHIKV challenge.

Furthermore, T-cell responses induced in animals injected with CVM1-IgG, CHIKV Env, or CVM1-IgG plus CHIKV Env was evaluated by a quantitative enzyme-linked immunospot assay, which measures IFN-γ levels (FIG. 6B). CHIKV Env elicited strong T-cell responses irrespective of codelivery with CVM1-IgG, showing the lack of interference of these approaches. Conversely, animals administered only CVM1-IgG did not develop T-cell responses, as would be expected. These findings demonstrate that both CVM1-IgG and CHIKV Env DNA vaccine can be administered simultaneously without reciprocal interference, providing immediate and long-lived protection via systemic humoral and cellular immunity.

### CVM1-IgG Administration Reduces CHIKV Viral Loads and Pro-Inflammatory Cytokine Levels

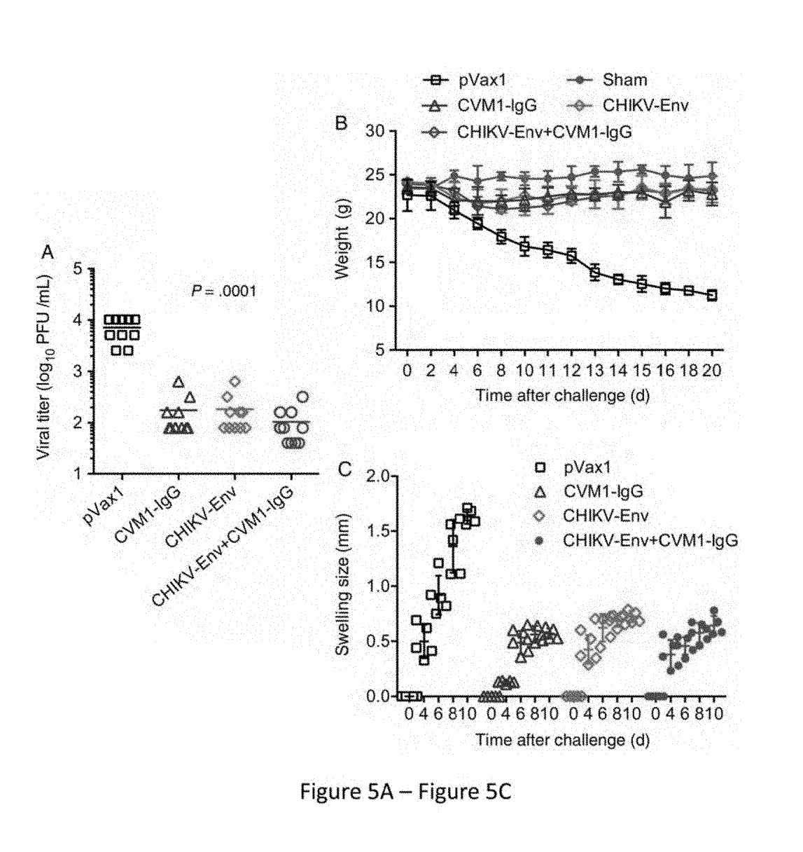

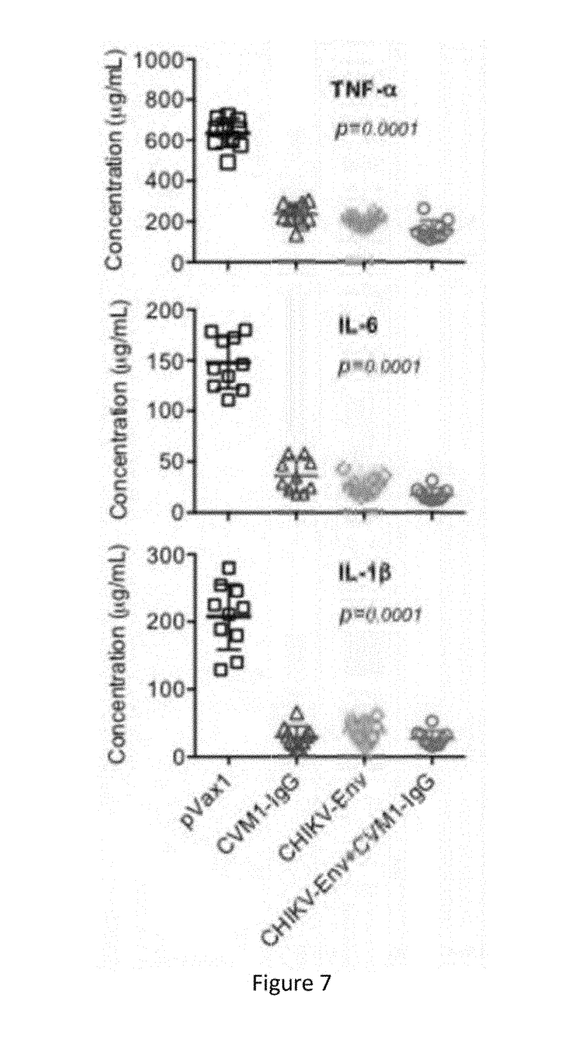

Previous studies identified molecular correlates of CHIKV-associated disease severity, including viral load and proinflammatory cytokine levels (Ng et al., 2009, PLoS One 4:e4261; Chaaitanya et al., 2011, Viral Immunol 24:265-71). Thus, the ability of CVM1 IgG to suppress these disease-associated markers at early and late time points after viral challenge was assessed. Mice immunized with CVM1 IgG, CVM1 Fab, CHIKV Env, or CVM1 IgG plus CHIKV Env DNA vaccine generated mAb and significantly reduced viral loads (FIG. 5A). In addition to viral load reduction, these mice did not exhibit footpad swelling, compared with control (pVax1) immunized mice, and consistently gained body weight during the 20-day experimental period (FIGS. 5B and 5C). Also the CVM1-IgG-generated mAb and the CHIKV Env DNA vaccine exhibited significantly reduced levels of CHIKV-mediated proinflammatory cytokines (ie, TNF-α, IL-6, and IL-β), compared with pVax1, 10 days after viral challenge (FIG. 7). These findings suggest that a single injection with CVM1-IgG suppresses CHIKV-associated pathology to an extent comparable to that induced by protective vaccination (Mallilankaraman et al., 2011, PLoS Negl Trop Dis 5:e928).

Electroporation-Mediated Delivery of Optimized DNA Plasmids for the In Vivo Rapid Production of Biologically Functional mAbs.

The results demonstrate that mice injected with a single dose of CVM1 IgG were fully protected from viral challenge 2 days after administration, whereas no mice survived infection following a single immunization with CHIKV Env DNA vaccine, owing presumably to an insufficient time to mount protective immunity. However, complete protection was observed with CHIKV Env after a immunization regimen followed by challenge at later time points. A similar level of protection occurred in mice administered a single dose of CVM1-IgG, although protection waned to 80% over time. Notably, the codelivery of CVM1-IgG and CHIKV Env produced rapid and persistent humoral and cellular immunity, demonstrating that a combination approach provides for synergistic, beneficial effects. Importantly, codelivery of CVM1-IgG and CHIKV Env were not antagonistic in terms of the development of short- or long-term protective immune responses.

### Example 2—Rapid and Long-Term Immunity Elicited by DNA Encoded Antibody Prophylaxis and DNA Vaccination Against Zika Virus

Vaccination is known to exhibit a lag phase before generation of immunity; thus, there is a gap of time during infection before an immune response is in effect. The following provides specific novel approaches that utilize the benefit of vaccines and the native immune response along with a rapid generation of effective immunity using the DNA synthetic antibodies or dMabs.

An antibody-based prophylaxis/therapy entailing the electroporation mediated delivery of synthetic plasmids, encoding biologically active anti-Zika virus envelope mAb (designated dMAb), is designed and evaluated for anti-viral efficacy as well as for the ability to overcome shortcomings inherent with conventional active vaccination by a novel passive immune-based strategy. One intramuscular injection of the ZIKV-dMAb produces antibodies in vivo more rapidly than active vaccination with an ZIKV-DNA vaccine. This dMAb neutralized diverse ZIKV clinical isolates and protected mice from viral challenge. Combinations of both afford rapid as well as long-lived protection.

A DNA based dMAb strategy induces rapid protection against an emerging viral infection, which can be combined with DNA vaccination providing a uniquely both short term and long-term protection against this emerging infectious disease. These studies have implications for pathogen treatment and control strategies.

dMAb IgG Quantification and Binding Assays

ELISA assays are performed with sera from subjects administered an ZIKV-dMAb to quantify expression kinetics and target antigen binding.

Analysis of dMAb Generated IgG

IgG expression of ZIKV infected cells are analyzed by western blot. For immunofluorescence analysis ZIKV infected cells are visually evaluated by confocal microscopy and quantitatively or semi-quantitatively analyzed.

dMAb DNA Plasmid Administration and In Vivo Analysis

Expression kinetics and functionality were evaluated in subjects following injection of control or ZIKV-dMAb. For studies that include the DNA vaccine, the ZIKV-DNA vaccine plasmid is administered.

### Challenge Study

Subjects receive electroporation-enhanced injection of ZIKV-dMAb or control plasmids. The ZIKV-DNA vaccine was delivered as described above. After DNA delivery, subjects are challenged with ZIKV. The animals are monitored for survival and signs of infection. Serum samples are collected for cytokine quantification and other immune analysis. Blood samples are collected from after infection and viremia levels are measured.

### Neutralizing Antibody Analysis

Anti-ZIKV neutralizing antibody titers from subjects administered ZIKV-dMAb are determined. Neutralization titers may be calculated as the reciprocal of the highest dilution mediating 100% reduction of the cytopathic effects in the cells.

### Cytokine Quantitative Analysis

Sera is collected from ZIKV-dMAb, and ZIKV-DNA vaccine injected subjects as well as ZIKV challenged subjects. TNF-α, IL-1β and IL-6 sera cytokine levels are measured.

Anti-ZIKV dMAbs Design and Confirmation of Expression

The optimized synthetic plasmids constructed from the anti-ZIKV-neutralizing mAb were designed for the IgG and Fab antibodies. Cells are transfected with either the ZIKV-IgG plasmid or the ZIKV-Fab (VL, VH, or combined) plasmids to validate expression in vitro. The ZIKV-Fab and ZIKV-IgG expressed antibodies in the muscle that appeared to be properly assembled and biologically functional in vitro.

In Vivo Expression and Quantification of Anti-ZIKV dMAb

Following confirmation of in vitro expression, the ability of ZIKV-Fab or ZIKV-IgG to produce anti-ZIKV antibodies in vivo is measured. Both constructs generate mAbs. Subjects are administered either ZIKV-IgG or ZIKV-Fab, and sera antibody levels are evaluated through a binding ELISA. Sera collected after injection from both ZIKV-IgG and ZIKV-Fab bind to ZIKV protein but not to an unrelated control antigen. These data indicate that in vivo produced anti-ZIKV antibodies from ZIKV-IgG or ZIKV-Fab constructs have similar biological characteristics to conventionally produced antigen specific antibodies.

In Vivo Specificity and Broadly Neutralizing Activity in Sera from Anti-ZIKV dMAb Injected Subjects

The anti-ZIKV dMAb generated mAbs are tested for binding specificity and anti-ZIKV neutralizing activity. Sera antibodies bind to ZIKV-infected cells. There is a strong specificity of the antibody generated from the anti-ZIKV dMAb plasmid.

Furthermore, the anti-ZIKV neutralizing activity in sera from subjects that received anti-ZIKV dMAb is measured against that in ZIKV strains. Sera from anti-ZIKV dMAb-injected subjects effectively neutralize ZIKV isolates, demonstrating that a single injection can produce significant neutralizing levels of human anti-ZIKV IgG. Thus, antibodies produced in vivo by anti-ZIKV dMAb constructs have relevant biological activity (ie, binding and neutralizing activity against ZIKV).

Anti-ZIKV dMAb Injection Protects Mice from Lethal ZIKV Challenge

To determine whether antibodies generated from anti-ZIKV dMAb provide protection against early exposure to ZIKV, groups of 10 subjects receive of a control or anti-ZIKV dMAb on day 0. Each group subsequently is challenged subcutaneously with virus to mimic natural ZIKV infection. Subject survival and weight changes are subsequently recorded. Anti-ZIKV dMAb plasmids confer protective immunity.

The longevity of immune protection is next evaluated. A second group of subjects are challenged with ZIKV after injection with anti-ZIKV dMAb, or control plasmid on day 0. Subjects are monitored for survival. Anti-ZIKV dMAb provides a more durable degree of immune protection.

Anti-ZIKV dMAb protects subjects from both subcutaneous viral challenge and intranasal viral challenge compared with control-injected subjects, demonstrating that anti-ZIKV dMAbs can protect against systemic and mucosal infection.

An efficacy study comparing the protective efficacy of anti-ZIKV dMAb administration vs a ZIKV-DNA vaccine (ZIKV-DNA) is next performed. A novel consensus-based DNA vaccine was developed by our laboratory and is capable of providing protection against ZIKV challenge. The DNA vaccine also induced both measurable cellular immune responses, as well as potent neutralizing antibody responses. Groups of subjects are administered a single injection of anti-ZIKV dMAb, ZIKV-DNA, or the pVax1, followed by viral challenge. Anti-ZIKV dMAb confers protective immunity more rapidly than the ZIKV-DNA vaccine.

Comparison Between In Vivo Protective Immunity Conferred by Anti-ZIKV dMAb Administration and ZIKV-DNA Vaccination

Next, a long-term ZIKV challenge protection study was performed following vaccination with the ZIKV-DNA vaccine or administration of anti-ZIKV dMAb on day 0. ZIKV-DNA confers longer protective immunity than anti-ZIKV dMAb.

Co-Delivery of Anti-ZIKV dMAb and the ZIKV-DNA Vaccine Produces Systemic Humoral Immunity, Cell-Mediated Immunity, and Protection In Vivo

One potential issue of combining antibody delivery with vaccination approaches is that the antibodies can neutralize many traditional vaccines and thus are incompatible platforms. The effect of co-administration of anti-ZIKV dMAb and ZIKV-DNA on subject survival in the context of ZIKV challenge was is evaluated. Subjects are administered at day 0 anti-ZIKV dMAb and ZIKV-DNA. Subsequently, some animals are challenged with ZIKV at day 2 and the others at day 35. Survival in these groups is followed as a function of time. Anti-ZIKV dMAb mediates protection from infection, with the survival percentage decreasing to approximately 30% by 4 days after challenge in control (pVax1) animals. Both IgG (induced by anti-ZIKV dMAb and ZIKV-DNA vaccine are detected. Anti-ZIKV dMAb mediates rapid protection from infection and death after ZIKV challenge.

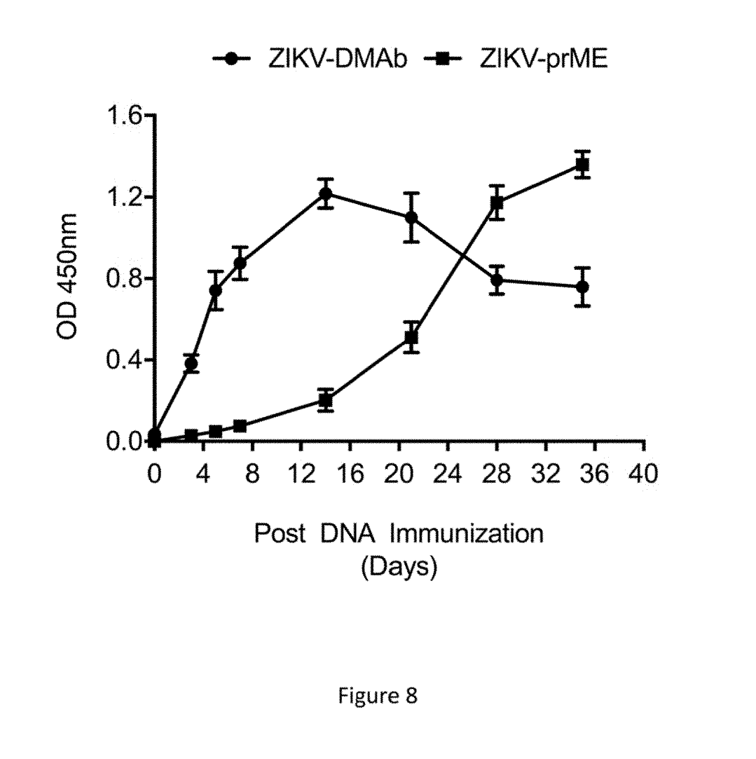

Furthermore, T-cell responses induced in subjects injected with Anti-ZIKV dMAb, ZIKV-DNA, or anti-ZIKV dMAb plus ZIKV-DNA are evaluated. ZIKV-DNA elicits strong T-cell responses irrespective of co-delivery with anti-ZIKV dMAb, showing the lack of interference of these approaches. Conversely, animals administered only anti-ZIKV dMAb do not develop T-cell responses. Both anti-ZIKV dMAb and ZIKV-DNA vaccine can be administered simultaneously without reciprocal interference, providing immediate and long-lived protection via systemic humoral and cellular immunity (FIG. 8).

Electroporation-Mediated Delivery of Optimized DNA Plasmids for the In Vivo Rapid Production of Biologically Functional mAbs

Subjects administered anti-ZIKV dMAbs are fully protected from viral challenge shortly after administration, whereas subjects do not survive infection following a single immunization with ZIKV-DNA vaccine, owing presumably to an insufficient time to mount protective immunity. However, ZIKV-DNA provides complete protection after an immunization regimen followed by challenge at later time points. A similar level of protection occurs in subjects administered a single dose of anti-ZIKV dMAbs, although protection wanes over time. Notably, the co-delivery of anti-ZIKV dMAbs and ZIKV-DNA produces rapid and persistent humoral and cellular immunity, suggesting that a combination approach can have additive or synergistic effects. Importantly, co-delivery of anti-ZIKV dMAbs and ZIKV-DNA are not antagonistic in terms of the development of short- or long-term protective immune responses.

### Example 3—Rapid and Long-Term Immunity Elicited by DNA Encoded Antibody Prophylaxis and DNA Vaccination Against Ebola Virus

Vaccination is known to exhibit a lag phase before generation of immunity; thus, there is a gap of time during infection before an immune response is in effect. The following provides specific novel approaches that utilize the benefit of vaccines and the native immune response along with a rapid generation of effective immunity using the DNA synthetic antibodies or dMabs.

An antibody-based prophylaxis/therapy entailing the electroporation mediated delivery of synthetic plasmids, encoding biologically active anti-Ebola virus envelope mAb (designated dMAb), is designed and evaluated for anti-viral efficacy as well as for the ability to overcome shortcomings inherent with conventional active vaccination by a novel passive immune-based strategy. One intramuscular injection of the EBOV-dMAb produces antibodies in vivo more rapidly than active vaccination with an EBOV-DNA vaccine. This dMAb neutralized diverse EBOV clinical isolates and protected mice from viral challenge. Combinations of both afford rapid as well as long-lived protection.

A DNA based dMAb strategy induces rapid protection against an emerging viral infection, which can be combined with DNA vaccination providing a uniquely both short term and long-term protection against this emerging infectious disease. These studies have implications for pathogen treatment and control strategies.

dMAb IgG Quantification and Binding Assays

ELISA assays are performed with sera from subjects administered an EBOV-dMAb to quantify expression kinetics and target antigen binding.

Analysis of dMAb Generated IgG

IgG expression of EBOV infected cells are analyzed by western blot. For immunofluorescence analysis EBOV infected cells are visually evaluated by confocal microscopy and quantitatively or semi-quantitatively analyzed.

dMAb DNA Plasmid Administration and In Vivo Analysis

Expression kinetics and functionality were evaluated in subjects following injection of control or EBOV-dMAb. For studies that include the DNA vaccine, the EBOV-DNA vaccine plasmid is administered.

### Challenge Study

Subjects receive electroporation-enhanced injection of EBOV-dMAb or control plasmids. The EBOV-DNA vaccine was delivered as described above. After DNA delivery, subjects are challenged with EBOV. The animals are monitored for survival and signs of infection. Serum samples are collected for cytokine quantification and other immune analysis. Blood samples are collected from after infection and viremia levels are measured.

### Neutralizing Antibody Analysis

Anti-EBOV neutralizing antibody titers from subjects administered EBOV-dMAb are determined. Neutralization titers may be calculated as the reciprocal of the highest dilution mediating 100% reduction of the cytopathic effects in the cells.

### Cytokine Quantitative Analysis

Sera is collected from EBOV-dMAb, and EBOV-DNA vaccine injected subjects as well as EBOV challenged subjects. TNF-α, IL-10 and IL-6 sera cytokine levels are measured.

Anti-EBOV dMAbs Design and Confirmation of Expression

The optimized synthetic plasmids constructed from the anti-EBOV-neutralizing mAb were designed for the IgG and Fab antibodies. Cells are transfected with either the EBOV-IgG plasmid or the EBOV-Fab (VL, VH, or combined) plasmids to validate expression in vitro. The EBOV-Fab and EBOV-IgG expressed antibodies in the muscle that appeared to be properly assembled and biologically functional in vitro.

In Vivo Expression and Quantification of Anti-EBOV dMAb

Following confirmation of in vitro expression, the ability of EBOV-Fab or EBOV-IgG to produce anti-EBOV antibodies in vivo is measured. Both constructs generate mAbs. Subjects are administered either EBOV-IgG or EBOV-Fab, and sera antibody levels are evaluated through a binding ELISA. Sera collected after injection from both EBOV-IgG and EBOV-Fab bind to EBOV protein but not to an unrelated control antigen. These data indicate that in vivo produced anti-EBOV antibodies from EBOV-IgG or EBOV-Fab constructs have similar biological characteristics to conventionally produced antigen specific antibodies.

In Vivo Specificity and Broadly Neutralizing Activity in Sera from Anti-EBOV dMAb Injected Subjects

The anti-EBOV dMAb generated mAbs are tested for binding specificity and anti-EBOV neutralizing activity. Sera antibodies bind to EBOV-infected cells. There is a strong specificity of the antibody generated from the anti-EBOV dMAb plasmid.

Furthermore, the anti-EBOV neutralizing activity in sera from subjects that received anti-EBOV dMAb is measured against that in EBOV strains. Sera from anti-EBOV dMAb-injected subects effectively neutralize EBOV isolates, demonstrating that a single injection can produce significant neutralizing levels of human anti-EBOV IgG. Thus, antibodies produced in vivo by anti-EBOV dMAb constructs have relevant biological activity (ie, binding and neutralizing activity against EBOV).

Anti-EBOV dMAb Injection Protects Mice from Lethal EBOV Challenge

To determine whether antibodies generated from anti-EBOV dMAb provide protection against early exposure to EBOV, groups of 10 subjects receive of a control or anti-EBOV dMAb on day 0. Each group subsequently is challenged subcutaneously with virus to mimic natural EBOV infection. Subject survival and weight changes are subsequently recorded. Anti-EBOV dMAb plasmids confer protective immunity.

The longevity of immune protection is next evaluated. A second group of subjects are challenged with EBOV after injection with anti-EBOV dMAb, or control plasmid on day 0. Subjects are monitored for survival. Anti-EBOV dMAb provides a more durable degree of immune protection.

Anti-EBOV dMAb protects subjects from both subcutaneous viral challenge and intranasal viral challenge compared with control-injected subjects, demonstrating that anti-EBOV dMAbs can protect against systemic and mucosal infection.

An efficacy study comparing the protective efficacy of anti-EBOV dMAb administration vs a EBOV-DNA vaccine (EBOV-DNA) is next performed. A novel consensus-based DNA vaccine was developed by our laboratory and is capable of providing protection against EBOV challenge. The DNA vaccine also induced both measurable cellular immune responses, as well as potent neutralizing antibody responses. Groups of subjects are administered a single injection of anti-EBOV dMAb, EBOV-DNA, or the pVax1, followed by viral challenge. Anti-EBOV dMAb confers protective immunity more rapidly than the EBOV-DNA vaccine.

Comparison Between In Vivo Protective Immunity Conferred by Anti-EBOV dMAb Administration and EBOV-DNA Vaccination

Next, a long-term EBOV challenge protection study was performed following vaccination with the EBOV-DNA vaccine or administration of anti-EBOV dMAb on day 0. EBOV-DNA confers longer protective immunity than anti-EBOV dMAb.

Co-Delivery of Anti-EBOV dMAb and the EBOV-DNA Vaccine Produces Systemic Humoral Immunity, Cell-Mediated Immunity, and Protection In Vivo

One potential issue of combining antibody delivery with vaccination approaches is that the antibodies can neutralize many traditional vaccines and thus are incompatible platforms. The effect of co-administration of anti-EBOV dMAb and EBOV-DNA on subject survival in the context of EBOV challenge was is evaluated. Subjects are administered at day 0 anti-EBOV dMAb and EBOV-DNA. Subsequently, some animals are challenged with EBOV at day 2 and the others at day 35. Survival in these groups is followed as a function of time. Anti-EBOV dMAb mediates protection from infection, with the survival percentage decreasing to approximately 30% by 4 days after challenge in control (pVax1) animals. Both IgG induced by anti-EBOV dMAb and EBOV-DNA vaccine are detected. Anti-EBOV dMAb mediates rapid protection from infection and death after EBOV challenge.

Furthermore, T-cell responses induced in subjects injected with Anti-EBOV dMAb, EBOV-DNA, or anti-EBOV dMAb plus EBOV-DNA are evaluated. EBOV-DNA elicits strong T-cell responses irrespective of co-delivery with anti-EBOV dMAb, showing the lack of interference of these approaches. Conversely, animals administered only anti-EBOV dMAb do not develop T-cell responses. Both anti-EBOV dMAb and EBOV-DNA vaccine can be administered simultaneously without reciprocal interference, providing immediate and long-lived protection via systemic humoral and cellular immunity.

Electroporation-Mediated Delivery of Optimized DNA Plasmids for the In Vivo Rapid Production of Biologically Functional mAbs

Subjects administered anti-EBOV dMAbs are fully protected from viral challenge shortly after administration, whereas subjects do not survive infection following a single immunization with EBOV-DNA vaccine, owing presumably to an insufficient time to mount protective immunity. However, EBOV-DNA provides complete protection after an immunization regimen followed by challenge at later time points. A similar level of protection occurs in subjects administered a single dose of anti-EBOV dMAbs, although protection wanes over time. Notably, the co-delivery of anti-EBOV dMAbs and EBOV-DNA produces rapid and persistent humoral and cellular immunity, suggesting that a combination approach can have additive or synergistic effects. Importantly, co-delivery of anti-EBOV dMAbs and EBOV-DNA are not antagonistic in terms of the development of short- or long-term protective immune responses.

### Example 4—Rapid and Long-Term Immunity Elicited by DNA Encoded Antibody Prophylaxis and DNA Vaccination Against Marburg Virus

Vaccination is known to exhibit a lag phase before generation of immunity; thus, there is a gap of time during infection before an immune response is in effect. The following provides specific novel approaches that utilize the benefit of vaccines and the native immune response along with a rapid generation of effective immunity using the DNA synthetic antibodies or dMabs.

An antibody-based prophylaxis/therapy entailing the electroporation mediated delivery of synthetic plasmids, encoding biologically active anti-Marburg virus (MARV) mAb (designated dMAb), is designed and evaluated for anti-viral efficacy as well as for the ability to overcome shortcomings inherent with conventional active vaccination by a novel passive immune-based strategy. One intramuscular injection of the MARV-dMAb produces antibodies in vivo more rapidly than active vaccination with an MARV-DNA vaccine. This dMAb neutralized diverse MARV clinical isolates and protected mice from viral challenge. Combinations of both afford rapid as well as long-lived protection.

A DNA based dMAb strategy induces rapid protection against an emerging viral infection, which can be combined with DNA vaccination providing a uniquely both short term and long-term protection against this emerging infectious disease. These studies have implications for pathogen treatment and control strategies.

dMAb IgG Quantification and Binding Assays

ELISA assays are performed with sera from subjects administered an MARV-dMAb to quantify expression kinetics and target antigen binding.

Analysis of dMAb Generated IgG

IgG expression of MARV infected cells are analyzed by western blot. For immunofluorescence analysis MARV infected cells are visually evaluated by confocal microscopy and quantitatively or semi-quantitatively analyzed.

dMAb DNA Plasmid Administration and In Vivo Analysis

Expression kinetics and functionality were evaluated in subjects following injection of control or MARV-dMAb. For studies that include the DNA vaccine, the MARV-DNA vaccine plasmid is administered.

### Challenge Study

Subjects receive electroporation-enhanced injection of MARV-dMAb or control plasmids. The MARV-DNA vaccine was delivered as described above. After DNA delivery, subjects are challenged with MARV. The animals are monitored for survival and signs of infection. Serum samples are collected for cytokine quantification and other immune analysis. Blood samples are collected from after infection and viremia levels are measured.

### Neutralizing Antibody Analysis

Anti-MARV neutralizing antibody titers from subjects administered MARV-dMAb are determined. Neutralization titers may be calculated as the reciprocal of the highest dilution mediating 100% reduction of the cytopathic effects in the cells.

### Cytokine Quantitative Analysis

Sera is collected from MARV-dMAb, and MARV-DNA vaccine injected subjects as well as MARV challenged subjects. TNF-α, IL-10 and IL-6 sera cytokine levels are measured.

Anti-MARV dMAbs Design and Confirmation of Expression

The optimized synthetic plasmids constructed from the anti-MARV-neutralizing mAb were designed for the IgG and Fab antibodies. Cells are transfected with either the MARV-IgG plasmid or the MARV-Fab (VL, VH, or combined) plasmids to validate expression in vitro. The MARV-Fab and MARV-IgG expressed antibodies in the muscle that appeared to be properly assembled and biologically functional in vitro.

In Vivo Expression and Quantification of Anti-MARV dMAb

Following confirmation of in vitro expression, the ability of MARV-Fab or MARV-IgG to produce anti-MARV antibodies in vivo is measured. Both constructs generate mAbs. Subjects are administered either MARV-IgG or MARV-Fab, and sera antibody levels are evaluated through a binding ELISA. Sera collected after injection from both MARV-IgG and MARV-Fab bind to MARV protein but not to an unrelated control antigen. These data indicate that in vivo produced anti-MARV antibodies from MARV-IgG or MARV-Fab constructs have similar biological characteristics to conventionally produced antigen specific antibodies.

In Vivo Specificity and Broadly Neutralizing Activity in Sera from Anti-MARV dMAb Injected Subjects

The anti-MARV dMAb generated mAbs are tested for binding specificity and anti-MARV neutralizing activity. Sera antibodies bind to MARV-infected cells. There is a strong specificity of the antibody generated from the anti-MARV dMAb plasmid.

Furthermore, the anti-MARV neutralizing activity in sera from subjects that received anti-MARV dMAb is measured against that in MARV strains. Sera from anti-MARV dMAb-injected subects effectively neutralize MARV isolates, demonstrating that a single injection can produce significant neutralizing levels of human anti-MARV IgG. Thus, antibodies produced in vivo by anti-MARV dMAb constructs have relevant biological activity (ie, binding and neutralizing activity against MARV).

Anti-MARV dMAb Injection Protects Mice from Lethal MARV Challenge

To determine whether antibodies generated from anti-MARV dMAb provide protection against early exposure to MARV, groups of 10 subjects receive of a control or anti-MARV dMAb on day 0. Each group subsequently is challenged subcutaneously with virus to mimic natural MARV infection. Subject survival and weight changes are subsequently recorded. anti-MARV dMAb plasmids confer protective immunity.

The longevity of immune protection is next evaluated. A second group of subjects was challenged with MARV after injection with anti-MARV dMAb, or control plasmid on day 0. Subjects are monitored for survival. Anti-MARV dMAb provides a more durable degree of immune protection.

Anti-MARV dMAb protects subjects from both subcutaneous viral challenge and intranasal viral challenge compared with control-injected subjects, demonstrating that anti-MARV dMAbs can protect against systemic and mucosal infection.

An efficacy study comparing the protective efficacy of anti-MARV dMAb administration vs a MARV-DNA vaccine (MARV-DNA) is next performed. A novel consensus-based DNA vaccine was developed by our laboratory and is capable of providing protection against MARV challenge. The DNA vaccine also induced both measurable cellular immune responses, as well as potent neutralizing antibody responses. Groups of subjects are administered a single injection of anti-MARV dMAb, MARV-DNA, or the pVax1, followed by viral challenge. Anti-MARV dMAb confers protective immunity more rapidly than the MARV-DNA vaccine.

Comparison Between In Vivo Protective Immunity Conferred by Anti-MARV dMAb Administration and MARV-DNA Vaccination

Next, a long-term MARV challenge protection study was performed following vaccination with the MARV-DNA vaccine or administration of anti-MARV dMAb on day 0. MARV-DNA confers longer protective immunity than anti-MARV dMAb.

Co-Delivery of Anti-MARV dMAb and the MARV-DNA Vaccine Produces Systemic Humoral Immunity, Cell-Mediated Immunity, and Protection In Vivo

One potential issue of combining antibody delivery with vaccination approaches is that the antibodies can neutralize many traditional vaccines and thus are incompatible platforms. The effect of co-administration of anti-MARV dMAb and MARV-DNA on subject survival in the context of MARV challenge was is evaluated. Subjects are administered at day 0 anti-MARV dMAb and MARV-DNA. Subsequently, some animals are challenged with MARV at day 2 and the others at day 35. Survival in these groups is followed as a function of time. Anti-MARV dMAb mediates protection from infection, with the survival percentage decreasing to approximately 30% by 4 days after challenge in control (pVax1) animals. Both IgG (induced by anti-MARV dMAb and MARV-DNA vaccine are detected. Anti-MARV dMAb mediates rapid protection from infection and death after MARV challenge.

Furthermore, T-cell responses induced in subjects injected with Anti-MARV dMAb, MARV-DNA, or anti-MARV dMAb plus MARV-DNA are evaluated. MARV-DNA elicits strong T-cell responses irrespective of co-delivery with anti-MARV dMAb, showing the lack of interference of these approaches. Conversely, animals administered only anti-MARV dMAb do not develop T-cell responses. Both anti-MARV dMAb and MARV-DNA vaccine can be administered simultaneously without reciprocal interference, providing immediate and long-lived protection via systemic humoral and cellular immunity.

Electroporation-Mediated Delivery of Optimized DNA Plasmids for the In Vivo Rapid Production of Biologically Functional mAbs

Subjects administered anti-MARV dMAbs are fully protected from viral challenge shortly after administration, whereas subjects do not survive infection following a single immunization with MARV-DNA vaccine, owing presumably to an insufficient time to mount protective immunity. However, MARV-DNA provides complete protection after an immunization regimen followed by challenge at later time points. A similar level of protection occurs in subjects administered a single dose of anti-MARV dMAbs, although protection wanes over time. Notably, the co-delivery of anti-MARV dMAbs and MARV-DNA produces rapid and persistent humoral and cellular immunity, suggesting that a combination approach can have additive or synergistic effects. Importantly, co-delivery of anti-MARV dMAbs and MARV-DNA are not antagonistic in terms of the development of short- or long-term protective immune responses.

### Example 5—Rapid and Long-Term Immunity Elicited by DNA Encoded Antibody Prophylaxis and DNA Vaccination Against Influenza

Vaccination is known to exhibit a lag phase before generation of immunity; thus, there is a gap of time during infection before an immune response is in effect. The following provides specific novel approaches that utilize the benefit of vaccines and the native immune response along with a rapid generation of effective immunity using the DNA synthetic antibodies or dMabs.

An antibody-based prophylaxis/therapy entailing the electroporation mediated delivery of synthetic plasmids, encoding biologically active anti-Influenza virus (Flu) mAb (designated dMAb), is designed and evaluated for anti-viral efficacy as well as for the ability to overcome shortcomings inherent with conventional active vaccination by a novel passive immune-based strategy. One intramuscular injection of the Flu-dMAb produces antibodies in vivo more rapidly than active vaccination with an Flu-DNA vaccine. This dMAb neutralized diverse Flu clinical isolates and protected mice from viral challenge. Combinations of both afford rapid as well as long-lived protection.

A DNA based dMAb strategy induces rapid protection against an emerging viral infection, which can be combined with DNA vaccination providing a uniquely both short term and long-term protection against this emerging infectious disease. These studies have implications for pathogen treatment and control strategies.

dMAb IgG Quantification and Binding Assays

ELISA assays are performed with sera from subjects administered an Flu-dMAb to quantify expression kinetics and target antigen binding.

Analysis of dMAb Generated IgG

IgG expression of Flu infected cells are analyzed by western blot. For immunofluorescence analysis Flu infected cells are visually evaluated by confocal microscopy and quantitatively or semi-quantitatively analyzed.

dMAb DNA Plasmid Administration and In Vivo Analysis

Expression kinetics and functionality were evaluated in subjects following injection of control or Flu-dMAb. For studies that include the DNA vaccine, the Flu-DNA vaccine plasmid is administered.

### Challenge Study

Subjects receive electroporation-enhanced injection of Flu-dMAb or control plasmids. The Flu-DNA vaccine was delivered as described above. After DNA delivery, subjects are challenged with Flu. The animals are monitored for survival and signs of infection. Serum samples are collected for cytokine quantification and other immune analysis. Blood samples are collected from after infection and viremia levels are measured.

### Neutralizing Antibody Analysis

Anti-Flu neutralizing antibody titers from subjects administered Flu-dMAb are determined. Neutralization titers may be calculated as the reciprocal of the highest dilution mediating 100% reduction of the cytopathic effects in the cells.

### Cytokine Quantitative Analysis

Sera is collected from Flu-dMAb, and Flu-DNA vaccine injected subjects as well as Flu challenged subjects. TNF-α, IL-10 and IL-6 sera cytokine levels are measured.

Anti-Flu dMAbs Design and Confirmation of Expression

The optimized synthetic plasmids constructed from the anti-Flu-neutralizing mAb were designed for the IgG and Fab antibodies. Cells are transfected with either the Flu-IgG plasmid or the Flu-Fab (VL, VH, or combined) plasmids to validate expression in vitro. The Flu-Fab and Flu-IgG expressed antibodies in the muscle that appeared to be properly assembled and biologically functional in vitro.

In Vivo Expression and Quantification of Anti-Flu dMAb

Following confirmation of in vitro expression, the ability of Flu-Fab or Flu-IgG to produce anti-Flu antibodies in vivo is measured. Both constructs generate mAbs. Subjects are administered either Flu-IgG or Flu-Fab, and sera antibody levels are evaluated through a binding ELISA. Sera collected after injection from both Flu-IgG and Flu-Fab bind to Flu protein but not to an unrelated control antigen. These data indicate that in vivo produced anti-Flu antibodies from Flu-IgG or Flu-Fab constructs have similar biological characteristics to conventionally produced antigen specific antibodies.

In Vivo Specificity and Broadly Neutralizing Activity in Sera from Anti-Flu dMAb Injected Subjects

The anti-Flu dMAb generated mAbs are tested for binding specificity and anti-Flu neutralizing activity. Sera antibodies bind to Flu-infected cells. There is a strong specificity of the antibody generated from the anti-Flu dMAb plasmid.

Furthermore, the anti-Flu neutralizing activity in sera from subjects that received anti-Flu dMAb is measured against that in Flu strains. Sera from anti-Flu dMAb-injected subects effectively neutralize Flu isolates, demonstrating that a single injection can produce significant neutralizing levels of human anti-Flu IgG. Thus, antibodies produced in vivo by anti-Flu dMAb constructs have relevant biological activity (ie, binding and neutralizing activity against Flu).

Anti-Flu dMAb Injection Protects Mice from Lethal Flu Challenge

To determine whether antibodies generated from anti-Flu dMAb provide protection against early exposure to Flu, groups of 10 subjects receive of a control or anti-Flu dMAb on day 0. Each group subsequently is challenged subcutaneously with virus to mimic natural Flu infection. Subject survival and weight changes are subsequently recorded. anti-Flu dMAb plasmids confer protective immunity.

The longevity of immune protection is next evaluated. A second group of subjects was challenged with Flu after injection with anti-Flu dMAb, or control plasmid on day 0. Subjects are monitored for survival. Anti-Flu dMAb provides a more durable degree of immune protection.

Anti-Flu dMAb protects subjects from both subcutaneous viral challenge and intranasal viral challenge compared with control-injected subjects, demonstrating that anti-Flu dMAbs can protect against systemic and mucosal infection.

An efficacy study comparing the protective efficacy of anti-Flu dMAb administration vs a Flu-DNA vaccine (Flu-DNA) is next performed. A novel consensus-based DNA vaccine was developed by our laboratory and is capable of providing protection against Flu challenge. The DNA vaccine also induced both measurable cellular immune responses, as well as potent neutralizing antibody responses. Groups of subjects are administered a single injection of anti-Flu dMAb, Flu-DNA, or the pVax1, followed by viral challenge. Anti-Flu dMAb confers protective immunity more rapidly than the Flu-DNA vaccine.

Comparison Between In Vivo Protective Immunity Conferred by Anti-Flu dMAb Administration and Flu-DNA Vaccination

Next, a long-term Flu challenge protection study was performed following vaccination with the Flu-DNA vaccine or administration of anti-Flu dMAb on day 0. Flu-DNA confers longer protective immunity than anti-Flu dMAb.

Co-Delivery of Anti-Flu dMAb and the Flu-DNA Vaccine Produces Systemic Humoral Immunity, Cell-Mediated Immunity, and Protection In Vivo

One potential issue of combining antibody delivery with vaccination approaches is that the antibodies can neutralize many traditional vaccines and thus are incompatible platforms. The effect of co-administration of anti-Flu dMAb and Flu-DNA on subject survival in the context of Flu challenge was is evaluated. Subjects are administered at day 0 anti-Flu dMAb and Flu-DNA. Subsequently, some animals are challenged with Flu at day 2 and the others at day 35. Survival in these groups is followed as a function of time. Anti-Flu dMAb mediates protection from infection, with the survival percentage decreasing to approximately 30% by 4 days after challenge in control (pVax1) animals. Both IgG (induced by anti-Flu dMAb and Flu-DNA vaccine are detected. Anti-Flu dMAb mediates rapid protection from infection and death after Flu challenge.

Furthermore, T-cell responses induced in subjects injected with Anti-Flu dMAb, Flu-DNA, or anti-Flu dMAb plus Flu-DNA are evaluated. Flu-DNA elicits strong T-cell responses irrespective of co-delivery with anti-Flu dMAb, showing the lack of interference of these approaches. Conversely, animals administered only anti-Flu dMAb do not develop T-cell responses. Both anti-Flu dMAb and Flu-DNA vaccine can be administered simultaneously without reciprocal interference, providing immediate and long-lived protection via systemic humoral and cellular immunity.

Electroporation-Mediated Delivery of Optimized DNA Plasmids for the In Vivo Rapid Production of Biologically Functional mAbs

Subjects administered anti-Flu dMAbs are fully protected from viral challenge shortly after administration, whereas subjects do not survive infection following a single immunization with Flu-DNA vaccine, owing presumably to an insufficient time to mount protective immunity. However, Flu-DNA provides complete protection after an immunization regimen followed by challenge at later time points. A similar level of protection occurs in subjects administered a single dose of anti-Flu dMAbs, although protection wanes over time. Notably, the co-delivery of anti-Flu dMAbs and Flu-DNA produces rapid and persistent humoral and cellular immunity, suggesting that a combination approach can have additive or synergistic effects. Importantly, co-delivery of anti-Flu dMAbs and Flu-DNA are not antagonistic in terms of the development of short- or long-term protective immune responses.

### Example 5—Functional Anti-Zika “DNA Monoclonal Antibodies” (DMAb)

The studies presented herein demonstrate the generation of functional anti-Zika “DNA monoclonal antibodies” (DMAb) via intramuscular electroporation of plasmid DNA. Codon-optimized variable region DNA sequences from anti-Zika monoclonal antibodies were synthesized onto a human IgG1 constant domain. Plasmid DNA encoding antibody was delivered to C3H mice mice. This study supports DMAb as an alternative to existing biologic therapies.

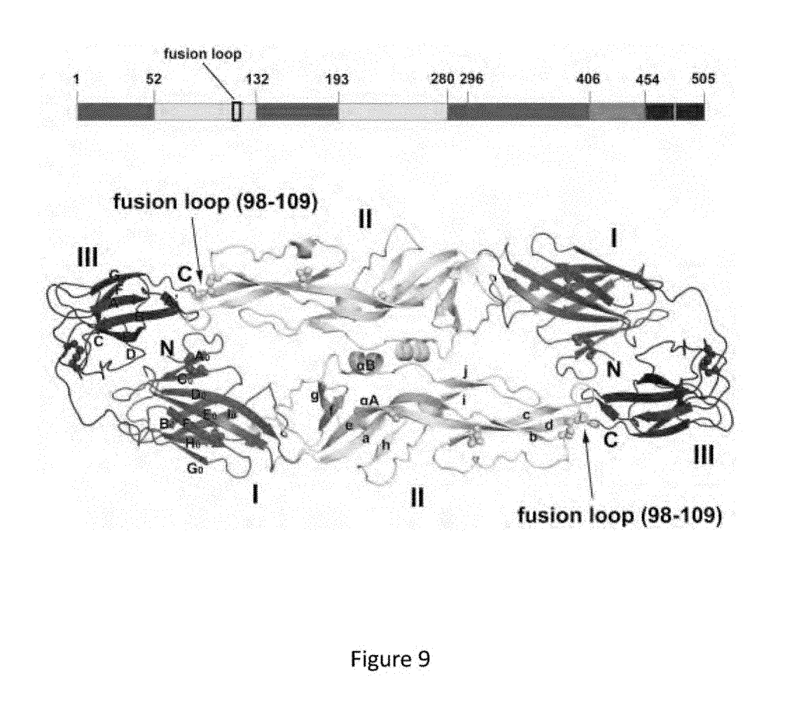

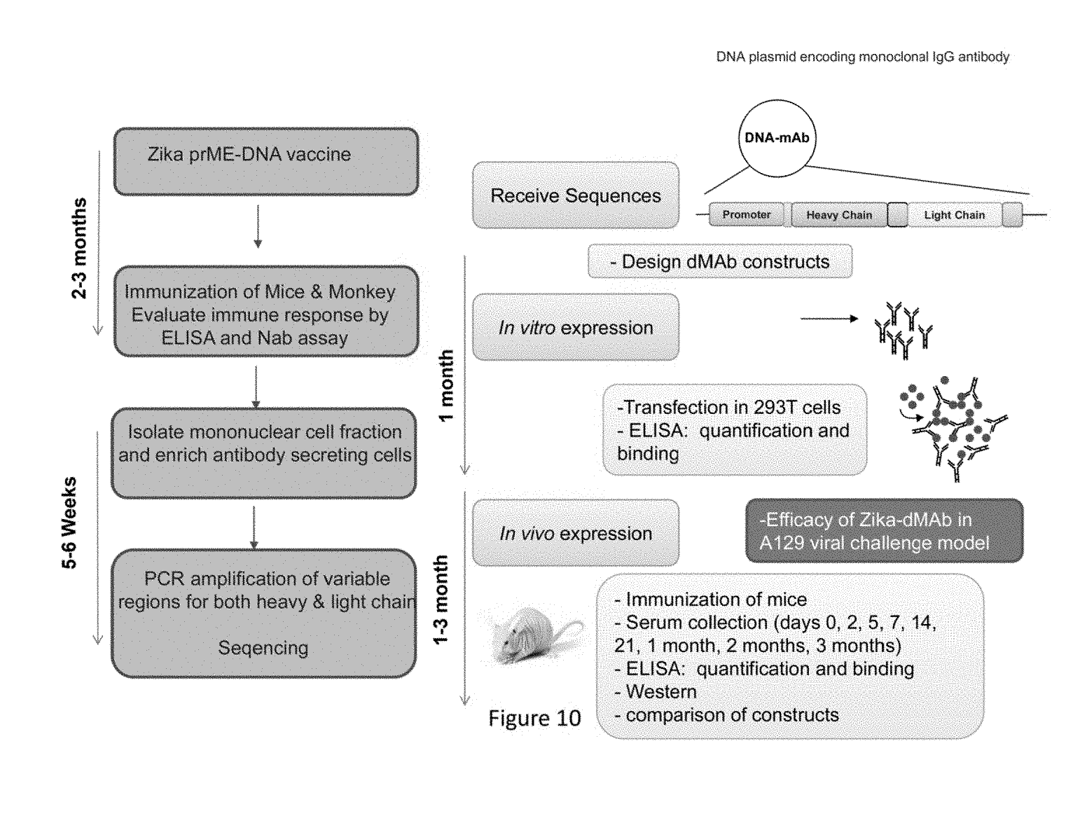

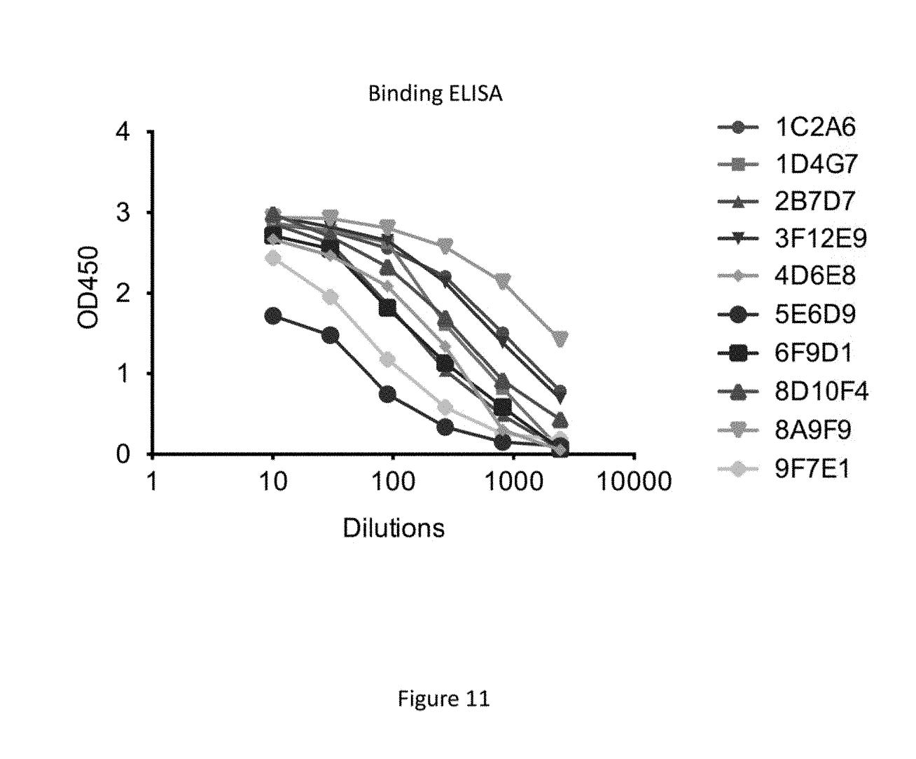

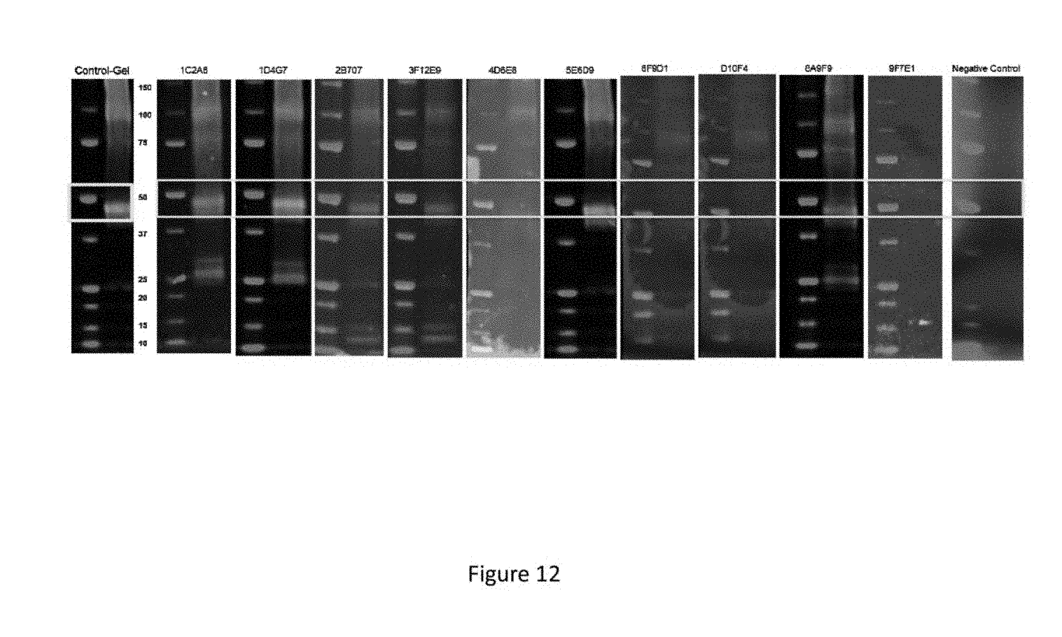

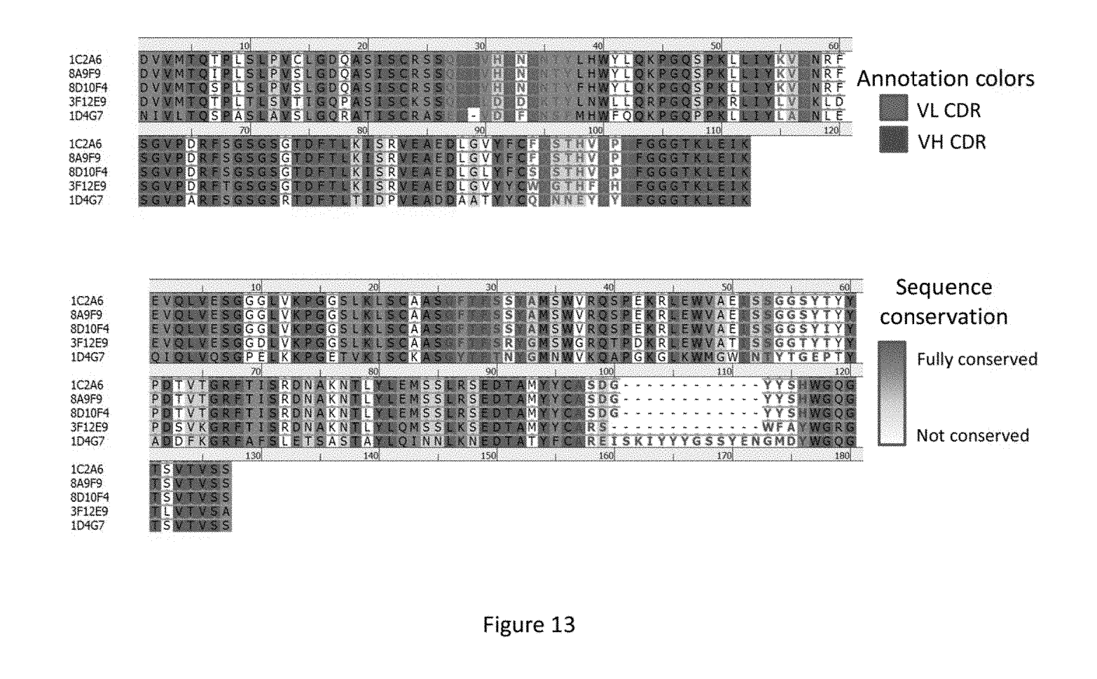

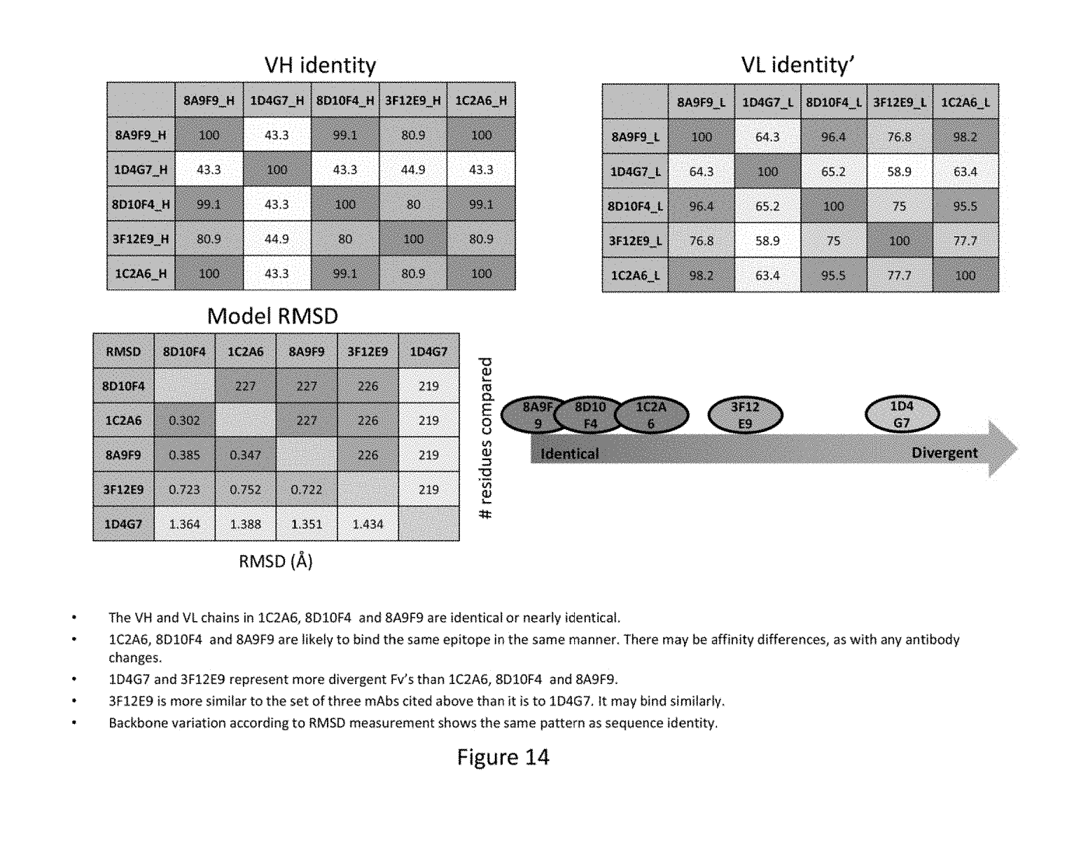

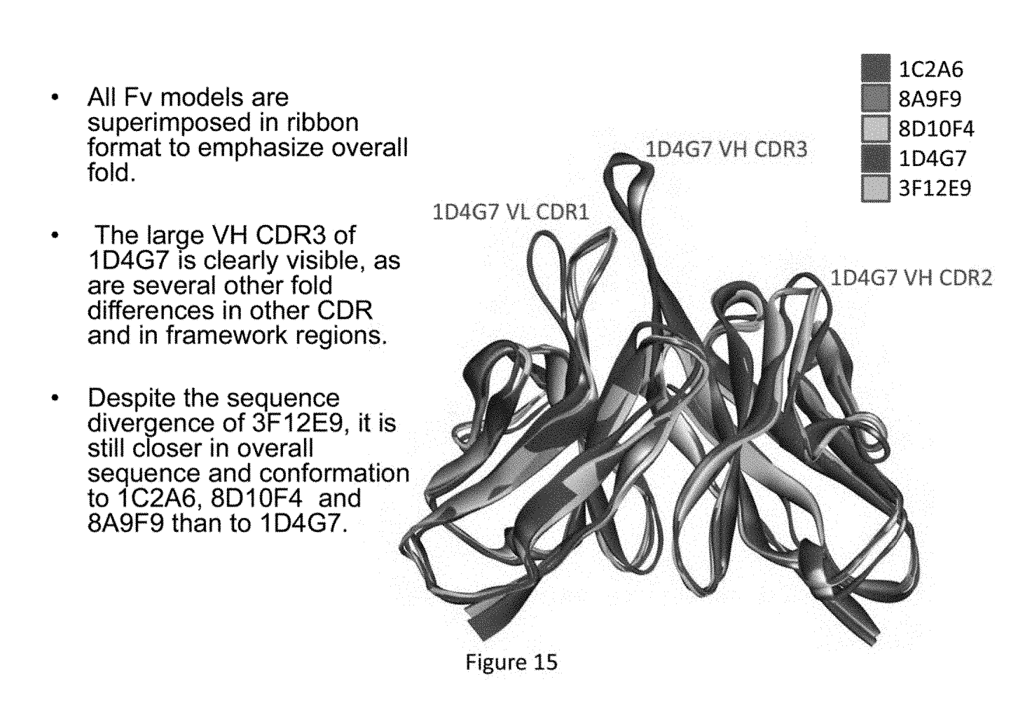

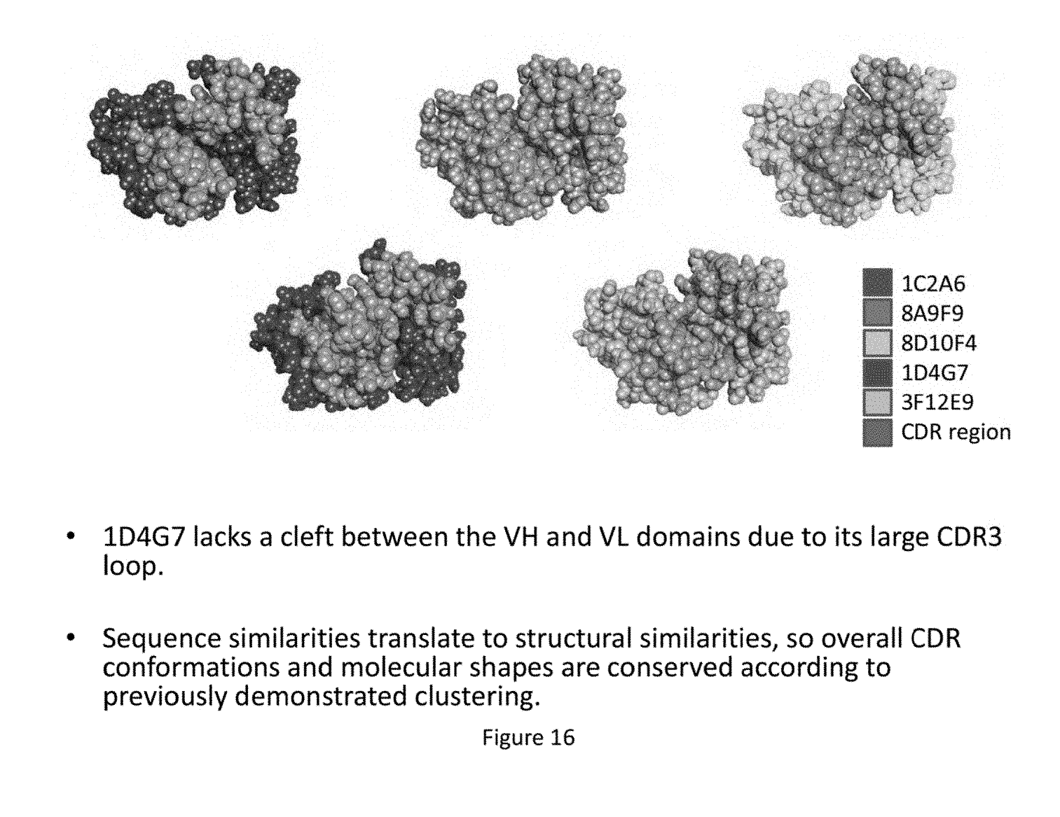

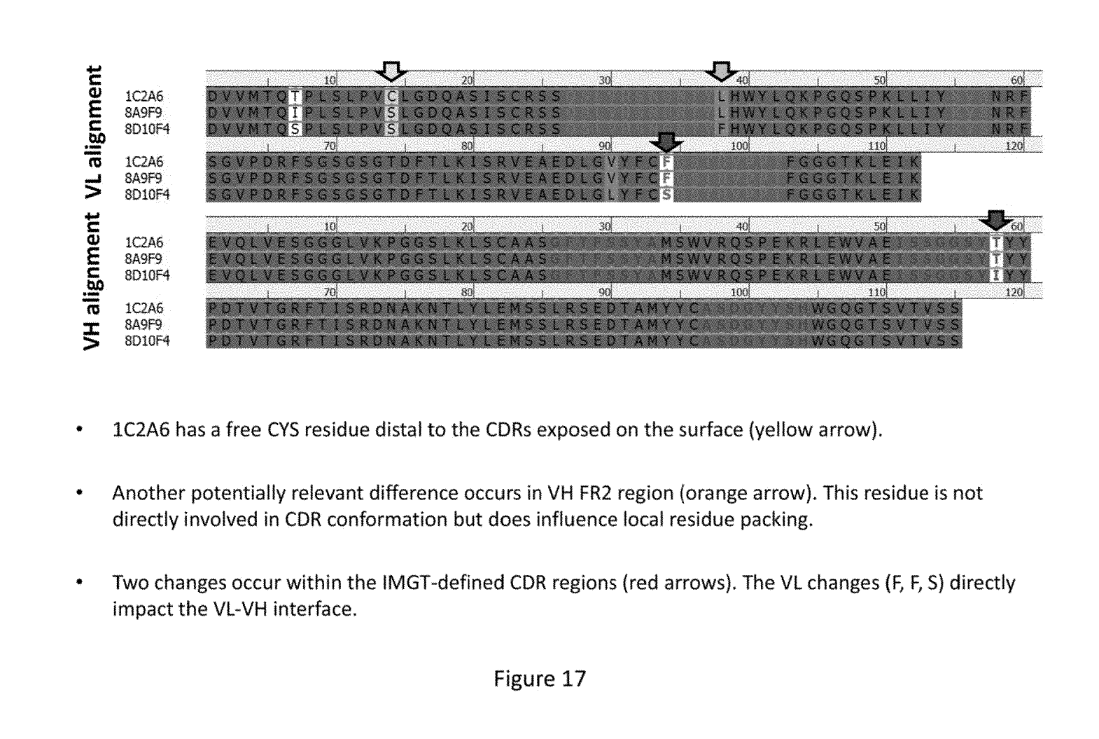

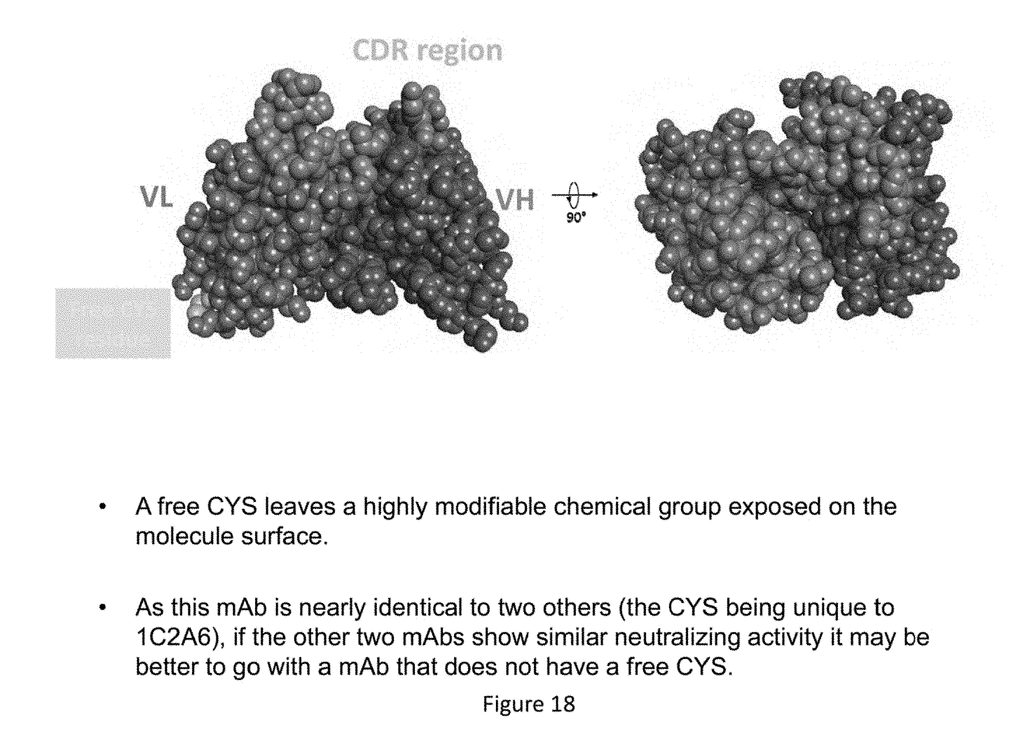

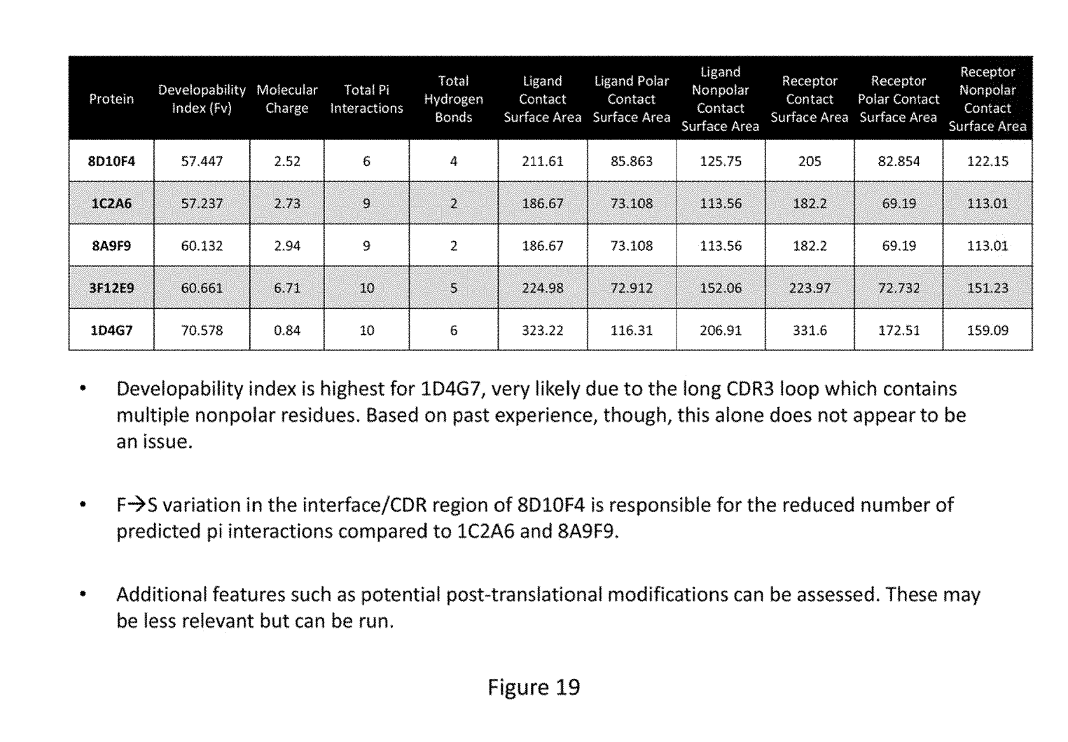

The ZIKV-Env (ZIKV-E) protein is a 505 amino acid protein having a fusion loop (FIG. 9). The antibodies against the ZIKV-E protein are expressed in vivo through DNA monoclonal antibodies (dMABs) which express a heavy and light chain (FIG. 10). ZIKV-Env specific monoclonal antibodies, 1C2A6, 1D4G7, 2B7D7, 3F12E9, 4D6E8, 5E6D9, 6F9D1, 9D10F4, 8A9F9, and 9F7E1, each bind ZIKV-Env in vitro (FIG. 11 and FIG. 12). The monoclonal antibodies show varying degrees of sequence homology among both the V_{H} and V_{L} chains (FIGS. 13-15). The large VH CDR3 of 1D4G7 is clearly visible, as are several other fold differences in other CDR and in framework regions. Despite the sequence divergence of 3F12E9, it is still closer in overall sequence and conformation to 1C2A6, 8D10F4 and 8A9F9 than to 1D4G7. (FIG. 15). 1D4G7 lacks a cleft between the VH and VL domains due to its large CDR3 loop. Sequence similarities translate to structural similarities, so overall CDR conformations and molecular shapes are conserved according to previously demonstrated clustering. (FIG. 16). 1C2A6 has a free CYS residue distal to the CDRs exposed on the surface Another potentially relevant difference occurs in VH FR2 region. This residue is not directly involved in CDR conformation but does influence local residue packing. Two changes occur within the IMGT-defined CDR regions. The VL changes (F, F, S) directly impact the VL-VH interface. (FIG. 17). A free CYS leaves a highly modifiable chemical group exposed on the molecule surface. (FIG. 18). Developability index is highest for 1D4G7, very likely due to the long CDR3 loop which contains multiple nonpolar residues. Based on past experience, though, this alone does not appear to be an issue (FIG. 19). Based on the high degrees of similarity, 1C2A6, 8D10F4 and 8A9F9 are likely to bind the same epitope in the same basic mode. Small differences between the three sequences include an exposed free CYS residue on 1C2A6 and a reduced number of predicted pi interactions at the VH-VL interface of 8D10F4. 3F12E9 has similarity to 1C2A6, 8D10F4 and 8A9F9 in the CDR regions, but also several important differences. mAb 1D4G7 is likely to bind in a different mode or to a completely different epitope than the other mAbs mentioned above.

### Example 6—In Vivo Protection Against ZIKV Infection and Pathogenesis Through Passive Antibody Transfer and Active Immunization with a prMEnv DNA Vaccine

In this study, novel, synthetic, DNA vaccine targeting the pre-membrane+envelope proteins (prMEnv) of ZIKV generated and evaluated for in vivo efficacy. Following initial in vitro development and evaluation studies of the plasmid construct, mice and non-human primates were immunized with this prMEnv DNA-based immunogen through electroporation-mediated enhanced DNA delivery. Vaccinated animals were found to generate antigen-specific cellular and humoral immunity and neutralization activity. In mice lacking receptors for interferon (IFN)-α/β (designated IFNAR^{-/-}) immunization with this DNA vaccine induced, following in vivo viral challenge, 100% protection against infection-associated weight loss or death in addition to preventing viral pathology in brain tissue. In addition, passive transfer of non-human primate anti-ZIKV immune serum protected IFNAR^{-/-} mice against subsequent viral challenge. This initial study of this ZIKV vaccine in a pathogenic mouse model supports the importance of immune responses targeting prME in ZIKV infection and suggests that additional research on this vaccine approach may have relevance for ZIKV control in humans.

### Cells, Virus and Animals

Human embryonic kidney 293T (American Type Culture Collection (ATCC) #CRL-N268, Manassas, Va., USA) and Vero CCL-81 (ATCC #CCL-81) cells were maintained in DMEM (Dulbecco's modified Eagle's medium; Gibco-Q3 Invitrogen) supplemented with 10% fetal bovine serum (FBS) and 1% penicillin and streptomycin and passaged upon confluence. Both ZIKV virus strains MR766 (a kind gift from Dr Susan Weiss) and PR209 (Bioqual, MD) were amplified in Vero cells and stocks were titred by standard plaque assay on Vero cells. Five- to six-week-old female C57BL/6 (The Jackson Laboratory) and IFNAR^{-/-} (MMRRC repository—The Jackson Laboratory) mice were housed and treated/vaccinated in a temperature-controlled, light-cycled facility in accordance with the National Institutes of Health, Wistar and the Public Health Agency of Canada IACUC (Institutional Animal Care and Use Committee) guidelines.

The RMs were housed and treated/vaccinated at Bioqual, MD, USA. This study was carried out in strict accordance with the recommendations described in the Guide for the Care and Use of Laboratory Animals of the NIH, the Office of Animal Welfare, and the U.S. Department of Agriculture. All animal immunization work was approved by the Bioqual Animal Care and Use Committee (IACUC). Bioqual is accredited by the American Association for Accreditation of Laboratory Animal Care. All the procedures were carried out under ketamine anesthesia by trained personnel under the supervision of veterinary staff, and all the efforts were made to protect the welfare of the animals and to minimize animal suffering in accordance with the ‘Weatherall report for the use of non-human primates’ recommendations. The animals were housed in adjoining individual primate cages allowing social interactions, under controlled conditions of humidity, temperature and light (12 h light/12 h dark cycles). Food and water were available ad libitum. The animals were monitored twice daily and fed commercial monkey chow, treats and fruits twice daily by trained personnel.

### Construction of ZIKV-prME DNA Vaccine

The ZIKV-prME plasmid DNA constructs encodes full-length precursor of membrane (prM) plus envelope (E) and Capsid proteins were synthesized. A consensus strategy was used and the consensus sequences were determined by the alignment of current ZIKV prME protein sequences. The vaccine insert was genetically optimized (i.e., codon and RNA optimization) for enhanced expression in humans and an IgE leader sequence was added to facilitate expression. The construct was synthesized commercially (Genscript, NJ, USA), and then subcloned into a modified pVax1 expression vector under the control of the cytomegalovirus immediate-early promoter as described before (Muthumani et al., 2016, Sci Transl Med 7:301ra132). The final construct is named ZIKV-prME vaccine and the control plasmid backbone is pVax1. In addition, a number of other matched DNA constructs encoding the prM and E genes from MR766 (DQ859059.1) and a 2016 Brazilin (AMA12084.1) outbreak strain were also designed, for further evaluation. Large-scale amplifications of DNA constructs were carried out by Inovio Pharmaceuticals Inc. (Plymouth Meeting, Pa., USA) and purified plasmid DNA was formulated in water for immunizations. The size of the DNA inserts was confirmed via agarose gel electrophoresis. Phylogenetic analysis was performed by multiple alignment with ClustalW using MEGA version 5 software (Muthumani et al., 2016, Sci Transl Med 7:301ra132).

DNA immunizations and electroporation-mediated delivery enhancement Female C57BL/6 mice (6-8 weeks old) and IFNAR^{-/-} mice (5-6 weeks old) were immunized with 25 μg of DNA in a total volume of 20 or 30 μl of water delivered into the tibialis anterior muscle with in vivo electroporation delivery. In vivo electroporation was delivered with the CELLECTRA adaptive constant current electroporation device (Inovio Pharmaceuticals) at the same site immediately following DNA injection. A three-pronged CELLECTRA minimally invasive device was inserted ˜2 mm into the muscle. Square-wave pulses were delivered through a triangular three-electrode array consisting of 26-gauge solid stainless steel electrodes and two constant current pulses of 0.1 Amps were delivered for 52 μs/pulse separated by a 1 s delay. Further protocols for the use of electroporation have been previously described in detail (Flingai et al., 2015, Sci Rep 5:12616). The mice were immunized three times at 2-week intervals and killed 1 week after the final immunization. The blood was collected after each immunization for the analysis of cellular and humoral immune responses (Muthumani et al., 2016, Sci Transl Med 7:301ra132). Rhesus macaque immunogenicity studies: five rhesus macaques were immunized intradermally at two sites two times at 5-week intervals with 2 mg ZIKV-prME vaccine. Electroporation was delivered immediately using the same device described for mouse immunizations.

### Western Blot Analysis

For in vitro expression studies, transfections were performed using the GeneJammer reagent, following the manufacturer's protocols (Agilent). Briefly, the cells were grown to 50% confluence in a 35 mm dish and transfected with 1 μg of ZIKV-prME vaccine. The cells were collected 2 days after transfection, washed twice with PBS and lysed with cell lysis buffer (Cell Signaling Technology). Western Blot was used to verify the expression of the ZIKV-prME protein from the harvested cell lysate and the immune specificity of the mouse and RM serum through the use of either anti-Flavivirus or immune sera from the ZIKV-prME vaccinated mice, as described previously (Muthumani et al., 2016, Sci Transl Med 7:301ra132). In brief, 3-12% Bis-Tris NuPAGE gels (Life Technologies) were loaded with 5 μg or 1 μg of ZIKV envelope recombinant protein (rZIKV-E); transfected cell lysates or supernatant and the Odyssey protein Molecular Weight Marker (Product #928-40000). The gels were run at 200 V for 50 min in MOPS buffer. The proteins were transferred onto nitrocellulose membranes using the iBlot 2 Gel Transfer Device (Life Technologies). The membranes were blocked in PBS Odyssey blocking buffer (LI-COR Biosciences) for 1 h at room temperature. To detect vaccine expression, the anti-Flavivirus group antigen (MAB10216-Clone D1-4G2-4-15) antibody was diluted 1:500 and the immune serum from mice and RM was diluted 1:50 in Odyssey blocking buffer with 0.2% Tween 20 (Bio-Rad) and incubated with the membranes overnight at 4° C. The membranes were washed with PBST and then incubated with the appropriate secondary antibody (goat anti-mouse IRDye680CW; LI-COR Biosciences) for mouse serum and flavivirus antibody; and goat anti-human IRDye800CW (LI-COR Biosciences) for RM sera at 1:15,000 dilution for mouse sera for 1 h at room temperature. After washing, the membranes were imaged on the Odyssey infrared imager (LI-COR Biosciences).

### Immunofluorescence Assays

For the immunofluorescence assay, the cells were grown on coverslips and transfected with 5 μg of ZIKV-prME vaccine. Two days after transfection, the cells were fixed with 4% paraformaldehyde for 15 min. Nonspecific binding was then blocked with normal goat serum diluted in PBS at room temperature for 1 h. The slides were then washed in PBS for 5 min and subsequently incubated with sera from immunized mice or RM at a 1:100 dilutions overnight at 4° C. The slides were washed as described above and incubated with appropriate secondary antibody (goat anti-mouse IgGAF488; for mouse serum and goat anti-human IgG-AF488 for RM serum; Sigma) at 1:200 dilutions at room temperature for 1 h. After washing, Flouroshield mounting media with DAPI (Abcam) was added to stain the nuclei of all cells. After which, coverslips were mounted and the slides were observed under a microscope (EVOS Cell Imaging Systems; Life Technologies) (Muthumani et al., 2016, Sci Transl Med 7:301ra132). In addition, Vero, SK-N-SH or U87-MB cells were grown on four-chamber tissue culture treated glass slides and infected at MOI of 0.01 with ZIKV-MR766 or PR209 that were preincubated with/without RM immune sera (1:200), and stained at 4 days post ZIKV infection using pan flavirus antibody as described (Rossi et al., 2016, J Rop Med Hyg 94:1362-9).

### Histopathology Analysis

For histopathology, formalin-fixed, paraffin-embedded brain tissue was sectioned into 5 μm thick sagittal sections, placed on Superfrost microscope slides (Fisher Scientific) and backed at 37° C. overnight. The sections were deparaffinised using two changes of xylene and rehydrated by immersing in 100%, 90% and then 70% ethanol. The sections were stained for nuclear structures using Harris haematoxylin (Surgipath) for 2 min followed by differentiation in 1% acid alcohol (Surgipath) and treatment with Scott's tap water for 2 min. Subsequently, the sections were counterstained for cytoplasmic structures using eosin (Surgipath) for 2 min. The slides were dehydrated with 70%, 90% and 100% ethanol, cleared in xylene and mounted using Permount (Fisher Scientific).

### Splenocyte and PBMC Isolation

Single-cell suspensions of splenocytes were prepared from all the mice. Briefly, the spleens from mice were collected individually in 5 ml of RPMI 1640 supplemented with 10% FBS (R10), then processed with a Stomacher 80 paddle blender (A.J. Seward and Co. Ltd.) for 30 s on high speed. The processed spleen samples were filtered through 45 mm nylon filters and then centrifuged at 1,500 g for 10 min at 4° C. The cell pellets were resuspended in 5 ml of ACK (ammonium-chloride-potassium) lysis buffer (Life Technologies) for 5 min at room temperature, and PBS was then added to stop the reaction. The samples were again centrifuged at 1,500 g for 10 min at 4° C. The cell pellets were resuspended in R10 and then passed through a 45 mm nylon filter before use in ELISpot assay and flow cytometric analysis (Muthumani et al., 2016, Sci Transl Med 7:301ra132). For RM, blood (20 ml at each time point) was collected in EDTA tubes and the PBMCs were isolated using a standard Ficoll-hypaque procedure with Accuspin tubes (Sigma-Aldrich, St. Louis, Mo., USA). Five millitres of blood was also collected into sera tubes at each time point for sera isolation.

### Flow Cytometry and Intracellular Cytokine Staining Assay

The splenocytes were added to a 96-well plate (2×10⁶/well) and were stimulated with ZIKV-prME pooled peptides for 5 h at 37° C./5% CO2 in the presence of Protein Transport Inhibitor Cocktail (brefeldin A and monensin; eBioscience). The cell stimulation cocktail (plus protein transport inhibitors; PMA (phorbol 12-myristate 13-acetate), ionomycin, brefeldin A and monensin; eBioscience) was used as a positive control and R10 media as the negative control. All the cells were then stained for surface and intracellular proteins as described by the manufacturer's instructions (BD Biosciences, San Diego, Calif., USA). Briefly, the cells were washed in FACS buffer (PBS containing 0.1% sodium azide and 1% FBS) before surface staining with flourochrome-conjugated antibodies. The cells were washed with FACS buffer, fixed and permeabilised using the BD Cytofix/Ctyoperm™ (BD Biosciences) according to the manufacturer's protocol followed by intracellular staining. The following antibodies were used for surface staining: LIVE/DEAD Fixable Violet Dead Cell stain kit (Invitrogen), CD19 (V450; clone 1D3; BD Biosciences) CD4 (FITC; clone RM4-5; eBioscience), CD8 (APC-Cy7; clone 53-6.7; BD Biosciences); CD44 (BV711; clone IM7; BioLegend). For intracellular staining, the following antibodies were used: IFN-γ (APC; clone XMG1.2; BioLegend), TNF-α (PE; clone MP6-XT22; eBioscience), CD3 (PerCP/Cy5.5; clone 145-2C11; BioLegend); IL-2 (PeCy7; clone JES6-SH4; eBioscience). All the data were collected using a LSRII flow cytometer (BD Biosciences) and analyzed using FlowJo software (Tree Star, Ashland, Oreg., USA).

### ELISpot Assay

Briefly, 96-well ELISpot plates (Millipore) were coated with anti-mouse IFN-γ capture Ab (R&D Systems) and incubated overnight at 4° C. The following day, the plates were washed with PBS and blocked for 2 h with PBST+1% BSA. Two hundred thousand splenocytes from immunized mice were added to each well and incubated overnight at 37° C. in 5% CO₂ in the presence of media alone (negative control), media with PMA/ionomycin (positive control) or media with peptide pools (1 μg/ml) consisting of 15-mers overlapping by nine amino acids and spanning the length of the ZIKV prME protein (Genscript). After 24 h, the cells were washed and then incubated overnight at 4° C. with biotinylated anti-mouse IFN-γ Ab (R&D Systems). Streptavidin-alkaline phosphatase (R&D Systems) was added to each well after washing and then incubated for 2 h at room temperature. The plate was washed, and then 5-bromo-4-chloro-3′-indolylphosphate p-toluidine salt and nitro blue tetrazolium chloride (chromogen colour reagent; R&D Systems) was added. Last, the plates were rinsed with distilled water, dried at room temperature and SFU were quantified by an automated ELISpot reader (CTL Limited), and the raw values were normalised to SFU per million splenocytes. For RM samples, the ELISPOT^{PRO} for monkey IFN-γ kit (MABTECH) was used as described by the manufacturer; two hundred thousand PBMCs were stimulated with peptide pools; and the plates were washed and spots were developed and counted as described before (Muthumani et al., 2016, Sci Transl Med 7:301ra132).

### Humoral Immune Response: Antibody-Binding ELISA

An ELISA was used to determine the titers of mouse and RM sera as previously described (Muthumani et al., 2016, Sci Transl Med 7:301ra132). Briefly, 1 μg of purified rZIKV-E protein was used to coat 96-well microtiter plates (Nalgene Nunc International, Naperville, Ill., USA) at 4° C. overnight. After blocking with 10% FBS in PBS for at least an hour, the plates were washed four times with 0.05% PBST (Tween20 in PBS). Serum samples from immunized mice and RMs were serially diluted in 1% FBS, added to the plates, then incubated for 1 h at room temperature. The plates were again washed four times in 0.05% PBST, then incubated with HRP-conjugated anti-mouse IgG (Sigma) at a 1:35,000 dilution for mouse sera for 1 h at room temperature. For RM sera, anti-monkey IgG HRP (Southern Biotech) was used at a 1:5,000 dilutions for 1 h at room temperature. The bound enzyme was detected by adding SIGMAFAST OPD (o-phenylenediamine dihydrochloride) substrate solution according to the manufacturer's instructions (Sigma-Aldrich). The reaction was stopped after 15 min with the addition of 1 N H₂SO₄. The optical density at 450 nm was read on a Synergy plate reader. All the mouse and RM serum samples were assayed in duplicate. End point titers were determined using the method described previously (Frey et al., 1998, J Immunol Methods 21:35-41).

### Neutralization (PRNT₅₀) Assay

The PRNT involving MR766 and Vero cells was described previously (Sun et al., 2006, J Infect Dis 193:1658-65). Briefly, heat-inactivated mouse or RM sera were serially diluted in serum-free DMEM (1:10 to 1:1280) and incubated with an equal volume of ZIKV MR766 (100 PFU) at 37° C. for 2 h. The mixtures were added to the confluent layers of Vero cells and left at 37° C. for adsorption for 2 h. A 2×DMEM media:soft-agar (1:1) overlay was added over cells and the plate was incubated for 5 days at 37° C. The agar overlay was removed and the cells were fixed with 4% paraformaldehyde, washed with 1×PBS, stained with crystal violet solution, washed with 1×PBS and the plates were left to dry. The plaques in assays done in 24-well plates were scanned with an automated Immunospot reader (CTL Limited), and the plaques in sample wells and in negative control (DMEM only) and positive control (100 PFU MR766 ZIKV virus only) wells were counted using the automated software provided with the ELISpot reader. The percentage plaque reduction was calculated as follows: % reduction=100×{1−(average number of plaques for each dilution/average number of plaques in positive control wells)}. GraphPad Prism software was used to perform nonlinear regression analysis of % plaque reduction versus a log transformation of each individual serum dilution to facilitate linear interpolation of actual 50% PRNT titers at peak post vaccination response. The medians and interquartile ranges at 50% neutralization were calculated for each neutralization target overall and by vaccine treatment group; the geometric mean titers were also calculated. The titers represent the reciprocal of the highest dilution resulting in a 50% reduction in the number of plaques.

### ZIKV Challenge Studies in IFNAR^{-/-} Mice

For the ZIKA challenge studies, IFNAR^{-/-} mice (n=10/group) were immunized once or twice with the ZIKA-prME vaccine or pVax1. The mice were with either 1×10⁶ PFU or 2×10⁶ PFU ZIKV-PR209 virus on day 15 (single immunization group) or day 21 one week after the second immunization (two immunization groups). Also, additional groups of IFNAR^{-/-} mice (n=10/group) were immunized once and challenged with 2×10⁶PFU ZIKV-PR209 virus on day 15. Post challenge, the animals were weighed and body temperature was measured daily by a subcutaneously located temperature chip. In addition, they were observed for clinical signs of disease twice daily (decreased mobility; hunched posture; hind-limb knuckle walking (partial paralysis), paralysis of one hind limb or both hind limbs) and the blood was drawn for viral load determination. The criteria for killing on welfare grounds consisted of 20% weight loss or paralysis in one or both hind limbs.

### Real-Time RT-PCR Assay for Measurement of ZIKV Load

The brains from treated mice were immersed in RNAlater (Ambion) 4° C. for 1 week, then stored at −80° C. The brain tissue was then weighed and homogenized in 600 μl RLT buffer in a 2 ml cryovial using a TissueLyser (Qiagen) with a stainless steel bead for 6 min at 30 cycles/s. Viral RNA was also isolated from blood with the RNeasy Plus mini kit (Qiagen). A ZIKV specific real-time RT-PCR assay was utilized for the detection of viral RNA from subject animals. RNA was reverse transcribed and amplified using the primers ZIKV 835 and ZIKV 911c and probe ZIKV 860FAM with the TaqMan Fast Virus 1-Step Master Mix (Applied Biosystems). A standard curve was generated in parallel for each plate and used for the quantification of viral genome copy numbers. The StepOnePlus Real-Time PCR System (ABI) software version 2.3 was used to calculate the cycle threshold (Ct) values, and a Ct value ≤38 for at least one of the replicates was considered positive, as previously described (Lanciotti et al., 2008, Emerg Infect Dis 14:1232-9). Pre-bleeds were negative in this assay.

### Statistical Analysis

Differences in fold increases in antibody titers were compared using Mann-Whitney analysis. Statistical analysis was performed using Graphpad, Prism 4 (Graphpad software, Inc. San Diego, Calif., USA). For all the analyses, P<0.05 was considered to be significant. Log₁₀ transformations were applied to end point binding ELISA titers and whole-virus PRNT₅₀ titers.

### Construction of the ZIKV-prME Consensus DNA Vaccine

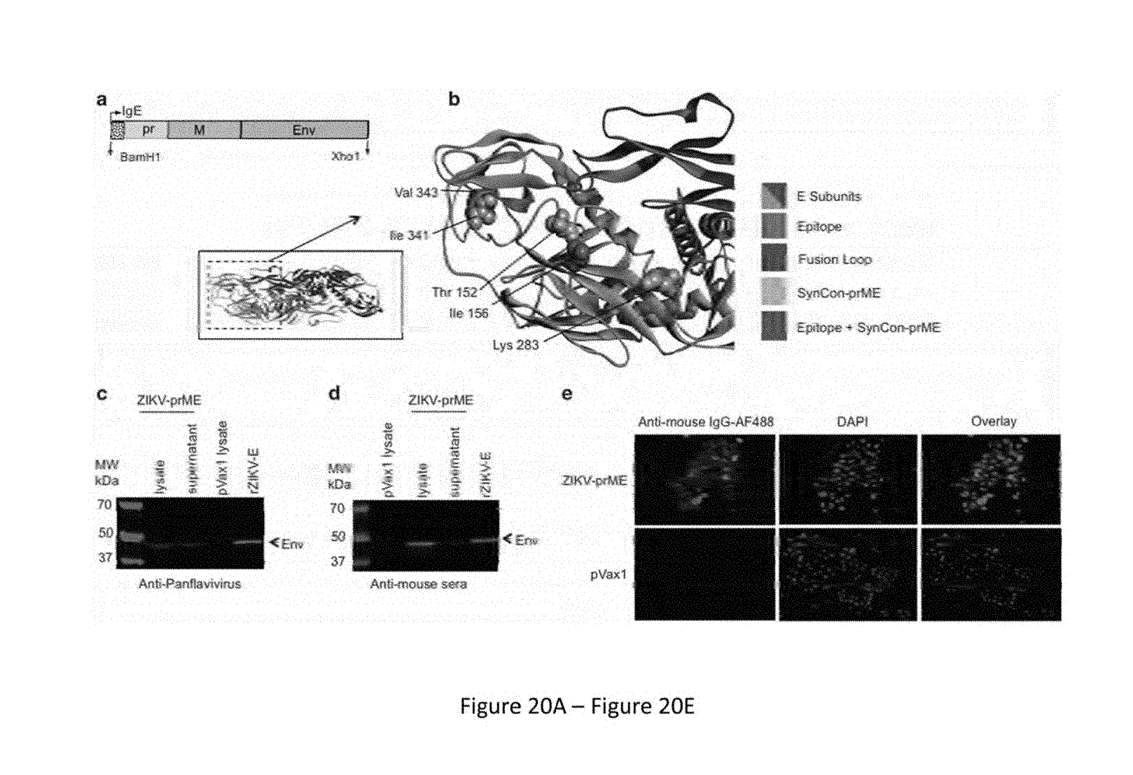

A consensus sequence of ZIKV prM (precursor membrane) and Env (envelope) genes (ZIKV-prME) was generated using prM and Env sequences from various ZIKV isolated between the years of 1952 and 2015, which caused infection in humans. The ZIKV-prME consensus sequence was cloned into the pVax1 vector after additional modifications and optimizations were made to improve its in vivo expression including the addition of a highly efficient immunoglobulin E (IgE) leader peptide sequence (FIG. 20A). Optimal alignment of ZIKV-envelope sequences was performed using homology models and visualization on Discovery Studio 4.5. Reference models included PDB 5JHM and PDB 5IZ7. Aligned residues corresponding to specific regions on the prME antigen were labelled in the models for visualization purposes (FIG. 20B). The optimized consensus vaccine selections are in general conservative or semi-conservative relative to multiple ZIKV strains analyzed in this study. Structural studies of EDE-specific neutralizing antibodies have revealed that these recognition determinants can be found at a serotype-invariant site at the envelope-dimer interface, which includes the exposed main chain of the fusion loop and two conserved glycan chains (N67- and N153-linked glycans) (Rouvinski et al., 2015, Nature 520:109-13). These two glycosylation sites are not highly conserved in other flaviviruses. Moreover, ZIKV does not possess the N67-linked glycosylation site, and the N154-linked glycosylation site (equivalent to the N153-linked glycosylation site in dengue) is absent in some of the isolated ZIKV strains. As part of the consensus design, therefore the construct was designed leaving out this glycosylation site. Lack of glycosylation at this site has been correlated with improved binding of EDE1 type broadly neutralizing antibodies (bnAbs) to ZIKV-envelope protein (Rouvinski et al., 2015, Nature 520:109-13).

Subsequent to construction, expression of the ZIKV-prME protein from the plasmid was confirmed by western blot analysis and an indirect immunofluorescence assay. The protein extracts prepared from the cells transiently transfected with ZIKV-prME were analyzed for expression by western blot using panflavivirus antibody (FIG. 20C) and sera collected from ZIKV-prME immunized mice (FIG. 20D). ZIKV-prME expression was further detected by IFA by the staining of 293T cells transfected with ZIKV-prME plasmid at 48 h post transfection with anti-ZIKV-prME specific antibodies (FIG. 20E).

ZIKV-prMEnv DNA Vaccine Induces Antigen-Specific T Cells in C57BL/6 Mice

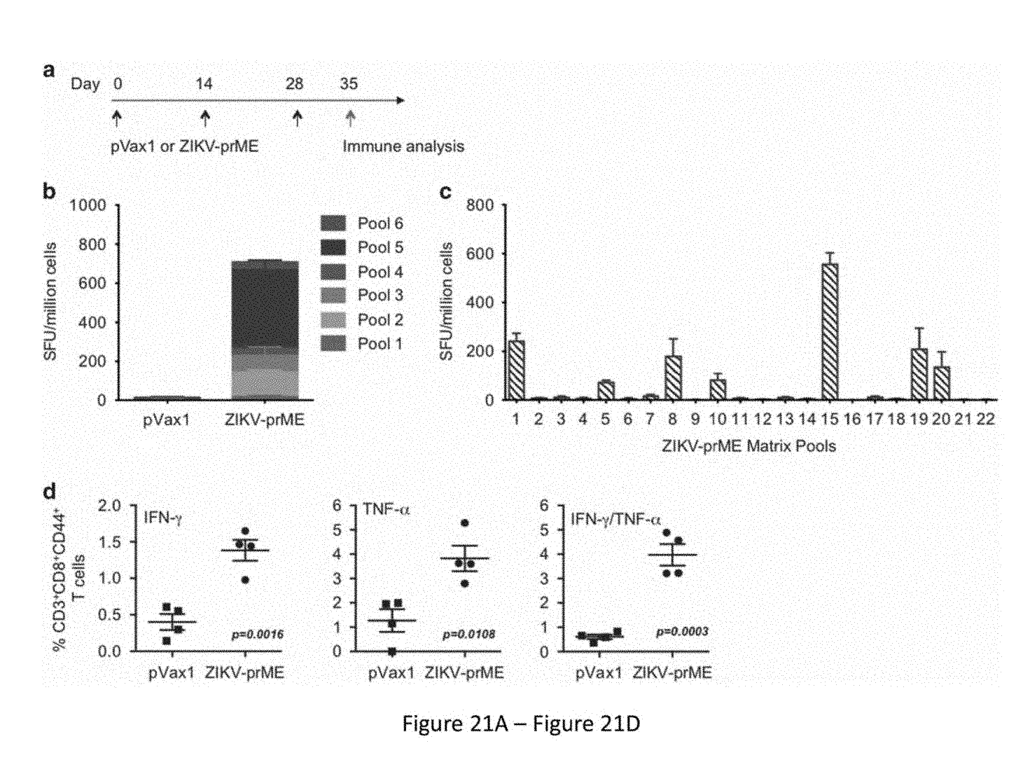

The ability of the ZIKV-prMEnv plasmid vaccine to induce cellular immune responses was evaluated. Groups of four female C57BL/6 mice were immunized with either the control plasmid backbone (pVax1) or the ZIKV-prME plasmid vaccine three times at 2 week intervals through intramuscular (i.m.) injection followed by electroporation at the site of delivery (FIG. 21A). The animals were killed 1 week after their third injection and bulk splenocytes harvested from each animal were evaluated in ELISpot assays for their ability to secrete interferon-γ (IFN-γ) after ex vivo exposure to peptide pools encompassing ZIKV-prME is included. The assay results show that splenocytes from ZIKV-prME immunized mice produced a cellular immune response after stimulation with multiple ZIKV-E peptide pools (FIG. 21B). The region(s) of ZIKVEnv, which elicited the strongest cellular response(s) were evaluated by ELISpot assay in a matrix format using 22 peptide pools consisting of 15-mers (overlapping by 11 amino acids) spanning the entire ZIKV-prME protein. Several pools demonstrated elevated T cell responses, with peptide pool 15 exhibiting the highest number of spot-forming units (SFU) (FIG. 21C). This matrix mapping analysis revealed a dominant prME epitope, ‘IRCIGVSNRDFVEGM (SEQ ID NO:17)’ (aa167-181). This peptide was confirmed to contain a H2-Db restricted epitope through analysis utilising the Immune Epitope Database Analysis Resource tool, which supports that in this haplotype the antigen is effectively processed.

Further evaluation of the cellular immunogenicity of the ZIKV-prMEnv vaccine entailed the determination of the polyfunctional properties of CD8⁺ T cells collected 1 week after the final immunization. The results show that the ZIKV-prMEnv vaccination increased the proportion of bifunctional vaccine-specific T cells expressing TNF-α (tumour necrosis factor-α) and IFN-γ. Importantly, ZIKV-prMEnv vaccination exhibited a strong ability to expand T cell functionality (FIG. 21D).

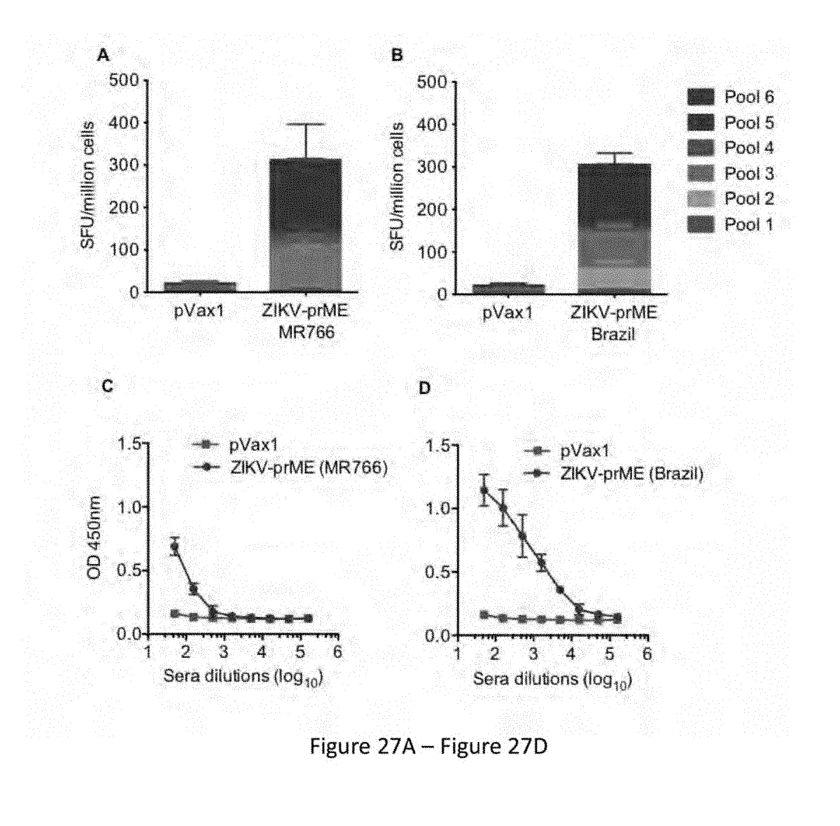

In addition, comparative immune studies were performed with optimized plasmids encoding the prMEnv sequence of either a recently identified Brazilian ZIKV strain or of the original MR766 ZIKV strain. Induction of cellular immune responses in mice immunized with either plasmid was measured 1 week after the third vaccination through IFN-γ ELISpot analysis after stimulating splenocytes with the ZIKV-prMEnv peptide pools. The results illustrate that the T-cell responses induced by the consensus ZIKVprME DNA vaccine construct were consistently higher than those generated by either of these two non-consensus plasmid vaccines (FIGS. 27A and 27B). Detailed mapping analysis of the cellular responses induced by either the Brazilian or MR766 prME vaccines revealed that both vaccines induced significant cellular response against the dominant Env-specific CTL epitope as identified in FIG. 21B and FIG. 21C for the consensus ZIKV-prMEnv plasmid (data not shown). The consensus immunogen consistently induced more robust responses in these T-cell assays at the same dose and was evaluated further in additional assays.

### Generation of a ZIKV Recombinant Envelope Protein

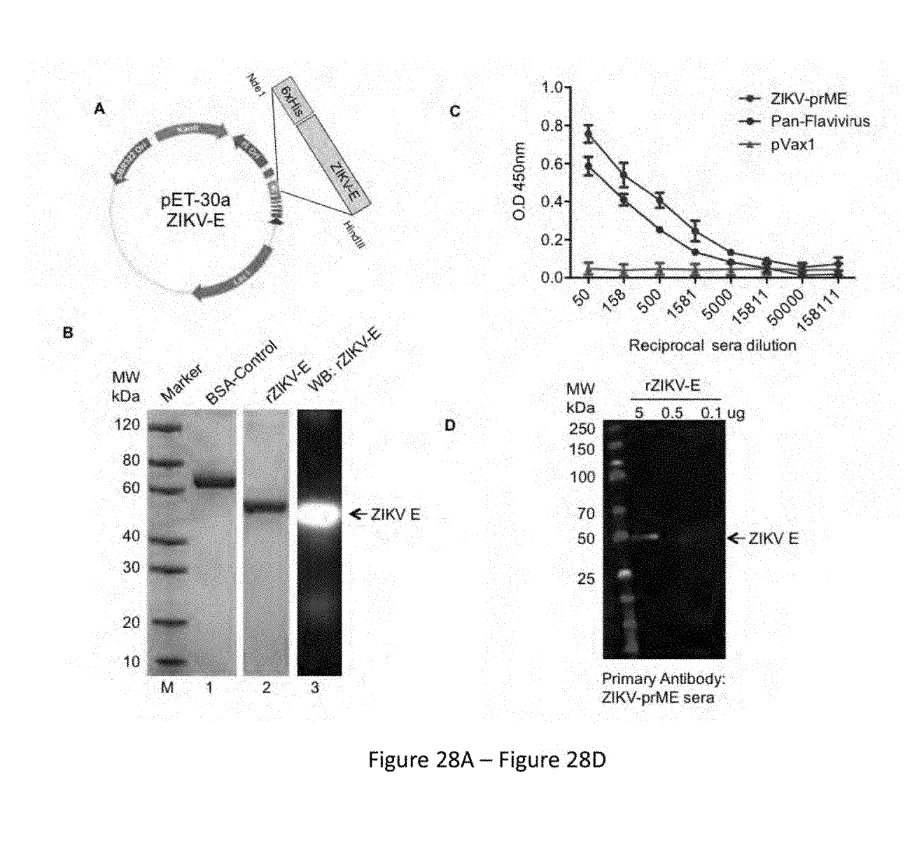

At the onset of these studies, there were no available commercial reagents to evaluate specific anti-ZIKV immune responses. Therefore, by necessity, recombinant ZIKV-envelope protein (rZIKV-E) was generated to support the assays performed in this study. To generate this reagent, a consensus ZIKV-Envelope sequence based on the ZIKV-prME vaccine consensus antigen was cloned into a pET30a Escherichia coli expression vector (FIG. 28A). The rZIKV-E antigen was produced in E. coli cultures, purified using nickel column chromatography and analyzed using SDS-PAGE, which showed overexpressed proteins of the predicted size in lysate from rZIKV-E transfected bacteria that could be detected by western analysis using an anti-His tag antibody (FIG. 28B). The sera from mice immunized with the ZIKV-prME vaccine bound to rZIKV-Env that was used as a capture antigen in an ELISA (enzyme-linked immunosorbent assay; FIG. 28C). A commercial antibody (designated panflavivirus) that reacts to the envelope protein of multiple flaviviruses, also bound to rZIKV-E. Western analysis demonstrated that immune sera from ZIKV-prMEnv immunized mice specifically recognized rZIKV-E (FIG. 28D). These data indicate that the generated rZIKV-E reacted specifically with immune sera from ZIKV-prMEnv vaccinated mice, thus this recombinant protein was used for further immunogenicity studies.

### Induction of Functional Humoral Responses in C57BL/6 Mice by the ZIKV-prME DNA Vaccine

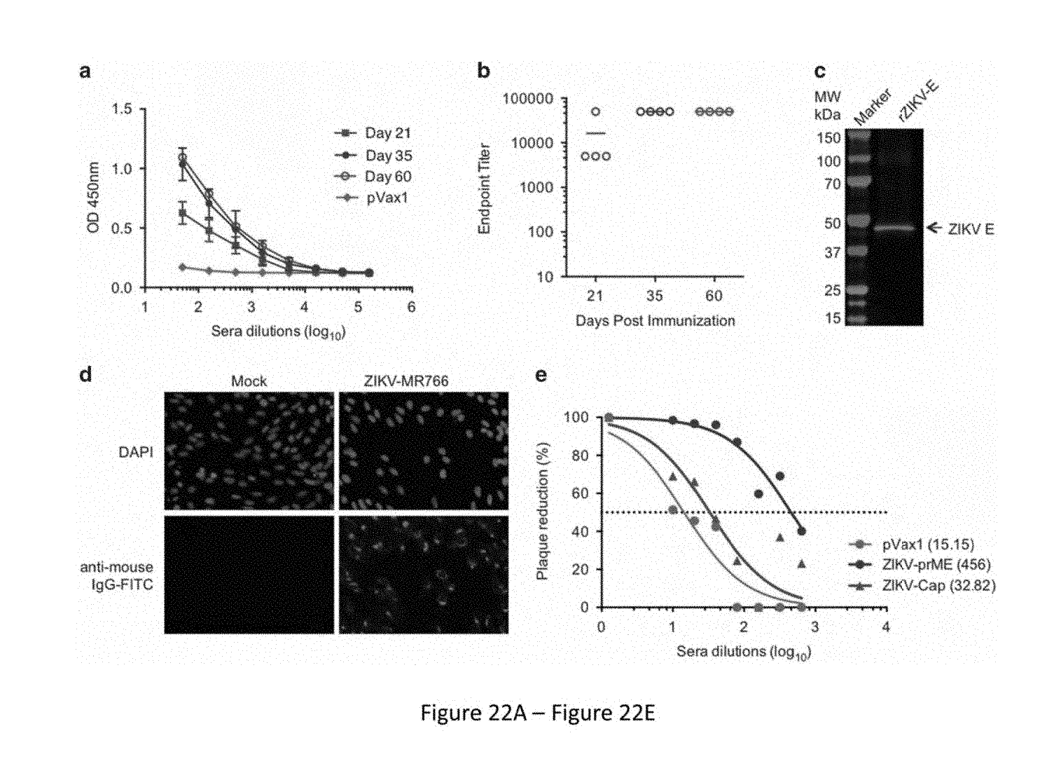

The ability of the consensus ZIKV-prMEnv vaccine to induce humoral immune responses in mice was evaluated. Groups of four C57BL/6 mice were immunized intramuscularly (i.m.) through electroporation-mediated delivery three times at 2-week intervals with 25 μg of either the empty control pVax1 or the consensus ZIKV-prMEnv vaccine plasmids. The sera were obtained from each immunized mouse and were tested by ELISA for ZIKV-specific IgG responses using immobilized rZIKV-E as the capture antigen. A significant increase in anti-ZIKV-specific IgG was observed on day 21 with a further boost in the sera IgG levels noted on day 35 (FIG. 22A). Day 60 sera from vaccinated animals show that elevated ZIKV-specific antibody responses were maintained long term following the final boost. Most importantly, the sera from vaccinated mice contained very high levels of rZIKV-E-specific antibodies as indicated by the end point titers (FIG. 22B). Additional assessment of the specificity of the vaccine-induced antibodies was performed by screening pooled sera from ZIKVprMEnv plasmid inoculated mice for its ability to detect rZIKV-E (envelope) by western analysis (FIG. 22C) and to stain ZIKV (MR766 strain)-infected cells by an immunofluorescence assay (FIG. 22D). The results from both these analyses confirmed specificity of the vaccine-induced humoral responses.

Furthermore, ZIKV-specific binding antibody responses were also assessed in mice immunized with plasmids encoding the prMEnv sequences from a Brazilian strain and the MR766 strain described above. Day 35 (1 week after third immunization) sera from pVax1- and both non-consensus vaccine-immunized mice were analyzed by ELISA for binding to rZIKV-E. This analysis indicates that both MR766 and Brazil vaccine plasmids induced significant antibody binding, and that immunization with the consensus ZIKV-prME DNA vaccine generates an effective humoral response against rZIKV-E (FIG. 27C and FIG. 27D).

A plaque reduction neutralization test (PRNT) assay was performed on pooled day 35 sera from mice immunized (3×) with either the control pVax1 plasmid, the consensus ZIKV-prMEnv plasmid vaccine or a consensus ZIKV-C (capsid) plasmid vaccine. The PRNT assay used was a method adapted from a previously described technique for analyzing dengue virus, West Nile virus and other flaviviruses (Davis et al., 2001, J Virol 75:4040-7). As shown in FIG. 22E, ZIKV-prME vaccination yielded significant neutralization response with anti-ZIKV reciprocal PRNT₅₀ dilution titers (inverse of the serum dilution at which 50% of the control ZIKV infection was inhibited) of 456±5, whereas mice vaccinated with the ZIKV-Cap DNA vaccine demonstrated titers (33±6) that were only minimally over pVax1 control plasmid vaccinated animals (titre=15±2).

Immune Responses and Protection Against ZIKV in Mice Lacking the Type I Interferon Receptor (IFNAR^{-/-}) Following Immunization with the ZIKV-prME DNA Vaccine

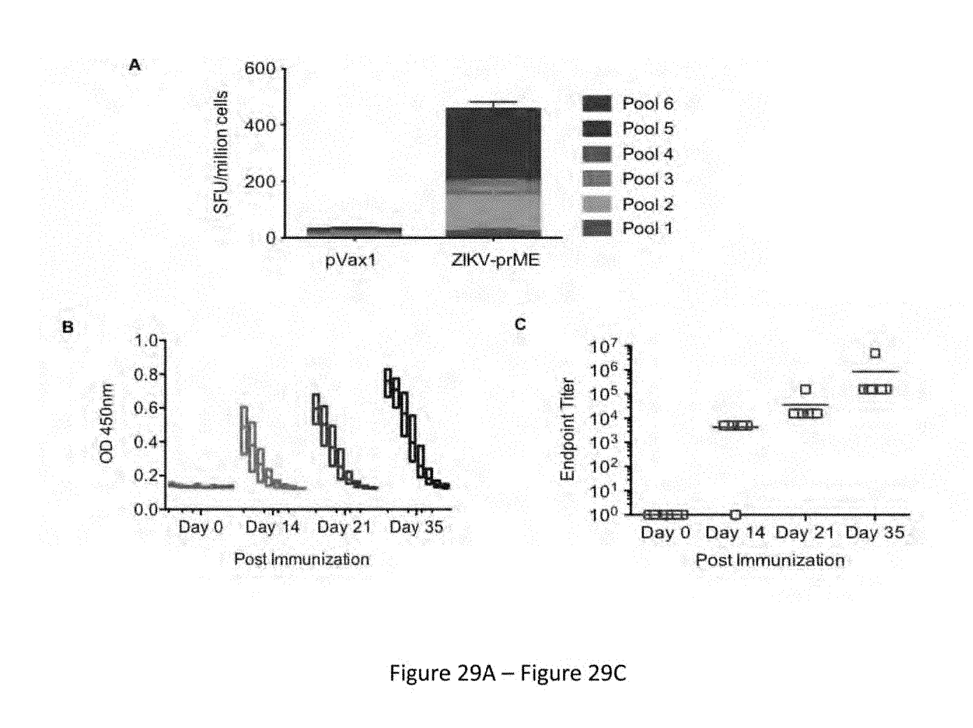

Mechanisms of ZIKV-induced disease and immunity are poorly defined, and the protective versus the hypothetical pathogenic nature of the immune response to ZIKV infection is as yet unclear (Rossi et al., 2016, J Rop Med Hyg 94:1362-9). Most strains of mice are resistant to ZIKV infection, however, mice lacking IFN-α/β receptor (IFNAR^{-/-}) were found to be susceptible to infection and disease with most succumbing within 6-7 days post challenge (Lazear et al., 2016, Cell Host Microbe 19:720-30). The ability of the consensus ZIKV-prME plasmid vaccine to induce cellular and humoral immune responses in this mouse strain was investigated. Five to six week old female IFNAR^{-/-} mice (n=4) were immunized i.m., with electroporation-mediated delivery, three times at 2-week intervals with either the control pVax1 plasmid or ZIKV prME vaccine plasmid vaccine. The serum was collected from immunized mice at days 0, 14, 21, and 35, and splenocytes were harvested from mice 1 week following the final immunization (day 35). The splenocytes from vaccine-immunized mice produced a clear cellular immune response as indicated by levels of SFU per 10⁶ cells in an ELISpot assay (FIG. 29A). The results from ELISA analysis, using rZIKV-E as a capture antigen, show detectable anti-ZIKV serum IgG by day 14 (titers of ˜1:1,000) and these levels were boosted with subsequent vaccinations with binding antibody titers reaching at least 1:100,000 (FIGS. 29B and 29C). By comparison, the PRNT₅₀ titer for the day 35 postimmunization samples was 1:60. The results indicate that IFNAR^{-/-} mice immunized with the consensus ZIKV-prMEnv vaccine are capable of generating anti-ZIKV cellular and humoral immune responses supporting further study in this model of putative vaccine effects in a pathogenic challenge.

### ZIKV-Specific Functional Cellular and Humoral Responses Elicited by the ZIKV-prMEnv DNA Vaccine in Non-Human Primates

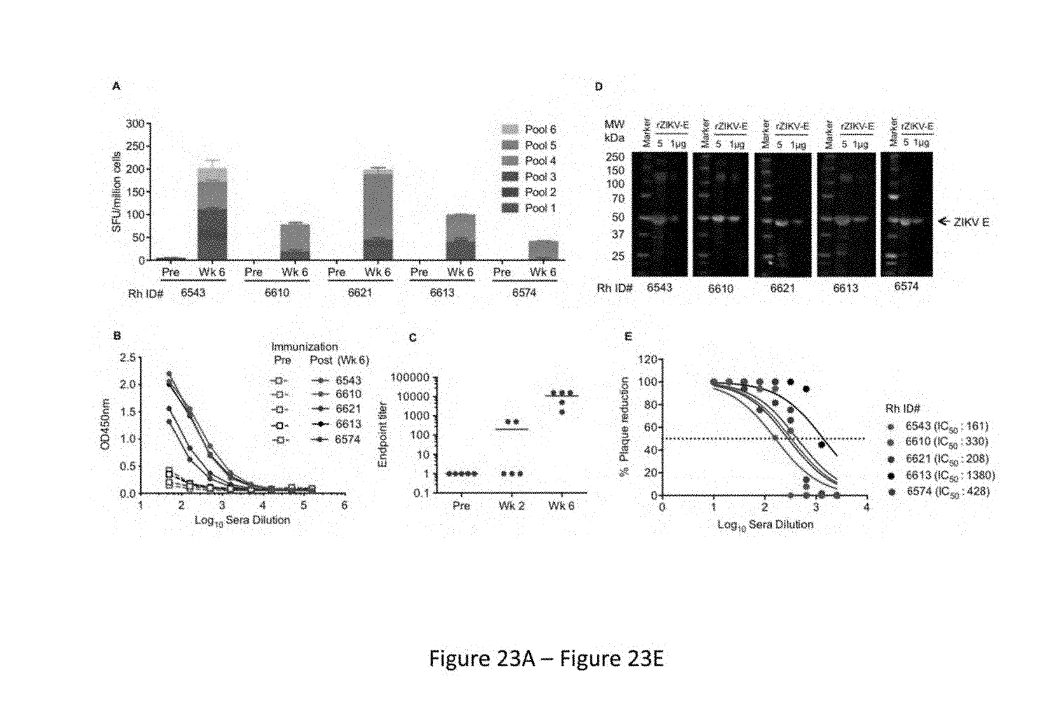

NHPs were immunized by intradermal immunization using intradermal electroporation, based on recent studies showing potent immune responses in a lower voltage intradermal format (Hutnick et al., 2012, Hum gene Ther 23:943-50; Broderick et al., Mol Ther Nucleic Acids 1:e11). Rhesus macaques (RM; n=5/group) were administered 2.0 mg of vaccine plasmid intradermally with electroporation, with each animal vaccinated twice 4 weeks apart. The sera and peripheral blood mononuclear cells (PBMCs) were collected at day 0 (pre-immunization) and week 6 (2 weeks post second immunization). ELISpot analysis of pre-immunization and week 6 PBMCs ex vivo stimulated with the ZIKV-prMEnv peptide pools showed that ZIKV-prMEnv immunization induced robust anti-ZIKV T cell responses in RM (FIG. 23A).

Specific anti-ZIKV antibody responses in sera from vaccinated RM were assessed by ELISA. At week 6, rZIKV-Env-specific binding antibodies were detectable in animals vaccinated with ZIKV-prMEnv (FIG. 23B). End point titers were determined for each animal at week 2 (after 1 immunization) and week 6 (after 2 immunizations; FIG. 23C). The ELISA results were confirmed by western blot analysis using RM sera from the individual vaccinated animals (FIG. 23D). The neutralization activity of the antibodies generated in RM at week 6 was evaluated by a PRNT₅₀ assay. All the vaccinated monkeys had significant neutralization activity with anti-ZIKV reciprocal PRNT₅₀ dilution titers ranging from 161 to 1380 (average 501±224 standard error of the mean; FIG. 23E). PRNT titers did not directly correlate with ELISA titer (data not shown).

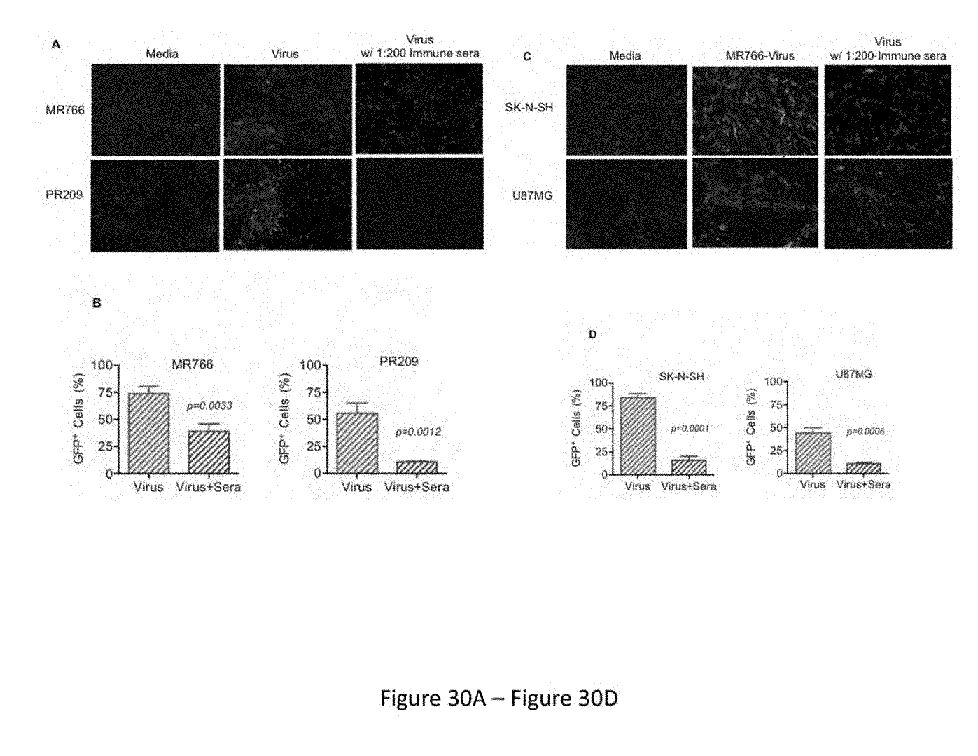

The ability of the NHP vaccine immune sera to block ZIKV infection of Vero cells, neuroblastoma (SK-N-SH) or neural progenitor (U-87MG) cells in vitro was examined by IFA. ZIKV Q2 strains (MR766 or PR209) were pre-incubated in sera or dilution of NHP-immune sera and added to monolayers of each cell type. Four days post infection, ZIKV-positive cells were identified by IFA using pan flavirus antibody (FIGS. 30A-30C) and quantified the ZIKV-positive cells (FIGS. 30B-30D). The sera from ZIKA-prME vaccinated RM inhibited the ZIKV infection in each cell type.

### Protection Against ZIKV Infection and Disease in IFNAR^{-/-} Mice Following ZIKV-prME Immunization

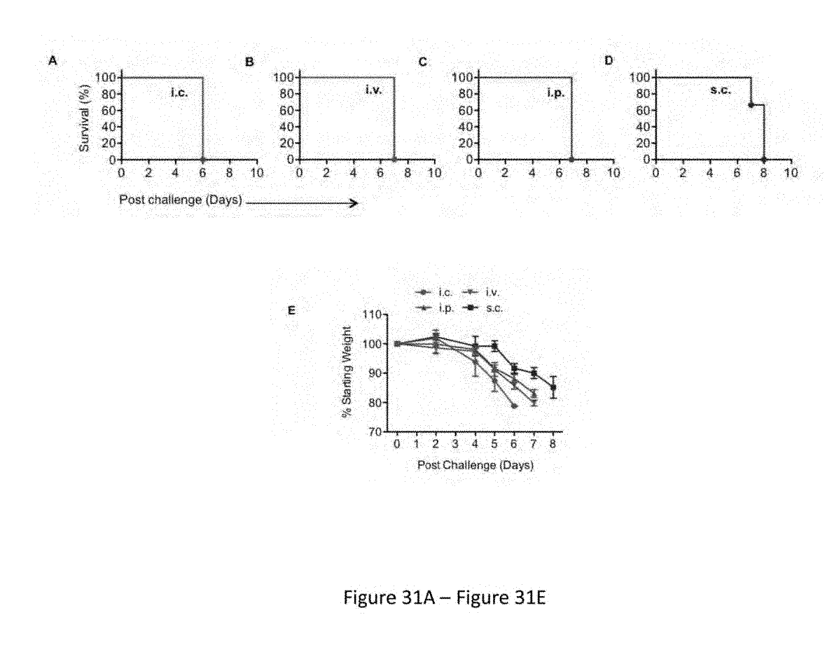

In exploratory studies, 5-6-week-old IFNAR^{(-/-)} mice (n=10) were challenged with 1×10⁶ plaque-forming units (PFU) of the ZIKV-PR209 isolate, administered by either subcutaneous (s.c.); intraperitoneal (i.p.); intracranial; or intravenous (i.v.) routes. After the challenge, all the animals were monitored for clinical signs of infection, which included routine measurement of body weight as well as inspection for other signs of a moribund condition such as hind limb weakness and paralysis. No change in the general appearance of the mice was observed during the first 4 days after inoculation. However, after the fourth day, the mice in each of the groups demonstrated reduced overall activity, decreased mobility and a hunched posture often accompanied by hind-limb weakness, decreased water intake and obvious weight loss. The animals succumbed to the infection between day 6 and day 8 regardless of the route of viral challenge (FIG. 31A-35E). On the basis of these data, the subsequent studies to evaluate ZIKV-prME-mediated protection in this model used the s.c. route for challenge.

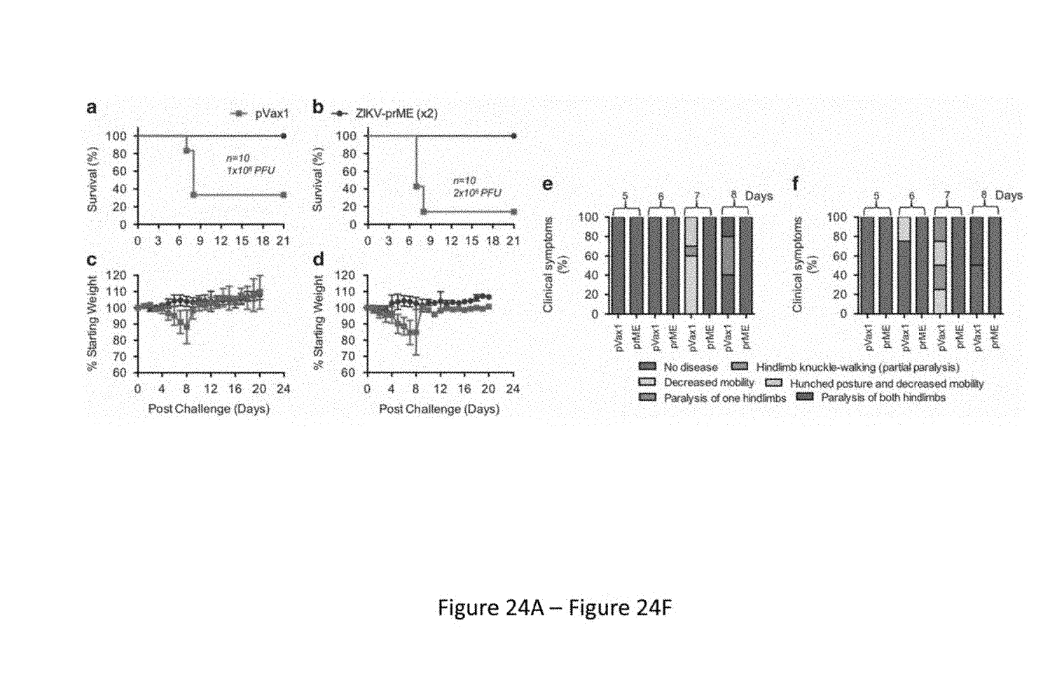

The protective efficacy of the ZIKV-prMEnv vaccine was next evaluated in this IFNAR⁻− mice model. Two groups of mice (n=10) were immunized (25 μg of vaccine) by the i.m. route, through electroporation-mediated delivery with the ZIKV-prME vaccine. Also, two groups of 10 mice were immunized by the i.m. route through electroporation-mediated delivery with the control pVax1 vector. The immunizations were performed two times, two weeks apart, and all the animals were challenged on day 21 (1 week post second immunization). One set of control and vaccinated mice received 1×10⁶ PFU of ZIKV-PR209 by the s.c. route and the other set of each group were challenged with a total of 2×10⁶ PFU ZIKV-PR209 by the s.c. route. At 3 weeks post challenge, 100% of all ZIKV-prME vaccinated animals survived, whereas only 30% of the single- or 10% of double-dose challenged controls survived (FIGS. 24A and 24B). In all the challenges, the vaccinated animals were without signs of disease including no evidence of weight loss (FIGS. 24C and 24D). The infection of control mice with ZIKV-PR209 virus produced a marked decrease in body weight along with decreased mobility, hunched posture, hindlimb knuckle walking and/or paralysis of one or both hind limbs (FIGS. 24E and 24F).

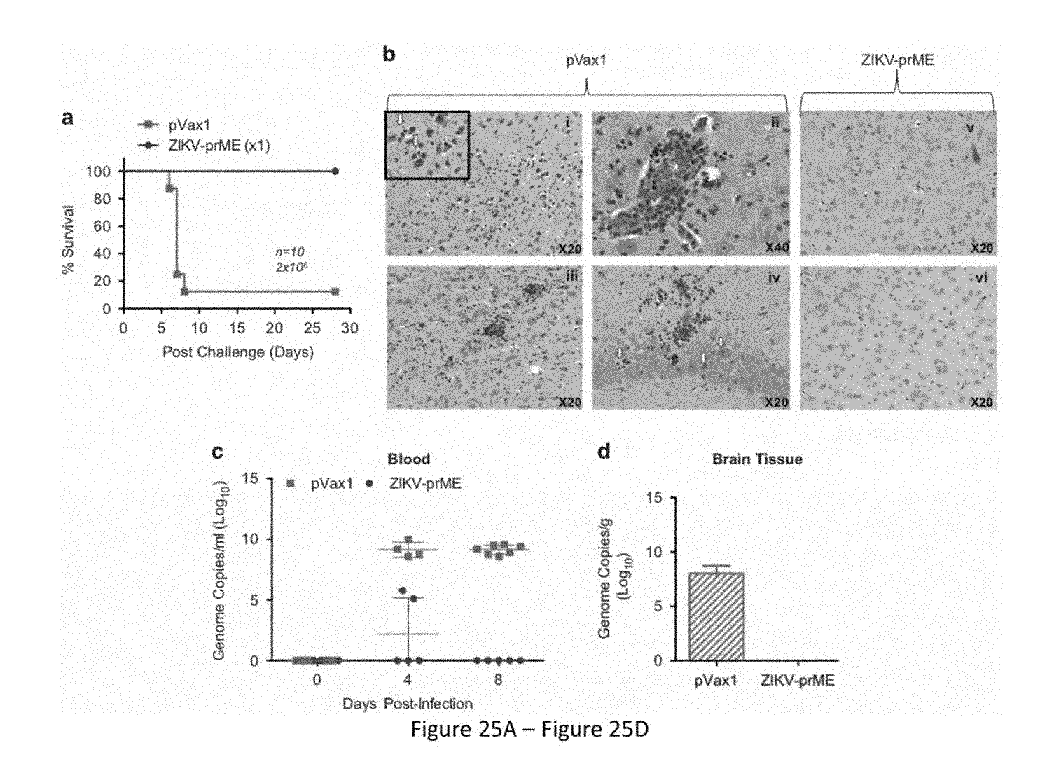

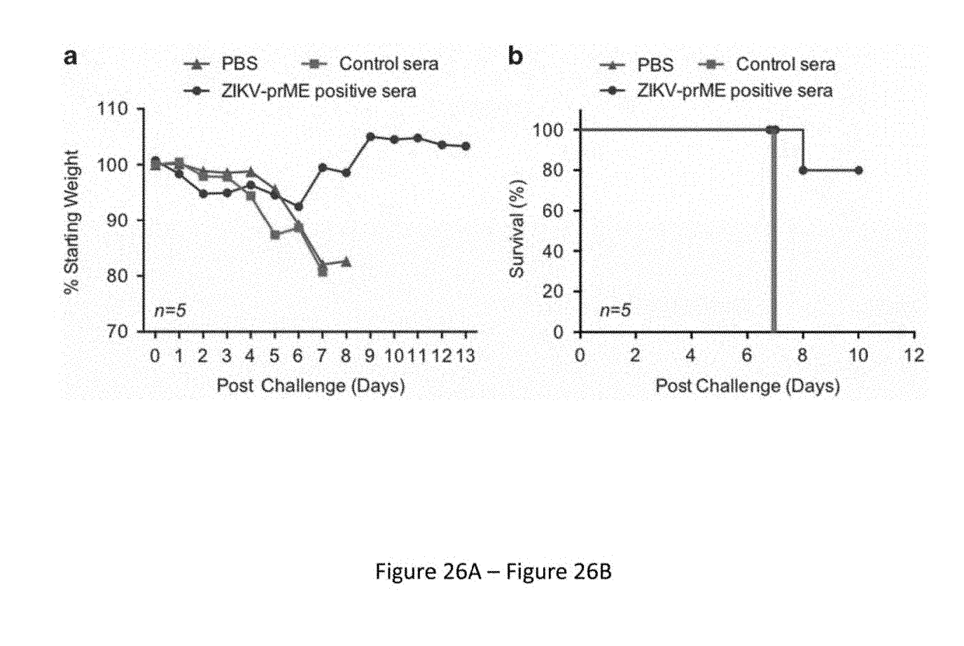

The potential ability of a single immunization with the ZIKVprME DNA vaccine to protect IFNAR^{-/-} mice from ZIKV challenge was evaluated. Groups of 10 mice were immunized i.m. with electroporation once with either control plasmid or ZIKV-prME vaccine and challenged 2 weeks later with a double total dose of 2×10⁶ PFU ZIKV-PR209 administration. Three weeks post challenge, 100% of the ZIKV-prME vaccinated animals survived, whereas only 10% of the control animals survived (FIG. 25A). To determine gross histopathological changes, brain tissue was sectioned into 5 μm-thick sagittal sections, stained for nuclear structures and counterstained for cytoplasmic structures using eosin (FIG. 25B). The mice were killed at day 7 or 8 post challenge for the analysis of histology and viral load. The ZIKV infection caused severe brain pathology in the mice. The unvaccinated control (pVax1) mice brain sections showed nuclear fragments within neutrophils (FIG. 25B); perivascular cuffing of vessel within the cortex, lymphocyte infiltration and degenerating cells of the cerebral cortex (FIG. 25B) and degenerating neurons within the hippocampus (FIG. 25B). In contrast, however, the ZIKV prME vaccinated animals presented with normal histopathology in brain tissues (FIG. 25B) supporting that protective antibodies induced by immunization with the synthetic ZIKA-prME vaccine could limit viral-induced disease in the brain. This observation demonstrates the potential for vaccination to protect the brain in this model. Consistent with the amelioration of body weight loss and mobility impairment in vaccinated mice following ZIKV challenge, a significantly lower viral load was noted in the blood (FIG. 25C) and brain (FIG. 25D) of the ZIKV-prME vaccinated animals compared with viral challenged pVax1 vaccinated animals in the high (2×10⁶ PFU) dose challenge groups. Taken together, these data illustrate that ZIKV-prME DNA vaccine-mediated immune responses can protect mice against ZIKV challenge.

### Passive Transfer of Anti-ZIKV Immune Sera Protects Mice Against ZIKV Infection

Next, whether transfer of immune sera from ZIKV-prMEnv vaccinated RM would prevent ZIKV-mediated pathogenesis in IFNAR^{-/-} mice was tested. To this end, 150 μg equivalent IgG (PRNT₅₀≈1/160) from week 6 RM were adoptively transferred into IFNAR^{-/-} mice 1 day after the ZIKV viral challenge. Two groups of control mice were included, one group receiving pre-immune sera from RM and the other group receiving phosphate-buffered saline (PBS). The mice that received PBS or control sera lost 15 to 25% of their original body weight during the course of infection, and all died 6-8 days post infection. When vaccine immune sera from RMs were transferred to infection-susceptible mice, the animals lost weight on day 3 and 4, but subsequently regained it beginning on day 5 and 80% ultimately survived infectious challenge (FIG. 26A) demonstrating the ability of the NHP sera transfer to confer protection against clinical manifestations of ZIKV infection following viral challenge (FIG. 26B). In repeated experiments performed to evaluate the efficacy of immune serum transfer in protection against challenge with ZIKV, the survival among ZIKV-prME immune sera recipients ranged from 80 to 100%. These studies show that anti-ZIKV vaccine immune sera had the ability to confer significant protection against ZIKV infection in the absence of an acquired adaptive anti-ZIKV immune response.

Vaccination with the ZIKV-prME Consensus Construct

Serious concerns have been raised by the recent spread of ZIKV and its associated pathogenesis in humans. Currently, there are no licensed vaccines or therapeutics for this emerging infectious agent. Very recently, a collection of experimental ZIKV vaccines have been shown to lower viral load post challenge in nonpathogenic animal infection models (Larocca et al., 2016, Nature 536:474-8; Abbink et al., 2016, Science 353:1192-32) These data are encouraging. In this regard, it is important to examine additional novel vaccine approaches targeting ZIKA in additional models. Here a synthetic DNA vaccine, designed to express a novel consensus ZIKV-prM and E antigen, was evaluated for immunogenicity following electroporation-enhanced immunization in mice and non-human primates. It was observed that ZIKV-prME DNA vaccination was immunogenic and generated antigen-specific T cells and binding and neutralizing antibodies in both mice and NHPs. Uniquely, the NHPs were immunized with ZIKV-prME through electroporation by the intradermal route, which uses lower voltage and a smaller transfection area than i.m. electroporation, as has been recently described (Trimble et al., 2016, Lancet 386:2078-88) Further study of such approaches may provide advantages in clinical settings.

The ZIKV-prME consensus construct includes a designed change of the potential NXS/T motif, which removes a putative glycosylation site. Deletion of glycosylation at this site has been correlated with improved binding of EDE1 type bnAbs (broadly neutralizing antibodies) against ZIKV-E protein (Muthumani et al., 2016, Sci Transl Med 7:301ra132). The antibody responses induced by the consensus ZIKV-prME appear as robust or in some cases superior in magnitude to those elicited by similarly developed ZIKV-prME-MR766 and ZIKV-prME-Brazil vaccines. These constructs were sequence matched with the original ZIKV-MR766 isolate or a recently circulating ZIKV strain from Brazil, respectively. While supportive, further study will provide more insight into the effects of such incorporated designed changes on induced immune responses.

As there are few pathogenic challenge models for ZIKV, the putative protective nature of the immune responses of the ZIKV-prME vaccine in C57BL/6 and IFNAR⁻− mice was compared. Both the strains of mice responded with a robust humoral immune response when immunized with ZIKV-prME. The T-cell responses were also induced, but appear to be more robust in wild-type C57BL/6 compared with those induced in the IFNAR⁻− animals, supporting a partial defect in innate to adaptive immunity transition as expected owing to the knock-out phenotype in the mouse. However, based on the induction of antigen specific immunity, the model was useful for evaluation of the impact of the vaccine on both infection and pathogenesis. A single vaccination with ZIKV-prME in IFNAR^{-/-} mice was protective against disease and death in this model, including protection of neuro-pathogenesis. Flavivirus-neutralizing antibodies directed against the Env antigen are thought to have a key role in protection against disease, an idea supported directly by passive antibody transfer experiments in animal models and indirectly by epidemiological data from prospective studies in geographical areas that are prone to mosquito-borne viral infections (Weaver et al., 2016, Antiviral Res 130:69-80; Roa et al., 2016, Lancet 387:843; Samarasekera et al., 2016, Lancet 387:521-4). Although immunization of IFNAR⁻− mice with the ZIKV-prME DNA vaccine as well as serum transfer from immunized NHPs were protective in this murine model, the IFNAR^{-/-} vaccinated as opposed to serum-transferred mice exhibited improved control of weight loss as an indication of control of pathogenesis. Although additional studies are needed, this result potentially suggests a role for the T-cell response in this aspect of protection in this model. In addition, it was observed that control IFNAR^{-/-} mice who recovered from challenge remain viral positive by PCR for at least several weeks, suggesting an additional benefit of vaccination. This study supports the potential of vaccination and, in this case this synthetic DNA vaccination, to impact prevention of disease in a susceptible host.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A composition comprising:
a) a first nucleic acid sequence wherein the nucleic acid sequence encodes an antigen; and
b) a second nucleic acid sequence encoding one or more antibodies or fragments thereof.

2. The composition of claim 1, wherein the antibody comprises a heavy chain polypeptide, or fragment thereof, and a light chain polypeptide, or fragment thereof.

3. The composition of claim 2, wherein the heavy chain polypeptide, or fragment thereof, is encoded by a third nucleic acid sequence and the light chain polypeptide, or fragment thereof, is encoded by a fourth nucleic acid sequence.

4. The composition of claim 3, wherein the second nucleic acid sequence comprises the third nucleic acid sequence and the fourth nucleic acid sequence.

5. The composition of claim 4, wherein the second nucleic acid sequence further comprises a promoter for expressing the third nucleic acid sequence and the fourth nucleic acid sequence as a single transcript.

6. The composition of claim 5, wherein the promoter is a cytomegalovirus (CMV) promoter.

7. The composition of claim 5, wherein the second nucleic acid sequence further comprises a fifth nucleic acid sequence encoding a protease cleavage site, wherein the fifth nucleic acid sequence is located between the third nucleic acid sequence and fourth nucleic acid sequence.

8. The composition of claim 7, wherein the protease of the subject recognizes and cleaves the protease cleavage site.

9. The composition of claim 2, wherein the heavy chain polypeptide comprises a variable heavy region and a constant heavy region 1.

10. The composition of claim 2, wherein the heavy chain polypeptide comprises a variable heavy region, a constant heavy region 1, a hinge region, a constant heavy region 2 and a constant heavy region 3.

11. The composition of claim 2, wherein the light chain polypeptide comprises a variable light region and a constant light region.

12. The composition of claim 1, wherein the second nucleic acid sequence further comprises a Kozak sequence.

13. The composition of claim 1, wherein the fourth nucleic acid sequence further comprises an immunoglobulin (Ig) signal peptide.

14. The composition of claim 13, wherein the Ig signal peptide comprises an IgE or IgG signal peptide.

15. The composition of claim 1, wherein the antibody is specific to the antigen.

16. The composition of claim 15, wherein the antigen is a foreign-antigen.

17. The composition of claim 16, wherein the foreign-antigen is selected from the group consisting of a viral antigen, a bacterial antigen and a parasitic antigen.

18. The composition of claim 17, wherein the viral antigen is selected from the group consisting of an HIV antigen, a Chickungunya antigen, a Dengue antigen, a Hepatitis antigen, a HPV antigen, a RSV antigen, an Influenza antigen, and an Ebola antigen.

19. The composition of claim 18, wherein the viral antigen is a Chickungunya antigen.

20. The composition of claim 19, wherein the first nucleic acid sequence encodes an antigen having an amino acid sequence having at least about 95% identity over an entire length of the amino acid sequence set forth in any of SEQ ID NOs: 81-88.

21. The composition of claim 20, wherein the first nucleic acid sequence comprises a nucleic acid sequence having at least about 95% identity over an entire length of the nucleic acid sequence set forth in any of SEQ ID NOs: 89-96.

22. The composition of claim 19, wherein the second nucleic acid sequence comprises a nucleic acid sequence encoding at least one amino acid sequence having at least about 95% identity over an entire length of the amino acid sequence set forth in SEQ ID NOs: 59 or 61.

23. The composition of claim 22, wherein the second nucleic acid sequence comprises a nucleic acid sequence having at least about 95% identity over an entire length of the nucleic acid sequence set forth in SEQ ID NOs: 58 or 60.

24. The composition of claim 15, wherein the antigen is a self-antigen.

25. A method of inducing an immune response comprising administering the composition of claim 1 to an individual in an amount effective to induce an immune response in said individual

26. The method of claim 25, wherein the immune response is persistent.

27. The method of claim 25, wherein the immune response is systemic.

28. A method of treating an individual who has been diagnosed with a disease or disorder comprising administering a therapeutically effective amount of the composition of claim 1 to an individual.


SEQUENCE LISTING (171 sequences)

SEQ ID NO: 1 - protein, 738 aa, Artificial Sequence
  Amino Acid Sequence of HIV-1 Env-4E10 Ig
MDWTWRILFLVAAATGTHAQVQLVQSGAEVKRPGSSVTVSCKASGGSFSTYALSWVRQAPGRGLEWMGGVIPLLTITNYAPRFQGRITITADRSTSTAYLELNSLRPEDTAVYYCAREGTTGWGWLGKPIGAFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVLTQSPGTQSLSPGERATLSCRASQSVGNNKLAWYQQRPGQAPRLLIYGASSRPSGVADRFSGSGSGTDFTLTISRLEPEDFAVYYCQQYGQSLSTFGQGTKVEKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 2 - protein, 768 aa, Artificial Sequence
  Amino Acid Sequence of HIV-1 Env-PG9 Ig
MDWTWRILFLVAAATGTHAEFGLSWVFLVAFLRGVQCQRLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYYDFYDGYYNYHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMAWTPLFLFLLTCCPGGSNSQSALTQPASVSGSPGQSITISCNGTSNDVGGYESVSWYQQHPGKAPKVVIYDVSKRPSGVSNRFSGSKSGNTASLTISGLQAEDEGDYYCKSLTSTRRRVFGTGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 3 - DNA, 792 bp, Artificial Sequence
  Nucleic Acid Sequence Encoding the Heavy Chain      (VH-CH1) of HIV-1 Env Fab
aagcttgccgccaccatggagactgatacactgctgctgtgggtgctgctgctgtgggtgccagggtcaaccggagatggggctcaggtccagctggtccagagcggcggacagatgaagaaacccggcgagagcatgaggatctcctgcagagcatctggatacgagttcatcgactgtaccctgaactggattaggctggctcctggaaagagaccagagtggatggggtggctgaaaccacgagggggagcagtgaattacgcccggcccctgcagggacgagtgaccatgaccagggacgtgtacagcgataccgccttcctggagctgcggtccctgacagtggacgatactgctgtctacttctgcacacgcggaaagaactgtgactataattgggattttgaacactggggccggggaacacccgtgatcgtcagctcccccagtactaagggaccttcagtgtttccactggccccctctagtaaatccacctctggagggacagccgctctgggatgcctggtgaaagattatttccccgaacctgtgaccgtcagttggaactcaggggctctgacttctggcgtgcacacctttcctgcagtcctgcagtcaagcgggctgtacagtctgtcctctgtggtcactgtgcctagttcaagcctgggcactcagacctatatttgtaacgtgaatcataagccatccaatacaaaagtggacaaaaaagccgaacccaaatcctgttacccttatgatgtgcccgactacgcctgactcgag

SEQ ID NO: 4 - DNA, 756 bp, Artificial Sequence
  Light Chain (VL-CL) of HIV-1 Env Fab
aagcttgccgccaccatggaaaccgatacactgctgctgtgggtgctgctgctgtgggtgccaggaagtaccggggatggggctcaggtccagattgtgctgactcagtcccctgggaccctgtctctgagtccaggcgagacagctatcatttcatgccgaactagccagtacggcagcctggcttggtatcagcagcgaccaggacaggcaccacgactggtcatctactcaggcagcacaagggccgctggcatccccgacaggttctccggcagcaggtgggggcctgattacaacctgactatctctaatctggagagtggggactttggcgtgtactattgccagcagtatgagttcttcggccagggaactaaggtgcaggtggacatcaaaagaaccgtggcagccccatccgtcttcatttttcccccttctgatgagcagctgaagtcaggcaccgccagcgtggtctgtctgctgaacaatttctacccccgggaagccaaggtgcagtggaaagtggacaacgctctgcagagtggaaattcacaggagagcgtgaccgaacaggactccaaggattctacatatagtctgagcagcaccctgaccctgagtaaagcagattacgagaagcacaaagtgtatgcctgtgaagtcacacatcagggcctgaggagccccgtgactaaaagtttcaaccgaggagagtgctacccttatgatgtgcccgactacgcctaactcgag

SEQ ID NO: 5 - protein, 731 aa, Artificial Sequence
  VRC01 IgG
MDWTWILFLVAAATRVHSQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMDWTWILFLVAAATRVHSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 6 - DNA, 819 bp, Artificial Sequence
  Optimized Nucleic Acid Sequence Encoding IgG      Heavy Chain
ggatccgccaccatggaaaccgacactctgctgctgtgggtgctgctgctgtgggtgcccggctcaacaggcgacggcgctcaggtccagctggtccagtctggagctgtgatcaagacccctggcagctccgtcaaaatttcttgcagagcaagtggctacaacttccgggactatagcatccactgggtgcggctgattcctgataagggatttgagtggatcggctggatcaagccactgtggggcgctgtgtcctacgcaaggcagctgcaggggcgcgtctccatgacacgacagctgtctcaggacccagacgatcccgattggggggtggcctacatggagttcagtggactgactcccgcagacaccgccgaatatttttgcgtgcggagaggctcctgcgactactgtggggatttcccatggcagtattggtgtcagggaactgtggtcgtggtctctagtgcatcaaccaagggccccagcgtgtttcctctggccccatcaagcaaaagtacatcaggaggaactgcagctctgggatgtctggtgaaggattacttccccgagcctgtgaccgtcagctggaactccggagcactgacctccggagtgcacacatttcccgctgtcctgcagtcctctgggctgtactctctgagttcagtggtcacagtgcctagctcctctctgggcacccagacatatatctgcaacgtcaatcataagccaagtaatactaaagtggacaagaaagtcgaacccaaatcatgttacccctatgacgtgcctgattatgcttgataactcgag

SEQ ID NO: 7 - DNA, 753 bp, Artificial Sequence
  Optimized Nucleic Acid Sequence Encoding IgG      Light Chain
ggatccgccaccatggagactgatacactgctgctgtgggtgctgctgctgtgggtgcctggctcaaccggcgacggggctcaggtccagattgtgctgacccagagccctggcatcctgtcactgagcccaggagagaccgcaacactgttctgcaaggcctcccagggcgggaacgctatgacatggtaccagaaacggagaggacaggtgccccgactgctgatctatgacacttcaaggcgagcaagcggagtgcctgatcgatttgtcggcagcggctctgggacagacttctttctgactattaataagctggacagagaggatttcgctgtgtactattgccagcagtttgaattctttggactgggcagcgagctggaagtgcacaggaccgtcgccgctccaagtgtgttcatttttccccctagcgatgagcagctgaaatccgggacagcctctgtggtctgtctgctgaacaatttctacccccgcgaagcaaaggtgcagtggaaagtcgacaacgccctgcagagtggcaattcacaggagagcgtgaccgaacaggactccaaggattctacatatagtctgagctccactctgaccctgtctaaagctgattacgagaagcacaaagtgtatgcatgcgaagtcactcatcagggcctgtctagtcctgtgaccaagagctttaaccgaggggagtgttacccatatgacgtccccgattacgcctgataactcgag

SEQ ID NO: 8 - protein, 18 aa, Artificial Sequence
  IgE1 Signal Peptide of VRC-1 IgG
MDWTWILFLVAAATRVHS

SEQ ID NO: 9 - protein, 126 aa, Artificial Sequence
  Variable Heavy Region of VRC01 IgG
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSPSTKG

SEQ ID NO: 10 - protein, 98 aa, Artificial Sequence
  Constant Heavy region 1 (CH1) of VRC01 IgG
PSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSC

SEQ ID NO: 11 - protein, 15 aa, Artificial Sequence
  Hinge Region of VRC01 IgG
EPKSCDKTHTCPPCP

SEQ ID NO: 12 - protein, 110 aa, Artificial Sequence
  Constant Heavy Region 2 (CH2) of VRC01 IgG
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 13 - protein, 107 aa, Artificial Sequence
  Constant Heavy Region 3 (CH3) of VRC01 IgG
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 7 aa, Artificial Sequence
  Furin Cleavage Site of VRC01 IgG
RGRKRRS

SEQ ID NO: 15 - protein, 22 aa, Artificial Sequence
  GSG Linker and P2A Peptide of VRC01 IgG
GSGATNFSLLKQAGDVEENPGP

SEQ ID NO: 16 - protein, 104 aa, Artificial Sequence
  Variable Light Region (VL) of VRC01 IgG
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKR

SEQ ID NO: 17 - protein, 106 aa, Artificial Sequence
  Constant Light Region (CL, kappa) of VRC01 IgG
TVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 18 - protein, 19 aa, Artificial Sequence
  Human IgG Heavy Chain Signal Peptide of HIV-1      Env-PG9 Ig
MDWTWRILFLVAAATGTHA

SEQ ID NO: 19 - protein, 154 aa, Artificial Sequence
  Variable Heavy Region of HIV-1 Env-PG9 Ig
EFGLSWVFLVAFLRGVQCQRLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYYDFYDGYYNYHYMDVWGKGTTVTVSS

SEQ ID NO: 20 - protein, 98 aa, Artificial Sequence
  Constant Heavy region 1 (CH1) of HIV-1      Env-PG9 Ig
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 21 - protein, 15 aa, Artificial Sequence
  Hinge Region of HIV-1 Env-PG9 Ig
EPKSCDKTHTCPPCP

SEQ ID NO: 22 - protein, 110 aa, Artificial Sequence; identical to SEQ ID NO: 12
  Constant Heavy Region 2 (CH2) of HIV-1      Env-PG9 Ig

SEQ ID NO: 23 - protein, 107 aa, Artificial Sequence; identical to SEQ ID NO: 13
  Constant Heavy Region 3 (CH3) of HIV-1      Env-PG9 Ig

SEQ ID NO: 24 - protein, 7 aa, Artificial Sequence
  Furin Cleavage Site of HIV-1 Env-PG9 Ig
RGRKRRS

SEQ ID NO: 25 - protein, 22 aa, Artificial Sequence
  GSG Linker and P2A Peptide of HIV-1 Env-PG9 Ig
GSGATNFSLLKQAGDVEENPGP

SEQ ID NO: 26 - protein, 20 aa, Artificial Sequence
  Human Lamba Light Chain Signal Peptide of HIV-1      Env-PG9 Ig
MAWTPLFLFLLTCCPGGSNS

SEQ ID NO: 27 - protein, 110 aa, Artificial Sequence
  Variable Light Region (VL) of HIV-1 Env-PG9 Ig
QSALTQPASVSGSPGQSITISCNGTSNDVGGYESVSWYQQHPGKAPKVVIYDVSKRPSGVSNRFSGSKSGNTASLTISGLQAEDEGDYYCKSLTSTRRRVFGTGTKLTVL

SEQ ID NO: 28 - protein, 106 aa, Artificial Sequence
  Constant Light Region (CL, lamba) of HIV-1      Env-PG9 Ig
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 29 - protein, 19 aa, Artificial Sequence
  Human IgG Heavy Chain Signal Peptide of HIV-1      Env-4E10 Ig
MDWTWRILFLVAAATGTHA

SEQ ID NO: 30 - protein, 127 aa, Artificial Sequence
  Variable Heavy Region of HIV-1 Env-4E10 Ig
QVQLVQSGAEVKRPGSSVTVSCKASGGSFSTYALSWVRQAPGRGLEWMGGVIPLLTITNYAPRFQGRITITADRSTSTAYLELNSLRPEDTAVYYCAREGTTGWGWLGKPIGAFAHWGQGTLVTVSS

SEQ ID NO: 31 - protein, 98 aa, Artificial Sequence
  Constant Heavy region 1 (CH1) of HIV-1      Env-4E10 Ig
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 32 - protein, 15 aa, Artificial Sequence
  Hinge Region of HIV-1 Env-4E10 Ig
EPKSCDKTHTCPPCP

SEQ ID NO: 33 - protein, 110 aa, Artificial Sequence; identical to SEQ ID NO: 12
  Constant Heavy Region 2 (CH2) of HIV-1      Env-4E10 Ig

SEQ ID NO: 34 - protein, 107 aa, Artificial Sequence; identical to SEQ ID NO: 13
  Constant Heavy Region 3 (CH3) of HIV-1      Env-4E10 Ig

SEQ ID NO: 35 - protein, 7 aa, Artificial Sequence
  Furin Cleavage Site of HIV-1 Env-4E10 Ig
RGRKRRS

SEQ ID NO: 36 - protein, 22 aa, Artificial Sequence
  GSG Linker and P2A Peptide of HIV-1 Env-4E10 Ig
GSGATNFSLLKQAGDVEENPGP

SEQ ID NO: 37 - protein, 20 aa, Artificial Sequence
  Human Kappa Light Chain Signal Peptide of HIV-1      Env-4E10 Ig
MVLQTQVFISLLLWISGAYG

SEQ ID NO: 38 - protein, 106 aa, Artificial Sequence
  Variable Light Region (VL) of HIV-1 Env-4E10 Ig
EIVLTQSPGTQSLSPGERATLSCRASQSVGNNKLAWYQQRPGQAPRLLIYGASSRPSGVADRFSGSGSGTDFTLTISRLEPEDFAVYYCQQYGQSLSTFGQGTKVE

SEQ ID NO: 39 - protein, 107 aa, Artificial Sequence
  Constant Light Region (CL, kappa) of HIV-1      Env-4E10 Ig
KRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 40 - DNA, 744 bp, Artificial Sequence
  Nucleic Acid Sequence Encoding the VH-CH1 of      anti-Her-2 Fab
ggatccgccaccatggactggacatggattctgtttctggtcgccgccgctacaagagtgcattccgaagtgcagctggtcgagagtggagggggactggtgcagcccggcggatctctgcgactgagttgcgccgcttcaggcttcacctttacagactacaccatggattgggtgagacaggcacctggcaagggactggagtgggtggctgatgtcaacccaaatagtgggggctcaatctacaaccagaggttcaagggcaggttcaccctgagcgtggacaggtccaaaaacactctgtatctgcagatgaattctctgcgggctgaagataccgcagtctactattgcgcccgcaatctgggcccaagcttctactttgactattgggggcagggcacactggtgactgtcagctccgcttctacaaagggaccaagcgtgttcccactggcaccctctagtaaatccacctctggagggacagcagccctgggctgtctggtgaaagactatttccccgagcctgtgactgtcagctggaactccggagcactgactagcggagtgcacacctttccagccgtcctgcagtcaagcggcctgtactccctgtcctctgtggtcacagtgcctagttcaagcctgggaactcagacctatatttgtaatgtgaaccataaaccaagcaatacaaaggtggacaagaaggtggaaccaaaatcctgctgataactcgag

SEQ ID NO: 41 - protein, 240 aa, Artificial Sequence
  Amino Acid Sequence of the VH-CH1 of anti-Her-2      Fab
MDWTWILFLVAAATRVHSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 42 - DNA, 720 bp, Artificial Sequence
  Nucleic Acid Sequence Encoding the VL-CL of      anti-Her-2 Fab
ggatccgccaccatggattggacttggattctgttcctggtcgccgccgctacccgcgtgcattccgatattcagatgactcagagcccctcctcactgtcagccagcgtgggcgaccgagtcaccatcacatgcaaagcttctcaggatgtgagtattggggtcgcatggtaccagcagaagccaggcaaagcacccaagctgctgatctattccgcctcttacaggtatacaggagtgcccagcagattcagtggctcaggaagcgggactgactttactctgaccatcagctccctgcagcctgaggatttcgctacctactattgccagcagtactatatctacccatatacctttggccagggaacaaaagtggagatcaagcggaccgtggccgctccctccgtcttcatttttcccccttctgacgaacagctgaagagcggaacagcaagcgtggtctgtctgctgaacaatttctaccctcgcgaggccaaagtgcagtggaaggtcgataacgctctgcagtccgggaattctcaggagagtgtgactgaacaggactcaaaagatagcacctattccctgtctagtacactgactctgagcaaggcagactacgaaaagcacaaagtgtatgcctgtgaggtcacccaccaggggctgtcaagtcccgtcaccaagtccttcaatagaggcgaatgctgataactcgag

SEQ ID NO: 43 - protein, 232 aa, Artificial Sequence
  Amino Acid Sequence of the VL-CL of anti-Her-2      Fab
MDWTWILFLVAAATRVHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 2241 bp, Artificial Sequence
  Nucleic Acid Sequence Encoding anti-DENV Human      IgG
ggatccgccaccatggactggacttggaggattctgtttctggtcgccgccgctactgggactcacgctcaggcacatctggtcgaatctggaggaggagtggtccagcctggccgatccctgcgactgtcttgcgcagctagcgccttcaacttcagcacaaacgcaatgcactgggtgcgacaggcaccaggcaagggactggagtgggtcgctgtgatctcatacgacggaagccataagtactatgcagattctgtgaaaggccggttcaccatttccagggacaattctaagaacaccctgtatctgcagatgaatagcctgcgcgcagccgataccgcagtgtactattgcgcaactgtcggcgtgctgacctggccagtgaacgccgaatactttcaccattggggacagggcagtctggtctcagtgagctccgcaagtactaagggaccatcagtgttcccactggcaccctctagtaaatctactagtggcgggaccgctgcactgggatgtctggtgaaggactatttccccgagcctgtcaccgtgagctggaattccggagccctgacaagcggcgtccacacttttcccgctgtgctgcagtcaagcggactgtactccctgtcctctgtggtcactgtgcctagttcaagcctgggcactcagacctatatctgcaatgtgaaccacaagccctctaacaccaaagtcgacaagaaagtggaacctaagagctgtgataaaacacatacttgcccaccttgtccagcaccagagctgctgggaggaccaagcgtgttcctgtttccacccaagcctaaagacacactgatgattagccggacacctgaagtcacttgcgtggtcgtggacgtgtcccacgaggaccccgaagtcaagtttaattggtacgtggatggcgtcgaggtgcataacgccaagaccaaaccccgggaggaacagtacaatagcacatatagagtcgtgtccgtcctgactgtgctgcatcaggattggctgaatgggaaggagtataagtgcaaagtgtctaacaaggctctgcctgcaccaatcgagaaaaccattagcaaggctaaaggccagcctagggaaccacaggtgtacacactgcctccaagtcgcgacgagctgaccaagaatcaggtctccctgacatgtctggtgaaaggcttctatccatcagatatcgccgtggagtgggaaagcaacgggcagcccgaaaacaattacaagaccacaccccctgtgctggactctgatggcagtttctttctgtattctaagctgaccgtggacaaaagtagatggcagcaggggaatgtcttttcatgtagcgtgatgcacgaggccctgcacaaccattacacacagaagtccctgtctctgagtcccggaaagaggggccgcaaacggagatcagggagcggagctactaatttcagcctgctgaaacaggcaggggatgtggaggaaaaccccggacctatggcttggaccccactgttcctgtttctgctgacatgctgtcccgggggcagcaattctcagagtgtcctgacacagccaccatcagtgagcggagcaccaggacagagggtgaccatctcctgcacaggcagcagcagcaacattggcgccgggtacgacgtgcattggtatcagcagctgcccggcaccgctcctaagctgctgatctgtggcaacaataaccgcccatctggggtgcccgatcgattctccggctctaaaagtgggacttcagccagcctggctattaccggcctgcaggccgaggacgaagctgattactattgccagagctacgactcaagcctgaccggagtcgtgttcggaggaggaaccaagctgacagtcctgggacagcctaaagccgctccaagcgtgacactgtttcctccatcctctgaggaactgcaggcaaacaaggccaccctggtgtgcctgatttccgacttctaccccggggcagtcactgtggcttggaaggcagatagttcacctgtcaaagccggagtggagactaccacaccatcaaagcagagcaataacaaatacgcagccagctcctatctgtccctgacccctgagcagtggaagtctcacaaatcctattcttgccaggtcactcacgaaggaagcactgtggagaaaactgtcgcaccaaccgaatgtagttgataactcgag

SEQ ID NO: 45 - protein, 739 aa, Artificial Sequence
  Amino Acid Sequence of anti-DENV Human IgG
MDWTWRILFLVAAATGTHAQAHLVESGGGVVQPGRSLRLSCAASAFNFSTNAMHWVRQAPGKGLEWVAVISYDGSHKYYADSVKGRFTISRDNSKNTLYLQMNSLRAADTAVYYCATVGVLTWPVNAEYFHHWGQGSLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMAWTPLFLFLLTCCPGGSNSQSVLTQPPSVSGAPGQRVTISCTGSSSNIGAGYDVHWYQQLPGTAPKLLICGNNNRPSGVPDRFSGSKSGTSASLAITGLQAEDEADYYCQSYDSSLTGVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 46 - protein, 265 aa, Artificial Sequence
  IgG Heavy Chain
METDTLLLWVLLLWVPGSTGDGAQVQLVQSGAVIKTPGSSVKISCRASGYNFRDYSIHWVRLIPDKGFEWIGWIKPLWGAVSYARQLQGRVSMTRQLSQDPDDPDWGVAYMEFSGLTPADTAEYFCVRRGSCDYCGDFPWQYWCQGTVVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCYPYDVPDYA

SEQ ID NO: 47 - protein, 243 aa, Artificial Sequence
  IgG Light Chain
METDTLLLWVLLLWVPGSTGDGAQVQIVLTQSPGILSLSPGETATLFCKASQGGNAMTWYQKRRGQVPRLLIYDTSRRASGVPDRFVGSGSGTDFFLTINKLDREDFAVYYCQQFEFFGLGSELEVHRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECYPYDVPDYA

SEQ ID NO: 48 - protein, 256 aa, Artificial Sequence
  Amino Acid Sequence of the Heavy Chain (VH-CH1)      of HIV-1 Env Fab
METDTLLLWVLLLWVPGSTGDGAQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCYPYDVPDYA

SEQ ID NO: 49 - protein, 244 aa, Artificial Sequence
  Amino Acid Sequence of the Light Chain (VL-CL)      of HIV-1 Env Fab
METDTLLLWVLLLWVPGSTGDGAQVQIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGECYPYDVPDYA

SEQ ID NO: 50 - DNA, 1536 bp, Artificial Sequence
  Nucleic Acid Sequence Encoding HIV-1 PG9 Fab
ggatccgccaccatggcaagacccctgtgcaccctgctgctgctgatggcaaccctggccggagccctggcacagagcgccctgacccagcccgcaagcgtctccggctcaccaggccagagcatcactattagttgcaacgggactagcaacgacgtgggaggctatgagagtgtcagctggtaccagcagcatcccggaaaagcaccaaaagtggtcatctacgatgtcagtaaaaggccaagtggggtctcaaataggttctcagggagtaaatctgggaatacagcatctctgaccatctccggactgggcgcagaagatgaaggcgactactattgcaaaagcctgacctcaaccagacggcgagtctttgggacaggcaccaagctgacagtcctgacagtcgctgccccctccgtcttcatttttccaccttcagatgagcagctgaaatctggcactgcatctgtggtctgcctgctgaacaacttctatccacgagaggccaaggtgcagtggaaagtggataacgcactgcagtccggcaatagtcaggaaagcgtgactgagcaggattccaaggacagtacctatagcctgtccagtacactgaccctgtccaaggctgactacgaaaaacataaggtgtatgcatgtgaagtgactcaccagggactgaggtcaccagtcactaagtcttttaacaggggagagtgcggcgggggaggatctggaggcggcggctctggagggggaggctcagggggcggaggaagcggcggaggagggtccggaggaggaggcagtcagagactggtcgaaagcgggggaggagtggtgcagcctgggtcctcactgagactgtcatgcgctgccagtggctttgatttttcacgacagggaatgcattgggtcaggcaggcacccggacagggcctggaatgggtcgccttcattaagtacgacggaagcgagaagtaccatgccgactcagtgtggggaaggctgagcatctcaagggacaactcaaaggacaccctgtacctgcagatgaatagcctgagagtggaagataccgctacttatttctgcgtgcgagaggccggagggccagattaccggaacgggtacaattactatgatttctacgacggctactacaattaccattatatggatgtctggggcaaaggaactacagtcaccgtgagctccgcaagtactaagggaccttccgtgtttcctctggctcccagttccaaaagtacatccggaggaacagccgctctgggatgtctggtcaaggactattttcccgagcccgtgactgtctcctggaacagcggggctctgacaagcggggtgcacacctttcctgccgtgctgcagtccagtgggctgtacagtctgtctagtgtcgtcactgtgccaagctcaagtctggggacccagacatacatttgtaatgtgaaccataaaccctcaaacaccaaagtggacaagaaagtggaacctaaaagctgataactcgag

SEQ ID NO: 51 - protein, 504 aa, Artificial Sequence
  Amino Acid Sequence of HIV-1 PG9 Fab
MARPLCTLLLLMATLAGALAQSALTQPASVSGSPGQSITISCNGTSNDVGGYESVSWYQQHPGKAPKVVIYDVSKRPSGVSNRFSGSKSGNTASLTISGLGAEDEGDYYCKSLTSTRRRVFGTGTKLTVLTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGECGGGGSGGGGSGGGGSGGGGSGGGGSGGGGSQRLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYYDFYDGYYNYHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 52 - DNA, 1503 bp, Artificial Sequence
  Nucleic Acid Sequence Encoding HIV-1 4E10 Fab
ggatccgccaccatggcaagacctctgtgcactctgctgctgctgatggctactctggccggggctctggctgagattgtcctgacccagtcccctggcactcagtcactgtcccccggcgagcgcgcaactctgtcctgcagagcaagccagtccgtcgggaacaacaagctggcatggtaccagcagcgcccaggacaggcacccaggctgctgatctacggagcaagctcccggcctagcggagtcgctgatagattctccggaagcggctccgggaccgatttcactctgaccatctccaggctggaacctgaggattttgccgtgtattactgtcagcagtacgggcagagcctgtcaactttcggccagggaactaaagtcgaaaagagaaccgtggccgcaccaagcgtctttatttttccccctagcgatgaacagctgaaatccgggactgcttccgtggtctgcctgctgaataacttctatccaagagaggcaaaggtgcagtggaaagtggacaacgccctgcagagcggaaactcacaggaatctgtgacagagcaggactccaaggatagcacatacagtctgtcctcaactctgaccctgtccaaagctgactatgagaagcataaagtctacgcatgtgaggtgacccaccagggactgaggtcccccgtcactaagtccttcaatagaggcgagtgcgggggcgggggcagtggcggagggggaagtgggggcggagggagtggcggcggcgggagtggcggcggcggctcagggggcggcggctcccaggtccagctggtccagagcggagccgaggtcaagagaccaggctcttcagtcaccgtgagctgcaaagccagcggaggctcctttagcacttacgccctgtcatgggtgcggcaggccccaggccgaggcctggagtggatgggcggcgtgatccccctgctgaccattactaactatgcccctagatttggaggccggatcaccatcacagctgacagatccacatccacagcttacctggagctgaacagtctgaggcccgaggacactgcagtctactactgtgcacgagaaggcaccactggatgggggtggctggggaagcccatcggggcttttgcacattggggcggagggacactggtgactgtgagctctgccagcactaaagggcccagtgtcttccctctggccccaagttccaagagtacatcagggggcaccgccgcactggggtgtctggtgaaggattacttcccagagcccgtgacagtcagttggaacagcggcgctctgaccagtggggtgcacactttcccagccgtgctgcagagttcagggctgtactccctgtcctcagtggtgactgtgccctcaagcagtctggggactcagacttacatttgtaatgtgaaccataaaccctcaaatactaaagtggacaaaaaagtggaaccaaagagctgataactcgag

SEQ ID NO: 53 - protein, 493 aa, Artificial Sequence
  Amino Acid Sequence of HIV-1 4E10 Fab
MARPLCTLLLLMATLAGALAEIVLTQSPGTQSLSPGERATLSCRASQSVGNNKLAWYQQRPGQAPRLLIYGASSRPSGVADRFSGSGSGTDFTLTISRLEPEDFAVYYCQQYGQSLSTFGQGTKVEKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGECGGGGSGGGGSGGGGSGGGGSGGGGSGGGGSQVQLVQSGAEVKRPGSSVTVSCKASGGSFSTYALSWVRQAPGRGLEWMGGVIPLLTITNYAPRFGGRITITADRSTSTAYLELNSLRPEDTAVYYCAREGTTGWGWLGKPIGAFAHWGGGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 54 - DNA, 1446 bp, Artificial Sequence
  Nucleic Acid Sequence Encoding the HIV-1 VRC01      IgG1 Heavy Chain (VH/CH1/Hinge/CH2/CH3)
ggatccgccaccatggattggacatggattctgttcctggtcgccgccgcaactagagtgcattcacaggtgcagctggtgcagtcaggcgggcagatgaagaaacccggcgagagtatgcgaatctcatgccgggctagcgggtacgaattcatcgactgtaccctgaactggattagactggcacctgggaagaggccagagtggatgggatggctgaaacctagaggcggggcagtgaattacgccagaccactgcagggcagggtcactatgacccgcgacgtgtattctgataccgcattcctggagctgcgaagtctgacagtcgacgatactgccgtgtacttctgcacacggggcaagaactgtgactataattgggattttgaacactggggcagggggacacctgtcattgtgagctccccaagtactaagggaccctcagtgtttcccctggccccttctagtaaaagtacctcaggaggcacagccgctctgggatgcctggtgaaggattacttccctgagccagtcaccgtgagttggaactcaggcgccctgacaagcggggtccatacttttccagctgtgctgcagtcaagcgggctgtactccctgtcctctgtggtcacagtgcccagttcaagcctgggaacacagacttatatctgtaacgtcaatcacaagcctagcaatactaaagtggacaagaaagccgagcctaagagctgcgaaccaaagtcctgtgataaaacccatacatgccctccctgtccagctcctgaactgctgggcggcccatccgtgttcctgtttccacccaagcccaaagacaccctgatgattagcaggactcctgaggtcacctgcgtggtcgtggacgtgtcccacgaggaccccgaagtcaagtttaactggtacgtggatggcgtcgaagtgcataatgccaagacaaaaccccgggaggaacagtacaactctacctatagagtcgtgagtgtcctgacagtgctgcaccaggactggctgaacgggaaggagtataagtgcaaagtgtctaataaggccctgccagctcccatcgagaaaacaatttccaaggcaaaaggccagccaagggaaccccaggtgtacactctgcctccatcccgcgacgagctgactaagaaccaggtctctctgacctgtctggtgaaaggattctatccaagcgatatcgccgtggagtgggaatccaatggccagcccgagaacaattacaagaccacaccccctgtgctggacagcgatggctccttctttctgtattcaaagctgaccgtggataaaagccgctggcagcaggggaacgtctttagctgctccgtgatgcacgaagctctgcacaatcattacacccagaagtctctgagtctgtcacctggcaagtgataactcgag

SEQ ID NO: 55 - protein, 474 aa, Artificial Sequence
  Amino Acid Sequence of the HIV-1 VRC01 IgG1      Heavy Chain (VH/CH1/CH2/CH3)
MDWTWILFLVAAATRVHSQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 708 bp, Artificial Sequence
  Nucleic Acid Sequence Encoding the HIV-1 VRC01      IgG Light Chain (VL/CL)
ggatccgccaccatggattggacttggattctgttcctggtggcagccgctaccagagtccattccgaaattgtgctgacccagtctcccggaacactgtctctgagtcctggcgagacagccatcatttcctgtaggacttctcagtacgggagtctggcatggtatcagcagcgaccaggacaggctcctcgactggtcatctactcaggaagcactcgggcagccggcattcccgaccgattctccgggtctcggtggggacctgattacaacctgaccatctcaaatctggaaagcggagactttggcgtgtactattgccagcagtatgagttctttgggcagggaaccaaggtccaggtggacatcaaacgcacagtcgctgcaccaagcgtgttcatctttccaccctcagatgaacagctgaagtccggcaccgcctctgtggtgtgcctgctgaacaatttctacccccgggaggcaaaggtccagtggaaagtggacaacgccctgcagtctggcaatagtcaggagtcagtgactgaacaggacagcaaggattccacctattctctgtcctctactctgaccctgagcaaagctgattacgagaagcacaaagtgtatgcatgtgaggtcacccaccagggactgcggtcacccgtcaccaagagcttcaatcgcggagagtgttgataactcgag

SEQ ID NO: 57 - protein, 228 aa, Artificial Sequence
  Amino Acid Sequence of the HIV-1 VRC01 IgG      Light Chain (VL/CL)
MDWTWILFLVAAATRVHSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 58 - DNA, 744 bp, Artificial Sequence
  Nucleic Acid Sequence Encoding the Heavy Chain      (VH-CH1) of the CHIKV-Env-Fab
ggatccgccaccatggattggacatggaggattctgtttctggtcgccgccgctactggaactcacgctcaggtgcagctggtgcagtcagggtccgaactgaagaaaccaggggcatctgtgaaggtcagttgcaaagcctcaggctacaccctgacacggtatgccatgacttgggtgcgccaggctcctggacagggactggagtggatgggctggatcaacacttacaccggaaatccaacttatgtgcaggggttcaccggccgattcgtgttttctctggacacttccgtctctaccgcctttctgcacattacaagtctgaaggcagaggacactgccgtgtacttctgcgctagggaaggcggagcaagaggctttgattattggggccagggaaccctggtgacagtcagctccgccagcacaaagggaccctccgtgttcccactggctccctctagtaaaagtacatcagggggcactgccgctctgggatgtctggtcaaagattacttccccgaacctgtgaccgtcagctggaactccggagctctgaccagcggggtgcatacatttcccgcagtcctgcagtcaagcggactgtactccctgtcctctgtggtcacagtgcctagttcaagcctggggacacagacttatatctgtaatgtgaaccataagccaagcaacaccaaagtggacaaaaaagtggaacctaagagctgctgataactcgag

SEQ ID NO: 59 - protein, 240 aa, Artificial Sequence
  Amino Acid Sequence of the Heavy Chain (VH-CH1)      of the CHIKV-Env-Fab
MDWTWRILFLVAAATGTHAQVQLVQSGSELKKPGASVKVSCKASGYTLTRYAMTWVRQAPGQGLEWMGWINTYTGNPTYVQGFTGRFVFSLDTSVSTAFLHITSLKAEDTAVYFCAREGGARGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 60 - DNA, 738 bp, Artificial Sequence
  Nucleic Acid Sequence Encoding the Light Chain      (VL-CL) of the CHIKV-Env-Fab
ggatccgccaccatggcatggaccccactgttcctgttcctgctgacttgttgtcctggcgggagcaattcacagagcgtcctgacccagcccccttctgtgtccggagcaccaggacagcgagtcacaatctcttgcactggaagctcctctaacattggggccagccacgacgtgcattggtaccagcagctgccagggaccgctcccacactgctgatctatgtgaactctaataggcctagtggcgtcccagatagattttcagggagcaagtccggcacctctgctagtctggcaattacaggactgcaggctgaggacgaagcagattactattgccagagttacgactcaaacctgtcaggcagcgcagtgttcggaggaggaactaagctgaccgtcctgggacagcccaaagccgctccttctgtgaccctgtttccccctagttcagaggaactgcaggccaacaaggctactctggtgtgtctgatctccgacttctaccctggagcagtgaccgtcgcatggaaggccgatagctccccagtgaaagctggggtcgagaccacaactcccagcaagcagtccaacaacaagtacgcagcctctagttatctgtcactgacacctgaacagtggaagagccacaaatcctattcttgccaagtgactcatgagggcagtaccgtggaaaagacagtcgccccaactgagtgttcctgataactcgag

SEQ ID NO: 61 - protein, 238 aa, Artificial Sequence
  Amino Acid Sequence of the Light Chain (VL-CL)      of the CHIKV-Env-Fab
MAWTPLFLFLLTCCPGGSNSQSVLTQPPSVSGAPGQRVTISCTGSSSNIGASHDVHWYQQLPGTAPTLLIYVNSNRPSGVPDRFSGSKSGTSASLAITGLQAEDEADYYCQSYDSNLSGSAVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 62 - DNA, 2238 bp, Artificial Sequence
  Nucleic Acid Sequence Encoding HIV-1 Env-4E10      Ig
ggatccgccaccatggattggacatggaggattctgtttctggtcgccgccgctacaggaactcacgcccaggtgcagctggtgcagtcaggagccgaagtgaagcgaccaggcagctccgtcactgtgtcctgcaaagcatctggcggatcattcagcacctacgccctgagctgggtgagacaggctcctggacgaggactggaatggatgggaggcgtcatcccactgctgacaattactaactacgccccccgatttcagggcaggatcaccattacagcagaccgctccacttctaccgcctatctggagctgaatagcctgagaccagaagataccgcagtgtactattgcgcccgggagggaaccacaggatggggatggctgggaaagcccatcggggctttcgcacactggggccagggaaccctggtcacagtgtctagtgccagcacaaagggcccctccgtgtttcccctggctccttcaagcaaaagtacttcaggagggaccgccgctctgggatgtctggtgaaggactacttccctgagccagtcaccgtgtcctggaactctggcgctctgacctccggagtgcatacatttcccgcagtcctgcagtcctctgggctgtactctctgagttcagtggtcactgtgcctagctcctctctgggcacacagacttatatctgcaacgtgaatcacaagccctccaataccaaagtcgacaagaaagtggaacctaagtcttgtgataaaacccatacatgcccaccttgtccagcacctgagctgctgggcggaccttccgtgttcctgtttccacccaagccaaaagacacactgatgattagccggacacctgaagtgacttgtgtggtcgtggacgtcagccacgaggaccccgaagtgaagttcaactggtacgtggatggcgtcgaggtgcataatgccaagaccaaacccagggaggaacagtacaactctacttatagggtcgtgagtgtcctgaccgtgctgcaccaggactggctgaacgggaaggagtataagtgcaaagtgtccaataaggccctgccagctcccatcgagaaaacaatttctaaggctaaaggccagccacgcgaaccccaggtgtacactctgcctcccagcagggacgagctgaccaagaaccaggtgagtctgacatgtctggtcaaaggcttctatccaagcgatatcgccgtggagtgggaatccaatggacagcccgaaaacaattacaagactaccccccctgtgctggacagtgatggatcattctttctgtattccaagctgaccgtggacaaatctcgctggcagcaggggaacgtctttagctgctccgtgatgcacgaggccctgcacaatcattacacacagaagtctctgagtctgtcaccaggcaagcggggacgcaaaaggagaagcgggtccggcgctactaacttcagcctgctgaaacaggcaggggatgtggaggaaaatcctggcccaatggtcctgcagacccaggtgtttatctcactgctgctgtggattagcggggcttatggcgaaatcgtgctgactcagagccccggaacccagtctctgagtcctggggagcgcgctacactgagctgtcgagcatcacagagcgtggggaacaataagctggcatggtaccagcagaggcctggccaggctccaagactgctgatctatggcgcaagttcacggcctagcggagtggcagaccgcttctccggatctgggagtggcaccgattttactctgaccattagcaggctggagccagaagacttcgctgtgtactattgccagcagtacggccagtcactgagcacatttggacaggggactaaggtcgaaaaaagaaccgtggcagccccaagtgtcttcatttttccaccctcagacgagcagctgaagagtggaacagcctcagtcgtgtgtctgctgaacaatttctaccccagggaggccaaggtccagtggaaagtggataacgctctgcagagcggcaattcccaggagtctgtgacagaacaggacagtaaggattcaacttatagcctgagctccacactgactctgtccaaagcagattacgagaagcacaaagtgtatgcctgcgaagtcacccatcagggactgtctagtcctgtgacaaagtcttttaacagaggggagtgataactcgag

SEQ ID NO: 63 - DNA, 2328 bp, Artificial Sequence
  Nucleic Acid Sequence Encoding HIV-1 Env-PG9 Ig
ggatccgccaccatggactggacttggaggattctgtttctggtcgccgccgcaactggaactcacgctgaatttggactgtcatgggtctttctggtggcctttctgcgaggggtccagtgccagaggctggtggagtccggaggaggagtggtccagccaggcagctccctgcgactgagttgtgccgcttcagggttcgacttttctagacagggcatgcactgggtgcggcaggcaccaggacagggactggagtgggtggctttcatcaagtacgacggaagtgaaaaatatcatgccgattcagtgtgggggcggctgtcaattagccgcgacaactccaaggataccctgtacctgcagatgaattctctgagggtcgaggacacagctacttatttctgcgtgagggaagcaggcggacctgattacagaaacgggtataattactatgacttttacgatggctactataactaccactatatggacgtgtggggcaagggaaccacagtcacagtgtctagtgcatcaactaaaggcccaagcgtgtttcccctggccccttcaagcaagtccacttctggaggaaccgcagcactgggatgtctggtgaaggattacttccctgagccagtcaccgtgagttggaactcaggcgccctgactagcggagtccatacctttcctgctgtgctgcagtcctctgggctgtacagcctgagttcagtggtcacagtgccaagctcctctctgggcacccagacatatatctgcaacgtgaatcacaagcctagcaatactaaggtcgacaaaagagtggaaccaaagagctgtgataaaactcatacctgcccaccttgtccagcacctgagctgctgggagggccttccgtgttcctgtttccacccaagccaaaagacaccctgatgattagccggacaccagaagtcacttgcgtggtcgtggacgtgagccacgaggaccccgaagtcaagtttaactggtacgtggatggcgtcgaggtgcataatgctaagacaaaaccacgggaggaacagtacaactccacatatcgcgtcgtgtctgtcctgactgtgctgcaccaggactggctgaacggcaaggagtataagtgcaaagtgtccaataaggcactgccagcccccatcgagaaaaccatttctaaggccaaaggccagccacgagaaccccaggtgtacacactgcctccaagtagggacgagctgactaagaaccaggtctctctgacctgtctggtgaaaggcttctatccctctgatatcgctgtggagtgggaaagtaatggacagcctgaaaacaattacaagactaccccccctgtgctggacagcgatggcagcttcttcctgtatagcaagctgaccgtggacaaatccagatggcagcaggggaacgtctttagttgctcagtgatgcacgaggcactgcacaatcattacacccagaaaagcctgtccctgtctcctggcaagaggggaagaaaaaggagaagtgggtcaggcgcaacaaacttcagcctgctgaagcaggccggagatgtggaggaaaatcctgggccaatggcttggacccccctgttcctgtttctgctgacatgctgtcctggcggaagcaactcccagtctgcactgacacagccagcaagtgtgtcagggagcccaggacagagcatcaccatttcctgtaacggcacaagcaatgacgtcgggggctacgagtccgtgtcttggtatcagcagcatcctggaaaggccccaaaagtcgtgatctacgatgtcagcaaacgcccctctggggtgagtaaccgattcagtggatcaaagagcgggaataccgcttctctgacaattagtggcctgcaggcagaggacgaaggagattactattgcaaatcactgacaagcactcggcgccgagtcttcggaaccgggacaaagctgactgtgctgggccagcccaaagctgcacctagcgtgaccctgtttccacccagttcagaggaactgcaggctaataaggcaacactggtgtgtctgatctccgacttctaccctggcgctgtcactgtggcctggaaggctgatagctccccagtcaaagcaggagtggaaacaactaccccctccaagcagtctaacaacaagtacgccgcttctagttatctgtcactgactcccgagcagtggaagagccacaaatcctattcttgccaggtgacccatgagggctccactgtcgaaaagaccgtggcccctacagagtgttcttgataactcgag

SEQ ID NO: 64 - DNA, 2217 bp, Artificial Sequence
  Nucleic Acid Sequence Encoding VRC01 IgG
ggatccgccaccatggattggacatggattctgttcctggtcgccgccgcaactagagtgcattcacaggtgcagctggtgcagtcaggcgggcagatgaagaaacccggcgagagtatgcgaatctcatgccgggctagcgggtacgaattcatcgactgtaccctgaactggattagactggcacctgggaagaggccagagtggatgggatggctgaaacctagaggcggggcagtgaattacgccagaccactgcagggcagggtcactatgacccgcgacgtgtattctgataccgcattcctggagctgcgaagtctgacagtcgacgatactgccgtgtacttctgcacacggggcaagaactgtgactataattgggattttgaacactggggcagggggacacctgtcattgtgagctccccaagtactaagggaccctcagtgtttcccctggccccttctagtaaaagtacctcaggaggcacagccgctctgggatgcctggtgaaggattacttccctgagccagtcaccgtgagttggaactcaggcgccctgacaagcggggtccatacttttccagctgtgctgcagtcaagcgggctgtactccctgtcctctgtggtcacagtgcccagttcaagcctgggaacacagacttatatctgtaacgtcaatcacaagcctagcaatactaaagtggacaagaaagccgagcctaagagctgcgaaccaaagtcctgtgataaaacccatacatgccctccctgtccagctcctgaactgctgggcggcccatccgtgttcctgtttccacccaagcccaaagacaccctgatgattagcaggactcctgaggtcacctgcgtggtcgtggacgtgtcccacgaggaccccgaagtcaagtttaactggtacgtggatggcgtcgaagtgcataatgccaagacaaaaccccgggaggaacagtacaactctacctatagagtcgtgagtgtcctgacagtgctgcaccaggactggctgaacgggaaggagtataagtgcaaagtgtctaataaggccctgccagctcccatcgagaaaacaatttccaaggcaaaaggccagccaagggaaccccaggtgtacactctgcctccatcccgcgacgagctgactaagaaccaggtctctctgacctgtctggtgaaaggattctatccaagcgatatcgccgtggagtgggaatccaatggccagcccgagaacaattacaagaccacaccccctgtgctggacagcgatggctccttctttctgtattcaaagctgaccgtggataaaagccgctggcagcaggggaacgtctttagctgctccgtgatgcacgaagctctgcacaatcattacacccagaagtctctgagtctgtcacctggcaagaggggacgaaaacggagaagcggcagcggagctacaaacttcagcctgctgaaacaggcaggcgacgtggaggaaaatcctgggccaatggattggacttggattctgttcctggtggcagccgctaccagagtccattccgaaattgtgctgacccagtctcccggaacactgtctctgagtcctggcgagacagccatcatttcctgtaggacttctcagtacgggagtctggcatggtatcagcagcgaccaggacaggctcctcgactggtcatctactcaggaagcactcgggcagccggcattcccgaccgattctccgggtctcggtggggacctgattacaacctgaccatctcaaatctggaaagcggagactttggcgtgtactattgccagcagtatgagttctttgggcagggaaccaaggtccaggtggacatcaaacgcacagtcgctgcaccaagcgtgttcatctttccaccctcagatgaacagctgaagtccggcaccgcctctgtggtgtgcctgctgaacaatttctacccccgggaggcaaaggtccagtggaaagtggacaacgccctgcagtctggcaatagtcaggagtcagtgactgaacaggacagcaaggattccacctattctctgtcctctactctgaccctgagcaaagctgattacgagaagcacaaagtgtatgcatgtgaggtcacccaccagggactgcggtcacccgtcaccaagagcttcaatcgcggagagtgttgataactcgag

SEQ ID NO: 65 - DNA, 2223 bp, Artificial Sequence
  CHIKV snapi nucleic acid sequence
ggatccgccaccatggactggacttggattctgtttctggtcgccgccgctacccgagtgcattcacaggtgcagctgcagcagcctggggccgctctggtgaagccaggagctagcgcaatgatgtcctgcaaagcctctggctacactttcacctcctattggatcacctgggtgaagcagcgacctggacagggactggagtggatcggcgacatctacccaggcaccgggagaacaatctacaaggaaaaattcaagacaaaagccacactgactgtggacaccagctcctctacagcttttatgcagctgaacagcctgacttccgaggatagcgccgtgtactattgcgcaagaggatacggctctccttactatgccctggactattgggggcagggaactagcgtcaccgtgagttcagcatctaccaagggaccaagcgtgttcccactggcacctagctccaaatccacttctggcgggaccgccgctctgggatgtctggtgaaggattacttccctgagccagtcacagtgagttggaactcaggggctctgaccagcggagtccacacatttcctgcagtgctgcagtctagtggactgtactccctgtcaagcgtggtcactgtcccatcctctagtctgggcacccagacatatatctgcaacgtgaatcacaagccatccaataccaaagtcgataagaaagtggagcccaagtcttgtgacaaaactcatacctgccctccctgtccagcacctgaactgctgggaggcccaagcgtgttcctgtttccacccaagcctaaagacaccctgatgattagcaggacaccagaggtcacttgcgtggtcgtggacgtgagccacgaagaccccgaggtcaagttcaactggtacgtggatggcgtcgaagtgcataatgccaagacaaaaccccgggaggaacagtacaactcaacctatcgggtcgtgagcgtcctgacagtgctgcaccaggactggctgaacggaaaggagtacaagtgcaaagtgtctaataaggccctgccagctcccatcgaaaaaaccattagcaaggctaaaggccagccaagagagccccaggtgtacacactgcctccatcaagggacgaactgacaaagaaccaggtcagcctgacttgtctggtgaaaggcttctatcccagcgatatcgcagtggaatgggagtccaatgggcagcctgagaacaattacaagaccacaccccctgtgctggacagcgatgggtccttctttctgtattccaagctgacagtggataaatctcggtggcagcagggaaacgtctttagttgctcagtgatgcacgaagccctgcacaatcattacactcagaagagcctgtccctgtctcccggaaagaggggccgcaaacggagaagtggctcaggggcaaccaacttctctctgctgaaacaggccggcgatgtggaggaaaatcctgggccaatggactggacatggattctgttcctggtggcagccgctacaagggtccattccgacattgtgctgactcagtctcctgcaagtctggccgtgtctcagggacagcgagcaaccatcagttgtaaggctagccagtccgtcgactacgatggggacagttacgtgaactggtatcagcagaagcctggacagtccccaaaactgctgatctatgatgctagtaatctggagtcaggcattcccgcacgattctctggaagtggctcagggacagacttcaccctgaacattcaccctgtcgaggaagaggacgtggctacctactattgccaggaaagcaatgaggacccccgcactttcgggggaggcaccaagctggagatcaaacgaactgtcgcagcccccagcgtgttcatctttccaccctcagacgaacagctgaagagcggaaccgcatccgtggtgtgcctgctgaacaacttctacccccgcgaggccaaggtccagtggaaagtggataacgctctgcagtcaggcaatagccaggaatccgtgactgagcaggattctaaggacagtacctattcactgtcaagcacactgactctgagcaaagcagactacgaaaagcataaagtgtatgcctgcgaagtcacccaccaggggctgaggtctccagtcactaagtctttcaacagaggggaatgctgataactcgag

SEQ ID NO: 66 - DNA, 2241 bp, Artificial Sequence; identical to SEQ ID NO: 44
  DVSF-1 WT nucleic acid sequence

SEQ ID NO: 67 - protein, 739 aa, Artificial Sequence; identical to SEQ ID NO: 45
  DVSF-1 WT amino acid sequence

SEQ ID NO: 68 - DNA, 2241 bp, Artificial Sequence
  DVSF-1 LALA nucleic acid sequence
ggatccgccaccatggactggacttggaggattctgtttctggtcgccgccgctactgggactcacgctcaggcacatctggtcgaatctggaggaggagtggtccagcctggccgatccctgcgactgtcttgcgcagctagcgccttcaacttcagcacaaacgcaatgcactgggtgcgacaggcaccaggcaagggactggagtgggtcgctgtgatctcatacgacggaagccataagtactatgcagattctgtgaaaggccggttcaccatttccagggacaattctaagaacaccctgtatctgcagatgaatagcctgcgcgcagccgataccgcagtgtactattgcgcaactgtcggcgtgctgacctggccagtgaacgccgaatactttcaccattggggacagggcagtctggtctcagtgagctccgcaagtactaagggaccatcagtgttcccactggcaccctctagtaaatctactagtggcgggaccgctgcactgggatgtctggtgaaggactatttccccgagcctgtcaccgtgagctggaattccggagccctgacaagcggcgtccacacttttcccgctgtgctgcagtcaagcggactgtactccctgtcctctgtggtcactgtgcctagttcaagcctgggcactcagacctatatctgcaatgtgaaccacaagccctctaacaccaaagtcgacaagaaagtggaacctaagagctgtgataaaacacatacttgcccaccttgtccagcaccagaggcagctggaggaccaagcgtgttcctgtttccacccaagcctaaagacacactgatgattagccggacacctgaagtcacttgcgtggtcgtggacgtgtcccacgaggaccccgaagtcaagtttaattggtacgtggatggcgtcgaggtgcataacgccaagaccaaaccccgggaggaacagtacaatagcacatatagagtcgtgtccgtcctgactgtgctgcatcaggattggctgaatgggaaggagtataagtgcaaagtgtctaacaaggctctgcctgcaccaatcgagaaaaccattagcaaggctaaaggccagcctagggaaccacaggtgtacacactgcctccaagtcgcgacgagctgaccaagaatcaggtctccctgacatgtctggtgaaaggcttctatccatcagatatcgccgtggagtgggaaagcaacgggcagcccgaaaacaattacaagaccacaccccctgtgctggactctgatggcagtttctttctgtattctaagctgaccgtggacaaaagtagatggcagcaggggaatgtcttttcatgtagcgtgatgcacgaggccctgcacaaccattacacacagaagtccctgtctctgagtcccggaaagaggggccgcaaacggagatcagggagcggagctactaatttcagcctgctgaaacaggcaggggatgtggaggaaaaccccggacctatggcttggaccccactgttcctgtttctgctgacatgctgtcccgggggcagcaattctcagagtgtcctgacacagccaccatcagtgagcggagcaccaggacagagggtgaccatctcctgcacaggcagcagcagcaacattggcgccgggtacgacgtgcattggtatcagcagctgcccggcaccgctcctaagctgctgatctgtggcaacaataaccgcccatctggggtgcccgatcgattctccggctctaaaagtgggacttcagccagcctggctattaccggcctgcaggccgaggacgaagctgattactattgccagagctacgactcaagcctgaccggagtcgtgttcggaggaggaaccaagctgacagtcctgggacagcctaaagccgctccaagcgtgacactgtttcctccatcctctgaggaactgcaggcaaacaaggccaccctggtgtgcctgatttccgacttctaccccggggcagtcactgtggcttggaaggcagatagttcacctgtcaaagccggagtggagactaccacaccatcaaagcagagcaataacaaatacgcagccagctcctatctgtccctgacccctgagcagtggaagtctcacaaatcctattcttgccaggtcactcacgaaggaagcactgtggagaaaactgtcgcaccaaccgaatgtagttgataactcgag

SEQ ID NO: 69 - protein, 739 aa, Artificial Sequence
  DVSF-1 LALA amino acid sequence
MDWTWRILFLVAAATGTHAQAHLVESGGGVVQPGRSLRLSCAASAFNFSTNAMHWVRQAPGKGLEWVAVISYDGSHKYYADSVKGRFTISRDNSKNTLYLQMNSLRAADTAVYYCATVGVLTWPVNAEYFHHWGQGSLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMAWTPLFLFLLTCCPGGSNSQSVLTQPPSVSGAPGQRVTISCTGSSSNIGAGYDVHWYQQLPGTAPKLLICGNNNRPSGVPDRFSGSKSGTSASLAITGLQAEDEADYYCQSYDSSLTGVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 70 - DNA, 2241 bp, Artificial Sequence
  DVSF-2 WT nucleic acid sequence
ggatccgccaccatggactggacatggagaatcctgttcctggtcgccgccgcaaccgggacacacgccgaagtgcagctggtggaatctggagggggatgggtgcagccaggagggtccctgcgactgtcttgcgccgctagtggcttcactttttccagatacgacatgcactgggtcaggcaggtgaccggaaagggcctggaatgggtgagcgcaatcaccacagccggagacacatactatcccgattctgtgaagggccggttcaccattagtcgggagaacgccaaaagctccctgtatctgcagatgaacaatctgagagctggcgacaccgcactgtactattgcgctaggggcccccctacagattgctctagtggacgatgtctgggagtcggagtgggactggacccatgggggcagggaacactggtcactgtgtcaagcgcctccacaaagggaccctctgtgttccctctggctccatcctctaaaagtacttcaggaggaaccgcagcactgggatgtctggtgaaggattacttcccagagcccgtcaccgtgagctggaactccggagctctgactagcggcgtccatacctttcctgcagtgctgcagagttcaggcctgtacagcctgagctccgtggtcaccgtgccatctagttcactggggacccagacatatatctgcaacgtgaatcacaagccatctaatacaaaagtcgacaagaaagtggaacccaagagttgtgataaaactcatacctgcccaccatgtcctgcaccagagctgctgggaggaccatccgtgttcctgtttcctccaaagcccaaagacacactgatgattagcaggacacccgaagtcacttgcgtggtcgtggacgtgagccacgaggaccccgaagtcaagtttaactggtacgtggatggcgtcgaggtgcataatgccaagaccaaaccccgggaggaacagtacaacagtacctatagagtcgtgtcagtcctgacagtgctgcaccaggactggctgaacgggaaagagtataagtgcaaagtgtccaataaggcactgcccgcccctatcgagaaaaccatttctaaggccaaaggacagccccgagaacctcaggtgtacacactgccccctagccgcgacgagctgacaaagaaccaggtctccctgacttgtctggtgaaagggttctatccttcagatatcgccgtggagtgggaaagcaatggacagccagaaaacaattacaagactaccccacccgtgctggactctgatggcagtttctttctgtatagcaagctgaccgtggacaaatcccgctggcagcaggggaacgtctttagctgctccgtgatgcatgaggccctgcacaatcattacactcagaagtctctgagtctgtcacctggaaagaggggacgaaaacgaagaagcggctccggagcaaccaacttcagcctgctgaaacaggccggggatgtggaggaaaatccaggacccatggcatggactcctctgttcctgtttctgctgacctgctgtccaggcgggagcaacagctcctacgaggtgacccagcctccatctgtcagtgtgtcacccggccagaccgcttcaatcacatgtagcggggacaagctgggaaagaaatacacaagttggtatcagcagaaaccaggacagtcacccctgctggtcatctaccaggatactaagcgccctagcggcattccagaacggttcagcggctccaactctgggaatacagctactctgaccatctccggcacccaggccatggacgaggctgattactattgccaggcatgggattctacaactcacgtcattttcggaggcgggaccaagctgacagtgctggggcagcccaaagctgcacctagcgtcaccctgtttcccccttctagtgaggaactgcaggctaataaggcaacactggtgtgtctgatttccgacttctacccaggagcagtcactgtggcatggaaggctgattcaagccccgtcaaagccggagtggaaaccacaactccttcaaagcagagcaacaacaagtacgccgcttcctcttatctgtccctgactcccgagcagtggaagtctcacaaaagttattcatgccaggtgacccatgagggctccactgtcgaaaagaccgtggcccctacagagtgttcttgataactcgag

SEQ ID NO: 71 - protein, 739 aa, Artificial Sequence
  DVSF-2 WT amino acid sequence
MDWTWRILFLVAAATGTHAEVQLVESGGGWVQPGGSLRLSCAASGFTFSRYDMHWVRQVTGKGLEWVSAITTAGDTYYPDSVKGRFTISRENAKSSLYLQMNNLRAGDTALYYCARGPPTDCSSGRCLGVGVGLDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMAWTPLFLFLLTCCPGGSNSSYEVTQPPSVSVSPGQTASITCSGDKLGKKYTSWYQQKPGQSPLLVIYQDTKRPSGIPERFSGSNSGNTATLTISGTQAMDEADYYCQAWDSTTHVIFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 72 - DNA, 2241 bp, Artificial Sequence
  DVSF-2 LALA nucleic acid sequence
ggatccgccaccatggactggacatggagaatcctgttcctggtcgccgccgctactgggactcacgccgaagtgcagctggtcgagagtggagggggatgggtgcagcccggcggcagcctgaggctgtcttgcgccgctagtggcttcactttttctagatacgacatgcactgggtccggcaggtgaccgggaagggactggaatgggtgagcgccatcaccacagcaggggacacatactatcccgattctgtgaagggcaggttcaccattagtagggagaacgcaaaaagctccctgtatctgcagatgaacaatctgagagccggcgacaccgctctgtactattgcgccaggggccctcccacagattgctctagtggacgctgtctgggagtcggagtgggactggacccatggggacaggggacactggtcaccgtgagcagcgcctccactaagggaccaagcgtgttccctctggcaccatcctctaaaagtacttcagggggcaccgcagccctgggatgtctggtgaaggattacttcccagagcccgtcacagtgagctggaactccggggccctgacttccggagtccacacctttcctgctgtgctgcagagttcaggcctgtactctctgagctccgtggtcacagtgccatctagttcactgggaacccagacatatatctgcaacgtgaatcacaagccaagtaatactaaagtcgacaagaaagtggaacccaagtcttgtgataaaactcatacctgcccaccctgtcctgcaccagaggctgcaggagggccatccgtgttcctgtttcctccaaagcccaaagacaccctgatgattagccggacacccgaagtcacttgcgtggtcgtggacgtgtcccacgaggaccccgaagtcaagtttaactggtacgtggatggcgtcgaggtgcataatgccaagacaaaacccagggaggaacagtacaacagtacctatagagtcgtgtcagtcctgacagtgctgcaccaggactggctgaacggaaaggagtataagtgcaaagtgtctaataaggctctgcccgcacctatcgagaaaaccattagcaaggccaaagggcagccccgagaacctcaggtgtacacactgcccccttcccgcgacgagctgacaaagaaccaggtctctctgacttgtctggtgaaaggattctatccttcagatatcgccgtggagtgggaaagcaatgggcagccagaaaacaattacaagactaccccacccgtgctggactctgatggcagtttctttctgtatagcaagctgaccgtggacaaatcccgctggcagcagggaaacgtctttagctgctccgtgatgcatgaggccctgcacaatcattacacccagaagtctctgagtctgtcacctgggaagcgaggacgaaaaaggagaagcggctccggagctacaaacttctccctgctgaaacaggcaggagatgtggaggaaaatccagggcccatggcctggactcctctgttcctgtttctgctgacctgctgtccaggcggaagcaacagctcctacgaggtgacccagcctccaagcgtgagcgtgagcccaggccagaccgcttcaatcacatgtagcggagacaagctggggaagaaatacactagttggtatcagcagaaaccagggcagtcacccctgctggtcatctaccaggataccaagcgccctagcggcattccagaacgattcagcggctccaactctggaaatacagccactctgaccatcagcggcacccaggcaatggacgaggccgattactattgccaggcttgggattccacaactcacgtcattttcgggggcggaaccaagctgacagtgctgggacagcccaaagccgctccttccgtcaccctgtttcccccttctagtgaggaactgcaggccaataaggccaccctggtgtgcctgattagcgacttctaccccggagctgtcactgtggcatggaaggccgattcaagccccgtcaaagcaggggtggaaaccacaactccttcaaagcagagcaacaacaagtacgcagcctcctcttatctgtccctgacccctgagcagtggaagtctcataaaagttattcatgtcaggtcacccatgagggcagcacagtggaaaaaaccgtggcaccaacagaatgtagctgataactcgag

SEQ ID NO: 73 - protein, 739 aa, Artificial Sequence
  DVSF-2 LALA amino acid sequence
MDWTWRILFLVAAATGTHAEVQLVESGGGWVQPGGSLRLSCAASGFTFSRYDMHWVRQVTGKGLEWVSAITTAGDTYYPDSVKGRFTISRENAKSSLYLQMNNLRAGDTALYYCARGPPTDCSSGRCLGVGVGLDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMAWTPLFLFLLTCCPGGSNSSYEVTQPPSVSVSPGQTASITCSGDKLGKKYTSWYQQKPGQSPLLVIYQDTKRPSGIPERFSGSNSGNTATLTISGTQAMDEADYYCQAWDSTTHVIFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 74 - DNA, 2229 bp, Artificial Sequence
  DVSF-3 WT nucleic acid sequence
ggatccgccaccatggactggacatggagaatcctgttcctggtcgccgccgcaaccgggacacacgccgaagtgcagctggtggaatcagggggagggctggtgcagcctggaagaagtctgaggctgtcatgcgccgctagcggcttcacctttgacgattacgccatgttctgggtgaggcaggctccaggcaagggactggaatggatcagcggcatttcctggaactctgcaactatcgggtatgccgactccgtgaaaggacggtttaccatttcaagagacaacgccaagaaaagcctggatctgcagatgaattccctgcggcccgacgataccgctctgtactattgcgcaaagggaggacctagaggcctgcagctgctgagctcctgggtggactactggggacagggcactctggtcaccgtgtctagtgcttccacaaagggaccttctgtgttcccactggcaccctcaagcaaatcaacaagcggaggaactgcagcactgggatgtctggtgaaggattatttccccgagcctgtcaccgtgagttggaactcaggagcactgacttccggagtccacacctttccagcagtgctgcagtcctctggactgtacagcctgagttcagtggtcacagtgcctagctcctctctgggcacacagacttatatctgcaacgtgaatcacaagcctagcaatactaaagtcgacaagaaagtggaaccaaagtcctgtgataaaacccatacatgcccaccttgtccagcaccagagctgctggggggaccaagcgtgttcctgtttccacccaagcccaaagacacactgatgatttctcggacccctgaagtcacatgtgtggtcgtggacgtgagccacgaggaccccgaagtcaagttcaactggtacgtggatggcgtcgaggtgcataatgctaagaccaaaccccgagaggaacagtacaacagcacttatcgggtcgtgtccgtcctgaccgtgctgcaccaggactggctgaacgggaaggagtataagtgcaaagtgtccaataaggccctgcctgctccaatcgagaaaacaatttctaaggcaaaaggacagcctcgcgaaccacaggtgtacactctgcctccatcccgagacgagctgaccaagaaccaggtctctctgacatgtctggtgaaaggcttctatccaagtgatatcgctgtggagtgggaaagcaatgggcagcccgaaaacaattacaagaccacaccccctgtgctggacagcgatggctccttctttctgtattctaagctgaccgtggataaaagtagatggcagcaggggaacgtcttttcctgctctgtgatgcatgaggccctgcacaatcattacacacagaagagtctgtcactgagcccagggaagcgaggacggaaacggagatccgggtctggagcaaccaacttctccctgctgaaacaggcaggcgacgtggaggaaaatccaggacctatggtcctgcagacccaggtgtttatctctctgctgctgtggattagtggcgcctacggggatatccagatgacacagtcccccagttcactgagtgcctcagtcggcgacagggtgactatcacctgtcgcgctagccaggatattaggcgctacctgaactggtatcagcagcgaccaggacgagtgcctcagctgctgatctacactacctccaccctgcagtctggagtcccaagtaggttcagcggctccgggtctgtgacagactttacactgactattagctccctgcagcccgaagatttcggcacttactattgccagcagagttattcaccaccccacacatttggacagggcactaagctggaaatcaaaactgtcgctgcaccctcagtgttcatttttcctccatctgacgagcagctgaagtcaggcaccgccagcgtcgtgtgtctgctgaacaatttctaccctcgcgaggctaaggtccagtggaaagtggataacgcactgcagtctgggaatagtcaggagtcagtgacagaacaggacagcaaggattccacttattctctgtctagtaccctgacactgagcaaagccgactacgagaagcacaaagtctatgcttgcgaagtgacccatcaggggctgagaagtcccgtgacaaagagcttcaacaggggagagtgttgataactcgag

SEQ ID NO: 75 - protein, 735 aa, Artificial Sequence
  DVSF-3 WT amino acid sequence
MDWTWRILFLVAAATGTHAEVQLVESGGGLVQPGRSLRLSCAASGFTFDDYAMFWVRQAPGKGLEWISGISWNSATIGYADSVKGRFTISRDNAKKSLDLQMNSLRPDDTALYYCAKGGPRGLQLLSSWVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSLSASVGDRVTITCRASQDIRRYLNWYQQRPGRVPQLLIYTTSTLQSGVPSRFSGSGSVTDFTLTISSLQPEDFGTYYCQQSYSPPHTFGQGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 76 - DNA, 2229 bp, Artificial Sequence
  DVSF-3 LALA nucleic acid sequence
ggatccgccaccatggactggacttggagaatcctgtttctggtcgccgccgcaactggaacccacgccgaggtgcagctggtcgaatcagggggaggcctggtgcagcctgggagaagtctgcggctgtcatgcgccgctagcggcttcacctttgacgattacgcaatgttctgggtgaggcaggcaccaggcaagggactggaatggatcagcggcatttcctggaactctgctaccatcggatatgcagacagcgtgaaagggaggtttacaatttctagagacaacgccaagaaaagtctggatctgcagatgaattcactgcgccccgacgataccgccctgtactattgcgctaagggcggacccaggggcctgcagctgctgagctcctgggtggactactgggggcagggcactctggtcaccgtgtctagtgcctccacaaagggccctagcgtgttcccactggctccctcaagcaaatcaacaagcgggggcactgcagccctgggatgtctggtgaaggattatttccccgagcctgtcaccgtgagttggaactcaggggctctgactagcggcgtccacacctttcccgcagtgctgcagtcctctggcctgtacagcctgagttcagtggtcactgtccctagctcctctctgggaacacagacttatatctgcaacgtgaatcacaagccttccaataccaaagtcgacaagaaagtggaaccaaagtcttgtgataaaacccatacatgccctccctgtccagcaccagaggctgcaggagggccaagcgtgttcctgtttccacccaagcccaaagacacactgatgattagccggacccctgaagtcacatgcgtggtcgtggacgtgagccacgaggaccccgaagtcaagtttaactggtacgtggatggcgtcgaggtgcataatgctaagaccaaaccccgagaggaacagtacaacagtacttatagggtcgtgtcagtcctgaccgtgctgcaccaggactggctgaacgggaaggagtataagtgcaaagtgtccaataaggcactgcctgccccaatcgagaaaactatttctaaggctaaaggccagcctagagaaccacaggtgtacaccctgcctccaagccgggacgagctgaccaagaaccaggtcagcctgacatgtctggtgaaaggattctatccatccgatatcgcagtggagtgggaatctaatgggcagcccgaaaacaattacaagaccacaccccctgtgctggacagcgatggcagcttcttcctgtatagcaagctgaccgtggataaatcccgctggcagcaggggaacgtcttttcctgctctgtgatgcatgaggccctgcacaatcattacacacagaagagtctgtcactgagcccaggaaagcgagggaggaaaaggagatccggatctggggctactaacttctccctgctgaagcaggcaggcgacgtggaggaaaatcccggacctatggtcctgcagacacaggtgtttatcagcctgctgctgtggatttccggcgcttacggagatatccagatgactcagtcccccagttcactgagtgcatcagtcggcgaccgggtgactatcacctgtcgcgcctctcaggatattcggcgctacctgaattggtatcagcagcgaccaggacgagtgcctcagctgctgatctacactacctccacactgcagtctggcgtcccaagtaggttcagcggctccggatctgtgactgactttacactgactattagctccctgcagcccgaggatttcggcacctactattgccagcagagttattcaccaccccacacatttgggcagggcactaagctggaaatcaaaaccgtcgccgctcccagcgtgttcatctttcctccaagtgacgagcagctgaagtcaggaacagccagcgtggtgtgcctgctgaacaatttctaccctagagaagccaaggtccagtggaaagtggataacgctctgcagtctgggaatagtcaggagtcagtgacagaacaggacagcaaggattccacttattctctgtctagtaccctgacactgagcaaagcagactacgagaagcataaagtgtatgcctgcgaagtcacccaccaggggctgcggtcaccagtcacaaaatcctttaacagaggcgaatgctgataactcgag

SEQ ID NO: 77 - protein, 735 aa, Artificial Sequence
  DSVF-3 LALA amino acid sequence
MDWTWRILFLVAAATGTHAEVQLVESGGGLVQPGRSLRLSCAASGFTFDDYAMFWVRQAPGKGLEWISGISWNSATIGYADSVKGRFTISRDNAKKSLDLQMNSLRPDDTALYYCAKGGPRGLQLLSSWVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSLSASVGDRVTITCRASQDIRRYLNWYQQRPGRVPQLLIYTTSTLQSGVPSRFSGSGSVTDFTLTISSLQPEDFGTYYCQQSYSPPHTFGQGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 78 - DNA, 2217 bp, Artificial Sequence
  Anti-PSMA nucleic acid sequence
ggatccgccaccatggattggacatggaggattctgtttctggtcgccgccgccacaggaacccacgccgaagtgcagctggtgcagtcaggagccgaggtgaagaaaccaggcgaaagtctgaaaatctcatgcaaaggaagtgggtactcattcactagcaactggattggatgggtgcggcagatgccaggcaagggactggagtggatgggaatcatctaccccggggactccgatacacgctatagtccttcatttcagggccaggtgacaatctctgccgacaaaagcatttccactgcttatctgcagtggagctccctgaaggcttccgataccgcaatgtactattgcgccaggcagacaggcttcctgtggtctagtgacctgtgggggagaggcaccctggtcacagtgtcaagcgcctctaccaaaggaccaagcgtgttcccactggctccttcctctaagtctactagtggcggaaccgccgctctgggatgtctggtgaaggattacttccctgagccagtcacagtgtcctggaactctggcgctctgaccagcggagtccacacatttcccgcagtgctgcagagttcaggcctgtactccctgagctccgtggtcacagtcccttctagttcactgggaactcagacctatatctgcaacgtgaatcacaaaccttccaatactaaggtcgacaagaaagtggaaccaaaatcttgtgataagacacatacttgccctccctgtccagcacctgagctgctgggcggcccaagcgtgttcctgtttccacccaagcccaaagataccctgatgattagcaggacaccagaagtcacttgcgtggtcgtggacgtgtcccacgaggaccccgaagtcaagttcaactggtacgtggacggcgtcgaggtgcataatgctaagaccaaaccaagagaggaacagtacaactcaacctatcgggtcgtgagcgtcctgacagtgctgcaccaggactggctgaacggaaaggagtataagtgcaaagtgtctaacaaggccctgccagctcccatcgagaagactattagcaaggctaaagggcagccacgcgaaccccaggtgtacaccctgcctccatcacgagatgagctgacaaaaaaccaggtctctctgacttgtctggtgaagggattctatccctctgacatcgcagtggagtgggaaagtaatgggcagcctgaaaacaattacaagaccacaccccctgtgctggacagtgatggatcattctttctgtatagtaaactgaccgtggataagtcaagatggcagcaggggaacgtcttttcatgcagcgtgatgcatgaggccctgcacaatcattacacccagaagtccctgtctctgagtcctggcaaacggggacgcaagaggagatcaggaagcggggctacaaacttctccctgctgaagcaggcaggggacgtggaggaaaatcctggcccaatggtcctgcagacccaggtgtttatctccctgctgctgtggatttctggggcatacggcgccatccagctgacacagtctcccagctccctgtccgcatctgtcggcgaccgagtgaccatcacatgtagggccagccaggatatttctagtgctctggcatggtaccagcagaagcctgggaaagcaccaaagctgctgatctatgatgcctcaagcctggaatccggagtgcctagccggttctccggatacggaagtgggacagactttactctgaccattaacagcctgcagcctgaggatttcgccacttactattgccagcagttcaatagctatccactgacttttggagggggcaccaaagtggaaatcaagcgaactgtcgcagcccctagcgtgttcatttttccaccctccgatgagcagctgaagagcggcaccgcttccgtggtgtgcctgctgaacaacttctacccacgcgaggccaaagtccagtggaaggtggacaacgctctgcagtctggaaatagtcaggagtcagtgactgaacaggacagcaaagattccacctattctctgtcctctacactgactctgagcaaggcagactacgagaagcataaagtgtatgcctgcgaagtcacccaccaggggctgtcctcaccagtcactaaatctttcaatcggggagaatgttgataactcgag

SEQ ID NO: 79 - protein, 731 aa, Artificial Sequence
  Anti-PSMA amino acid sequence
MDWTWRILFLVAAATGTHAEVQLVQSGAEVKKPGESLKISCKGSGYSFTSNWIGWVRQMPGKGLEWMGIIYPGDSDTRYSPSFQGQVTISADKSISTAYLQWSSLKASDTAMYYCARQTGFLWSSDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGAIQLTQSPSSLSASVGDRVTITCRASQDISSALAWYQQKPGKAPKLLIYDASSLESGVPSRFSGYGSGTDFTLTINSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 733 aa, Artificial Sequence
  CHIKV-snapi amino acid sequence
MDWTWILFLVAAATRVHSQVQLQQPGAALVKPGASAMMSCKASGYTFTSYWITWVKQRPGQGLEWIGDIYPGTGRTIYKEKFKTKATLTVDTSSSTAFMQLNSLTSEDSAVYYCARGYGSPYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMDWTWILFLVAAATRVHSDIVLTQSPASLAVSQGQRATISCKASQSVDYDGDSYVNWYQQKPGQSPKLLIYDASNLESGIPARFSGSGSGTDFTLNIHPVEEEDVATYYCQESNEDPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 81 - protein, 457 aa, Artificial Sequence
  CHIKV E1 consensus
MDWTWILFLVAAATRVHSYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAVGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 82 - protein, 441 aa, Artificial Sequence
  CHIKV E2 consensus
MDWTWILFLVAAATRVHSSTKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKA

SEQ ID NO: 83 - protein, 279 aa, Artificial Sequence
  CHIKV capsid consensus
MDWTWILFLVAAATRVHSMEFIPTQTFYNRRYQPRPWTPRPTIQVIRPRPRPQRQAGQLAQLISAVNKLTMRAVPQQKPRRNRKNKKQKQKQQAPQNNTNQKKQPPKKKPAQKKKKPGRRERMCMKIENDCIFEVKHEGKVTGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEW

SEQ ID NO: 84 - protein, 439 aa, Artificial Sequence
  CHIKV E1 consensus
YEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAVGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 85 - protein, 423 aa, Artificial Sequence
  CHIKV E2 consensus
STKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKA

SEQ ID NO: 86 - protein, 261 aa, Artificial Sequence
  CHIKV capsid consensus
MEFIPTQTFYNRRYQPRPWTPRPTIQVIRPRPRPQRQAGQLAQLISAVNKLTMRAVPQQKPRRNRKNKKQKQKQQAPQNNTNQKKQPPKKKPAQKKKKPGRRERMCMKIENDCIFEVKHEGKVTGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEW

SEQ ID NO: 87 - protein, 998 aa, Artificial Sequence
  CHIKV Env consensus
MDWTWILFLVAAATRVHSSLAIPVMCLLANTTFPCSQPPCTPCCYEKEPEETLRMLEDNVMRPGYYQLLQASLTCSPHRQRRRGRKRRSATMDWTWILFLVAAATRVHSSTKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKARGRKRRSATMDWTWILFLVAAATRVHSYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAVGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 88 - protein, 980 aa, Artificial Sequence
  CHIKV Env consensus
SLAIPVMCLLANTTFPCSQPPCTPCCYEKEPEETLRMLEDNVMRPGYYQLLQASLTCSPHRQRRRGRKRRSATMDWTWILFLVAAATRVHSSTKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKARGRKRRSATMDWTWILFLVAAATRVHSYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAVGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 89 - DNA, 1374 bp, Artificial Sequence
  CHIKV E1 consensus
atggactggacctggatcctgtttctggtcgctgctgccacccgggtgcacagctacgagcacgtgaccgtgatccccaacaccgtgggcgtgccctacaagaccctggtgaacaggcccggctacagccccatggtgctggaaatggaactgctgtccgtgaccctggaacccaccctgagcctggactacatcacctgcgagtacaagacagtgatccccagcccctacgtgaagtgctgcggcaccgccgagtgcaaggacaagaacctgcccgactacagctgcaaggtgttcaccggcgtgtaccccttcatgtggggcggagcctactgcttctgcgacgccgagaacacccagctgtccgaggcccacgtggagaagagcgagagctgcaagaccgagttcgccagcgcctaccgggcccacacagccagcgccagcgccaagctgcgggtgctgtaccagggcaacaacatcaccgtgaccgcctacgccaacggcgaccacgccgtgacagtgaaggacgccaagttcatcgtgggccccatgagcagcgcctggacccccttcgacaacaagatcgtggtgtacaagggcgacgtgtacaacatggactacccccccttcggagccggcagacccggccagttcggcgacatccagagccggacccccgagagcaaggacgtgtacgccaatacccagctggtgctgcagagacccgccgtgggcaccgtgcacgtgccttacagccaggcccccagcggcttcaagtactggctgaaagagaggggcgccagcctgcagcacaccgcccccttcggctgccagatcgccaccaaccccgtgcgggccgtgaattgtgccgtgggcaacatgcccatcagcatcgacatccccgaggccgccttcaccagggtggtggacgcccccagcctgaccgacatgagctgcgaggtgcccgcctgcacccacagcagcgacttcggcggcgtggccatcatcaagtacgccgccagcaagaaaggcaagtgcgccgtgcacagcatgaccaatgccgtgaccatccgggaggccgagatcgaggtggagggcaacagccagctgcagatcagcttcagcaccgccctggccagcgccgagttccgggtgcaggtctgcagcacccaggtgcactgtgccgccgagtgtcacccccccaaggaccacatcgtgaactaccccgccagccacaccaccctgggcgtgcaggacatcagcgccaccgccatgagctgggtgcagaagatcacaggcggcgtcggcctggtggtggccgtggccgccctgatcctgatcgtggtgctgtgcgtgagcttcagccggcactga

SEQ ID NO: 90 - DNA, 1326 bp, Artificial Sequence
  CHIKV E2 consensus
atggactggacctggatcctgttcctggtcgctgctgccacaagagtgcacagcagcaccaaggacaacttcaacgtgtacaaggccacccggccctacctggcccactgccccgattgcggcgagggccacagctgccacagccccgtggccctggaacggatccggaacgaggccaccgacggcaccctgaagatccaggtgtccctgcagatcggcatcaagaccgacgacagccacgactggaccaagctgcggtacatggacaaccacatgcccgccgacgccgagagagccggcctgttcgtccggaccagcgccccctgcaccatcaccggcaccatgggccacttcatcctggcccggtgccccaagggcgagacactgaccgtgggcttcaccgacagccggaagatcagccactcctgcacccaccccttccaccacgacccccccgtgatcggccgggagaagttccacagcaggccccagcacggcaaagagctgccctgcagcacctacgtgcagagcaccgccgccacaaccgaggaaatcgaggtgcacatgccccccgatacccccgaccggaccctgatgagccagcagagcggcaacgtgaagatcaccgtgaacggccagaccgtgcggtacaagtgcaactgcggcggcagcaacgagggcctgaccaccaccgacaaggtgatcaacaactgcaaggtggaccagtgccacgccgccgtgaccaaccacaagaagtggcagtacaacagccccctggtgccccggaatgccgagctgggcgaccggaagggcaagatccacatccccttccccctggccaacgtgacctgccgggtgcccaaggcccggaaccccaccgtgacctacggcaagaaccaggtgatcatgctgctgtaccccgaccaccccaccctgctgtcctaccggaacatgggcgaggaacccaactaccaagaggagtgggtcatgcacaagaaagaagtggtgctgaccgtccccaccgagggcctggaagtgacctggggcaacaacgagccctacaagtactggccccagctgtccaccaacggcaccgcccacggccacccccacgagatcatcctgtactactacgagctgtaccctaccatgaccgtggtggtggtgtccgtggccacctttatcctgctgtccatggtcggcatggccgctggcatgtgcatgtgcgccaggaggcgctgtatcaccccctacgagctgacacctggcgccaccgtgccctttctgctgtccctgatctgctgcatccggaccgccaaggcctga

SEQ ID NO: 91 - DNA, 840 bp, Artificial Sequence
  CHIKV capsid consensus
atggactggacctggatcctgttcctggtggccgctgccacccgggtgcacagcatggaattcatccccacccagaccttctacaaccggcgctaccagcccagaccctggacccccaggcccaccatccaggtgatccggcccaggcccagaccccagaggcaggccgggcagctggcacagctgatcagcgccgtgaacaagctgaccatgagagccgtgccccagcagaagcccaggcggaaccggaagaacaagaagcagaagcagaaacagcaggccccccagaacaacaccaaccagaagaagcagccccccaagaagaagcctgcccagaagaagaagaaacccggcaggcgggagcggatgtgcatgaagatcgagaacgactgcatcttcgaggtgaagcacgagggcaaggtgaccggctacgcctgcctggtcggcgacaaagtgatgaagcccgcccacgtgaagggcaccatcgacaacgccgacctggccaagctggccttcaagcggagcagcaagtacgacctggaatgcgcccagatccccgtgcacatgaagagcgacgccagcaagttcacccacgagaagcccgagggctactacaactggcaccacggagccgtgcagtacagcggcggcaggttcaccatccccacaggcgccggaaagcccggcgacagcggcaggcccatcttcgacaacaagggccgggtggtggccatcgtgctgggcggagccaacgagggcgccaggaccgccctgagcgtggtgacctggaacaaggacatcgtgaccaagatcacccccgagggcgccgaagagtggtga

SEQ ID NO: 92 - DNA, 1320 bp, Artificial Sequence
  CHIKV E1 consensus
tacgagcacgtgaccgtgatccccaacaccgtgggcgtgccctacaagaccctggtgaacaggcccggctacagccccatggtgctggaaatggaactgctgtccgtgaccctggaacccaccctgagcctggactacatcacctgcgagtacaagacagtgatccccagcccctacgtgaagtgctgcggcaccgccgagtgcaaggacaagaacctgcccgactacagctgcaaggtgttcaccggcgtgtaccccttcatgtggggcggagcctactgcttctgcgacgccgagaacacccagctgtccgaggcccacgtggagaagagcgagagctgcaagaccgagttcgccagcgcctaccgggcccacacagccagcgccagcgccaagctgcgggtgctgtaccagggcaacaacatcaccgtgaccgcctacgccaacggcgaccacgccgtgacagtgaaggacgccaagttcatcgtgggccccatgagcagcgcctggacccccttcgacaacaagatcgtggtgtacaagggcgacgtgtacaacatggactacccccccttcggagccggcagacccggccagttcggcgacatccagagccggacccccgagagcaaggacgtgtacgccaatacccagctggtgctgcagagacccgccgtgggcaccgtgcacgtgccttacagccaggcccccagcggcttcaagtactggctgaaagagaggggcgccagcctgcagcacaccgcccccttcggctgccagatcgccaccaaccccgtgcgggccgtgaattgtgccgtgggcaacatgcccatcagcatcgacatccccgaggccgccttcaccagggtggtggacgcccccagcctgaccgacatgagctgcgaggtgcccgcctgcacccacagcagcgacttcggcggcgtggccatcatcaagtacgccgccagcaagaaaggcaagtgcgccgtgcacagcatgaccaatgccgtgaccatccgggaggccgagatcgaggtggagggcaacagccagctgcagatcagcttcagcaccgccctggccagcgccgagttccgggtgcaggtctgcagcacccaggtgcactgtgccgccgagtgtcacccccccaaggaccacatcgtgaactaccccgccagccacaccaccctgggcgtgcaggacatcagcgccaccgccatgagctgggtgcagaagatcacaggcggcgtcggcctggtggtggccgtggccgccctgatcctgatcgtggtgctgtgcgtgagcttcagccggcactga

SEQ ID NO: 93 - DNA, 1273 bp, Artificial Sequence
  CHIKV E1 consensus
cagcaccaaggacaacttcaacgtgtacaaggccacccggccctacctggcccactgccccgattgcggcgagggccacagctgccacagccccgtggccctggaacggatccggaacgaggccaccgacggcaccctgaagatccaggtgtccctgcagatcggcatcaagaccgacgacagccacgactggaccaagctgcggtacatggacaaccacatgcccgccgacgccgagagagccggcctgttcgtccggaccagcgccccctgcaccatcaccggcaccatgggccacttcatcctggcccggtgccccaagggcgagacactgaccgtgggcttcaccgacagccggaagatcagccactcctgcacccaccccttccaccacgacccccccgtgatcggccgggagaagttccacagcaggccccagcacggcaaagagctgccctgcagcacctacgtgcagagcaccgccgccacaaccgaggaaatcgaggtgcacatgccccccgatacccccgaccggaccctgatgagccagcagagcggcaacgtgaagatcaccgtgaacggccagaccgtgcggtacaagtgcaactgcggcggcagcaacgagggcctgaccaccaccgacaaggtgatcaacaactgcaaggtggaccagtgccacgccgccgtgaccaaccacaagaagtggcagtacaacagccccctggtgccccggaatgccgagctgggcgaccggaagggcaagatccacatccccttccccctggccaacgtgacctgccgggtgcccaaggcccggaaccccaccgtgacctacggcaagaaccaggtgatcatgctgctgtaccccgaccaccccaccctgctgtcctaccggaacatgggcgaggaacccaactaccaagaggagtgggtcatgcacaagaaagaagtggtgctgaccgtccccaccgagggcctggaagtgacctggggcaacaacgagccctacaagtactggccccagctgtccaccaacggcaccgcccacggccacccccacgagatcatcctgtactactacgagctgtaccctaccatgaccgtggtggtggtgtccgtggccacctttatcctgctgtccatggtcggcatggccgctggcatgtgcatgtgcgccaggaggcgctgtatcaccccctacgagctgacacctggcgccaccgtgccctttctgctgtccctgatctgctgcatccggaccgccaaggcctga

SEQ ID NO: 94 - DNA, 786 bp, Artificial Sequence
  CHIKV capsid consensus
atggaattcatccccacccagaccttctacaaccggcgctaccagcccagaccctggacccccaggcccaccatccaggtgatccggcccaggcccagaccccagaggcaggccgggcagctggcacagctgatcagcgccgtgaacaagctgaccatgagagccgtgccccagcagaagcccaggcggaaccggaagaacaagaagcagaagcagaaacagcaggccccccagaacaacaccaaccagaagaagcagccccccaagaagaagcctgcccagaagaagaagaaacccggcaggcgggagcggatgtgcatgaagatcgagaacgactgcatcttcgaggtgaagcacgagggcaaggtgaccggctacgcctgcctggtcggcgacaaagtgatgaagcccgcccacgtgaagggcaccatcgacaacgccgacctggccaagctggccttcaagcggagcagcaagtacgacctggaatgcgcccagatccccgtgcacatgaagagcgacgccagcaagttcacccacgagaagcccgagggctactacaactggcaccacggagccgtgcagtacagcggcggcaggttcaccatccccacaggcgccggaaagcccggcgacagcggcaggcccatcttcgacaacaagggccgggtggtggccatcgtgctgggcggagccaacgagggcgccaggaccgccctgagcgtggtgacctggaacaaggacatcgtgaccaagatcacccccgagggcgccgaagagtggtga

SEQ ID NO: 95 - DNA, 3008 bp, Artificial Sequence
  CHIKV Env consensus
atggactggacctggatcctgttcctggtggctgctgccacccgcgtgcacagcagcctggccatccccgtgatgtgcctgctggccaacaccaccttcccttgcagccagcccccctgcaccccctgctgctacgagaaagagcccgaggaaaccctgcggatgctggaagataacgtgatgaggcccggctactaccagctgctccaggccagcctgacctgctccccccaccggcagcggcggcgcgggcgcaaacgccgctctgccaccatggactggacctggatcctgttcctggtcgctgctgccacaagagtgcacagcagcaccaaggacaacttcaacgtgtacaaggccacccggccctacctggcccactgccccgattgcggcgagggccacagctgccacagccccgtggccctggaacggatccggaacgaggccaccgacggcaccctgaagatccaggtgtccctgcagatcggcatcaagaccgacgacagccacgactggaccaagctgcggtacatggacaaccacatgcccgccgacgccgagagagccggcctgttcgtccggaccagcgccccctgcaccatcaccggcaccatgggccacttcatcctggcccggtgccccaagggcgagacactgaccgtgggcttcaccgacagccggaagatcagccactcctgcacccaccccttccaccacgacccccccgtgatcggccgggagaagttccacagcaggccccagcacggcaaagagctgccctgcagcacctacgtgcagagcaccgccgccacaaccgaggaaatcgaggtgcacatgccccccgatacccccgaccggaccctgatgagccagcagagcggcaacgtgaagatcaccgtgaacggccagaccgtgcggtacaagtgcaactgcggcggcagcaacgagggcctgaccaccaccgacaaggtgatcaacaactgcaaggtggaccagtgccacgccgccgtgaccaaccacaagaagtggcagtacaacagccccctggtgccccggaatgccgagctgggcgaccggaagggcaagatccacatccccttccccctggccaacgtgacctgccgggtgcccaaggcccggaaccccaccgtgacctacggcaagaaccaggtgatcatgctgctgtaccccgaccaccccaccctgctgtcctaccggaacatgggcgaggaacccaactaccaagaggagtgggtcatgcacaagaaagaagtggtgctgaccgtccccaccgagggcctggaagtgacctggggcaacaacgagccctacaagtactggccccagctgtccaccaacggcaccgcccacggccacccccacgagatcatcctgtactactacgagctgtaccctaccatgaccgtggtggtggtgtccgtggccacctttatcctgctgtccatggtcggcatggccgctggcatgtgcatgtgcgccaggaggcgctgtatcaccccctacgagctgacacctggcgccaccgtgccctttctgctgtccctgatctgctgcatccggaccgccaaggcccgcgggcgcaaacgccgctctgccaccatggactggacctggatcctgtttctggtcgctgctgccacccgggtgcacagctacgagcacgtgaccgtgatccccaacaccgtgggcgtgccctacaagaccctggtgaacaggcccggctacagccccatggtgctggaaatggaactgctgtccgtgaccctggaacccaccctgagcctggactacatcacctgcgagtacaagacagtgatccccagcccctacgtgaagtgctgcggcaccgccgagtgcaaggacaagaacctgcccgactacagctgcaaggtgttcaccggcgtgtaccccttcatgtggggcggagcctactgcttctgcgacgccgagaacacccagctgtccgaggcccacgtggagaagagcgagagctgcaagaccgagttcgccagcgcctaccgggcccacacagccagcgccagcgccaagctgcgggtgctgtaccagggcaacaacatcaccgtgaccgcctacgccaacggcgaccacgccgtgacagtgaaggacgccaagttcatcgtgggccccatgagcagcgcctggacccccttcgacaacaagatcgtggtgtacaagggcgacgtgtacaacatggactacccccccttcggagccggcagacccggccagttcggcgacatccagagccggacccccgagagcaaggacgtgtacgccaatacccagctggtgctgcagagacccgccgtgggcaccgtgcacgtgccttacagccaggcccccagcggcttcaagtactggctgaaagagaggggcgccagcctgcagcacaccgcccccttcggctgccagatcgccaccaaccccgtgcgggccgtgaattgtgccgtgggcaacatgcccatcagcatcgacatccccgaggccgccttcaccagggtggtggacgcccccagcctgaccgacatgagctgcgaggtgcccgcctgcacccacagcagcgacttcggcggcgtggccatcatcaagtacgccgccagcaagaaaggcaagtgcgccgtgcacagcatgaccaatgccgtgaccatccgggaggccgagatcgaggtggagggcaacagccagctgcagatcagcttcagcaccgccctggccagcgccgagttccgggtgcaggtctgcagcacccaggtgcactgtgccgccgagtgtcacccccccaaggaccacatcgtgaactaccccgccagccacaccaccctgggcgtgcaggacatcagcgccaccgccatgagctgggtgcagaagatcacaggcggcgtcggcctggtggtggccgtggccgccctgatcctgatcgtggtgctgtgcgtgagcttcagccggcactgatgagcggccgc

SEQ ID NO: 96 - DNA, 2954 bp, Artificial Sequence
  CHIKV Env consensus
agcctggccatccccgtgatgtgcctgctggccaacaccaccttcccttgcagccagcccccctgcaccccctgctgctacgagaaagagcccgaggaaaccctgcggatgctggaagataacgtgatgaggcccggctactaccagctgctccaggccagcctgacctgctccccccaccggcagcggcggcgcgggcgcaaacgccgctctgccaccatggactggacctggatcctgttcctggtcgctgctgccacaagagtgcacagcagcaccaaggacaacttcaacgtgtacaaggccacccggccctacctggcccactgccccgattgcggcgagggccacagctgccacagccccgtggccctggaacggatccggaacgaggccaccgacggcaccctgaagatccaggtgtccctgcagatcggcatcaagaccgacgacagccacgactggaccaagctgcggtacatggacaaccacatgcccgccgacgccgagagagccggcctgttcgtccggaccagcgccccctgcaccatcaccggcaccatgggccacttcatcctggcccggtgccccaagggcgagacactgaccgtgggcttcaccgacagccggaagatcagccactcctgcacccaccccttccaccacgacccccccgtgatcggccgggagaagttccacagcaggccccagcacggcaaagagctgccctgcagcacctacgtgcagagcaccgccgccacaaccgaggaaatcgaggtgcacatgccccccgatacccccgaccggaccctgatgagccagcagagcggcaacgtgaagatcaccgtgaacggccagaccgtgcggtacaagtgcaactgcggcggcagcaacgagggcctgaccaccaccgacaaggtgatcaacaactgcaaggtggaccagtgccacgccgccgtgaccaaccacaagaagtggcagtacaacagccccctggtgccccggaatgccgagctgggcgaccggaagggcaagatccacatccccttccccctggccaacgtgacctgccgggtgcccaaggcccggaaccccaccgtgacctacggcaagaaccaggtgatcatgctgctgtaccccgaccaccccaccctgctgtcctaccggaacatgggcgaggaacccaactaccaagaggagtgggtcatgcacaagaaagaagtggtgctgaccgtccccaccgagggcctggaagtgacctggggcaacaacgagccctacaagtactggccccagctgtccaccaacggcaccgcccacggccacccccacgagatcatcctgtactactacgagctgtaccctaccatgaccgtggtggtggtgtccgtggccacctttatcctgctgtccatggtcggcatggccgctggcatgtgcatgtgcgccaggaggcgctgtatcaccccctacgagctgacacctggcgccaccgtgccctttctgctgtccctgatctgctgcatccggaccgccaaggcccgcgggcgcaaacgccgctctgccaccatggactggacctggatcctgtttctggtcgctgctgccacccgggtgcacagctacgagcacgtgaccgtgatccccaacaccgtgggcgtgccctacaagaccctggtgaacaggcccggctacagccccatggtgctggaaatggaactgctgtccgtgaccctggaacccaccctgagcctggactacatcacctgcgagtacaagacagtgatccccagcccctacgtgaagtgctgcggcaccgccgagtgcaaggacaagaacctgcccgactacagctgcaaggtgttcaccggcgtgtaccccttcatgtggggcggagcctactgcttctgcgacgccgagaacacccagctgtccgaggcccacgtggagaagagcgagagctgcaagaccgagttcgccagcgcctaccgggcccacacagccagcgccagcgccaagctgcgggtgctgtaccagggcaacaacatcaccgtgaccgcctacgccaacggcgaccacgccgtgacagtgaaggacgccaagttcatcgtgggccccatgagcagcgcctggacccccttcgacaacaagatcgtggtgtacaagggcgacgtgtacaacatggactacccccccttcggagccggcagacccggccagttcggcgacatccagagccggacccccgagagcaaggacgtgtacgccaatacccagctggtgctgcagagacccgccgtgggcaccgtgcacgtgccttacagccaggcccccagcggcttcaagtactggctgaaagagaggggcgccagcctgcagcacaccgcccccttcggctgccagatcgccaccaaccccgtgcgggccgtgaattgtgccgtgggcaacatgcccatcagcatcgacatccccgaggccgccttcaccagggtggtggacgcccccagcctgaccgacatgagctgcgaggtgcccgcctgcacccacagcagcgacttcggcggcgtggccatcatcaagtacgccgccagcaagaaaggcaagtgcgccgtgcacagcatgaccaatgccgtgaccatccgggaggccgagatcgaggtggagggcaacagccagctgcagatcagcttcagcaccgccctggccagcgccgagttccgggtgcaggtctgcagcacccaggtgcactgtgccgccgagtgtcacccccccaaggaccacatcgtgaactaccccgccagccacaccaccctgggcgtgcaggacatcagcgccaccgccatgagctgggtgcagaagatcacaggcggcgtcggcctggtggtggccgtggccgccctgatcctgatcgtggtgctgtgcgtgagcttcagccggcactgatgagcggccgc

SEQ ID NO: 97 - DNA, 2223 bp, Artificial Sequence; identical to SEQ ID NO: 65
  Nucleic acid sequence encoding CHIKV1-IgG

SEQ ID NO: 98 - DNA, 2217 bp, Artificial Sequence
  Nucleic Acid Sequence encoding CHIKV2-IgG
ggatccgccaccatggattggacatggattctgtttctggtcgccgccgccacaagggtccactctcaggtgcagctggtgcagtcaggctccgaactgaagaaacccggagcttctgtcaaggtgagttgcaaagcatcagggtacactctgaccagatatgcaatgacctgggtgcggcaggcacctggacagggactggagtggatgggctggatcaacacatacactggaaatccaacttatgtgcagggcttcaccggacggttcgtcttttctctggacacaagcgtgtccactgcctttctgcacattacaagcctgaaggcagaggacactgccgtgtacttctgcgcccgagaaggcggagctaggggctttgattattgggggcagggcacactggtcactgtgagctccgcttctacaaagggacctagcgtgttcccactggcaccctctagtaaatctaccagtgggggcacagccgctctgggatgtctggtgaaggattacttccccgagcctgtcaccgtgtcatggaacagcggggcactgacctccggagtccatacatttcccgccgtgctgcagtcaagcggactgtacagcctgtcctctgtggtcactgtgcctagttcaagcctggggacccagacatatatctgcaacgtgaatcacaagccttctaataccaaggtcgacaaaagggtggaaccaaagagctgtgataaaactcatacctgccctccctgtccagctccagagctgctgggagggccatccgtgttcctgtttccacccaagcctaaagacacactgatgatttccagaactccagaagtcacctgcgtggtcgtggacgtgtctcacgaggaccccgaagtcaagttcaactggtacgtggatggcgtcgaggtgcataatgccaagaccaaacccagggaggaacagtacaactcaacatatagagtcgtgagcgtcctgactgtgctgcaccaggactggctgaacggcaaggagtataagtgcaaagtgagcaataaggccctgcctgctccaatcgagaaaacaatttccaaggctaaaggccagcctcgggaaccacaggtgtacactctgcctccaagtcgcgacgagctgaccaagaaccaggtctctctgacatgtctggtgaaaggcttttatcccagtgatatcgccgtggagtgggaatcaaatggacagcctgaaaacaattacaagaccacaccccctgtgctggactccgatggatctttctttctgtattccaagctgacagtggataaatctcgctggcagcaggggaacgtcttctcatgcagcgtgatgcacgaggccctgcacaatcattacactcagaagtccctgtctctgagtcctgggaagcggggccgcaaaaggagatcaggaagcggggccaccaactttagcctgctgaaacaggctggggacgtggaggaaaatcccggccctatggactggacctggattctgttcctggtggcagccgctacccgagtccattcccagtctgtgctgacacagccacccagtgtctcaggagcaccaggacagcgagtgacaatcagctgtactggcagcagcagcaacattggggcctcccacgacgtgcattggtaccagcagctgcccggcaccgctcctacactgctgatctatgtcaacagcaatcgcccatccggagtgcccgatcgattcagcggctccaagtctggaactagtgcatcactggccattaccggcctgcaggctgaggacgaagcagattactattgccagagttacgactcaaacctgagcgggtccgccgtcttcggcggagggactaagctgaccgtgctgggccagcctaaagcagccccatccgtgaccctgtttcctccatcaagcgaggaactgcaggctaataaggccaccctggtgtgcctgatcagcgacttctacccaggagcagtcaccgtggcatggaaggctgattcctctccagtcaaagccggcgtggaaactaccacacccagcaagcagtccaacaacaagtacgctgcaagttcatatctgtccctgacccccgagcagtggaagtctcacaaatcttatagttgccaggtgacccatgaaggctcaactgtggaaaaaactgtcgctcctactgaatgctcatgataactcgag

SEQ ID NO: 99 - DNA, 2220 bp, Artificial Sequence
  Nucleic Acid Sequence encoding CHIKV3-IgG
ggatccgccaccatggattggacatggattctgtttctggtcgctgctgccacacgggtgcattctcaggtccagctgcaggagtcagggccagggctggtcaagccttccgaaaccctgtctctgacatgcaccgtgagcggcgacagtatcagctcctactcatggagctggattcggcagccccctggcaaaggactggagtggatcggctacattcactatacaggaagcactaactataatccatcactgaagagccgcctgaccatcagtgtggacgcctcaaaaaaccagttctccctgaagctgtctagtgtcactgccgctgacaccgccgtgtactattgcgcacgggattggggcggatactcaagctcctggacatatgggatggacgtgtgggggcagggcaccacagtcacagtgtctagtgcaagcactaaaggcccctccgtgtttccactggccccctcaagcaagtccacatctgggggcactgcagccctgggatgtctggtgaaggattacttccccgagcctgtcaccgtgtcctggaactctggagccctgacttccggggtccatacctttcccgctgtgctgcagtcctctgggctgtactctctgagttcagtggtcacagtgcctagctcctctctgggcacccagacatatatctgcaacgtgaatcacaagcctagcaatactaaggtcgacaaaagggtggaaccaaaaagctgtgataagactcatacctgcccaccctgtccagcaccagagctgctgggagggccaagcgtgttcctgtttcctccaaagcccaaagacaccctgatgatttccagaaccccagaagtcacatgcgtggtcgtggacgtgtctcacgaggaccccgaagtcaagttcaactggtacgtggatggcgtcgaggtgcataatgctaagacaaaaccacgcgaggaacagtacaattctacatatcgagtcgtgagtgtcctgactgtgctgcaccaggactggctgaacggaaaggagtacaagtgcaaagtgagcaacaaggccctgcctgccccaatcgagaagacaattagcaaggctaaagggcagcctagggaaccacaggtgtacactctgcccccttccagagacgagctgaccaaaaaccaggtctctctgacatgtctggtgaagggcttctatcccagcgatatcgcagtggagtgggaaagcaatggacagcctgaaaacaattacaagactaccccacccgtgctggacagtgatggctcattctttctgtattcaaaactgaccgtggataagagccggtggcagcagggaaacgtctttagttgctcagtgatgcacgaggccctgcacaatcattacacacagaaaagcctgtccctgtctcctgggaaacggggccgcaagaggagaagtggatcaggggccactaacttcagcctgctgaagcaggctggggacgtggaggaaaatcccggccctatggattggacctggattctgttcctggtcgctgcagccactcgcgtgcattccgacattcagatgacccagtctcccagttcactgagcgcctccgtcggagatcgagtgacaatcacttgtagggcaagccaggggatttctaacagtctggcctggtaccagcagaagcccggaaaagctcctaagctgctgctgtatgctgcaagcagactggaatccggggtgccttctcggttctcaggcagcggcagcggaaccgactacaccctgacaattagctccctgcagcctgaggatttcgccacatactattgccagcagtactattccaccccatatacatttggccagggaactaaactggaaatcaagcgaaccgtcgccgctccaagcgtgttcatctttcctccatctgacgagcagctgaagagtggaaccgcttcagtggtgtgcctgctgaacaacttctacccccgcgaggcaaaagtccagtggaaggtggataacgccctgcagagcggcaattcccaggagtctgtgaccgaacaggacagtaaagattcaacatatagcctgtctagtactctgaccctgagcaaggctgactacgagaagcacaaagtgtatgcatgcgaagtgacccaccaggggctgagttctccagtgactaaatctttcaacagaggcgaatgttgataactcgag

SEQ ID NO: 100 - DNA, 2208 bp, Artificial Sequence
  Nucleic Acid Sequence encoding CHIKV4-IgG
ggatccgccaccatggactggacatggattctgttcctggtcgctgctgctactcgggtgcatagcgaagtgaggctggtcgaaagtgggggcgggctggagcagcccggcggcagcctgaagctgtcttgcgccgctagtggcttcacctttagcgactacttcatgtattgggtcagacagacacctgagaagcggctggaatgggtggcctacatcagcaacggcggaatttcaacattctatagcgacgccgtgaaaggccggtttaccatctcccgagataacgctaggaatacactgtacctgcagatgtcccggctgaagtctgaagacacagcaatctactattgcgtccgccaggtgtatgggcagggctacttcgattattggggacaggggaccacactggctgtcagctccgcaagcactaagggaccctccgtgtttcccctggccccttctagtaaaagtacctcaggagggacagcagccctggggtgtctggtgaaggactacttccctgagccagtcaccgtgtcatggaacagcggagctctgacatccggggtccacacttttcctgcagtgctgcagtcaagcggactgtactctctgtcctctgtggtcaccgtgccaagttcaagcctggggacacagacttatatctgcaacgtgaatcacaagccatccaatacaaaagtcgacaagaaagtggaacccaagtcttgtgataaaacccatacatgccctccctgtccagctcctgagctgctgggcggaccatccgtgttcctgtttccacccaagcctaaagacacactgatgattagccggactcccgaagtcacctgcgtggtcgtggacgtgtcccacgaggaccccgaagtcaagttcaactggtacgtggatggcgtcgaggtgcataatgccaagaccaaacctcgcgaggaacagtacaactctacttatcgagtcgtgagtgtcctgaccgtgctgcaccaggactggctgaacggcaaggagtataagtgcaaagtgtctaataaggcactgccagcccccatcgagaaaactattagcaaggctaaaggacagccaagggaaccccaggtgtacaccctgcctccatctagagacgagctgaccaagaaccaggtcagcctgacatgtctggtgaaagggttctatccttctgatatcgcagtggagtgggaaagtaatggccagccagaaaacaattacaagactacccctcccgtgctggactccgatgggtctttctttctgtattcaaagctgaccgtggataaaagccgctggcagcagggcaacgtctttagctgctccgtgatgcatgaggccctgcacaatcattacactcagaaatctctgagtctgtcaccagggaagcgaggacgaaaaaggagaagcggctccggagctaccaacttcagcctgctgaagcaggcaggcgacgtggaggaaaatcctggaccaatggattggacttggattctgttcctggtggctgcagccacccgagtccactcccagatcgtgctgattcagtctccagccattatgtctgctagtctgggcgagcgcgtcactatgacctgtacagccagcagcagcgtgagcagcagctacctgcattggtatcagcagaagcctggctctagtccaaaactgtggatctactcaagcttcagtctggcatcaggagtgccagcaaggttttcagggagcggctccggaacatcttacagtctgacaattagcactatggaggctgaagacgctgcaacttactattgccaccagtatctgagaagcccatggaccttcggcggcggcagcaagctggaaatcaaaactgtcgccgctcccagcgtgttcatctttccaccctcagacgagcagctgaagagtgggaccgcctcagtggtgtgcctgctgaacaacttctaccctagagaagccaaggtccagtggaaagtggataacgctctgcagagcggaaattcccaggagtctgtgacagaacaggacagtaaggattcaacttatagcctgtcctctactctgaccctgtccaaagcagattacgagaagcataaagtgtatgcctgcgaggtcacccaccaggggctgcggtctcccgtcacaaagagcttcaacaggggcgaatgttgataactcgag

SEQ ID NO: 101 - protein, 133 aa, Artificial Sequence
  ZIKV-3F12E9-VH
MNFGLSLIFLALILKGVQCEVQLVESGGDLVKPGGSLKLSCAASGFTFSRYGMSWGRQTPDKRLEWVATISSGGTYTYYPDSVKGRFTISRDNAKNTLYLQMSSLKSEDTAMYYCARSWFAYWGRGTLVTVSA

SEQ ID NO: 102 - protein, 132 aa, Artificial Sequence
  ZIKV-3F12E9-VL
MMSPAQFLFLLVLWIRETNGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPHTFGGGTKLEIK

SEQ ID NO: 103 - protein, 134 aa, Artificial Sequence
  ZIKV-8A9F9-VH
MNFGLSLIFLVLILKGVQCEVQLVESGGGLVKPGGSLKLSCAASGFTFSSYAMSWVRQSPEKRLEWVAEISSGGSYTYYPDTVTGRFTISRDNAKNTLYLEMSSLRSEDTAMYYCASDGYYSHWGQGTSVTVSS

SEQ ID NO: 104 - protein, 131 aa, Artificial Sequence
  ZIKV-8A9F9-VL
MKLPVRLLVLMFWIPASRSDVVMTQIPLSLPVSLGDQASISCRSSQSLVHSNGNTYLHWYLQKPGQSPKLLIYKVSNRFSGVPDRFSGSGSGTDFTLKISRVEAEDLGVYFCFQSTHVPPTFGGGTKLEIK

SEQ ID NO: 105 - protein, 82 aa, Artificial Sequence
  ZIKV-8D10F4-VH
MNFGLSLIFLVLILKGVKCEVQLVESGGGLVKPGGSLKLSCAASGFTFSSYAMSWVRQSPEKRLEWVAEISSGGSYIYYPDT

SEQ ID NO: 106 - protein, 131 aa, Artificial Sequence
  ZIKV-8D10F4-VL
MKLPVRLLVLMFWIPASSSDVVMTQSPLSLPVSLGDQASISCRSSQSLVHSNGNTYFHWYLQKPGQSPKLLIYKVSNRFSGVPDRFSGSGSGTDFTLKISRVEAEDLGLYFCSQSTHVPPTFGGGTKLEIK

SEQ ID NO: 107 - protein, 134 aa, Artificial Sequence; identical to SEQ ID NO: 103
  ZIKV-IC2A6-VH

SEQ ID NO: 108 - protein, 131 aa, Artificial Sequence
  ZIKV-IC2A6-VH
MKLPVRLLVLMFWIPASSSDVVMTQTPLSLPVCLGDQASISCRSSQSLVHSNGNTYLHWYLQKPGQSPKLLIYKVSNRFSGVPDRFSGSGSGTDFTLKISRVEAEDLGVYFCFQSTHVPPTFGGGTKLEIK

SEQ ID NO: 109 - protein, 146 aa, Artificial Sequence
  ZIKV-ID4G7-VH
MAWVWTLLFLMAAAQSAQAQIQLVQSGPELKKPGETVKISCKASGYTFTNYGMNWVKQAPGKGLKWMGWINTYTGEPTYADDFKGRFAFSLETSASTAYLQINNLKNEDTATYFCAREISKIYYYGSSYENGMDYWGQGTSVTVSS

SEQ ID NO: 110 - protein, 131 aa, Artificial Sequence
  ZIKV-ID4G7-VL
METDTLLLWVLLLWVPGSTGNIVLTQSPASLAVSLGQRATISCRASESVDSFGNSFMHWFQQKPGQPPKLLIYLASNLESGVPARFSGSGSRTDFTLTIDPVEADDAATYYCQQNNEYPYTFGGGTKLEIK

SEQ ID NO: 111 - protein, 728 aa, Artificial Sequence
  Human anti-Zika (3F12E9)-IgG4 Human IgG heavy      signal peptide-VH-CH1-Hinge Region-CH2-CH3-custom Furin cleavage site-′GSG′ Linker and P2A Peptide-human kappa light chain signal peptide-VL-CL (kappa)
MDWTWRILFLVAAATGTHAEVQLVESGGDLVKPGGSLKLSCAASGFTFSRYGMSWGRQTPDKRLEWVATISSGGTYTYYPDSVKGRFTISRDNAKNTLYLQMSSLKSEDTAMYYCARSWFAYWGRGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVSPNMVPHAHHAQAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 731 aa, Artificial Sequence
  Human IgG heavy signal peptide-VH-CH1-Hinge      Region-CH2-CH3-custom Furin cleavage site-′GSG′ Linker and P2A      Peptide-human kappa light chain signal peptide-VL-CL (kappa)
MDWTWRILFLVAAATGTHAEVQLVESGGDLVKPGGSLKLSCAASGFTFSRYGMSWGRQTPDKRLEWVATISSGGTYTYYPDSVKGRFTISRDNAKNTLYLQMSSLKSEDTAMYYCARSWFAYWGRGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 729 aa, Artificial Sequence
  Human IgG heavy signal peptide-VH-CH1-Hinge      Region-CH2-CH3-custom Furin cleavage site-′GSG′ Linker and P2A      Peptide-human kappa light chain signal peptide-VL-CL (kappa)
MDWTWRILFLVAAATGTHAEVQLVESGGGLVKPGGSLKLSCAASGFTFSSYAMSWVRQSPEKRLEWVAEISSGGSYTYYPDTVTGRFTISRDNAKNTLYLEMSSLRSEDTAMYYCASDGYYSHWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVSPNMVPHAHHAQAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGDVVMTQIPLSLPVSLGDQASISCRSSQSLVHSNGNTYLHWYLQKPGQSPKLLIYKVSNRFSGVPDRFSGSGSGTDFTLKISRVEAEDLGVYFCFQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 732 aa, Artificial Sequence
  Human anti-Zika (8A9F9)-IgG1 Human IgG heavy      signal peptide-VH-CH1-Hinge Region-CH2-CH3-custom Furin cleavage site-′GSG′ Linker and P2A Peptide-human kappa light chain signal peptide-VL-CL (kappa)
MDWTWRILFLVAAATGTHAEVQLVESGGGLVKPGGSLKLSCAASGFTFSSYAMSWVRQSPEKRLEWVAEISSGGSYTYYPDTVTGRFTISRDNAKNTLYLEMSSLRSEDTAMYYCASDGYYSHWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDVVMTQIPLSLPVSLGDQASISCRSSQSLVHSNGNTYLHWYLQKPGQSPKLLIYKVSNRFSGVPDRFSGSGSGTDFTLKISRVEAEDLGVYFCFQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 728 aa, Artificial Sequence
  Human anti-Zika (8D10F4)-IgG4 Human IgG heavy      signal peptide-VH-CH1-Hinge Region-CH2-CH3-custom Furin cleavage site-′GSG′ Linker and P2A Peptide-human kappa light chain signal peptide-VL-CL (kappa)
MDWTWRILFLVAAATGTHAEVQLVESGGGLVKPGGSLKLSCAASGFTFSSYAMSWVRQSPEKRLEWVAEISSGGSYIYYPDTVTGRFTISRDNAKNTLYLEMSSLRSEDTAMYYCASDGYYSHWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVSPNMVPHAHHAQAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGDVVMTQSPLSLPVSLGDQASISCRSSQSLVHSNGNTYFHWYLQKPGQSPKLLIYKVSNRFSGVPDRFSGSGSGTDFTLKISRVEAEDLGLYFCSQSTHVPPTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 732 aa, Artificial Sequence
  Human anti-Zika (8D10F4)-IgG1 Human IgG heavy      signal peptide-VH-CH1-Hinge Region-CH2-CH3-custom Furin cleavage site-′GSG′ Linker and P2A Peptide-human kappa light chain signal peptide-VL-CL (kappa)
MDWTWRILFLVAAATGTHAEVQLVESGGGLVKPGGSLKLSCAASGFTFSSYAMSWVRQSPEKRLEWVAEISSGGSYIYYPDTVTGRFTISRDNAKNTLYLEMSSLRSEDTAMYYCASDGYYSHWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDVVMTQSPLSLPVSLGDQASISCRSSQSLVHSNGNTYFHWYLQKPGQSPKLLIYKVSNRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 740 aa, Artificial Sequence
  Human anti-Zika (1D4G7)-IgG4 Human IgG heavy      signal peptide-VH-CH1-Hinge Region-CH2-CH3-custom Furin cleavage site-′GSG′ Linker and P2A Peptide-human kappa light chain signal peptide-VL-CL (kappa)
MDWTWRILFLVAAATGTHAQIQLVQSGPELKKPGETVKISCKASGYTFTNYGMNWVKQAPGKGLKWMGWINTYTGEPTYADDFKGRFAFSLETSASTAYLQINNLKNEDTATYFCAREISKIYYYGSSYENGMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVSPNMVPHAHHAQAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGNIVLTQSPASLAVSLGQRATISCRASESVDSFGNSFMHWFQQKPGQPPKLLIYLASNLESGVPARFSGSGSRTDFTLTIDPVEADDAATYYCQQNNEYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 743 aa, Artificial Sequence
  Human anti-Zika (1D4G7)-IgG1 Human IgG heavy      signal peptide-VH-CH1-Hinge Region-CH2-CH3-custom Furin cleavage site-′GSG′ Linker and P2A Peptide-human kappa light chain signal peptide-VL-CL (kappa)
MDWTWRILFLVAAATGTHAQIQLVQSGPELKKPGETVKISCKASGYTFTNYGMNWVKQAPGKGLKWMGWINTYTGEPTYADDFKGRFAFSLETSASTAYLQINNLKNEDTATYFCAREISKIYYYGSSYENGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGNIVLTQSPASLAVSLGQRATISCRASESVDSFGNSFMHWFQQKPGQPPKLLIYLASNLESGVPARFSGSGSRTDFTLTIDPVEADDAATYYCQQNNEYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 732 aa, Artificial Sequence
  Human anti-Zika (8A9F9)-IgG1 Human IgG heavy      signal peptide-VH-CH1-Hinge Region-CH2 (with LALA variant at 4th      and 5th residue)-CH3-custom Furin cleavage site-′GSG′ Linker and      P2A Peptide-human kappa light chain signal peptide-VL-CL (kappa)
MDWTWRILFLVAAATGTHAEVQLVESGGGLVKPGGSLKLSCAASGFTFSSYAMSWVRQSPEKRLEWVAEISSGGSYTYYPDTVTGRFTISRDNAKNTLYLEMSSLRSEDTAMYYCASDGYYSHWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDVVMTQIPLSLPVSLGDQASISCRSSQSLVHSNGNTYLHWYLQKPGQSPKLLIYKVSNRFSGVPDRFSGSGSGTDFTLKISRVEAEDLGVYFCFQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 731 aa, Artificial Sequence
  Human anti-Zika (3F12E9)-IgG1 Human IgG heavy      signal peptide-VH-CH1-Hinge Region-CH2 (with LALA variant at 4th      and 5th residue)-CH3-custom Furin cleavage site-′GSG′ Linker and      P2A Peptide-human kappa light chain signal peptide-VL-CL (kappa)
MDWTWRILFLVAAATGTHAEVQLVESGGDLVKPGGSLKLSCAASGFTFSRYGMSWGRQTPDKRLEWVATISSGGTYTYYPDSVKGRFTISRDNAKNTLYLQMSSLKSEDTAMYYCARSWFAYWGRGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 780 aa, Artificial Sequence
  Human anti-Zika (IC2A6)-IgG4 Human IgG heavy      signal peptide-VH-CH1-Hinge Region-CH2-CH3-custom Furin cleavage site-′GSG′ Linker and P2A Peptide-human kappa light chain signal peptide-VL-CL (kappa)
MDWTWRILFLVAAATGTHAEVQLVESGGGLVKPGGSLKLSCAASGFTFSSYAMSWVRQSPEKRLEWVAEISSGGSYTYYPDTVTGRFTISRDNAKNTLYLEMSSLRSEDTAMYYCASDGYYSHWGQGTSVTVSSVTGRFTISRDNAKNTLYLEMSSLRSEDTAMYYCASDGYYSHWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVSPNMVPHAHHAQAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGDVVMTQTPLSLPVCLGDQASISCRSSQSLVHSNGNTYLHWYLQKPGQSPKLLIYKVSNRFSGVPDRFSGSGSGTDFTLKISRVEAEDLGVYFCFQSTHVPPTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 784 aa, Artificial Sequence
  Human anti-Zika (IC2A6)-IgG1 Human IgG heavy      signal peptide-VH-CH1-Hinge Region-CH2-CH3-custom Furin cleavage site-′GSG′ Linker and P2A Peptide-human kappa light chain signal peptide-VL-CL (kappa)
MDWTWRILFLVAAATGTHAEVQLVESGGGLVKPGGSLKLSCAASGFTFSSYAMSWVRQSPEKRLEWVAEISSGGSYTYYPDTVTGRFTISRDNAKNTLYLEMSSLRSEDTAMYYCASDGYYSHWGQGTSVTVSSVTGRFTISRDNAKNTLYLEMSSLRSEDTAMYYCASDGYYSHWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDVVMTQTPLSLPVCLGDQASISCRSSQSLVHSNGNTYLHWYLQKPGQSPKLLIYKVSNRFSGVPDRFSGSGSGTDFTLKISRVEAEDLGVYFCFQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 702 aa, Artificial Sequence
  consensus Zika IgE Leader-prME
MDWTWILFLVAAATRVHSGIIGLLLTTAMAAEITRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIVNDIGHETDENRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKVPAQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

SEQ ID NO: 124 - DNA, 2112 bp, Artificial Sequence
  consensus Zika IgE Leader-prME (construct 1)
atggactggacctggattctgtttctggtcgctgctgctacaagagtgcattctgggattattggactgctgctgactactgccatggcagcagagatcaccaggagaggcagcgcctactatatgtacctggaccggtctgatgccggcaaggccatcagctttgccaccacactgggcgtgaataagtgccacgtgcagatcatggacctgggccacatgtgcgatgccaccatgtcctacgagtgtccaatgctggacgagggcgtggagcccgacgatgtggattgctggtgtaacaccacatctacatgggtggtgtatggcacctgtcaccacaagaagggagaggcacggcgcagcaggagagcagtgacactgccctctcacagcaccaggaagctgcagacaagaagccagacctggctggagtcccgggagtatacaaagcacctgatcaaggtggagaactggatctttcgcaatccaggattcgcactggtggcagtggcaatcgcatggctgctgggcagctccacctcccagaaagtgatctacctggtcatgatcctgctgatcgcccctgcctattccatcaggtgcatcggcgtgtctaatagagacttcgtggagggcatgtctggcggcacctgggtggatgtggtgctggagcacggcggatgcgtgacagtgatggcccaggacaagccaaccgtggatatcgagctggtgaccacaaccgtgagcaacatggccgaggtgaggtcctactgctatgaggcctccatctctgacatggccagcgattccagatgtcccacccagggcgaggcctacctggacaagcagtccgatacacagtacgtgtgcaagcggaccctggtggacaggggatggggaaatggatgtggcctgtttggcaagggctctctggtgacatgcgccaagttcacctgttctaagaagatgaccggcaagagcatccagcccgagaacctggagtacaggatcatgctgagcgtgcacggcagccagcactccggcatgacagtgaacgacatcggctatgagaccgatgagaatagggccaaggtggaggtgacacctaacagcccaagagccgaggccaccctgggcggctttggctccctgggactggactgcgagcctagaacaggcctggacttctccgatctgtactatctgaccatgaacaataagcactggctggtgcacaaggagtggtttcacgacatcccactgccatggcacgcaggagcagatacaggaaccccacactggaacaataaggaggccctggtggagttcaaggatgcccacgccaagcgccagacagtggtggtgctgggcagccaggagggagcagtgcacaccgccctggcaggcgccctggaggccgagatggacggcgccaagggcaagctgttttccggccacctgaagtgccggctgaagatggataagctgcgcctgaagggcgtgtcttacagcctgtgcacagccgccttcaccttcaccaaggtgcctgccgagaccctgcacggcacagtgaccgtggaggtgcagtatgccggcacagacggcccctgtaagatccctgtgcagatggccgtggatatgcagacactgacccctgtgggccggctgatcaccgcaaatccagtgatcacagagtccaccgagaactctaagatgatgctggagctggaccctcccttcggcgacagctacatcgtgatcggcgtgggcgacaagaagatcacacaccactggcaccgctccggctctacaatcggcaaggccttcgaggcaaccgtgcggggcgccaagaggatggccgtgctgggcgacaccgcatgggattttggctccgtgggcggcgtgttcaactctctgggcaagggcatccaccagatcttcggcgccgcctttaagtctctgttcggcggaatgtcttggttcagccagatcctgatcggcacactgctggtgtggctgggcctgaacaccaagaatggcagcatctctctgacttgtctggccctgggaggcgtgatgattttcctgtccactgccgtgtctgcctgataa

SEQ ID NO: 125 - protein, 702 aa, Artificial Sequence
  consensus Zika IgE Leader-prME (construct 1)
MDWTWILFLVAAATRVHSGIIGLLLTTAMAAEITRRGSAYYMYLDRSDAGKAISFATTLGVNKCHVQIMDLGHMCDATMSYECPMLDEGVEPDDVDCWCNTTSTWVVYGTCHHKKGEARRSRRAVTLPSHSTRKLQTRSQTWLESREYTKHLIKVENWIFRNPGFALVAVAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMTVNDIGYETDENRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGKLFSGHLKCRLKMDKLRLKGVSYSLCTAAFTFTKVPAETLHGTVTVEVQYAGTDGPCKIPVQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

SEQ ID NO: 126 - DNA, 1119 bp, Artificial Sequence
  consensus Zika IgE Leader-NS1
atggactggacttggattctgttcctggtggctgccgctacaagagtgcatagcgtgggatgcagcgtggacttcagcaagaaggagacccgctgcggaacaggcgtgttcgtgtacaacgacgtggaggcttggagagaccggtacaagtaccaccccgatagccctagaagactggccgcagccgtgaaacaggcttgggaagagggaatttgcggcatcagcagcgtgtcccggatggagaacatcatgtggaagagcgtggagggcgagctgaacgctatcctggaggagaacggcgtgcagctgacagtggtcgtgggatcagtgaagaaccccatgtggagaggccctcagaggctgccagtgccagtgaacgaactgcctcacggttggaaggcttggggcaagagctacttcgtgagggccgccaagaccaacaacagcttcgtggtggacggcgataccctcaaggagtgtcctctgaagcaccgggcttggaacagcttcctggtggaagaccacggctttggcgtgttccacacaagcgtctggctgaaggtccgcgaagactacagcctggagtgcgatccagcagtgatcggcacagccgtgaagggaaaagaggccgctcacagcgacctgggctattggatcgagagcgagaagaacgacacttggaggctgaagcgggcccacctgatcgagatgaagacttgcgagtggcccaagagccacactctgtggacagacggcgtggaagagagcgacctgatcatccctaagagcctggccggacctctgtctcatcacaacaccagggagggctacagaacccaggtgaagggaccttggcacagcgaagagctggagatccgcttcgaggagtgtccaggaaccaaggtgcacgtggaggagacttgcggaaccagaggccctagcctgagaagcacaacagccagcggacgcgtgatcgaggagtggtgttgtagggagtgcaccatgcctcctctgagcttcagggccaaggacggttgttggtacggcatggagatcaggcccagaaaggagccagagagcaacctcgtgcggtctatggtgacagccggaagctgataa

SEQ ID NO: 127 - protein, 371 aa, Artificial Sequence
  consensus Zika IgE Leader-NS1
MDWTWILFLVAAATRVHSVGCSVDFSKKETRCGTGVFVYNDVEAWRDRYKYHPDSPRRLAAAVKQAWEEGICGISSVSRMENIMWKSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAAHSDLGYWIESEKNDTWRLKRAHLIEMKTCEWPKSHTLWTDGVEESDLIIPKSLAGPLSHHNTREGYRTQVKGPWHSEELEIRFEECPGTKVHVEETCGTRGPSLRSTTASGRVIEEWCCRECTMPPLSFRAKDGCWYGMEIRPRKEPESNLVRSMVTAGS

SEQ ID NO: 128 - DNA, 438 bp, Artificial Sequence
  consensus Zika IgE Leader-capsid
atggactggacttggatcctgtttctggtggccgccgccacaagagtgcatagcaagaaccccaagaagaagagcggcggcttcaggatcgtgaacatgctgaagcggggcgtggctagagtgaaccctctgggaggcggactgaagagactgccagcaggactgctcctgggacacggacctattcgcatggtgctggccatcctggctttcctgaggttcaccgccatcaagcccagcctgggactgatcaaccgctggggttcagtcggcaagaaggaggccatggagatcatcaagaagttcaagaaggacctggccgccatgctgaggatcatcaacgcccggaaggagcggaagagaagaggagccgacaccagcatcggcatcatcggactgctgctgacaaccgccatggctgccgagatctgatgatga

SEQ ID NO: 129 - protein, 143 aa, Artificial Sequence
  consensus Zika IgE Leader-capsid
MDWTWILFLVAAATRVHSKNPKKKSGGFRIVNMLKRGVARVNPLGGGLKRLPAGLLLGHGPIRMVLAILAFLRFTAIKPSLGLINRWGSVGKKEAMEIIKKFKKDLAAMLRIINARKERKRRGADTSIGIIGLLLTTAMAAEI

SEQ ID NO: 130 - DNA, 2130 bp, Artificial Sequence
  Zika IgE Leader-prME MR766
atggactggacttggattctgttcctggtggctgccgctacaagagtgcattcaggagccgacacctctatcggcatcgtgggactgctgctgaccacagccatggccgccgaaattaccaggaggggcagcgcctactacatgtacctggacagaagcgacgccggaaaagccatcagcttcgccacaaccctgggcgtcaacaagtgccacgtgcagatcatggacctgggccacatgtgcgacgccacaatgagctacgagtgccctatgctggacgagggagtggaaccagacgacgtcgactgttggtgcaacaccacctccacttgggtcgtgtacggcacttgccaccacaagaagggcgaggccagaagaagcagaagagccgtgaccctgcctagccacagcaccagaaagctgcagaccaggagccagacttggctggaaagccgcgagtacaccaagcacctgatcaaggtggagaattggatcttccggaaccccggcttcacactggtggccgtggcaatcgcttggctgctgggatctagcaccagccagaaagtgatctacctggtcatgatcctgctgatcgccccagcctacagcatccgctgtatcggagtgagcaaccgggacttcgtggagggaatgagcggaggaacttgggtggacgtggtgctggaacacggaggttgcgtgacagtgatggctcaggacaagcccaccgtggatatcgagctggtgaccaccaccgtgtccaacatggccgaagtgcgcagctactgctacgaggccagtatctccgacatggccagcgatagccgctgtcctacacagggagaggcctatctggacaagcagagcgacacccagtacgtctgcaagaggaccctcgtggatagaggctggggaaacggttgcggactgttcggaaagggcagcctcgtgacttgcgccaagttcacttgcagcaagaagatgaccggcaagtctatccagcccgagaacctggagtaccggatcatgctgagcgtgcacggaagccagcacagcggcatgatcgtgaacgacgagggatacgagaccgacgagaacagggccaaggtggaagtgacccctaacagccctagagccgaagccacactgggaggatttggcagcctgggactggattgcgagcctagaacaggcctggacttcagcgacctgtactacctgaccatgaacaacaagcattggctggtgcacaaggagtggttccacgacatccctctgccttggcacgcaggagccgatacaggcacacctcattggaacaacaaggaggccctggtggagttcaaggacgctcacgccaagagacagacagtggtggtgctgggaagccaggaaggagcagtgcatacagccctggcaggagctctggaagcagaaatggacggcgctaagggcagactgttcagcggacacctcaagtgccggctgaagatggacaagctgcggctgaagggcgtgtcttacagcctctgcaccgcagccttcaccttcaccaaggtgccagcagagacactgcacggaacagtgaccgtggaagtgcagtacgccggaacagacggaccttgcaaagtgccagcccagatggcagtggacatgcagacactgaccccagtgggaaggctgatcaccgctaaccccgtcatcaccgagagcaccgagaacagcaagatgatgctggagctggacccccccttcggcgatagctacatcgtgatcggcgtgggcgacaagaagatcacccaccattggcacagaagcggcagcacaatcggcaaggctttcgaggccaccgtgagaggagctaagagaatggccgtgctgggagacaccgcttgggattttggcagcgtgggaggagtgttcaacagcctgggcaagggcatccaccagatcttcggagccgccttcaagagcctgttcggcggcatgtcttggttcagccagatcctgatcggaacactcctcgtctggctgggactgaacaccaagaacggcagcatcagcctgacttgtctggccctgggaggcgtgatgatcttcctgagcaccgccgtgtccgcttgataa

SEQ ID NO: 131 - protein, 708 aa, Artificial Sequence
  Zika IgE Leader-prME MR766
MDWTWILFLVAAATRVHSGADTSIGIVGLLLTTAMAAEITRRGSAYYMYLDRSDAGKAISFATTLGVNKCHVQIMDLGHMCDATMSYECPMLDEGVEPDDVDCWCNTTSTWVVYGTCHHKKGEARRSRRAVTLPSHSTRKLQTRSQTWLESREYTKHLIKVENWIFRNPGFTLVAVAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIVNDEGYETDENRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKVPAQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

SEQ ID NO: 132 - DNA, 2130 bp, Artificial Sequence
  Zika IgE Leader-prME Brazil
atggactggacttggattctgttcctggtggctgccgctacaagagtgcattcaggagccgacacatcagtgggcatcgtgggactgctgctgacaacagctatggccgccgaagtgaccagaagaggcagcgcctactacatgtacctggaccggaacgacgccggagaggccattagctttcctaccaccctgggcatgaacaagtgctacatccagatcatggacctgggccacatgtgcgacgctacaatgagctacgagtgccccatgctggacgaaggagtggagccagacgacgtggattgttggtgcaacaccacctccacttgggtcgtgtacggcacctgtcaccacaaaaagggcgaagccaggagaagcagaagagccgtgaccctgcctagccactctaccaggaagctgcagaccaggagccagacttggctggagagcagggagtacaccaagcacctgatccgcgtggagaattggatcttcagaaaccccggcttcgccctggcagccgcagcaattgcttggctgctgggatctagcaccagccagaaggtcatctacctggtcatgatcctgctgatcgcccccgcttacagcatccgctgtatcggcgtgtccaacagggacttcgtggagggcatgagcggaggaacttgggtggacgtggtgctggaacacggaggttgtgtgaccgtgatggctcaggacaagcctaccgtggacatcgagctggtgaccacaaccgtgtccaacatggccgaggtccgcagctattgctacgaggccagcatcagcgatatggccagcgatagcaggtgtcccacccagggtgaagcttacctggacaagcagagcgacacccagtacgtgtgcaagcggacactggtggatagaggctggggaaacggttgcggcctgtttggcaagggaagcctggtgacctgcgccaagttcgcatgcagcaagaagatgaccggcaagagcatccagcccgagaacctggagtaccggatcatgctgagcgtgcacggatctcagcatagcggaatgatcgtgaacgacaccggccacgagaccgacgaaaacagggccaaggtggaaatcacccccaactctcctagagccgaggccacactgggaggttttggaagcctgggcctggattgcgagcctagaacaggcctggacttcagcgacctgtactacctgaccatgaacaacaagcattggctggtgcacaaggagtggttccacgacatccctctgccttggcacgcaggagcagatacaggaaccccccattggaacaacaaggaggccctggtggagttcaaggacgctcacgccaagagacagacagtggtggtgctgggaagccaggaaggagcagtgcacacagctctggcaggagctctggaagccgaaatggacggagccaagggcagactgtcctccggacacctcaagtgccggctgaagatggacaagctgcggctgaagggcgtgtcttatagcctctgcacagccgctttcaccttcaccaagatccccgcagagaccctgcacggaacagtgaccgtggaagtgcagtacgccggaacagacggaccttgcaaggtgccagctcagatggcagtggacatgcagaccctgaccccagtgggaagactgatcaccgctaaccccgtcatcaccgagagcaccgagaacagcaagatgatgctggagctggacccccccttcggcgatagctacatcgtgatcggcgtgggcgagaaaaagatcacccaccattggcacaggagcggcagcacaatcggcaaggcctttgaggccaccgtgagaggagccaagagaatggccgtgctgggagataccgcttgggatttcggcagcgtgggaggcgccctgaacagcctgggcaagggcattcaccagatcttcggagccgccttcaagagcctgttcggcggcatgtcttggttcagccagatcctgatcggcacactgctcatgtggctgggcctgaacaccaagaacggcagcatcagcctgatgtgtctggctctgggaggcgtgctgatcttcctgagcaccgctgtgtccgcttgataa

SEQ ID NO: 133 - protein, 708 aa, Artificial Sequence
  Zika IgE Leader-prME Brazil
MDWTWILFLVAAATRVHSGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 134 - protein, 18 aa, Artificial Sequence
  IgE leader
MDWTWILFLVAAATRVHS

SEQ ID NO: 135 - protein, 732 aa, Artificial Sequence
  DMAb-2G4
MDWTWRILFLVAAATGTHAEVQLQESGGGLMQPGGSMKLSCVASGFTFSNYWMNWVRQSPEKGLEWVAEIRLKSNNYATHYAESVKGRFTISRDDSKRSVYLQMNTLRAEDTGIYYCTRGNGNYRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPASLSVSVGETVSITCRASENIYSSLAWYQQKQGKSPQLLVYSATILADGVPSRFSGSGSGTQYSLKINSLQSEDFGTYYCQHFWGTPYTFGGGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 136 - DNA, 2202 bp, Artificial Sequence
  PGX9226, DMAb-2G4
atggattggacatggaggattctgtttctggtcgccgccgcaactggaacccacgctgaagtgcagctgcaggagtcaggaggaggactgatgcagcccggcggaagcatgaagctgtcctgcgtggcatctggcttcacctttagtaactactggatgaattgggtccgccagtcacctgagaagggactggaatgggtggctgagatccgactgaaaagcaacaattacgccacccactatgctgagtcagtgaaggggcgattcacaattagcagggacgattctaaaagaagtgtgtatctgcagatgaacactctgagagccgaagacaccggaatctactattgcacacggggcaacggaaattaccgcgctatggattattgggggcagggcacttccgtcaccgtgagctccgcaagcacaaagggaccctccgtgtttcccctggccccttctagtaaaagcacctccggaggaacagcagctctgggatgtctggtgaaggactacttccctgagccagtcaccgtgtcatggaacagcggagccctgacctctggggtccatacatttcctgctgtgctgcagtcaagcgggctgtactccctgtcctctgtggtcactgtgccaagttcaagcctgggcactcagacctatatctgcaacgtgaatcacaagcccagcaataccaaagtcgacaagaaagtggagcctaagtcctgtgataaaacacatacttgcccaccttgtccagcacctgaactgctgggaggacctagcgtgttcctgtttccacccaagccaaaagacacactgatgatttcccgcactcctgaggtcacctgtgtggtcgtggacgtgtctcacgaggaccccgaagtcaagttcaactggtacgtggatggcgtcgaagtgcataatgccaagaccaaacccagggaggaacagtacaactctacctatagggtcgtgagtgtcctgacagtgctgcaccaggactggctgaacggcaaggagtataagtgcaaagtgagcaataaggctctgccagcacccatcgaaaaaactatttccaaggccaaaggacagccaagagagccccaggtgtacaccctgcctccatctcgggacgaactgacaaagaaccaggtcagtctgacttgtctggtgaaaggcttctatccatccgatatcgctgtggagtgggaatctaatggacagcccgagaacaattacaagaccacaccccctgtgctggactccgatgggtctttctttctgtatagtaagctgaccgtggataaatcacggtggcagcagggcaacgtcttttcttgcagtgtgatgcatgaagccctgcacaatcattacacacagaagtcactgagcctgtccccaggcaagcgaggacgaaaaaggagatctggaagtggggctactaacttcagcctgctgaaacaggcaggcgacgtggaggaaaatcctggaccaatggtcctgcagacccaggtgtttatctcactgctgctgtggattagcggggcttacggcgatattcagatgacacagtccccagcatcactgagcgtctccgtgggagaaacagtgtccatcacttgtcgcgcctctgagaacatctacagcagcctggcttggtatcagcagaagcagggaaaaagcccccagctgctggtctactccgcaacaatcctggccgacggggtgccttctaggttctctggcagtggatcagggacacagtatagcctgaagattaatagtctgcagtcagaggattttgggacttactattgccagcacttctggggcacaccatacacttttggcggagggactaagctggagatcaaaaccgtcgcagccccctctgtgttcatttttccacccagtgacgaacagctgaagagtggcaccgcctcagtcgtgtgtctgctgaacaatttctaccctagagaggcaaaggtccagtggaaagtggataacgccctgcagagcggcaattcccaggaatctgtgactgagcaggacagtaaggattcaacctatagcctgtccaacaccctgacactgagcaaagctgactacgaaaagcacaaagtctatgcatgcgaggtgacacatcagggactgagttcaccagtgactaagtcctttaatcggggggagtgttgataa

SEQ ID NO: 137 - protein, 711 aa, Artificial Sequence
  DMAb-4G7
QVQLQQSGPELEMPGASVKISCKASGSSFTGFSMNWVKQSNGKSLEWIGNIDTYYGGTTYNQKFKGKATLTVDKSSSTAYMQLKSLTSEDSAVYYCARSAYYGSTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPASLSASVGETVTITCRASENIYSYLAWYQQKQGKSPQLLVYNAKTLIEGVPSRFSGSGSGTQFSLKINSLQPEDFGSYFCQHHFGTPFTFGSGTELEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 138 - DNA, 2196 bp, Artificial Sequence
  pGX9229, DMAb-4G7
atggattggacatggaggattctgtttctggtcgccgccgccactggaacccacgcccaggtgcagctgcagcagtcagggcctgagctggaaatgcctggcgcttctgtgaaaatcagttgcaaggcatcaggaagctccttcacagggtttagcatgaactgggtgaaacagagcaatgggaagtccctggagtggatcggcaacattgacacctactatggcggaaccacatacaatcagaagttcaaaggcaaggctacactgactgtggacaaatctagttcaaccgcatatatgcagctgaagagcctgacatccgaggattctgcagtgtactattgcgctagatcagcatactatggcagcactttcgcctactggggccagggaaccctggtcacagtgagctccgcctccaccaaaggaccatctgtgtttcccctggctccttctagtaagagtacatcaggaggaactgcagctctgggatgtctggtgaaggattatttccctgagccagtcaccgtgagttggaactcaggcgcactgacttctggagtccacacctttcctgccgtgctgcagtcaagcggcctgtacagcctgtcctctgtggtcaccgtgccaagttcaagcctgggaacccagacatatatctgcaacgtgaatcacaaaccctctaatacaaaggtcgacaagaaagtggaacctaaaagctgtgataagactcatacctgcccaccttgtccagcacctgagctgctgggagggccttccgtgttcctgtttccacccaaaccaaaggacacactgatgattagcagaacccctgaagtcacatgtgtggtcgtggacgtgtcccacgaggaccccgaagtcaagttcaactggtacgtggatggcgtcgaggtgcataatgctaaaaccaagccccgagaggaacagtacaactctacttatagggtcgtgagtgtcctgaccgtgctgcaccaggactggctgaacggcaaggagtataaatgcaaggtgtctaacaaggccctgccagctcccatcgagaagacaattagcaaagctaagggacagccaagagaaccccaggtgtacactctgcctccatctcgggacgagctgaccaaaaaccaggtcagtctgacatgtctggtgaagggattctatccaagcgatatcgcagtggagtgggaatccaatgggcagcccgaaaacaattacaagactaccccccctgtgctggacagcgatggcagcttcttcctgtattccaaactgacagtggataagtctcggtggcagcaggggaacgtctttagctgctccgtgatgcatgaggccctgcacaatcattacactcagaagtctctgagtctgtcaccaggcaaacgaggacgaaagaggagaagcgggtccggagcaaccaacttctccctgctgaagcaggctggagacgtggaggaaaatcctgggccaatggtcctgcagacacaggtgtttatctcactgctgctgtggattagcggggcctacggcgatattcagatgactcagagccccgcatctctgagtgcctcagtcggcgagacagtgactatcacctgtcgcgcaagtgaaaacatctactcatatctggcctggtaccagcagaaacaggggaagagcccccagctgctggtctataatgctaaaaccctgatcgaaggagtgccttcccgattcagcggcagcgggtctggcacacagtttagcctgaagattaactccctgcagccagaggacttcggcagctacttttgccagcaccatttcggaactcccttcacctttggcagcgggacagagctggaaatcaaaactgtcgcagcccccagtgtgttcatttttccaccctcagacgaacagctgaagtctgggaccgccagtgtcgtgtgtctgctgaacaatttttaccctcgggaggctaaagtccagtggaaggtggataacgcactgcagtctggaaatagtcaggagtcagtgacagaacaggacagcaaagattccacttatagtctgtcaaacacactgactctgtctaaggccgactacgagaaacacaaggtctatgcttgcgaagtgactcatcaggggctgtcctctcctgtgaccaagagcttcaatcgcggcgagtgttgataa

SEQ ID NO: 139 - protein, 733 aa, Artificial Sequence
  DMAB4
MDWTWRILFLVAAATGTHAQVTLKESGPGILKPSQTLSLTCSLSGFSLSTSGVGVGWFRQPSGKGLEWLALIWWDDDKYYNPSLKSQLSISKDFSRNQVFLKISNVDIADTATYYCARRDPFGYDNAMGYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSQKFMSTSVGDRVSLTCKASQNVGTAVAWYQQKPGQSPKLLIYSASNRYTGVPDRFTGSGSGTDFTLTISNMQSEDLADYFCQQYSSYPLTFGAGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 140 - DNA, 2226 bp, Artificial Sequence
  pGX9230, DMAb4
ggatccgccgccaccatggactggacttggagaattctgttcctggtcgcagcagccactgggacacacgcacaggtgacactgaaagagagcggacccggaatcctgaaaccaagccagactctgtccctgacctgcagcctgtccggcttctctctgagtacctcaggagtcggagtgggatggtttcgacagccaagcggaaagggactggagtggctggccctgatctggtgggacgatgacaagtactataacccttcactgaaaagccagctgagcatttccaaggatttctctcgcaaccaggtctttctgaagatcagtaatgtggatattgccgacactgctacctactattgcgctaggagagatccattcggctacgacaatgcaatgggatattggggccagggaacctccgtcacagtgagctccgcatccacaaaagggccctctgtgtttcccctggccccttctagtaagtctacaagtggcggaactgccgctctgggctgtctggtgaaggactacttccctgagccagtcaccgtgtcctggaactctggagccctgacttctggggtccacacctttcctgctgtgctgcagtcaagcggactgtacagcctgtcctctgtggtcaccgtgccaagttcaagcctggggacacagacttatatctgcaacgtgaatcacaagccatctaatacaaaagtcgataagaaagtggaacccaagagctgtgacaaaacccatacatgcccaccttgtccagcacctgagctgctgggaggaccaagcgtgttcctgtttccacccaagcctaaagatacactgatgatttccaggacccccgaagtgacatgtgtggtcgtggatgtgtctcacgaggaccctgaagtcaagttcaactggtacgtggacggcgtcgaggtgcataatgctaagaccaaacctcgcgaggaacagtacaacagtacatatcgagtcgtgtcagtgctgaccgtcctgcaccaggactggctgaacggaaaggagtataagtgcaaagtgagcaacaaggcactgccagcccccatcgagaagactatttccaaggcaaaagggcagccaagggaaccccaggtgtacaccctgcctccaagcagagatgagctgactaaaaaccaggtctccctgacctgtctggtgaaggggttctatcctagtgacatcgctgtggagtgggaatcaaatggccagccagaaaacaattacaagaccacaccccctgtgctggatagtgacggctcattctttctgtattcaaagctgaccgtggataaaagcagatggcagcagggaaacgtcttctcatgcagcgtgatgcatgaggccctgcacaatcattacactcagaaatccctgtctctgagtcccggcaagcgaggaaggaaacggcgctcagggagcggcgctacaaacttttccctgctgaagcaggcaggggacgtggaggaaaatcctggcccaatggtcctgcagacccaggtgttcatcagcctgctgctgtggatttccggggcctacggcgatattgtgatgacccagagccagaagttcatgtccacatctgtcggcgaccgggtgtctctgacctgtaaggccagtcagaacgtcggaactgctgtggcatggtatcagcagaagcctgggcagtccccaaaactgctgatctacagtgcttcaaacagatataccggcgtgcctgatcggttcaccggaagcgggtccggcacagactttactctgaccatttctaatatgcagagtgaagatctggctgactacttctgccagcagtactcctcttatccactgacatttggagcagggactaagctggaaatcaaaacagtcgcagccccctccgtgttcatttttccaccctctgatgagcagctgaagtcaggcactgccagcgtcgtgtgtctgctgaacaatttctaccccagggaggccaaggtccagtggaaagtggacaacgctctgcagagcggaaattcccaggagtctgtgactgaacaggatagtaaagactcaacctattctctgagtaacacactgactctgtccaaggcagactacgagaagcacaaagtctatgcctgcgaagtgacccatcagggcctgagttcaccagtgacaaagtcttttaatcgcggagagtgttgataactcgag

SEQ ID NO: 141 - protein, 729 aa, Artificial Sequence
  DMAb-10
MDWTWRILFLVAAATGTHAEVQLVESGGGLVQPGGSLRLSCAASGFTFRSYDMHWVRQATGKGLEWVSAIGTAGDTYYPGSVKGRFTISRENAKNSLYLQMNSLRAEDTAVYYCARVRFGDTAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSLSASVGDRVTITCRASQSISSFLNWHQQKPGKAPKLLIYAASSLQSGVPSRFSGSGSGTDFTLTISSLQPEDFAIYYCQQSYISPFTFGPGTKVDIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - DNA, 2214 bp, Artificial Sequence
  pGX9244, DMAb-10
ggatccgccgccaccatggactggacttggagaatcctgttcctggtcgccgccgctactgggactcatgccgaggtgcagctggtcgaatctggagggggcctggtgcagcctggcggcagcctgaggctgtcctgcgcagcatctggcttcacctttaggagctacgacatgcactgggtgcgccaggcaacaggcaagggactggagtgggtgtctgccatcggaaccgcaggcgatacatactatccaggctccgtgaagggcaggttcaccatctcccgcgagaacgccaagaattctctgtacctgcagatgaacagcctgagagccgaggacaccgccgtgtactattgcgccagggtgcgcttcggcgacacagcagtggattattggggccagggcaccctggtgacagtgagctccgcctccaccaagggaccaagcgtgttcccactggcaccttctagcaagtccacctctggcggcacagccgccctgggctgtctggtgaaggattacttccctgagccagtgacagtgtcctggaactctggcgccctgaccagcggagtgcacacatttcctgccgtgctgcagtcctctggcctgtactccctgagctccgtggtgaccgtgccatctagctccctgggcacccagacatatatctgcaacgtgaatcacaagcctagcaatacaaaggtggacaagaaggtggagccaaagtcctgtgataagacccacacatgccctccctgtccagcacctgagctgctgggcggcccaagcgtgttcctgtttccacccaagcccaaggacaccctgatgatcagccggaccccagaggtgacatgcgtggtggtggacgtgtcccacgaggaccccgaggtgaagtttaactggtacgtggatggcgtggaggtgcacaatgccaagaccaagccccgggaggagcagtacaacagcacctatagagtggtgtccgtgctgacagtgctgcaccaggactggctgaacggcaaggagtataagtgcaaggtgagcaataaggccctgccagcccccatcgagaagaccatctccaaggcaaagggacagccaagggagccacaggtgtacacactgcctccatcccgcgacgagctgaccaagaaccaggtgtctctgacatgtctggtgaagggcttctatccctctgatatcgccgtggagtgggagagcaatggccagcctgagaacaattacaagaccacaccccctgtgctggacagcgatggctccttctttctgtattccaagctgaccgtggacaagtctcggtggcagcagggcaacgtgtttagctgctccgtgatgcacgaggccctgcacaatcactacacccagaagtctctgagcctgtccccaggcaagaggggaagaaagcggagatctggcagcggcgccacaaacttcagcctgctgaagcaggccggcgatgtggaggagaatcctggcccaatggtgctgcagacccaggtgtttatctctctgctgctgtggatcagcggcgcctatggcgacatccagatgacacagtccccttctagcctgtccgcctctgtgggcgatcgggtgaccatcacatgtagagccagccagtccatctcctctttcctgaactggcaccagcagaagcctggcaaggccccaaagctgctgatctacgcagccagctccctgcagagcggagtgccctccaggttctctggcagcggctccggaaccgactttaccctgacaatctctagcctgcagcctgaggattttgccatctactattgccagcagtcttatatcagccccttcacctttggccctggcacaaaggtggacatcaagaccgtggccgccccaagcgtgttcatctttccaccctccgatgagcagctgaagtctggcacagccagcgtggtgtgcctgctgaacaatttctacccccgcgaggccaaggtgcagtggaaggtggacaacgccctgcagtccggcaattctcaggagagcgtgaccgagcaggactccaaggattctacatattctctgagctctaccctgacactgagcaaggccgattacgagaagcacaaggtgtatgcctgcgaggtcacccaccaggggctgtcaagtccagtcactaagtccttcaatcggggcgaatgctgataactcgag

SEQ ID NO: 143 - protein, 739 aa, Artificial Sequence
  DMAb-11
MDWTWRILFLVAAATGTHAEVQLVESGGGLIQPGGSLRLSCAASGFAVRSNYLSWVRQAPGKGLEWVSLIYSGGLTAYADSVEGRFTISRDNSKNTLYLQMNSLRVEDTALYYCARVASSAGTFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPRSLSVTPGEPASISCRSSQSLLHRNGYNYLDWYLQKPGQSPQLLIYLGSNRASGVPDRFSGSGSGTDFTLKISRVEAEDVGVYYCMQALQTPSWTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - DNA, 2244 bp, Artificial Sequence
  pGX9256, DMAb-11
ggatccgccgccaccatggactggacctggagaatcctgttcctggtggcagcagcaaccggaacacacgcagaggtgcagctggtggagagcggcggcggcctgatccagccaggcggcagcctgaggctgtcctgcgcagcatctggatttgccgtgaggagcaactacctgtcctgggtgagacaggcaccaggcaagggactggagtgggtgtctctgatctacagcggcggcctgaccgcatatgcagacagcgtggagggcaggttcaccatctccagagataactctaagaatacactgtatctgcagatgaattccctgcgggtggaggacaccgccctgtactattgcgcccgcgtggccagctccgccggcacattctactatggcatggacgtgtggggccagggcaccacagtgaccgtgtctagcgcctccacaaagggaccaagcgtgttcccactggcaccttcctctaagtccacctctggcggcacagccgccctgggctgtctggtgaaggattacttccctgagccagtgaccgtgtcttggaacagcggcgccctgaccagcggagtgcacacatttcctgccgtgctgcagagctccggcctgtactccctgtctagcgtggtgaccgtgccatcctctagcctgggcacccagacatatatctgcaacgtgaatcacaagcctagcaatacaaaggtggacaagaaggtggagccaaagtcctgtgataagacccacacatgccctccctgtccagcacctgagctgctgggcggcccaagcgtgttcctgtttccacccaagcccaaggacacactgatgatctctaggaccccagaggtgacatgcgtggtggtggacgtgagccacgaggaccccgaggtgaagtttaactggtacgtggatggcgtggaggtgcacaatgccaagaccaagccaagggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggcaaagggacagccaagggagccacaggtgtacacactgcctccatccagagacgagctgaccaagaaccaggtgtctctgacatgtctggtgaagggcttctatccctctgatatcgccgtggagtgggagagcaatggccagcctgagaacaattacaagaccacaccccctgtgctggactccgatggctctttctttctgtattccaagctgaccgtggataagtctcggtggcagcagggcaacgtgtttagctgctccgtgatgcacgaggccctgcacaatcactacacccagaagtctctgagcctgtcccctggcaagaggggaaggaagaggagatctggcagcggcgccacaaacttcagcctgctgaagcaggcaggcgacgtggaggagaatcctggaccaatggtgctgcagacccaggtgtttatctctctgctgctgtggatcagcggcgcctacggcgatatcgtgatgacccagtcccctcgctccctgtctgtgacacctggcgagccagccagcatctcctgtcggtcctctcagtctctgctgcaccgcaacggctacaattatctggactggtacctgcagaagcccggccagtcccctcagctgctgatctatctgggcagcaacagggcatccggagtgccagaccgcttctctggcagcggctccggaaccgacttcaccctgaagatcagcagggtggaggccgaggatgtgggcgtgtactattgcatgcaggccctgcagaccccctcctggacattcggccagggcaccaaggtggagatcaagacagtggccgcccctagcgtgttcatctttccaccctccgacgagcagctgaagtctggcaccgccagcgtggtgtgcctgctgaacaacttctaccccagagaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtgacccaccagggcctgtctagccctgtgacaaagtccttcaacagaggcgagtgttgataactcgag

SEQ ID NO: 145 - protein, 741 aa, Artificial Sequence
  DMAb12
MDWTWRILFLVAAATGTHAQVQLVQSGAEVKKPGSSVKVSCKASGATFGSDTVTWVRQAPGQGLEWMGGIIPFFGEANYAQRFQGRVTITADKSTNTAYMELSSLRSEDTAVYFCARQINEMATFGEIHYYTYMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGGSELTQDPAVSVALGQTVRITCQGDSLRNYYASWYQQKPRQAPVLVFYGKNNRPSGIPDRFSGSSSGNTASLTISGAQAEDEADYYCNSRDSSSNHLVFGGGTKLTVLSTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 146 - DNA, 2232 bp, Artificial Sequence
  pGX9260, DMAb12
catggactggacctggagaatcctgttcctggtggcagcagcaaccggaacacacgcacaggtgcagctggtgcagagcggagcagaggtgaagaagccaggcagctccgtgaaggtgtcctgcaaggcatctggagccaccttcggctccgataccgtgacatgggtgcgccaggcaccaggacagggactggagtggatgggcggcatcatccctttctttggcgaggccaactacgcccagcggtttcagggcagagtgaccatcacagccgacaagtccaccaatacagcctacatggagctgtctagcctgcggtctgaggataccgccgtgtatttctgcgccagacagatcaacgagatggccacctttggcgagatccactactatacatacatggacgtgtggggacagggcaccctggtgacagtgtcctctgcctccaccaagggacctagcgtgttcccactggcacctagctccaagtctaccagcggcggcacagccgccctgggatgtctggtgaaggattatttccctgagccagtgacagtgtcctggaactctggcgccctgaccagcggagtgcacacatttcccgccgtgctgcagtctagcggcctgtactccctgtcctctgtggtgaccgtgcctagctcctctctgggcacccagacatatatctgcaacgtgaatcacaagccttctaatacaaaggtggacaagaaggtggagccaaagagctgtgataagacccacacatgccctccctgtccagcacctgagctgctgggcggcccaagcgtgttcctgtttccacccaagcccaaggacaccctgatgatcagcaggacccctgaggtgacatgcgtggtggtggacgtgtcccacgaggaccccgaggtgaagttcaactggtacgtggatggcgtggaggtgcacaatgccaagaccaagccccgggaggagcagtacaacagcacctatagagtggtgtccgtgctgacagtgctgcaccaggactggctgaacggcaaggagtataagtgcaaggtgtctaataaggccctgccagcccccatcgagaagaccatctccaaggcaaagggacagccaagggagccacaggtgtacacactgcctccaagccgcgacgagctgaccaagaaccaggtgtccctgacatgtctggtgaagggcttctatccatccgatatcgccgtggagtgggagtctaatggccagcccgagaacaattacaagaccacaccccctgtgctggacagcgatggctccttctttctgtattccaagctgaccgtggacaagtctcggtggcagcagggcaacgtgttttcctgctctgtgatgcacgaggccctgcacaatcactacacccagaagagcctgtccctgtctcctggcaagaggggaaggaagcggagaagcggctccggagccacaaacttcagcctgctgaagcaggccggcgatgtggaggagaatcctggcccaatggtgctgcagacccaggtgtttatctctctgctgctgtggatcagcggagcatacggcggctccgagctgacacaggacccagccgtgagcgtggccctgggacagaccgtgaggatcacatgtcagggcgatagcctgcgcaactactatgcctcctggtaccagcagaagcctcggcaggccccagtgctggtgttctatggcaagaacaataggccctctggcatccctgaccgctttagcggcagctcctctggcaataccgcaagcctgacaatctccggagcacaggcagaggacgaggcagattactattgcaacagcagagatagctcctctaatcacctggtgttcggcggcggaaccaagctgacagtgctgtctaccgtggccgccccaagcgtgttcatctttccaccctccgacgagcagctgaagtctggcacagccagcgtggtgtgcctgctgaacaacttctacccccgggaggccaaggtgcagtggaaggtggataacgccctgcagtctggcaatagccaggagtccgtgaccgagcaggactctaaggatagcacatattctctgagcaacaccctgacactgagcaaggccgactacgagaagcacaaggtgtatgcatgcgaggtgacccaccagggactgagctccccagtgacaaagtccttcaatagaggcgagtgttgataact

SEQ ID NO: 147 - protein, 735 aa, Artificial Sequence
  DMAb13
MDWTWRILFLVAAATGTHAQVQLVESGGGVVQPGGSLRLSCEVSGLTFSNFGMQWVRQAPGKGLEWVAFIRFDGSNKYYADSVKGRFTISRDNSKNTVYLQMGSLRAEDTAVYFCGRVLYGAAADFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCTSSHSLLYSSDNKNYLTWYQQKAGQPPKLLIYWASTRQSGVPDRFSGSGSGTEFTLTISSLQAEDVAVYYCQQYYTKSFTFGQGTKVEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 148 - DNA, 2214 bp, Artificial Sequence
  pGX9261, DMAb13
catggactggacctggagaatcctgttcctggtggcagcagcaaccggaacacacgcacaggtgcagctggtggagagcggcggcggcgtggtgcagcctggcggctctctgagactgagctgcgaggtgtccggcctgaccttcagcaactttggaatgcagtgggtgaggcaggcaccaggcaagggactggagtgggtggccttcatccgctttgacggctctaataagtactatgccgatagcgtgaagggccggttcaccatctctagagacaacagcaagaatacagtgtacctgcagatgggcagcctgagggcagaggacaccgccgtgtacttctgcggacgcgtgctgtatggagcagcagcagatttttggggacagggcaccctggtgacagtgagctccgcctctacaaagggaccaagcgtgtttccactggcaccctctagcaagtccacctctggcggcacagccgccctgggctgtctggtgaaggattacttccccgagcctgtgaccgtgagctggaactccggcgccctgacctccggagtgcacacatttccagccgtgctgcagtcctctggcctgtacagcctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtccagcaccagagctgctgggcggcccaagcgtgttcctgtttccacccaagcctaaggacaccctgatgatctctagaacccccgaggtgacatgcgtggtggtggacgtgagccacgaggaccccgaggtgaagttcaactggtacgtggatggcgtggaggtgcacaatgccaagacaaagcctcgggaggagcagtacaactccacctatagagtggtgtctgtgctgacagtgctgcaccaggactggctgaacggcaaggagtataagtgcaaggtgtccaataaggccctgcctgccccaatcgagaagaccatctctaaggcaaagggacagcctcgggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtattccaagctgaccgtggacaagtctcggtggcagcagggcaacgtgtttagctgctccgtgatgcacgaggccctgcacaatcactacacccagaagtctctgagcctgtccccaggcaagaggggaaggaagcggagatctggcagcggagccacaaacttctccctgctgaagcaggcaggcgatgtggaggagaatccaggacctatggtgctgcagacccaggtgtttatcagcctgctgctgtggatctccggcgcctatggcgacatcgtgatgacacagtccccagattctctggccgtgtccctgggagagagggcaaccatcaactgtacatctagccacagcctgctgtactcctctgacaacaagaattacctgacctggtatcagcagaaggccggccagccacccaagctgctgatctattgggcatccaccaggcagtctggagtgccagaccgcttctccggctctggcagcggcacagagtttaccctgacaatcagctccctgcaggccgaggatgtggccgtgtactattgccagcagtactataccaagagcttcacatttggccagggcaccaaggtggagatcaagacagtggccgcccccagcgtgttcatctttcctccatccgacgagcagctgaagagcggaaccgcatccgtggtgtgcctgctgaacaatttctaccctagggaggccaaggtgcagtggaaggtggataacgccctgcagagcggcaattcccaggagtctgtgaccgagcaggacagcaaggattccacatattccctgtctaacaccctgacactgtccaaggccgattacgagaagcacaaggtgtatgcctgcgaggtgacccaccagggcctgtctagccctgtgacaaagagctttaatcgcggcgagtgttgataact

SEQ ID NO: 149 - DNA, 2067 bp, Artificial Sequence
  Marburg GP1 Consensus
ggatccgccaccatgaagaccacatgcctgttcatcagcctgatcctgatccagggcgtgaagaccctgccaatcctggagatcgcctccaacaatcagccccagaacgtggacagcgtgtgctctggcaccctgcagaagacagaggatgtgcacctgatgggcttcacactgagcggacagaaggtggcagactccccactggaggcctctaagaggtgggcctttagaaccggcgtgccccctaagaacgtggagtacaccgagggcgaggaggccaagacatgctataatatctccgtgaccgatcccagcggcaagtccctgctgctggacccacccacaaacatcagggattaccctaagtgtaagaccatccaccacatccagggccagaatccacacgcacagggaatcgccctgcacctgtggggcgccttctttctgtacgacaggatcgccagcaccacaatgtatagaggcaaggtgttcacagagggcaacatcgccgccatgatcgtgaataagaccgtgcacaagatgatcttttctaggcagggccagggctacagacacatgaacctgacaagcaccaataagtattggacaagctccaacggcacacagaccaatgacaccggatgcttcggcgccctgcaggagtacaactccaccaagaatcagacatgtgccccatctaagatccctctgccactgccaacagcaaggccagaggtgaagctgacatctaccagcacagacgccaccaagctgaacaccacagaccccaattccgacgatgaggatctgaccacatccggctctggcagcggagagcaggagccttataccacatctgatgccgccaccaagcagggcctgtctagcaccatgcctccaacacctagcccacagccctccacacctcagcaggagggcaacaataccaaccactctcagggagcagtgaccgagcctggcaagacaaacaccacagcccagccaagcatgccccctcacaataccacagccatcagcaccaacaatacatccaagcacaacttttctacccctagcgtgccactgcagaatgccaccaactacaatacacagtccaccgccacagagaacgagcagacatccgccccctctaagaccacactgccacccaccgagaaccctaccacagccaagagcaccaattccacaaagtctccaaccacaaccgtgcccaacacaaccaataagcactccacctctcctagcccaacccccaaccctacagcccagcacctggtgtatttcaggagaaagcggaatatcctgtggcgcgagggcgacatgttcccctttctggatggcctgatcaacgcccctatcgacttcgatccagtgcccaataccaagacaatctttgacgagtcctctagctccggagcaagcgccgaggaggatcagcacgcctctcctaacatcagcctgacactgtcctactttccaaagatcaacgagaataccgcctattccggcgagaacgagaatgactgcgatgccgagctgaggatctggagcgtgcaggaggacgatctggcagcaggactgtcctggattcccttcttcggacctggaatcgagggactgtacaccgcaggactgatcaagaaccagaacaacctggtgtgcagactgcggcgcctggccaatcagacagccaagtccctggagctgctgctgcgggtgacaaccgaggagcgcaccttctctctgatcaaccggcacgccatcgactttctgctggcaagatggggcggcacctgcaaggtgctgggaccagactgctgtatcggcatcgaggatctgtctcggaatatcagcgagcagatcgaccagatcaagaaggatgagcagaaggagggaaccggatggggactgggcggcaagtggtggacaagcgattggggcgtgctgaccaacctgggcatcctgctgctgctgtctatcgccgtgctgatcgccctgagctgcatctgtcgcatcttcaccaagtatatcggctgataactcgag

SEQ ID NO: 150 - protein, 681 aa, Artificial Sequence
  Marburg GP1 Consensus
MKTTCLFISLILIQGVKTLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPLPLPTARPEVKLTSTSTDATKLNTTDPNSDDEDLTTSGSGSGEQEPYTTSDAATKQGLSSTMPPTPSPQPSTPQQEGNNTNHSQGAVTEPGKTNTTAQPSMPPHNTTAISTNNTSKHNFSTPSVPLQNATNYNTQSTATENEQTSAPSKTTLPPTENPTTAKSTNSTKSPTTTVPNTTNKHSTSPSPTPNPTAQHLVYFRRKRNILWREGDMFPFLDGLINAPIDFDPVPNTKTIFDESSSSGASAEEDQHASPNISLTLSYFPKINENTAYSGENENDCDAELRIWSVQEDDLAAGLSWIPFFGPGIEGLYTAGLIKNQNNLVCRLRRLANQTAKSLELLLRVTTEERTFSLINRHAIDFLLARWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 151 - DNA, 2067 bp, Artificial Sequence
  Marburg GP2 Consensus
ggatccgccaccatgcggaccacatgcttctttatcagcctgatcctgatccagggcatcaagaccctgccaatcctggagatcgcctccaacgaccagccccagaatgtggatagcgtgtgctccggcaccctgcagaagacagaggacgtgcacctgatgggcttcacactgtctggccagaaggtggccgatagccccctggaggcctctaagaggtgggcctttagaaccggcgtgccccctaagaacgtggagtacaccgagggcgaggaggccaagacatgctataatatcagcgtgaccgacccttctggcaagagcctgctgctggacccacccacaaacgtgcgcgattaccctaagtgtaagaccatccaccacatccagggccagaatccacacgcacagggaatcgccctgcacctgtggggcgccttctttctgtacgataggatcgcctccaccacaatgtatagaggcaaggtgttcacagagggcaacatcgccgccatgatcgtgaataagaccgtgcacaagatgatcttttccaggcagggccagggctacagacacatgaacctgacatctaccaataagtattggaccagctccaacggcacacagaccaatgacaccggctgcttcggcacactgcaggagtacaactccaccaagaatcagacatgtgccccttctaagacccctccaccccctccaacagcaaggccagagatcaagcctacaagcaccccaacagacgccaccagactgaacaccacaaaccccaattccgacgatgaggatctgaccacaagcggctccggctctggagagcaggagccttataccacatccgacgccgtgaccaagcagggcctgtctagcaccatgccccctacaccttccccacagccaggaacacctcagcagggcggcaacaataccaaccactctcaggacgccaccacagagctggataacacaaataccacagcccagccacccatgccaagccacaataccacaaccatcagcaccaacaatacatccaagcacaacctgtccaccctgtctgagcctccacagaatacaaccaacccaaatacccagagcatggtgacagagaacgagaagaccagcgcccctcccaaggccaccctgccacccacagagaatcccacaaccgagaagtctaccaacaatacaaagagccctacaaccctggagccaaacaagacaaatggccacttcaccagcccctcctctacacctaactccacaacccagcacctgatctactttaggagaaagcggtccatcctgtggcgcgagggcgacatgttcccctttctggatggcctgatcaacgcccctatcgacttcgatcctgtgccaaataccaagacaatctttgacgagagctcctctagcggagcaagcgccgaggaggatcagcacgcctcctctaacatcagcctgaccctgtcctacctgccacacacctccgagaatacagcctattctggcgagaacgagaatgactgcgatgccgagctgcggatctggtctgtgcaggaggacgatctggcagcaggactgagctggattcccctgttcggaccaggaatcgagggactgtataccgccggcctgatcaagaaccagaacaacctggtgtgcaggctgcggcgcctggcaaatcagacagccaagtctctggagctgctgctgcgggtgacaaccgaggagcgcaccttcagcctgatcaaccggcacgccatcgactttctgctgacccgctggggcggcacatgcaaggtgctgggaccagactgctgtatcggcatcgaggatctgtctaggaacatcagcgagcagatcgaccagatcaagaaggatgagcagaaggagggaaccggatggggactgggcggcaagtggtggacaagcgattggggcgtgctgaccaacctgggcatcctgctgctgctgtctatcgccgtgctgatcgccctgagctgcatctgtagaatctttaccaagtatatcggctgataactcgag

SEQ ID NO: 152 - protein, 681 aa, Artificial Sequence
  Marburg GP2 Consensus
MRTTCFFISLILIQGIKTLPILEIASNDQPQNVDSVCSGTLQKTEDVHLMGFTLSGQKVADSPLEASKRWAFRTGVPPKNVEYTEGEEAKTCYNISVTDPSGKSLLLDPPTNVRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCAPSKTPPPPPTARPEIKPTSTPTDATRLNTTNPNSDDEDLTTSGSGSGEQEPYTTSDAVTKQGLSSTMPPTPSPQPGTPQQGGNNTNHSQDATTELDNTNTTAQPPMPSHNTTTISTNNTSKHNLSTLSEPPQNTTNPNTQSMVTENEKTSAPPKATLPPTENPTTEKSTNNTKSPTTLEPNKTNGHFTSPSSTPNSTTQHLIYFRRKRSILWREGDMFPFLDGLINAPIDFDPVPNTKTIFDESSSSGASAEEDQHASSNISLTLSYLPHTSENTAYSGENENDCDAELRIWSVQEDDLAAGLSWIPLFGPGIEGLYTAGLIKNQNNLVCRLRRLANQTAKSLELLLRVTTEERTFSLINRHAIDFLLTRWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 153 - DNA, 2067 bp, Artificial Sequence
  Marburg GP3 Consensus
ggatccgccaccatgaagacaatctacttcctgatctctctgatcctgatccagagcatcaagaccctgccagtgctggagatcgcctccaactctcagccccaggacgtggatagcgtgtgctctggcaccctgcagaagacagaggacgtgcacctgatgggcttcacactgtccggccagaaggtggccgattcccctctggaggcctctaagaggtgggcctttagaaccggcgtgccccctaagaacgtggagtacaccgagggcgaggaggccaagacatgctataatatctccgtgaccgacccaagcggcaagtccctgctgctggacccaccctctaacatcagggattaccccaagtgtaagaccgtgcaccacatccagggccagaatcctcacgcacagggaatcgccctgcacctgtggggcgccttctttctgtacgatcgggtggccagcaccacaatgtatcgcggcaaggtgttcacagagggcaacatcgccgccatgatcgtgaataagaccgtgcaccggatgatcttttctcgccagggccagggctacaggcacatgaacctgaccagcacaaataagtattggacaagctccaacgagacccagagaaatgacacaggctgctttggcatcctgcaggagtataactccaccaacaatcagacatgtcctccaagcctgaagcccccttccctgcctaccgtgacaccatctatccacagcaccaacacacagatcaataccgccaagagcggcacaatgaacccttctagcgacgatgaggacctgatgatcagcggctccggctctggagagcagggaccacacaccacactgaatgtggtgaccgagcagaagcagtcctctaccatcctgtccacaccatctctgcacctgagcacatcccagcacgagcagaactctaccaatcccagccggcacgcagtgaccgagcacaacggcacagaccccaccacacagcctgccaccctgctgaacaatacaaataccacacctacctacaacacactgaagtataatctgagcacaccatccccacccaccaggaacatcacaaacaatgatacccagagagagctggccgagtccgagcagaccaacgcccagctgaataccacactggacccaacagagaaccccaccacaggccaggataccaactctaccacaaatatcatcatgaccacatccgacatcacctctaagcacccaacaaatagctcccctgattctagcccaaccacacgccctccaatctacttcaggaagaagaggagcatcttttggaaggagggcgacatcttcccctttctggatggcctgatcagcaccgagatcgacttcgatccaatccccaacaccgagacaatcttcgacgagtctcccagctttaacacctccacaaatgaggagcagcacacaccccctaacatcagcctgaccttctcctactttcctgacaagaatggcgataccgcctatagcggcgagaacgagaatgactgcgatgccgagctgcggatctggagcgtgcaggaggacgatctggcagcaggactgtcctggattcccttcttcggccctggcatcgagggcctgtataccgccggcctgatcaagaaccagaacaacctggtgtgccgcctgaggagactggccaatcagacagccaagagcctggagctgctgctgagggtgaccacagaggagagaaccttctccctgatcaaccggcacgccatcgactttctgctgacccgctggggcggcacatgcaaggtgctgggaccagactgctgtatcggcatcgaggatctgtctaagaatatcagcgagcagatcgacaagatcaggaaggatgagcagaaggaggagaccggatggggactgggcggcaagtggtggacaagcgattggggcgtgctgaccaacctgggcatcctgctgctgctgtccatcgccgtgctgatcgccctgtcttgcatctgtagaatcttcaccaagtacatcggctgataactcgag

SEQ ID NO: 154 - protein, 681 aa, Artificial Sequence
  Marburg GP3 Consensus
MKTIYFLISLILIQSIKTLPVLEIASNSQPQDVDSVCSGTLQKTEDVHLMGFTLSGQKVADSPLEASKRWAFRTGVPPKNVEYTEGEEAKTCYNISVTDPSGKSLLLDPPSNIRDYPKCKTVHHIQGQNPHAQGIALHLWGAFFLYDRVASTTMYRGKVFTEGNIAAMIVNKTVHRMIFSRQGQGYRHMNLTSTNKYWTSSNETQRNDTGCFGILQEYNSTNNQTCPPSLKPPSLPTVTPSIHSTNTQINTAKSGTMNPSSDDEDLMISGSGSGEQGPHTTLNVVTEQKQSSTILSTPSLHLSTSQHEQNSTNPSRHAVTEHNGTDPTTQPATLLNNTNTTPTYNTLKYNLSTPSPPTRNITNNDTQRELAESEQTNAQLNTTLDPTENPTTGQDTNSTTNIIMTTSDITSKHPTNSSPDSSPTTRPPIYFRKKRSIFWKEGDIFPFLDGLISTEIDFDPIPNTETIFDESPSFNTSTNEEQHTPPNISLTFSYFPDKNGDTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSKNISEQIDKIRKDEQKEETGWGLGGKWWTSDWGVLTNLGILLLLSIAVLIALSCICRIFTKYIG

SEQ ID NO: 155 - protein, 735 aa, Artificial Sequence
  Influenza A DMAb
MDWTWRILFLVAAATGTHAQVQLQQSGPGLVKPSQTLSLTCAISGDSVSSNNAVWNWIRQSPSRGLEWLGRTYYRSKWYNDYAESVKSRITINPDTSKNQFSLQLNSVTPEDTAVYYCARSGHITVFGVNVDAFDMWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSLSASVGDRVTITCRTSQSLSSYLHWYQQKPGKAPKLLIYAASSLQSGVPSRFSGSGSGTDFTLTISSLQPEDFATYYCQQSRTFGQGTKVEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 156 - protein, 745 aa, Artificial Sequence
  Influenza B DMAb
MDWTWRILFLVAAATGTHAEVQLVESGGGLVKPGGSLRLSCAASGLTFLNAWMSWVRQAPGKGLEWVGRIKSNTDGGTTDYAAPVKGRFTISRDDSKNTLYLQMSSLKTEDTAVYYCTTDGPYSDDFRSGYAARYRYFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSVSASVGDRVTITCRASQDISTWLAWYQQKPGKAPKLLIYAASSLQSGVPSRFSGSGSGTDFTLTISSLQPEDFATYYCQQANSFPPTFGQGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 157 - protein, 736 aa, Artificial Sequence
  Influenza A DMAb
MDWTWRILFLVAAATGTHAEVQLVESGPGLVKPSDILSLTCAVSGYSISSNYYWGWIRQPPGKGLEWIGSIYHSGSTYYKPSLESRLGISVDTSKNQFSLKLSFVSAADTAVYYCARHVRSGYPDTAYYFDKWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGSYVLTQPPSVSVAPGETARISCGGNNIGTKVLHWYQQTPGQAPVLVVYDDSDRPSGIPERFSGSNSGNTATLTISRVEVGDEADYYCQVWDISTDQAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 158 - protein, 744 aa, Artificial Sequence
  Influenza A DMAb
MDWTWRILFLVAAATGTHAQVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 159 - protein, 477 aa, Artificial Sequence
  Influenza A DMAb
MDWTWRILFLVAAATGTHAQVQLQQSGPGLVKPSQTLSLTCAISGDSVSSNNAVWNWIRQSPSRGLEWLGRTYYRSKWYNDYAESVKSRITINPDTSKNQFSLQLNSVTPEDTAVYYCARSGHITVFGVNVDAFDMWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 229 aa, Artificial Sequence
  Influenza A DMAb
MVLQTQVFISLLLWISGAYGDIQMTQSPSSLSASVGDRVTITCRTSQSLSSYLHWYQQKPGKAPKLLIYAASSLQSGVPSRFSGSGSGTDFTLTISSLQPEDFATYYCQQSRTFGQGTKVEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 161 - protein, 477 aa, Artificial Sequence; identical to SEQ ID NO: 159
  Influenza A DMAb

SEQ ID NO: 162 - protein, 229 aa, Artificial Sequence; identical to SEQ ID NO: 160
  Influenza A DMAb

SEQ ID NO: 163 - DNA, 2211 bp, Artificial Sequence
  Influenza A DMAb
atggattggacttggaggattctgtttctggtcgccgccgctactggaactcacgctcaggtgcagctgcagcagtctggacccggactggtgaagccttcacagactctgagcctgacctgcgccatctccggcgactctgtgagctccaacaatgctgtctggaactggattagacagtccccatctcgggggctggaatggctgggacgaacatactataggagcaaatggtacaatgactatgctgagagtgtgaagtcacgaatcacaattaacccagatactagcaagaatcagttctccctgcagctgaactctgtgacacccgaggatactgcagtctactattgcgcacgctccggacacatcaccgtgttcggagtcaatgtggacgcctttgatatgtggggacaggggaccacagtcacagtgtctagtgcaagtactaaaggcccatcagtgtttcccctggccccttcaagcaagagtacctcaggcggaacagccgctctgggatgtctggtgaaggactacttccctgagccagtcaccgtgagctggaactccggagctctgaccagcggggtgcatacatttcctgcagtcctgcagtcctctggcctgtacagcctgagttcagtggtcaccgtgccaagctcctctctgggaacacagacttatatctgcaacgtgaatcacaaaccatccaatacaaaggtcgacaagaaagtggaacccaaatcttgtgataagacccatacatgccctccctgtccagcacctgagctgctgggcggcccatccgtgttcctgtttccacccaagcctaaagacacactgatgattagccggactcccgaagtgacctgcgtggtcgtggacgtgagccacgaggaccccgaagtgaagttcaactggtacgtggatggcgtcgaggtgcataatgccaagaccaaacctagggaggaacagtacaacagcacttatagagtcgtgtccgtcctgaccgtgctgcaccaggattggctgaacgggaaggagtataagtgcaaagtgtccaacaaggccctgccagctcccatcgagaagaccatttctaaggccaaaggccagccacgggaaccccaggtgtacacactgcctccaagccgcgacgagctgaccaaaaaccaggtgagcctgacatgtctggtcaagggattctatcctagtgatatcgctgtggagtgggaatctaatgggcagccagaaaacaattacaagactacccctcccgtgctggactctgatggaagtttctttctgtattcaaaactgaccgtggacaagagccgctggcagcaggggaacgtctttagctgctccgtgatgcacgaggccctgcacaatcattacactcagaaatctctgagtctgtcacccggaaaacgaggacgaaagaggagaagcggctccggagctaccaacttctccctgctgaagcaggcaggggatgtggaggaaaatcctggcccaatggtcctgcagacacaggtgtttatctctctgctgctgtggattagtggcgcttacggagacatccagatgactcagtctcctagttcactgtctgcaagtgtcggcgatcgcgtgactattacctgtcgaacctcacagagcctgagctcctacctgcattggtatcagcagaagcctgggaaagcaccaaagctgctgatctatgcagcctctagtctgcagtccggcgtgccctctaggttctccgggtctggcagtggaactgactttacactgactatttcaagcctgcagcctgaggatttcgctacctactattgccagcagagcagaacttttgggcagggcaccaaagtcgaaatcaagacagtggctgcaccatccgtcttcatttttccaccctctgacgagcagctgaagagtggaactgcctcagtggtgtgcctgctgaacaatttctacccccgggaagccaaagtccagtggaaggtggataacgctctgcagtcaggcaatagccaggagtccgtgacagaacaggactctaaagatagtacttattcactgagcaacaccctgacactgagcaaggcagactacgagaagcacaaagtgtatgcctgcgaagtgacccaccaggggctgagcagtccagtgaccaaatctttcaacaggggagaatgttgataa

SEQ ID NO: 164 - DNA, 2241 bp, Artificial Sequence
  Influenza B DMAb
atggactggacttggaggattctgtttctggtggccgccgcaactggcactcatgccgaggtgcagctggtggaatcagggggaggactggtgaagcctggcggatcactgcgactgagctgcgcagcttccggactgaccttcctgaacgcttggatgagctgggtgcgacaggcaccagggaaaggcctggaatgggtcgggcgcatcaagagcaatacagacggcggaaccacagattacgcagcccccgtgaaaggcaggttcaccatttctcgggacgatagtaagaacacactgtatctgcagatgagctccctgaaaaccgaggacacagccgtgtactattgcactaccgatggcccctacagcgacgatttccgctccggatatgctgcacggtaccgctattttgggatggacgtgtggggacaggggacaactgtcacagtgtctagtgcatctactaagggacctagcgtgttcccactggccccctcaagcaaatcaactagcggagggaccgccgctctgggatgtctggtgaaggattacttccccgagcctgtcaccgtgagctggaactccggggccctgacctccggagtgcacacatttcctgctgtcctgcagtcctctgggctgtactctctgagttcagtggtcacagtgccaagctcctctctgggcactcagacctatatctgcaacgtgaatcacaaacctagcaatactaaggtcgacaagaaagtggaaccaaaaagctgtgataagacacatacttgccctccctgtccagctccagagctgctgggcggaccatccgtgttcctgtttccacccaagcccaaagacaccctgatgatttcccggacaccagaagtgacttgcgtggtcgtggacgtgagccacgaggaccccgaagtgaagttcaactggtacgtggatggcgtcgaggtgcataatgccaagacaaaacccagggaggaacagtacaactcaacttatagagtcgtgagcgtcctgaccgtgctgcaccaggactggctgaacggcaaggagtataagtgcaaagtgagcaacaaggccctgcctgctccaatcgagaagactattagcaaggctaaaggacagcctcgggaaccacaggtgtacaccctgcctccatcccgcgacgagctgaccaaaaaccaggtgtctctgacatgtctggtcaagggcttctatccctctgatatcgccgtggagtgggaaagtaatggacagcctgaaaacaattacaagaccacaccccctgtgctggactctgatggcagtttctttctgtatagtaaactgaccgtggacaagtcaagatggcagcagggaaacgtgttttcctgctctgtcatgcatgaggccctgcacaatcattacacccagaagagtctgtcactgagcccaggaaaacgagggaggaagaggagatccggctctggagccacaaacttctccctgctgaagcaggctggagacgtggaggaaaatcccgggcctatggtgctgcagacccaggtctttatctccctgctgctgtggatttctggcgcttacggagatatccagatgacacagtctcccagttcagtcagtgcatcagtgggcgaccgcgtcaccatcacatgtcgagcatcacaggatattagcacctggctggcctggtaccagcagaagcccggaaaagctcctaagctgctgatctatgcagccagctccctgcagtccggagtgccctctaggttcagcgggtccggctctggaacagactttactctgaccatttctagtctgcagcctgaggatttcgcaacttactattgccagcaggccaacagcttcccacccacttttgggcagggcaccaaactggaaatcaagactgtggctgcacctagcgtcttcatttttcctccatccgacgagcagctgaagagtggcaccgcctcagtggtgtgcctgctgaacaacttctacccaagagaagcaaaagtgcagtggaaggtcgataacgccctgcagtcaggcaatagccaggagtccgtgacagaacaggactctaaggatagtacttatagtctgtcaaatacactgactctgagcaaagctgactacgagaagcataaagtgtatgcatgcgaggtcactcaccagggactgtcttcacccgtcaccaaatctttcaatagaggagaatgctgataa

SEQ ID NO: 165 - DNA, 2214 bp, Artificial Sequence
  Influenza A DMAb
atggactggacatggagaatcctgttcctggtcgccgccgctactgggactcacgcagaagtgcagctggtcgaatcagggcctgggctggtgaagccctcagacatcctgagcctgacctgcgccgtgtctggctacagtatcagctccaactactattggggatggattcggcagccccctggcaagggactggaatggatcgggtccatctaccactcaggcagcacctactataaaccttcactggagagccgcctgggaatttccgtggacacatctaagaatcagttcagcctgaaactgtcctttgtctctgccgctgatactgcagtgtactattgcgcccgacatgtcaggtccggctacccagacaccgcttactattttgataagtgggggcagggcaccctggtcacagtgtctagtgctagcaccaagggcccctccgtgttccctctggcaccatcaagcaaatccacatctggcggaactgcagccctgggatgtctggtgaaggattacttcccagagcccgtcacagtgagttggaactcaggcgcactgacttctggagtccacacctttcccgccgtgctgcagtcctctggcctgtacagcctgagttcagtggtcacagtgcctagctcctctctgggaactcagacctatatctgcaacgtgaatcacaagccctcaaatactaaagtcgacaagaaagtggaacctaagtcttgtgataaaacacatacttgcccaccatgtcctgcaccagagctgctgggaggaccaagcgtgttcctgtttcctccaaagcccaaagacaccctgatgatctccagaacccctgaagtgacatgtgtggtcgtggacgtctctcacgaggaccccgaagtcaagtttaactggtacgtggatggcgtcgaggtgcataatgctaagacaaaaccccgcgaggaacagtacaactcaacctatcgagtcgtgagcgtcctgacagtgctgcaccaggactggctgaacggaaaggagtataagtgcaaagtgagcaataaggcactgcccgcccctatcgagaaaactatttccaaggctaaagggcagcccagggaacctcaggtgtacaccctgcccccttctagagacgagctgacaaagaaccaggtcagtctgacttgtctggtgaaaggattttatccaagtgatatcgcagtggagtgggaatcaaatgggcagcccgaaaacaattacaagaccacaccacccgtgctggacagcgatggcagcttcttcctgtattccaagctgaccgtggacaaatctcggtggcagcaggggaacgtcttcagttgctcagtgatgcacgaggccctgcacaatcattacacccagaagagcctgtccctgtctccaggcaagcggggacgcaaaaggagaagtggatcaggggccacaaacttttccctgctgaaacaggctggagatgtggaggaaaatccagggcccatggtcctgcagactcaggtgttcatcagcctgctgctgtggatttctggggcctacggcagttatgtgctgacacagcctccaagcgtctccgtggctcctggcgaaactgcacgaatctcctgtggagggaacaatattgggactaaggtgctgcattggtaccagcagaccccaggacaggctccagtgctggtcgtgtatgacgatagtgacagaccttcaggcatccccgagcggttctctggaagtaactcagggaataccgccacactgactatttcccgcgtcgaagtgggcgacgaagctgattactattgccaagtgtgggacatctctaccgatcaggccgtcttcggcggagggactaagctgaccgtgctgggccagcccaaagctgcaccttccgtgacactgtttccccctagttcagaggaactgcaggctaacaaggcaaccctggtgtgtctgattagcgacttctacccaggagcagtcacagtggcatggaaggctgatagctcccctgtcaaagccggcgtggaaactaccacaccatctaagcagagtaacaacaagtacgccgcttctagttatctgagcctgacacctgagcagtggaagtcccacaggagctattcctgccaagtgactcatgagggcagtactgtcgaaaaaaccgtggccccaacagagtgtagctgataa

SEQ ID NO: 166 - DNA, 2238 bp, Artificial Sequence
  Influenza A DMAb
atggactggacttggaggattctgtttctggtcgccgccgctactgggacacacgctcaggtgcagctggtcgagagtggggggggagtggtccagccagggcgatctctgaggctgagttgcgccgcttcaggcttcaccttcagcacttacgcaatgcactgggtgcggcaggctccaggaaagggactggagtgggtcgccgtgatctcttacgacgctaactataagtactatgcagatagtgtgaaaggcagattcaccattagccgggacaactccaagaatacactgtacctgcagatgaattccctgcgagcagaagacaccgccgtgtactattgcgccaaagattctcagctgcgcagtctgctgtatttcgagtggctgtctcaggggtactttgactattggggccagggaaccctggtcacagtgagctccgccagtaccaagggcccatcagtgtttcctctggctccatctagtaaatctacaagtggcggaactgcagccctgggctgtctggtgaaggattacttcccagagcccgtcacagtgtcctggaactctggagctctgacttccggggtgcatacctttcctgcagtcctgcagtcaagcgggctgtactctctgtcctctgtggtcaccgtgccaagttcaagcctgggcactcagacctatatctgcaacgtgaatcacaagccttccaatacaaaagtcgacaagaaagtggaaccaaagtcttgtgataaaacacatacttgccccccttgtcctgctccagagctgctgggaggaccaagcgtgttcctgtttccacccaagcccaaagacaccctgatgattagcaggaccccagaagtgacatgtgtggtcgtggacgtcagccacgaggaccccgaagtgaagttcaactggtacgtggatggcgtcgaggtgcataatgccaagacaaaacctagggaggaacagtacaacagcacttatagagtcgtgtccgtcctgaccgtgctgcaccaggactggctgaacggaaaggagtataagtgcaaagtgtccaataaggccctgcccgctcctatcgagaaaaccatttctaaggctaaagggcagccccgggaacctcaggtgtacacactgcctccaagccgcgacgagctgaccaagaaccaggtgtccctgacatgtctggtcaaaggcttctatcccagtgatatcgccgtggagtgggaatcaaatggacagcctgaaaacaattacaagaccacaccccctgtgctggacagtgatggctcattctttctgtattcaaagctgaccgtggacaaaagccggtggcagcagggaaacgtcttttcatgcagcgtgatgcatgaggctctgcacaatcattacactcagaagtccctgtctctgagtcccggcaagcggggacgcaaaaggagatcagggagcggcgctacaaacttctccctgctgaagcaggcaggcgatgtggaggaaaatccaggacccatggtcctgcagacacaggtgtttatctctctgctgctgtggattagtggggcctatggcgacatcgtgatgactcagagccctgattccctggcagtgagcctgggagagcgagcaacaattaactgtaagtcctctcagagcgtgactttcaactacaaaaattatctggcatggtaccagcagaagcccggacagccacccaaactgctgatctattgggcctcaactcgcgaaagcggggtgcctgaccgattctccggatctgggagtggcaccgattttaccctgacaattagttcactgcaggctgaggacgtcgcagtgtactattgccagcagcactacaggactcctccaaccttcggacaggggacaaaggtcgaaatcaaaactgtggctgcaccttccgtcttcatttttcccccttctgacgagcagctgaagtccggcaccgcctctgtcgtgtgtctgctgaacaatttttacccaagagaagccaaggtccagtggaaagtggataacgctctgcagtctggaaatagtcaggagtcagtgacagaacaggacagcaaggattccacttattcactgagcaacactctgaccctgagcaaagcagattacgagaagcacaaagtgtatgcctgcgaagtcactcatcagggactgagctcccccgtgaccaagagctttaatagaggggagtgttgataa

SEQ ID NO: 167 - DNA, 1437 bp, Artificial Sequence
  Influenza A DMAb
atggattggacttggaggattctgtttctggtcgccgccgctactggaactcacgctcaggtgcagctgcagcagtctggacccggactggtgaagccttcacagactctgagcctgacctgcgccatctccggcgactctgtgagctccaacaatgctgtctggaactggattagacagtccccatctcgggggctggaatggctgggacgaacatactataggagcaaatggtacaatgactatgctgagagtgtgaagtcacgaatcacaattaacccagatactagcaagaatcagttctccctgcagctgaactctgtgacacccgaggatactgcagtctactattgcgcacgctccggacacatcaccgtgttcggagtcaatgtggacgcctttgatatgtggggacaggggaccacagtcacagtgtctagtgcaagtactaaaggcccatcagtgtttcccctggccccttcaagcaagagtacctcaggcggaacagccgctctgggatgtctggtgaaggactacttccctgagccagtcaccgtgagctggaactccggagctctgaccagcggggtgcatacatttcctgcagtcctgcagtcctctggcctgtacagcctgagttcagtggtcaccgtgccaagctcctctctgggaacacagacttatatctgcaacgtgaatcacaaaccatccaatacaaaggtcgacaagaaagtggaacccaaatcttgtgataagacccatacatgccctccctgtccagcacctgagctgctgggcggcccatccgtgttcctgtttccacccaagcctaaagacacactgatgattagccggactcccgaagtgacctgcgtggtcgtggacgtgagccacgaggaccccgaagtgaagttcaactggtacgtggatggcgtcgaggtgcataatgccaagaccaaacctagggaggaacagtacaacagcacttatagagtcgtgtccgtcctgaccgtgctgcaccaggattggctgaacgggaaggagtataagtgcaaagtgtccaacaaggccctgccagctcccatcgagaagaccatttctaaggccaaaggccagccacgggaaccccaggtgtacacactgcctccaagccgcgacgagctgaccaaaaaccaggtgagcctgacatgtctggtcaagggattctatcctagtgatatcgctgtggagtgggaatctaatgggcagccagaaaacaattacaagactacccctcccgtgctggactctgatggaagtttctttctgtattcaaaactgaccgtggacaagagccgctggcagcaggggaacgtctttagctgctccgtgatgcacgaggccctgcacaatcattacactcagaaatctctgagtctgtcacccggaaaatgataa

SEQ ID NO: 168 - DNA, 693 bp, Artificial Sequence
  Influenza A DMAb
atggtcctgcagacacaggtgtttatctctctgctgctgtggattagtggcgcttacggagacatccagatgactcagtctcctagttcactgtctgcaagtgtcggcgatcgcgtgactattacctgtcgaacctcacagagcctgagctcctacctgcattggtatcagcagaagcctgggaaagcaccaaagctgctgatctatgcagcctctagtctgcagtccggcgtgccctctaggttctccgggtctggcagtggaactgactttacactgactatttcaagcctgcagcctgaggatttcgctacctactattgccagcagagcagaacttttgggcagggcaccaaagtcgaaatcaagacagtggctgcaccatccgtcttcatttttccaccctctgacgagcagctgaagagtggaactgcctcagtggtgtgcctgctgaacaatttctacccccgggaagccaaagtccagtggaaggtggataacgctctgcagtcaggcaatagccaggagtccgtgacagaacaggactctaaagatagtacttattcactgagcaacaccctgacactgagcaaggcagactacgagaagcacaaagtgtatgcctgcgaagtgacccaccaggggctgagcagtccagtgaccaaatctttcaacaggggagaatgttgataa

SEQ ID NO: 169 - DNA, 1437 bp, Artificial Sequence
  Influenza A DMAb
atggattggacatggaggattctgtttctggtcgccgccgcaactggaactcacgctcaggtgcagctgcagcagtcagggcctggcctggtgaagcccagccagaccctgtccctgacatgcgccatctccggcgactctgtgagctccaacaatgccgtgtggaactggatcaggcagtccccttctcgcggcctggagtggctgggaaggacctactatagaagcaagtggtacaatgactatgccgagagcgtgaagtccaggatcaccatcaacccagatacatctaagaatcagttcagcctgcagctgaactccgtgacccccgaggatacagccgtgtactattgcgccagatccggccacatcaccgtgttcggcgtgaatgtggacgcctttgatatgtggggccagggcaccacagtgaccgtgtctagcgcctctacaaagggcccaagcgtgtttccactggcaccctcctctaagagcacctccggcggcacagccgccctgggctgtctggtgaaggactacttcccagagcccgtgaccgtgtcttggaacagcggcgccctgaccagcggagtgcacacatttcctgccgtgctgcagagctccggcctgtactccctgtctagcgtggtgaccgtgccatcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccaagcgtgttcctgtttccacccaagcctaaggacaccctgatgatctctcggacccccgaggtgacatgcgtggtggtggacgtgagccacgaggaccccgaggtgaagttcaactggtacgtggatggcgtggaggtgcacaatgccaagacaaagcctagggaggagcagtacaactccacctatagagtggtgtctgtgctgacagtgctgcaccaggattggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggactccgatggctctttctttctgtatagcaagctgaccgtggacaagtcccgctggcagcagggcaacgtgttttcttgtagcgtgatgcacgaagcactgcacaaccattacacccagaagtcactgtcactgtccccaggaaaatgataa

SEQ ID NO: 170 - DNA, 693 bp, Artificial Sequence
  Influenza A DMAb
atggtgctgcagacccaggtgtttatttccctgctgctgtggattagcggcgcatacggcgacattcagatgactcagagcccttcaagcctgtccgcctctgtgggcgacagggtgaccatcacatgcagaaccagccagtccctgagctcctacctgcactggtatcagcagaagccaggcaaggcccccaagctgctgatctacgcagcctctagcctgcagagcggcgtgccttcccggttctctggcagcggctccggcaccgactttaccctgacaatctcctctctgcagccagaggatttcgccacatactattgccagcagtcccgcacctttggccagggcacaaaggtggagatcaagaccgtggccgccccctccgtgttcatctttcccccttctgacgagcagctgaagtctggcacagccagcgtggtgtgcctgctgaacaatttctaccctagggaggccaaggtgcagtggaaggtggataacgccctgcagtccggcaattctcaggagagcgtgaccgagcaggactccaaggattctacatattctctgagcaacaccctgacactgagcaaggccgattacgagaagcacaaggtgtatgcctgtgaggtcactcaccaggggctgtcatcacccgtcaccaaatcctttaataggggagaatgttgataa

SEQ ID NO: 171 - protein, 47 aa, Artificial Sequence
  amyloid beta  (X-Y) peptide
DAEFRHDSGYEVHHQKLVFFAEDVGSNKGAIIGLMVGGVVIATVIVI